SEQUENCE LISTING (161 sequences)

SEQ ID NO: 1 - protein, 227 aa, Homo sapiens
  misc_feature (1)..(227): hIgG1 Fc - Shorter (No N-terminal EPKSC)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 227 aa, Artificial Sequence
  Synthetic hIgG1 Fc - Hole (Y407T) - Shorter
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 3 - protein, 227 aa, Artificial Sequence
  Synthetic hIgG1 Fc - Knob (T366Y) - Shorter
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 227 aa, Artificial Sequence
  Synthetic hIgG1 Fc - N297G - Shorter
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 232 aa, Homo sapiens
  misc_feature (1)..(232): hIgG1 Fc - Longer (N-terminal EPKSC)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Artificial Sequence
  Synthetic hIgG1 Fc - Knob (T366Y) - Longer
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 232 aa, Artificial Sequence
  Synthetic hIgG1 Fc - Hole (Y407T) - Longer
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 8 - protein, 232 aa, Artificial Sequence
  Synthetic hIgG1 Fc - N297G - Longer
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 10 aa, Artificial Sequence
  Synthetic linker
GGGGSGGGGS

SEQ ID NO: 10 - protein, 5 aa, Artificial Sequence
  Synthetic linker
GGGGS

SEQ ID NO: 11 - protein, 15 aa, Artificial Sequence
  Synthetic linker
GGGGSGGGGSGGGGS

SEQ ID NO: 12 - protein, 5 aa, Artificial Sequence
  Synthetic linker
EPKSS

SEQ ID NO: 13 - protein, 15 aa, Artificial Sequence
  Synthetic linker
  REPEAT (1)..(15): “Gly Gly Pro” is present at least once and may repeat up to five times
GGPGGPGGPGGPGGP

SEQ ID NO: 14 - protein, 25 aa, Artificial Sequence
  Synthetic linker
  REPEAT (1)..(25): “Gly Gly Gly Gly Ser” is present at least once and may repeat up to five times
GGGGSGGGGSGGGGSGGGGSGGGGS

SEQ ID NO: 15 - protein, 1709 aa, Homo sapiens
  misc_feature (1)..(1709): Siglec 1 FL
MGFLPKLLLLASFFPAGQASWGVSSPQDVQGVKGSCLLIPCIFSFPADVEVPDGITAIWYYDYSGQRQVVSHSADPKLVEARFRGRTEFMGNPEHRVCNLLLKDLQPEDSGSYNFRFEISEVNRWSDVKGTLVTVTEEPRVPTIASPVELLEGTEVDFNCSTPYVCLQEQVRLQWQGQDPARSVTFNSQKFEPTGVGHLETLHMAMSWQDHGRILRCQLSVANHRAQSEIHLQVKYAPKGVKILLSPSGRNILPGELVTLTCQVNSSYPAVSSIKWLKDGVRLQTKTGVLHLPQAAWSDAGVYTCQAENGVGSLVSPPISLHIFMAEVQVSPAGPILENQTVTLVCNTPNEAPSDLRYSWYKNHVLLEDAHSHTLRLHLATRADTGFYFCEVQNVHGSERSGPVSVVVNHPPLTPVLTAFLETQAGLVGILHCSVVSEPLATLVLSHGGHILASTSGDSDHSPRFSGTSGPNSLRLEIRDLEETDSGEYKCSATNSLGNATSTLDFHANAARLLISPAAEVVEGQAVTLSCRSGLSPTPDARFSWYLNGALLHEGPGSSLLLPAASSTDAGSYHCRARDGHSASGPSSPAVLTVLYPPRQPTFTTRLDLDAAGAGAGRRGLLLCRVDSDPPARLQLLHKDRVVATSLPSGGGCSTCGGCSPRMKVTKAPNLLRVEIHNPLLEEEGLYLCEASNALGNASTSATFNGQATVLAIAPSHTLQEGTEANLTCNVSREAAGSPANFSWFRNGVLWAQGPLETVTLLPVARTDAALYACRILTEAGAQLSTPVLLSVLYPPDRPKLSALLDMGQGHMALFICTVDSRPLALLALFHGEHLLATSLGPQVPSHGRFQAKAEANSLKLEVRELGLGDSGSYRCEATNVLGSSNTSLFFQVRGAWVQVSPSPELQEGQAVVLSCQVHTGVPEGTSYRWYRDGQPLQESTSATLRFAAITLTQAGAYHCQAQAPGSATTSLAAPISLHVSYAPRHVTLTTLMDTGPGRLGLLLCRVDSDPPAQLRLLHGDRLVASTLQGVGGPEGSSPRLHVAVAPNTLRLEIHGAMLEDEGVYICEASNTLGQASASADFDAQAVNVQVWPGATVREGQLVNLTCLVWTTHPAQLTYTWYQDGQQRLDAHSIPLPNVTVRDATSYRCGVGPPGRAPRLSRPITLDVLYAPRNLRLTYLLESHGGQLALVLCTVDSRPPAQLALSHAGRLLASSTAASVPNTLRLELRGPQPRDEGFYSCSARSPLGQANTSLELRLEGVRVILAPEAAVPEGAPITVTCADPAAHAPTLYTWYHNGRWLQEGPAASLSFLVATRAHAGAYSCQAQDAQGTRSSRPAALQVLYAPQDAVLSSFRDSRARSMAVIQCTVDSEPPAELALSHDGKVLATSSGVHSLASGTGHVQVARNALRLQVQDVPAGDDTYVCTAQNLLGSISTIGRLQVEGARVVAEPGLDVPEGAALNLSCRLLGGPGPVGNSTFAWFWNDRRLHAEPVPTLAFTHVARAQAGMYHCLAELPTGAAASAPVMLRVLYPPKTPTMMVFVEPEGGLRGILDCRVDSEPLASLTLHLGSRLVASSQPQGAPAEPHIHVLASPNALRVDIEALRPSDQGEYICSASNVLGSASTSTYFGVRALHRLHQFQQLLWVLGLLVGLLLLLLGLGACYTWRRRRVCKQSMGENSVEMAFQKETTQLIDPDAATCETSTCAPPLG

SEQ ID NO: 16 - DNA, 6736 bp, Homo sapiens
  misc_feature (1)..(6736): Siglec 1 FL
atgggcttcttgcccaagcttctcctcctggcctcattcttcccagcaggccaggcctcatggggcgtctccagtccccaggacgtgcagggtgtgaaggggtcttgcctgcttatcccctgcatcttcagcttccctgccgacgtggaggtgcccgacggcatcacggccatctggtactacgactactcgggccagcggcaggtggtgagccactcggcggaccccaagctggtggaggcccgcttccgcggccgcaccgagttcatggggaaccccgagcacagggtgtgcaacctgctgctgaaggacctgcagcccgaggactctggttcctacaacttccgcttcgagatcagtgaggtcaaccgctggtcagatgtgaaaggcaccttggtcacagtaacagaggagcccagggtgcccaccattgcctccccggtggagcttctcgagggcacagaggtggacttcaactgctccactccctacgtatgcctgcaggagcaggtcagactgcagtggcaaggccaggaccctgctcgctctgtcaccttcaacagccagaagtttgagcccaccggcgtcggccacctggagaccctccacatggccatgtcctggcaggaccacggccggatcctgcgctgccagctctccgtggccaatcacagggctcagagcgagattcacctccaagtgaagtatgcccccaagggtgtgaagatcctcctcagcccctcggggaggaacatccttccaggtgagctggtcacactcacctgccaggtgaacagcagctaccctgcagtcagttccattaagtggctcaaggatggggtacgcctccaaaccaagactggtgtgctgcacctgccccaggcagcctggagcgatgctggcgtctacacctgccaagctgagaacggcgtgggctctttggtctcaccccccatcagcctccacatcttcatggctgaggtccaggtgagcccagcaggtcccatcctggagaaccagacagtgacactagtctgcaacacacccaatgaggcacccagtgatctccgctacagctggtacaagaaccatgtcctgctggaggatgcccactcccataccctccggctgcacttggccactagggctgatactggcttctacttctgtgaggtgcagaacgtccatggcagcgagcgctcgggccctgtcagcgtggtagtcaaccacccgcctctcactccagtcctgacagccttcctggagacccaggcgggacttgtgggcatccttcactgctctgtggtcagtgagcccctggccacactggtgctgtcacatgggggtcatatcctggcctccacctccggggacagtgatcacagcccacgcttcagtggtacctctggtcccaactccctgcgcctggagatccgagacctggaggaaactgacagtggggagtacaagtgctcagccaccaactcccttggaaatgcaacctccaccctggacttccatgccaatgccgcccgtctcctcatcagcccggcagccgaggtggtggaaggacaggcagtgacactgagctgcagaagcggcctaagccccacacctgatgcccgcttctcctggtacctgaatggagccctgcttcacgagggtcccggcagcagcctcctgctccccgcggcctccagcactgacgccggctcataccactgccgggcccgggacggccacagtgccagtggcccctcttcgccagctgttctcactgtgctctacccccctcgacaaccaacattcaccaccaggctggaccttgatgccgctggggccggggctggacggcgaggcctccttttgtgccgtgtggacagcgacccccccgccaggctgcagctgctccacaaggaccgtgttgtggccacttccctgccatcagggggtggctgcagcacctgtgggggctgttccccacgcatgaaggtcaccaaagcccccaacttgctgcgtgtggagattcacaaccctttgctggaagaggagggcttgtacctctgtgaggccagcaatgccctgggcaacgcctccacctcagccaccttcaatggccaggccactgtcctggccattgcaccatcacacacacttcaggagggcacagaagccaacttgacttgcaacgtgagccgggaagctgctggcagccctgctaacttctcctggttccgaaatggggtgctgtgggcccagggtcccctggagaccgtgacactgctgcccgtggccagaactgatgctgccctttacgcctgccgcatcctgactgaggctggtgcccagctctccactcccgtgctcctgagtgtactctatcccccggaccgtccaaagctgtcagccctcctagacatgggccagggccacatggctctgttcatctgcactgtggacagccgccccctggccttgctggccttgttccatggggagcacctcctggccaccagcctgggtccccaggtcccatcccatggtcggttccaggctaaagctgaggccaactccctgaagttagaggtccgagaactgggccttggggactctggcagctaccgctgtgaggccacaaatgttcttggatcatccaacacctcactcttcttccaggtccgaggagcctgggtccaggtgtcaccatcacctgagctccaagagggccaggctgtggtcctgagctgccaggtacacacaggagtcccagaggggacctcatatcgttggtatcgggatggccagcccctccaggagtcgacctcggccacgctccgctttgcagccataactttgacacaagctggggcctatcattgccaagcccaggccccaggctcagccaccacgagcctagctgcacccatcagcctccacgtgtcctatgccccacgccacgtcacactcactaccctgatggacacaggccctggacgactgggcctcctcctgtgccgtgtggacagtgaccctccggcccagctgcggctgctccacggggatcgccttgtggcctccaccctacaaggtgtggggggacccgaaggcagctctcccaggctgcatgtggctgtggcccccaacacactgcgtctggagatccacggggctatgctggaggatgagggtgtctatatctgtgaggcctccaacaccctgggccaggcctcggcctcagctgacttcgacgctcaagctgtgaatgtgcaggtgtggcccggggctaccgtgcgggaggggcagctggtgaacctgacctgccttgtgtggaccactcacccggcccagctcacctacacatggtaccaggatgggcagcagcgcctggatgcccactccatccccctgcccaacgtcacagtcagggatgccacctcctaccgctgcggtgtgggcccccctggtcgggcaccccgcctctccagacctatcaccttggacgtcctctacgcgccccgcaacctgcgcctgacctacctcctggagagccatggcgggcagctggccctggtactgtgcactgtggacagccgcccgcccgcccagctggccctcagccacgccggtcgcctcttggcctcctcgacagcagcctctgtccccaacaccctgcgcctggagctgcgagggccacagcccagggatgagggtttctacagctgctctgcccgcagccctctgggccaggccaacacgtccctggagctgcggctggagggtgtgcgggtgatcctggctccggaggctgccgtgcctgaaggtgcccccatcacagtgacctgtgcggaccctgctgcccacgcacccacactctatacttggtaccacaacggtcgttggctgcaggagggtccagctgcctcactctcattcctggtggccacgcgggctcatgcaggcgcctactcttgccaggcccaggatgcccagggcacccgcagctcccgtcctgctgccctgcaagtcctctatgcccctcaggacgctgtcctgtcctccttccgggactccagggccagatccatggctgtgatacagtgcactgtggacagtgagccacctgctgagctggccctatctcatgatggcaaggtgctggccacgagcagcggggtccacagcttggcatcagggacaggccatgtccaggtggcccgaaacgccctacggctgcaggtgcaagatgtgcctgcaggtgatgacacctatgtttgcacagcccaaaacttgctgggctcaatcagcaccatcgggcggttgcaggtagaaggtgcacgcgtggtggcagagcctggcctggacgtgcctgagggcgctgccctgaacctcagctgccgcctcctgggtggccctgggcctgtgggcaactccacctttgcatggttctggaatgaccggcggctgcacgcggagcctgtgcccactctcgccttcacccacgtggctcgtgctcaagctgggatgtaccactgcctggctgagctccccactggggctgctgcctctgctccagtcatgctccgtgtgctctaccctcccaagacgcccaccatgatggtcttcgtggagcctgagggtggcctccggggcatcctggattgccgagtggacagcgagccgctcgccagcctgactctccaccttggcagtcgactggtggcctccagtcagccccagggtgctcctgcagagccacacatccatgtcctggcttcccccaatgccctgagggtggacatcgaggcgctgaggcccagcgaccaaggggaatacatctgttctgcctcaaatgtcctgggctctgcctctacctccacctactttggggtcagagccctgcaccgcctgcatcagttccagcagctgctctgggtcctgggactgctggtgggcctcctgctcctgctgttgggcctgggggcctgctacacctggagaaggaggcgtgtttgtaagcagagcatgggcgagaattcggtggagatggcttttcagaaagagaccacgcagctcattgatcctgatgcagccacatgtgagacctcaacctgtgccccacccctgggctgaccagtggtgttgcctgccctccggaggagaaagtggccagaatctgtgatgactccagcctatgaatgtgaatgaggcagtgttgagtcctgcccgcctctacgaaaacagctctgtgacatctgactttttatgacctggccccaagcctcttgcccccccaaaaatgggtggtgagaggtctgcccaggagggtgttgaccctggaggacactgaagagcactgagctgatctcgctctctcttctctggatctcctcccttctctccatttctccctcaaaggaagccctgccctttcacatccttctcctcgaaagtcaccctggactttggttggattgcagcatcctgcatcctcagaggctcaccaaggcattctgtattcaacagagtatcagtcagcctgctctaacaagagaccaaatacagtgacttcaacatgatagaattttatttttctctcccacgctagtctggctgttacgatggtttatgatgttggggctcaggatccttctatcttccttttctctatccctaaaatgatgcctttgattgtgaggctcaccatggccccgctttgtccacatgccctccagccagaagaaggaagagtggaggtagaagcacacccatgcccatggtggacgcaactcagaagctgcacaggacttttccactcacttcccattggctggagtattgtcacatggctactgcaagctacaagggagactgggaaatgtagtttttattttgagtccagaggacatttggaattggacttccaaaggactcccaactgtgagctcatccctgagacttttgacattgttgggaatgccaccagcaggccatgttttgtctcagtgcccatctactgagggccagggtgtgcccctggccattctggttgtgggcttcctggaagaggtgatcactctcacactaagactgaggaaataaaaaaggtttggtgttttcctagggagagagcatgccaggcagtggagttgcctaagcagacatccttgtgccagatttggcccctgaaagaagagatgccctcattcccaccaccaccccccctacccccagggactgggtactaccttactggcccttacaagagtggagggcagacacagatgttgtcagcatccttattcctgctccagatgcatctctgttcatgactgtgtgagctcctgtccttttcctggagaccctgtgtcgggctgttaaagagaatgagttaccaagaaggaatgacgtgcccctgcgaatcagggaccaacaggagagagctcttgagtgggctagtgactccccctgcagcctggtggagatggtgtgaggagcgaagagccctctgctctaggatttgggttgaaaaacagagagagaagtggggagttgccacaggagctaacacgctgggaggcagttgggggcgggtgaactttgtgtagccgaggccgcaccctccctcattccaggctcattcattttcatgctccattgccagactcttgctgggagcccgtccagaatgtcctcccaataaaactccatcctatgacgcaaaaaaaaaaaaaaaaaa

SEQ ID NO: 17 - protein, 301 aa, Homo sapiens
  misc_feature (1)..(301): Siglec 1 Fragment (20-320)
SWGVSSPQDVQGVKGSCLLIPCIFSFPADVEVPDGITAIWYYDYSGQRQVVSHSADPKLVEARFRGRTEFMGNPEHRVCNLLLKDLQPEDSGSYNFRFEISEVNRWSDVKGTLVTVTEEPRVPTIASPVELLEGTEVDFNCSTPYVCLQEQVRLQWQGQDPARSVTFNSQKFEPTGVGHLETLHMAMSWQDHGRILRCQLSVANHRAQSEIHLQVKYAPKGVKILLSPSGRNILPGELVTLTCQVNSSYPAVSSIKWLKDGVRLQTKTGVLHLPQAAWSDAGVYTCQAENGVGSLVSPPIS

SEQ ID NO: 18 - protein, 301 aa, Artificial Sequence
  Synthetic Siglec 1 Fragment (20-320) SAX (R97A)
SWGVSSPQDVQGVKGSCLLIPCIFSFPADVEVPDGITAIWYYDYSGQRQVVSHSADPKLVEARFRGRTEFMGNPEHRVCNLLLKDLQPEDSGSYNFAFEISEVNRWSDVKGTLVTVTEEPRVPTIASPVELLEGTEVDFNCSTPYVCLQEQVRLQWQGQDPARSVTFNSQKFEPTGVGHLETLHMAMSWQDHGRILRCQLSVANHRAQSEIHLQVKYAPKGVKILLSPSGRNILPGELVTLTCQVNSSYPAVSSIKWLKDGVRLQTKTGVLHLPQAAWSDAGVYTCQAENGVGSLVSPPIS

SEQ ID NO: 19 - protein, 538 aa, Artificial Sequence
  Synthetic Siglec 1 Fragment (20-320) / (G4S)2 / hIgG1 Fc N297G
SWGVSSPQDVQGVKGSCLLIPCIFSFPADVEVPDGITAIWYYDYSGQRQVVSHSADPKLVEARFRGRTEFMGNPEHRVCNLLLKDLQPEDSGSYNFRFEISEVNRWSDVKGTLVTVTEEPRVPTIASPVELLEGTEVDFNCSTPYVCLQEQVRLQWQGQDPARSVTFNSQKFEPTGVGHLETLHMAMSWQDHGRILRCQLSVANHRAQSEIHLQVKYAPKGVKILLSPSGRNILPGELVTLTCQVNSSYPAVSSIKWLKDGVRLQTKTGVLHLPQAAWSDAGVYTCQAENGVGSLVSPPISGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1614 bp, Artificial Sequence
  Synthetic Siglec 1 Fragment (20-320) / (G4S)2 / hIgG1 Fc N297G
tcttggggcgttagcagccctcaggatgtgcagggcgtgaagggaagctgtctgctgatcccctgcatcttcagcttccccgccgatgtggaagtgcccgatggaatcacagccatctggtactacgactacagcggccagagacaggtggtgtcccatagcgccgatcctaagctggtggaagcccggtttagaggccggaccgagttcatgggaaaccccgagcacagagtgtgcaacctgctgctgaaggacctgcagcctgaggatagcggcagctacaacttcagattcgagatcagcgaagtgaaccgttggagcgacgtgaagggcaccctggtcacagtgaccgaggaacccagagtgcccacaatcgcctctccagtggaactgctggaaggcaccgaggtggacttcaactgcagcaccccttacgtgtgcctgcaagagcaagttcggctgcagtggcagggccaagatcctgccagaagcgtgaccttcaacagccagaagttcgagcctacaggcgtgggccacctggaaacactgcatatggccatgagctggcaggaccacggcagaatcctgagatgccagctgagcgtggccaaccacagagcccagtctgagatccatctgcaagtgaagtacgcccctaaaggcgtgaagattctgctgagccccagcggcagaaacatcctgcctggcgaactcgtgaccctgacctgccaagtgaacagcagctaccctgccgtgtccagcatcaagtggctgaaagacggcgtgcggctgcagacaaaaaccggcgttctgcatctgcctcaggccgcttggagtgacgctggcgtgtacacttgtcaggccgagaatggcgtgggcagcctggtgtctcctcctatctctggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 21 - protein, 538 aa, Artificial Sequence
  Synthetic Siglec 1 Fragment (20-320) SAX / (G4S)2 / hIgG1 FcN297G
SWGVSSPQDVQGVKGSCLLIPCIFSFPADVEVPDGITAIWYYDYSGQRQVVSHSADPKLVEARFRGRTEFMGNPEHRVCNLLLKDLQPEDSGSYNFAFEISEVNRWSDVKGTLVTVTEEPRVPTIASPVELLEGTEVDFNCSTPYVCLQEQVRLQWQGQDPARSVTFNSQKFEPTGVGHLETLHMAMSWQDHGRILRCQLSVANHRAQSEIHLQVKYAPKGVKILLSPSGRNILPGELVTLTCQVNSSYPAVSSIKWLKDGVRLQTKTGVLHLPQAAWSDAGVYTCQAENGVGSLVSPPISGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 1614 bp, Artificial Sequence
  Synthetic Siglec 1 Fragment (20-320) SAX / (G4S)2 / hIgG1 Fc N297G
tcttggggcgttagcagccctcaggatgtgcagggcgtgaagggaagctgtctgctgatcccctgcatcttcagcttccccgccgatgtggaagtgcccgatggaatcacagccatctggtactacgactacagcggccagagacaggtggtgtcccatagcgccgatcctaagctggtggaagcccggtttagaggccggaccgagttcatgggaaaccccgagcacagagtgtgcaacctgctgctgaaggacctgcagcctgaggatagcggcagctacaacttcgccttcgagatcagcgaagtgaaccgttggagcgacgtgaagggcaccctggtcacagtgaccgaggaacccagagtgcccacaatcgcctctccagtggaactgctggaaggcaccgaggtggacttcaactgcagcaccccttacgtgtgcctgcaagagcaagttcggctgcagtggcagggccaagatcctgccagaagcgtgaccttcaacagccagaagttcgagcctacaggcgtgggccacctggaaacactgcatatggccatgagctggcaggaccacggcagaatcctgagatgccagctgagcgtggccaaccacagagcccagtctgagatccatctgcaagtgaagtacgcccctaaaggcgtgaagattctgctgagccccagcggcagaaacatcctgcctggcgaactcgtgaccctgacctgccaagtgaacagcagctaccctgccgtgtccagcatcaagtggctgaaagacggcgtgcggctgcagacaaaaaccggcgttctgcatctgcctcaggccgcttggagtgacgctggcgtgtacacttgtcaggccgagaatggcgtgggcagcctggtgtctcctcctatctctggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 23 - protein, 847 aa, Homo sapiens
  misc_feature (1)..(847): Siglec 2 FL
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 24 - DNA, 2544 bp, Homo sapiens
  misc_feature (1)..(2544): Siglec 2 FL
atgcatctcctcggcccctggctcctgctcctggttctagaatacttggctttctctgactcaagtaaatgggtttttgagcaccctgaaaccctctacgcctgggagggggcctgcgtctggatcccctgcacctacagagccctagatggtgacctggaaagcttcatcctgttccacaatcctgagtataacaagaacacctcgaagtttgatgggacaagactctatgaaagcacaaaggatgggaaggttccttctgagcagaaaagggtgcaattcctgggagacaagaataagaactgcacactgagtatccacccggtgcacctcaatgacagtggtcagctggggctgaggatggagtccaagactgagaaatggatggaacgaatacacctcaatgtctctgaaaggccttttccacctcatatccagctccctccagaaattcaagagtcccaggaagtcactctgacctgcttgctgaatttctcctgctatgggtatccgatccaattgcagtggctcctagagggggttccaatgaggcaggctgctgtcacctcgacctccttgaccatcaagtctgtcttcacccggagcgagctcaagttctccccacagtggagtcaccatgggaagattgtgacctgccagcttcaggatgcagatgggaagttcctctccaatgacacggtgcagctgaacgtgaagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcaggggcttcaggagaattccagcggccagagcttctttgtgaggaataaaaaggttagaagggcccccctctctgaaggcccccactccctgggatgctacaatccaatgatggaagatggcattagctacaccaccctgcgctttcccgagatgaacataccacgaactggagatgcagagtcctcagagatgcagagacctcccccggactgcgatgacacggtcacttattcagcattgcacaagcgccaagtgggcgactatgagaacgtcattccagattttccagaagatgaggggattcattactcagagctgatccagtttggggtcggggagcggcctcaggcacaagaaaatgtggactatgtgatcctcaaacattga

SEQ ID NO: 25 - protein, 364 aa, Homo sapiens
  misc_feature (1)..(364): Siglec 3 FL
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 26 - DNA, 1466 bp, Homo sapiens
  misc_feature (1)..(1466): Siglec 3 FL
tctgctcacacaggaagccctggaagctgcttcctcagacatgccgctgctgctactgctgcccctgctgtgggcaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctatgttccacagaacccaacaactggtatctttccaggagatggctcagggaaacaagagaccagagcaggagtggttcatggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccagtgaggaacccacaagagcatcaggctcagctagaagatccacatcctctacaggtcggggaccaaaggctgattcttggagatttaacaccccacaggcaatgggtttatagacattatgtgagtttcctgctatattaacatcatcttagactttgcaagcagagagtcgtggaatcaaatctgtgctctttcatttgctaagtgtatgatgtcacacaagctccttaaccttccatgtctccattttcttctctgtgaagtaggtataagaagtcctatctcatagggatgctgtgagcattaaataaaggtacacatggaaaacaccagtc

SEQ ID NO: 27 - protein, 117 aa, Homo sapiens
  misc_feature (1)..(117): Siglec 3 V-Set (23-139)
LQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVT

SEQ ID NO: 28 - protein, 205 aa, Homo sapiens
  misc_feature (1)..(205): Siglec 3 V-Set Domain + 1 C2-Set Domain (23-227)
LQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTI

SEQ ID NO: 29 - protein, 242 aa, Homo sapiens
  misc_feature (1)..(242): Siglec 3 Fragment (18-259)
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVH

SEQ ID NO: 30 - protein, 242 aa, Artificial Sequence
  Synthetic Siglec 3 Fragment (18-259) SAX
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFKMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVH

SEQ ID NO: 31 - protein, 471 aa, Artificial Sequence
  Synthetic Siglec 3 Fragment (18-259) SAX / AS / hIgG1 Fc N297G
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFKMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 1413 bp, Artificial Sequence
  Synthetic Siglec 3 Fragment (18-259) SAX / AS / hIgG1 Fc N297G
gatcccaatttctggctgcaagtgcaagagtccgtgaccgtgcaagagggcctgtgtgtgctggtgccctgcaccttctttcaccccattccttactacgacaagaacagccctgtgcacggctactggtttagagagggcgccatcatcagcagagatagccctgtggccaccaacaagctggaccaagaggtgcaagaagagacacagggcagattcagactgctgggcgaccccagcagaaacaactgcagcctgtctatcgtggacgccaggcggagagacaacggcagctacttcttcaagatggaacggggcagcaccaagtacagctacaagagccctcagctgtccgtgcacgtgaccgacctgacacacagacccaagattctgatccccggcacactggaacctggccacagcaagaatctgacctgctccgtgtcctgggcctgcgaacagggaacccctcctatctttagctggctgagcgccgctcctacatctctgggccctagaacaacacacagcagcgtgctgatcatcacccctagacctcaggaccacggcaccaacctgacctgccaagtgaaatttgctggcgctggcgtgaccaccgagagaaccatccagctgaacgtgacctacgtgccacagaaccctaccaccggcatctttccaggcgacggctctggcaagcaagaaacaagagctggcgtggtgcacgctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 33 - protein, 626 aa, Homo sapiens
  misc_feature (1)..(626): Siglec 4 FL
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAVVAFAILIAIVCYITQTRRKKNVTESPSFSAGDNPPVLFSSDFRISGAPEKYESERRLGSERRLLGLRGEPPELDLSYSHSDLGKRPTKDSYTLTEELAEYAEIRVK

SEQ ID NO: 34 - DNA, 1881 bp, Homo sapiens
  misc_feature (1)..(1881): Siglec 4 FL
atgatattcctcacggcactgcctctgttctggattatgatttcagcctcccgagggggtcactggggtgcctggatgccctcgtccatctcggccttcgaaggcacgtgcgtctccatcccctgccgctttgacttcccggatgagctgcggcccgctgtggtgcatggtgtctggtacttcaatagcccctaccccaagaactaccccccggtggtcttcaagtcgcgcacccaagtagtccacgagagcttccagggccgcagccgcctcctgggggacctgggcctgcgaaactgcaccctcctgctcagcaacgtcagccccgagctgggcgggaagtactacttccgtggggacctgggcggctacaaccagtacaccttctcagagcacagcgtcctggatatcgtcaacacccccaacatcgtggtgcccccagaggtggtggcaggcacggaggtggaggtcagctgcatggtgccggacaactgcccagagctgcgccctgagctgagctggctgggccacgaggggctgggggagcccgctgtgctgggccggctgcgggaggacgagggcacctgggtgcaggtgtcactgctgcacttcgtgcccacgagggaggccaacggccacaggctgggctgccaggcctccttccccaacaccaccctgcagttcgagggctacgccagcatggacgtcaagtaccccccggtgattgtggagatgaactcctcggtggaggccatcgagggctcccacgtgagcctgctctgtggggctgacagcaaccccccgccgctgctgacctggatgcgggacgggacagtcctccgggaggcggtggccgagagcctgctcctggagctggaggaggtgacccccgccgaagacggcgtctatgcctgcctggccgagaatgcctatggccaggacaaccgcaccgtggggctcagtgtcatgtatgcaccctggaagccaacagtgaacgggacaatggtggccgtagagggggagacggtctctatcttgtgctccacacagagcaacccggaccctattctcaccatcttcaaggagaagcagatcctgtccacggtcatctacgagagcgagctgcagctggagctgccggccgtgtcacccgaggatgatggagagtactggtgtgtggctgagaaccagtatggccagagggccaccgccttcaacctgtctgtggagttcgcccctgtgctcctcctggagtcccactgcgcggcagcccgagacacggtgcagtgcctgtgcgtggtgaagtccaacccggagccgtccgtggcctttgagctgccatcgcgcaatgtgaccgtgaacgagagcgagcgggagttcgtgtactcggagcgcagcggcctcgtgctcaccagcatcctcacgctgcgggggcaggcccaggccccgccccgcgtcatctgcaccgcgaggaacctctatggcgccaagagcctggagctgcccttccagggagcccatcgactgatgtgggccaagatcgggcctgtgggcgccgtggtcgcctttgccatcctgattgccatcgtctgctacattacccagacacgcaggaaaaagaacgtgacagagagccccagcttctcggcaggggacaaccctcccgtcctgttcagcagcgacttccgcatctctggggcaccagagaagtacgagagcgagaggcgcctgggatctgagaggaggctgctgggccttcggggtgagcccccagagctggacctgagctattctcactcggacctggggaaacggcccaccaaggacagctacacgctgacggaggagctagctgagtatgctgaaatccgggtcaagtga

SEQ ID NO: 35 - protein, 497 aa, Homo sapiens
  misc_feature (1)..(497): Siglec 4 Fragment (20-516)
GHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGP

SEQ ID NO: 36 - protein, 497 aa, Artificial Sequence
  Synthetic Siglec 4 Fragment (20-516) SAX
GHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFAGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGP

SEQ ID NO: 37 - protein, 734 aa, Artificial Sequence
  Synthetic Siglec 4 Fragment (20-516) / (G4S)2 / hIgG1 Fc N297G
GHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 2202 bp, Artificial Sequence
  Synthetic Siglec 4 Fragment (20-516) / (G4S)2 / hIgG1 Fc N297G
ggacattggggagcctggatgcctagcagcatcagcgcctttgagggaacctgtgtgtcaatcccctgcagattcgacttccccgacgaactgaggcctgctgtggttcacggcgtgtggtacttcaacagcccctatcctaagaactaccctcctgtggtgttcaagagcagaacccaggtggtgcacgagagcttccagggcagatctagactgctgggcgatctgggcctgagaaactgtacactgctgctgagcaacgtgtcccctgaactcggcggcaagtactacttcagaggcgatctcggcggctacaaccagtacacctttagcgagcacagcgtgctggacatcgtgaacacccctaacatcgtggtgcctccagaagtggtggccggcacagaggtggaagtgtcctgtatggtgcccgacaactgccctgagctgaggccagaactgtcttggctgggacacgaaggactgggagaacctgctgttctgggcagactgagagaggacgaaggcacctgggtgcaagtgtccctgctgcactttgtgcctaccagagaggccaacggccacagactgggttgtcaggccagctttcccaacaccacactgcagtttgagggctacgccagcatggacgtgaagtaccctccagtgatcgtggaaatgaacagcagcgtggaagccatcgagggctctcatgtgtctctgctgtgtggcgccgacagcaatcctcctcctctgctgacctggatgagagatggcaccgtgctgagagaagccgtggccgaatctctgctgctggaactggaagaagtgacccctgccgaggatggcgtgtacgcttgtctggccgagaatgcctacggccaggacaatagaaccgtgggcctgtccgtgatgtacgccccttggaagcctaccgtgaacggcacaatggtggccgtggaaggcgagacagtgtccatcctgtgtagcacccagagcaaccccgatcctatcctgaccatcttcaaagagaagcagatcctgagcaccgtgatctacgagagcgaactgcagctcgaactgcccgctgtgtccccagaggatgatggcgaatattggtgcgtggcagagaaccagtacggccagagagccaccgccttcaacctgagcgtggaatttgctcccgtgctgctgctcgagagccattgtgctgccgccagagataccgtgcagtgcctgtgtgtggtcaagtctaaccccgagcctagcgtggcctttgagctgcccagcagaaacgtgaccgtgaatgagagcgagcgcgagttcgtgtacagcgagagatctggactggtgctgaccagcatcctgacactgagaggacaggctcaggcccctcctagagtgatctgcaccgccagaaatctgtacggcgccaagagcctggaactgccatttcagggcgcccacagactcatgtgggccaagattggacctggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 39 - protein, 734 aa, Artificial Sequence
  Synthetic Siglec 4 Fragment (20-516) SAX / (G4S)2 / hIgG1 Fc N297G
GHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFAGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 2202 bp, Artificial Sequence
  Synthetic Siglec 4 Fragment (20-516) SAX / (G4S)2 / hIgG1 Fc N297G
ggacattggggagcctggatgcctagcagcatcagcgcctttgagggaacctgtgtgtcaatcccctgcagattcgacttccccgacgaactgaggcctgctgtggttcacggcgtgtggtacttcaacagcccctatcctaagaactaccctcctgtggtgttcaagagcagaacccaggtggtgcacgagagcttccagggcagatctagactgctgggcgatctgggcctgagaaactgtacactgctgctgagcaacgtgtcccctgaactcggcggcaagtactacttcgccggcgatctcggcggctacaaccagtacacctttagcgagcacagcgtgctggacatcgtgaacacccctaacatcgtggtgcctccagaagtggtggccggcacagaggtggaagtgtcctgtatggtgcccgacaactgccctgagctgaggccagaactgtcttggctgggacacgaaggactgggagaacctgctgttctgggcagactgagagaggacgaaggcacctgggtgcaagtgtccctgctgcactttgtgcctaccagagaggccaacggccacagactgggttgtcaggccagctttcccaacaccacactgcagtttgagggctacgccagcatggacgtgaagtaccctccagtgatcgtggaaatgaacagcagcgtggaagccatcgagggctctcatgtgtctctgctgtgtggcgccgacagcaatcctcctcctctgctgacctggatgagagatggcaccgtgctgagagaagccgtggccgaatctctgctgctggaactggaagaagtgacccctgccgaggatggcgtgtacgcttgtctggccgagaatgcctacggccaggacaatagaaccgtgggcctgtccgtgatgtacgccccttggaagcctaccgtgaacggcacaatggtggccgtggaaggcgagacagtgtccatcctgtgtagcacccagagcaaccccgatcctatcctgaccatcttcaaagagaagcagatcctgagcaccgtgatctacgagagcgaactgcagctcgaactgcccgctgtgtccccagaggatgatggcgaatattggtgcgtggcagagaaccagtacggccagagagccaccgccttcaacctgagcgtggaatttgctcccgtgctgctgctcgagagccattgtgctgccgccagagataccgtgcagtgcctgtgtgtggtcaagtctaaccccgagcctagcgtggcctttgagctgcccagcagaaacgtgaccgtgaatgagagcgagcgcgagttcgtgtacagcgagagatctggactggtgctgaccagcatcctgacactgagaggacaggctcaggcccctcctagagtgatctgcaccgccagaaatctgtacggcgccaagagcctggaactgccatttcagggcgcccacagactcatgtgggccaagattggacctggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 41 - protein, 551 aa, Homo sapiens
  misc_feature (1)..(551): Siglec 5 FL
MLPLLLLPLLWGGSLQEKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQMKRQGAQVTTERTVQLNVSYAPQTITIFRNGIALEILQNTSYLPVLEGQALRLLCDAPSNPPAHLSWFQGSPALNATPISNTGILELRRVRSAEEGGFTCRAQHPLGFLQIFLNLSVYSLPQLLGPSCSWEAEGLHCRCSFRARPAPSLCWRLEEKPLEGNSSQGSFKVNSSSAGPWANSSLILHGGLSSDLKVSCKAWNIYGSQSGSVLLLQGRSNLGTGVVPAALGGAGVMALLCICLCLIFFLIVKARRKQAAGRPEKMDDEDPIMGTITSGSRKKPWPDSPGDQASPPGDAPPLEEQKELHYASLSFSEMKSREPKDQEAPSTTEYSEIKTSK

SEQ ID NO: 42 - DNA, 2523 bp, Homo sapiens
  misc_feature (1)..(2523): Siglec 5 FL
gtgcgcgtccacagctctcactcaccctccggcttcctgtcggggctttctcagccccaccccacgtttggacatttggagcatttccttccctgacagccggacctgggactgggctggggccctggcggatggagacatgctgcccctgctgctgctgcccctgctgtggggggggtccctgcaggagaagccagtgtacgagctgcaagtgcagaagtcggtgacggtgcaggagggcctgtgcgtccttgtgccctgctccttctcttacccctggagatcctggtattcctctcccccactctacgtctactggttccgggacggggagatcccatactacgctgaggttgtggccacaaacaacccagacagaagagtgaagccagagacccagggccgattccgcctccttggggatgtccagaagaagaactgctccctgagcatcggagatgccagaatggaggacacgggaagctatttcttccgcgtggagagaggaagggatgtaaaatatagctaccaacagaataagctgaacttggaggtgacagccctgatagagaaacccgacatccactttctggagcctctggagtccggccgccccacaaggctgagctgcagccttccaggatcctgtgaagcgggaccacctctcacattctcctggacggggaatgccctcagccccctggaccccgagaccacccgctcctcggagctcaccctcacccccaggcccgaggaccatggcaccaacctcacctgtcagatgaaacgccaaggagctcaggtgaccacggagagaactgtccagctcaatgtctcctatgctccacagaccatcaccatcttcaggaacggcatagccctagagatcctgcaaaacacctcataccttccggtcctggagggccaggctctgcggctgctctgtgatgctcccagcaacccccctgcacacctgagctggttccagggctcccctgccctgaacgccacccccatctccaataccgggatcttggagcttcgtcgagtaaggtctgcagaagaaggaggcttcacctgccgcgctcagcacccgctgggcttcctgcaaatttttctgaatctctcagtttactccctcccacagttgctgggcccctcctgctcctgggaggctgagggtctgcactgcagatgctcctttcgagcccggccggccccctccctgtgctggcggcttgaggagaagccgctggaggggaacagcagccagggctcattcaaggtcaactccagctcagctgggccctgggccaacagctccctgatcctccacggggggctcagctccgacctcaaagtcagctgcaaggcctggaacatctatgggtcccagagcggctctgtcctgctgctgcaagggagatcgaacctcgggacaggagtggttcctgcagcccttggtggtgctggtgtcatggccctgctctgtatctgtctgtgcctcatcttctttttaatagtgaaagcccgcaggaagcaagcagctgggagaccagagaaaatggatgatgaagaccccattatgggtaccatcacctcgggttccaggaagaagccctggccagacagccccggagatcaagcatctcctcctggggatgcccctcccttggaagaacaaaaggagctccattatgcctcccttagtttttctgagatgaagtcgagggagcctaaggaccaggaggccccaagcaccacggagtactcggagatcaagacaagcaagtgaggatttgcccagagttcagtcctggctggaggagccacagcctgtctgggggaaaggacaagtcagggaccacttgctgaagcacgaagagcccttgtggcaatgttaacattaactgatgtttaagtgctccaagcagatggaattagagaggtgggctcaaatctaggccctggcactgtcatcaagcaattcactgcatccctctgtgcctcagtttcccattctgtaaatcagagatcatgcatgctacctcaaaggttgttgtgaacattaaagaaatcaacacatggaaatcaaccaacatgggtcctggaacagggcgttgtgctcagtgctttctggtctctcttccttgaatagaaaggtcctgctggcaagttctctcaaggctggggatgaccaggcacaaaaaacagggcagcaatatgttggtgtcactccccttcccaaaactcttcgaagactccctaggaaagaccagcccctcagcctggcacttggttcatgatgtgggatcttatatccttgccagagtcatatctttgcccacttttacctgcaatccttgcatcatattcctttggctccagtccttcatttatgagacccataggaatccttccaacagccaaagagttgagtctaactctttcctgcccaaacccattcacggccccctggccttagacaatatatcacaagcatctcccctgacacataaagtc

SEQ ID NO: 43 - protein, 453 aa, Homo sapiens
  misc_feature (1)..(453): Siglec 6 FL
MQGAQEASASEMLPLLLPLLWAGALAQERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGVLGAVWGASITTLVFLCVCFIFRVKTRRKKAAQPVQNTDDVNPVMVSGSRGHQHQFQTGIVSDHPAEAGPISEDEQELHYAVLHFHKVQPQEPKVTDTEYSEIKIHK

SEQ ID NO: 44 - DNA, 3815 bp, Homo sapiens
  misc_feature (1)..(3815): Siglec 6 FL
gcgggacacagtctcttctcctctgctcttctttgggcaggtctctgggtctcaaagtttccgtctgctctgtgcagagggagtggagctccgagggcttgtggcttcgcagttcctcttctgtgaacagccgagatcacgcgctcctccccagccacccgttcctccccgcagtccttcccctccactcccttccccttctctgctcatgcagggagcccaggaagcctccgcctcagagatgctaccgctgctgctgcccctgctgtgggcaggggccctggctcaggagcggagattccagctggaggggccagagtcactgacggtgcaggagggtctgtgcgtcctcgtaccctgcagattgcccactacccttccagcctcgtactatggttatggctactggttcctggaaggggctgatgttccagtggccacaaacgacccagacgaagaagtgcaggaggagacccggggccgattccacctcctctgggatcccagaaggaagaactgctccctgagcatcagagatgcccggaggagggacaatgctgcatacttctttcggttgaagtccaaatggatgaaatacggttatacatcttccaagctctctgtgcgtgtgatggccctgacccacaggcccaacatctccatcccagggaccctggagtctggccatcccagcaatctgacctgctctgtgccctgggtctgtgagcaggggacgccccccatcttctcctggatgtcagctgcccccacctccctgggccccaggaccacccagtcctcggtgctcacaatcaccccacggccccaggaccacagcaccaacctcacctgtcaggtgacgttccctggagccggtgtgaccatggagagaaccatccagctcaatgtctcctccttcaaaatcctgcaaaacacctcgtccctccctgtcctggagggccaggctctgcggctgctctgtgatgctgacggcaacccccctgcacacctgagctggttccagggcttccccgccctgaacgccacccccatctccaataccggggtcctggagctgcctcaagtagggtctgcagaagaaggagatttcacctgccgtgctcagcatcctctgggctccctgcaaatctctctgagtctctttgtgcattggaaaccagaaggcagggctggtggtgtcctgggagcagtctggggagctagcatcacaaccctggttttcctctgtgtttgcttcatcttcagagtgaagactagaaggaagaaagcagcccagccagtgcaaaacacggatgatgtgaaccccgtcatggtctcaggctccaggggtcatcagcaccagttccagacaggcatagtttcagaccaccctgctgaggctggccccatctcagaagatgagcaggagctccactacgctgtcctacacttccacaaggtgcaacctcaggaaccaaaggtcaccgacactgagtactcagaaatcaagatacacaagtgaggaattgtccaaagccataaccttgattggagagaacatggtacctctcagtgtattggttactagggctgccacagcaatgtaccacaaaccgagtgacataaacacagaactttattttcgtatagtttcagatgttagaggtctgagaacaaggtgttatcagggttggtcccttctaaggcctctcttgttggcttgtagatggctgtctcctccttgtgtcttcacatggtctttcctctgagtgtgtttgtgtcctaatcttctcttcttataaagacactagtcatattggattagggcctccccatgacctaatttaaataaattaactatttaaagaccctccaaatacagtaaccttctgagatattgagatttaggacttccaacatatgaattttagaagggaacaatttagcccataacactgtgtccaattcttttaaaattaatgtttttgttgtaaatggactatataaataccttcgtatatatggcagaccacaggacttctgtccaagagaactgagttcaactccatctatgccagctattgagcaagtcgctttatgtccctgctctgtaaggcagggaaataatttccatctaaccagattattgtgaaaggtcaaagaaagcatacagctaacatacagctttgttagctgtaaaacagctaacaaaggccctgacacaaaggttttcataaagtctgtatatttttgtaaatgaatgccttgtatctggctttggctagctttttttttttttttttttttctgagatggagtcttgctgtatctcccaggctggagtgcagtggtgcgatcttggctcagtgcaagcttcgccccctgggttcacaccattctcctgcctcagcctcccaagtagctgggactacaggcacccaccaccacacccggctaattttttgtatttttagtagagacggggtttcaccgtgttagccaggatggtcttgatctcctgacctcgtgatctgccctcctcggcctcccaaagtgctgagattacaggcatgagccaccgcacccggctttgattagctgttttaacagacggtttctgctggcaatttcttctaaggctgaaaaggagtaggcccagaggccacagcaccaggtgtctgtgtcaccccctgttcagaactcttcacgactccccaaaataaggtttccatccatcatctttctgctcaaggctctctacatgatcttgtccatccagatttccccagatcaattcctcaccagtcaccaataccccatgtccatttccacagcttcctcttacaaatcccagtctccactattccagtgaaattgaagaaagcatttaatgaagaccaactattaaggaaaattcttaagagtagccatgaaaaataaaaagatagattacacttaaaagaccacagttagcagtggctcacgcctgtaatcccagcactttgggaggctgaggtgggtggatcactggaggtcaggagttcgagactggtctggccaacatggtgaaaccccatctctactaaaatacaaaaattagctcggtgtggtggcacttgcctgtaatcccaggtacttctgcaggttgaagcaggagaattgcttgaacctgggaggtggaggttgaagtgagctgagattgcatcactgcactccagcctgggcaacagagcgagactccaactcagaaaaagcaaaacaaaacaaacaaacaagcaaaaaaccacaattagactgacagctgacttttttaggagcaatattggaaggctaaatgcaatagaaagatgtctttgatggcttaagagaaataaatgttgttttagaaagcctactcaatgaaaacacattttaagactgaaagtgaaatatagatattttaaggaaaaccaaaatatgtgagtgttaataaagaaaagatttctcaaataaattctaaaacatataattcaggtattaggaaagtgatcccagattagatttttgagatccaaaaaaaatgaaaacctaggaaagtagcaaatatgtgagcaaaatgaaacaaatacttgttgtaaaaatgatggtttgtagaggggtcaaacatcaaatgtaatattgaaataccaatattatatagcccagaaactataataacataaagttcagaagagtgtaaatagaatttatattacataaagtctttatatttttccagagaaaattaaatgttatgatgaatgttaaaaaaaaaaaaaaaaaaaaaaaaaaaaaaa

SEQ ID NO: 45 - protein, 321 aa, Homo sapiens
  misc_feature (1)..(321): Siglec 6 Fragment (27-347)
QERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGV

SEQ ID NO: 46 - protein, 321 aa, Artificial Sequence
  Synthetic Siglec 6 Fragment (27-347) SAX
QERRFQLEGPESLTVQEGLCVLVPCRLPTTLPASYYGYGYWFLEGADVPVATNDPDEEVQEETRGRFHLLWDPRRKNCSLSIRDARRRDNAAYFFALKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGV

SEQ ID NO: 47 - protein, 558 aa, Artificial Sequence
  Synthetic Siglec 6 Fragment (27-347) / (G4S)2 / hIgG1 Fc N297G
QERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGVGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 1674 bp, Artificial Sequence
  Synthetic Siglec 6 Fragment (27-347) / (G4S)2 / hIgG1 Fc N297G
caagagcggcggtttcagctggaaggacctgagagcctgacagtgcaagagggcctgtgtgtgctggtgccttgtagactgcctaccacactgcccgccagctactacggctacggctattggtttctggaaggcgccgatgtgcccgtggccaccaatgatcctgacgaggaagtgcaagaagagacacggggcagattccatctgctgtgggaccccagacggaagaactgcagcctgagcatcagagatgccagaaggcgggataacgccgcctacttcttccggctgaagtccaagtggatgaagtacggctacaccagcagcaagctgagcgtcagagtgatggccctgacacacagacccaacatcagcatccccggcacactggaaagcggacaccctagcaatctgacctgcagcgtgccatgggtttgcgagcagggaacccctcctatcttctcctggatgtctgccgctcctacctctctgggccctagaacaacacagagcagcgtgctgaccatcacacccagacctcaggaccacagcaccaacctgacctgccaagtgacctttcctggcgctggcgtgaccatggaaagaaccatccagctcaacgtgtcctacgctccccagaaggtggccatcagcattttccagggcaacagcgccgccttcaagatcctgcagaataccagcagcctgcctgtgctggaaggccaggctctgagactgctgtgtgacgccgatggcaatcctcctgctcacctgagctggttccagggcttcccagctctgaacgccacacctatcagcaacaccggcgtgctggaactgcctcaagtgggatctgccgaggaaggcgacttcacctgtagagcccaacatcctctgggcagcctgcagatctccctgagcctgtttgtgcactggaagcccgaaggcagagctggcggagtgggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 49 - protein, 558 aa, Artificial Sequence
  Synthetic Siglec 6 Fragment (27-347) SAX / (G4S)2 / hIgG1 Fc N297G
QERRFQLEGPESLTVQEGLCVLVPCRLPTTLPASYYGYGYWFLEGADVPVATNDPDEEVQEETRGRFHLLWDPRRKNCSLSIRDARRRDNAAYFFALKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGVGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 1674 bp, Artificial Sequence
  Synthetic Siglec 6 Fragment (27-347) SAX / (G4S)2 / hIgG1 Fc N297G
caagagcggcggtttcagctggaaggacctgagagcctgacagtgcaagagggcctgtgtgtgctggtgccttgtagactgcctaccacactgcccgccagctactacggctacggctattggtttctggaaggcgccgatgtgcccgtggccaccaatgatcctgacgaggaagtgcaagaagagacacggggcagattccatctgctgtgggaccccagacggaagaactgcagcctgagcatcagagatgccagaaggcgggataacgccgcctacttcttcgccctgaagtccaagtggatgaagtacggctacaccagcagcaagctgagcgtcagagtgatggccctgacacacagacccaacatcagcatccccggcacactggaaagcggacaccctagcaatctgacctgcagcgtgccatgggtttgcgagcagggaacccctcctatcttctcctggatgtctgccgctcctacctctctgggccctagaacaacacagagcagcgtgctgaccatcacacccagacctcaggaccacagcaccaacctgacctgccaagtgacctttcctggcgctggcgtgaccatggaaagaaccatccagctcaacgtgtcctacgctccccagaaggtggccatcagcattttccagggcaacagcgccgccttcaagatcctgcagaataccagcagcctgcctgtgctggaaggccaggctctgagactgctgtgtgacgccgatggcaatcctcctgctcacctgagctggttccagggcttcccagctctgaacgccacacctatcagcaacaccggcgtgctggaactgcctcaagtgggatctgccgaggaaggcgacttcacctgtagagcccaacatcctctgggcagcctgcagatctccctgagcctgtttgtgcactggaagcccgaaggcagagctggcggagtgggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 51 - protein, 467 aa, Homo sapiens
  misc_feature (1)..(467): Siglec 7 FL
MLLLLLLPLLWGRERVEGQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGGAGATALVFLSFCVIFIVVRSCRKKSARPAADVGDIGMKDANTIRGSASQGNLTESWADDNPRHHGLAAHSSGEEREIQYAPLSFHKGEPQDLSGQEATNNEYSEIKIPK

SEQ ID NO: 52 - DNA, 1769 bp, Homo sapiens
  misc_feature (1)..(1769): Siglec 7 FL
gcagttcctgagagaagaaccctgaggaacagacgttccctcgcggccctggcacctccaaccccagatatgctgctgctgctgctgctgcccctgctctgggggagggagagggtggaaggacagaagagtaaccggaaggattactcgctgacgatgcagagttccgtgaccgtgcaagagggcatgtgtgtccatgtgcgctgctccttctcctacccagtggacagccagactgactctgacccagttcatggctactggttccgggcagggaatgatataagctggaaggctccagtggccacaaacaacccagcttgggcagtgcaggaggaaactcgggaccgattccacctccttggggacccacagaccaaaaattgcaccctgagcatcagagatgccagaatgagtgatgcggggagatacttctttcgtatggagaaaggaaatataaaatggaattataaatatgaccagctctctgtgaacgtgacagccttgacccacaggcccaacatccttatccccggtaccctggagtctggctgcttccagaatctgacctgctctgtgccctgggcctgtgagcaggggacgccccctatgatctcctggatggggacctctgtgtcccccctgcacccctccaccacccgctcctcagtgctcaccctcatcccacagccccagcaccacggcaccagcctcacctgtcaggtgaccttgcctggggccggcgtgaccacgaacaggaccatccaactcaatgtgtcctaccctcctcagaacttgactgtgactgtcttccaaggagaaggcacagcatccacagctctggggaacagctcatctctttcagtcctagagggccagtctctgcgcttggtctgtgctgttgacagcaatccccctgccaggctgagctggacctggaggagtctgaccctgtacccctcacagccctcaaaccctctggtactggagctgcaagtgcacctgggggatgaaggggaattcacctgtcgagctcagaactctctgggttcccagcacgtttccctgaacctctccctgcaacaggagtacacaggcaaaatgaggcctgtatcaggagtgttgctgggggcggtcgggggagctggagccacagccctggtcttcctctccttctgtgtcatcttcattgtagtgaggtcctgcaggaagaaatcggcaaggccagcagcggacgtgggagacataggcatgaaggatgcaaacaccatcaggggctcagcctctcagggtaacctgactgagtcctgggcagatgataacccccgacaccatggcctggctgcccactcctcaggggaggaaagagagatccagtatgcacccctcagctttcataagggggagcctcaggacctatcaggacaagaagccaccaacaatgagtactcagagatcaagatccccaagtaagaaaatgcagaggctcgggcttgtttgagggttcacgacccctccagcaaaggagtctgaggctgattccagtagaattagcagccctcaatgctgtgcaacaagacatcagaacttattcctcttgtctaactgaaaatgcatgcctgatgaccaaactctccctttccccatccaatcggtccacactccccgccctggcctctggtacccaccattctcctctgtacttctctaaggatgactactttagattccgaatatagtgagattgtaacgtgaaaaaaaaaaaaaaa

SEQ ID NO: 53 - protein, 119 aa, Homo sapiens
  misc_feature (1)..(119): Siglec 7 V-Set (26-144)
YSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVT

SEQ ID NO: 54 - protein, 92 aa, Homo sapiens
  misc_feature (1)..(92): Siglec 7 V-Set (Alt. boundary) (31-122)
QSSVTVQEGMCVHVRCSFSYPVDSQTDSDPVHGYWFRAGNDISWKAPVATNNPAWAVQEETRDRFHLLGDPQTKNCTLSIRDARMSDAGRYF

SEQ ID NO: 55 - protein, 221 aa, Homo sapiens
  misc_feature (1)..(221): Siglec 7 V-Set + 1 C2-Set Domain (19-239)
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYP

SEQ ID NO: 56 - protein, 339 aa, Homo sapiens
  misc_feature (1)..(339): Siglec 7 V-Set Domain + 2 C2-Set Domains (Full ECD) (19-357)
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVG

SEQ ID NO: 57 - protein, 339 aa, Homo sapiens; identical to SEQ ID NO: 56
  misc_feature (1)..(339): Siglec 7 Fragment (19-357)

SEQ ID NO: 58 - protein, 339 aa, Artificial Sequence
  Synthetic Siglec 7 Fragment (19-357) SAX
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFKMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVG

SEQ ID NO: 59 - protein, 568 aa, Artificial Sequence
  Synthetic Siglec 7 Fragment (19-357) / AS / hIgG1 Fc N297G
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - DNA, 1704 bp, Artificial Sequence
  Synthetic Siglec 7 Fragment (19-357) / AS / hIgG1 Fc N297G
cagaagtccaacagaaaggactacagcctgaccatgcagagcagcgtgacagtgcaagaggggatgtgcgtccacgtccggtgcagctttagctaccctgtggacagccagaccgacagcgatcctgtgcacggctactggttcagagccggcaacgacatctcttggaaagccccagtggccaccaacaatcctgcctgggctgtgcaagaagagacacgggacagattccatctgctgggcgaccctcagaccaagaactgcacactgagcatccgggacgccagaatgtctgacgccggcagatacttcttcagaatggaaaagggcaacatcaagtggaactataagtacgaccagctgagcgtgaacgtgacagccctgacacacagacccaacattctgatccccggcacactggaaagcggctgcttccagaatctgacctgctctgtgccttgggcctgcgagcagggaacacctcctatgatcagctggatgggaaccagcgtgtcccctctgcaccctagcaccacaagatccagcgtgctgacactgatccctcagcctcagcaccacggcacaagcctgacctgtcaagttacacttcctggcgctggcgtgaccaccaacagaacaatccagctcaacgtgtcctatcctcctcagaacctgaccgtgaccgtgttccaaggcgagggcacagcttctacagccctgggcaatagcagcagcctgtctgtgctggaaggccagtctctgagactcgtgtgcgccgtggatagcaaccctcctgctagactgagctggacttggcggagcctgacactgtaccctagccagcctagcaatcccctggtgctggaactgcaagtgcacctgggagatgagggcgagttcacctgtagagcccagaatagcctgggcagccagcacgtgtccctgaacctgtctctgcagcaagagtacaccggcaagatgaggcctgtgtctggcgttctgctgggagccgtgggagctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 61 - protein, 568 aa, Artificial Sequence
  Synthetic Siglec 7 Fragment (19-357) SAX / AS / hIgG1 Fc N297G
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFKMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - DNA, 1704 bp, Artificial Sequence
  Synthetic Siglec 7 Fragment (19-357) SAX / AS / hIgG1 Fc N297G
cagaagtccaacagaaaggactacagcctgaccatgcagagcagcgtgacagtgcaagaggggatgtgcgtccacgtccggtgcagctttagctaccctgtggacagccagaccgacagcgatcctgtgcacggctactggttcagagccggcaacgacatctcttggaaagccccagtggccaccaacaatcctgcctgggctgtgcaagaagagacacgggacagattccatctgctgggcgaccctcagaccaagaactgcacactgagcatccgggacgccagaatgtctgacgccggcagatacttcttcaagatggaaaagggcaacatcaagtggaactataagtacgaccagctgagcgtgaacgtgacagccctgacacacagacccaacattctgatccccggcacactggaaagcggctgcttccagaatctgacctgctctgtgccttgggcctgcgagcagggaacacctcctatgatcagctggatgggaaccagcgtgtcccctctgcaccctagcaccacaagatccagcgtgctgacactgatccctcagcctcagcaccacggcacaagcctgacctgtcaagttacacttcctggcgctggcgtgaccaccaacagaacaatccagctcaacgtgtcctatcctcctcagaacctgaccgtgaccgtgttccaaggcgagggcacagcttctacagccctgggcaatagcagcagcctgtctgtgctggaaggccagtctctgagactcgtgtgcgccgtggatagcaaccctcctgctagactgagctggacttggcggagcctgacactgtaccctagccagcctagcaatcccctggtgctggaactgcaagtgcacctgggagatgagggcgagttcacctgtagagcccagaatagcctgggcagccagcacgtgtccctgaacctgtctctgcagcaagagtacaccggcaagatgaggcctgtgtctggcgttctgctgggagccgtgggagctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 63 - protein, 499 aa, Homo sapiens
  misc_feature (1)..(499): Siglec 8 FL
MLLLLLLLPLLWGTKGMEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 64 - DNA, 2967 bp, Homo sapiens
  misc_feature (1)..(2967): Siglec 8 FL
agtttctgagagaagaaccctgaggaacagacgttccctggcggccctggcgccttcaaacccagacatgctgctgctgctgctgctgctgcccctgctctgggggacaaaggggatggagggagacagacaatatggggatggttacttgctgcaagtgcaggagctggtgacggtgcaggagggcctgtgtgtccatgtgccctgctccttctcctacccccaggatggctggactgactctgacccagttcatggctactggttccgggcaggagacagaccataccaagacgctccagtggccacaaacaacccagacagagaagtgcaggcagagacccagggccgattccaactccttggggacatttggagcaacgactgctccctgagcatcagagacgccaggaagagggataaggggtcatatttctttcggctagagagaggaagcatgaaatggagttacaaatcacagttgaattacaaaactaagcagctgtctgtgtttgtgacagccctgacccataggcctgacatcctcatcctagggaccctagagtctggccactccaggaacctgacctgctctgtgccctgggcctgtaagcaggggacaccccccatgatctcctggattggggcctccgtgtcctccccgggccccactactgcccgctcctcagtgctcacccttaccccaaagccccaggaccacggcaccagcctcacctgtcaggtgaccttgcctgggacaggtgtgaccacgaccagtaccgtccgcctcgatgtgtcctaccctccttggaacttgaccatgactgtcttccaaggagatgccacagcatccacagccctgggaaatggctcatctctttcagtccttgagggccagtctctgcgcctggtctgtgctgtcaacagcaatccccctgccaggctgagctggacccgggggagcctgaccctgtgcccctcacggtcctcaaaccctgggctgctggagctgcctcgagtgcacgtgagggatgaaggggaattcacctgccgagctcagaacgctcagggctcccagcacatttccctgagcctctccctgcagaatgagggcacaggcacctcaagacctgtatcacaagtgacactggcagcagtcgggggagctggagccacagccctggccttcctgtccttctgcatcatcttcatcatagtgaggtcctgcaggaagaaatcggcaaggccagcagcgggcgtgggggatacaggcatggaagatgcaaaggccatcaggggctcggcctctcagggacccctgactgaatcctggaaagatggcaaccccctgaagaagcctcccccagctgttgccccctcgtcaggggaggaaggagagctccattatgcaaccctcagcttccataaagtgaagcctcaggacccgcagggacaggaggccactgacagtgaatactcggagatcaagatccacaagcgagaaactgcagagactcaggcctgtttgaggaatcacaacccctccagcaaagaagtcagaggctgattctcatagaacaagaaccctctagagccccatgctatgcagtaggtcaccagggctccctcctcctgtctaaccaaaacttggaccaatgtctcccctttccccggctaccagggacccatccctgcctctagcttctactacccaccattctcctctcgacctctctgaggttgactattttagattccacatagagatgaggtcatgtggtacttgcctctctgtgtgtggctcattttacacaaaaaaatatcccctaggttcatccatgttctctcaaatgacagaatcaagcactgaatatttttttttctttgagagatggagtttcgctctgttgcccaggctggagtgcagtggttcaatctctgctcactgcaacctccacctcctgggttcaaacgattctcctgcctcagcttcccaagtagctggtactacaggcgtgtgtcaccacgcccagctaatttttgtattttttagtagagacggggtttcactataagtgggccaggctagtctcaaactcctgacctcaagtgatctgcctgccttggcctcccaaagtgctgggatttcaggcatgagccaccgcacccagcttgcattgaatattttcaaggagctaaaagaagattttaaatggtctcacaaaaacagataaatatttgcacagatgggtgtgctaatcattgtgccttgatggttccacgatgtatccgggtgtggaaatctcactgggtctctctcaaggccactcggctactcaggacagggctggaatttaaagcctgtccgattctgaggtctcttctctcatctagcactgagtcaagcaatcagcaggctgggcaccccttagccataagttttcaggaaataaattccttgagggcattgacttttacaaaagagggagcagcaatggcctagagtctcaggaacaagacaggtgcactgaggagatgaaggccgggaccccctgcccaacctgtatggcgggtctgtacttattttgtttacccccaatttaaaacgtttttttttattgcaggttgtttgtttgatatggtttggctgtgtccccacccaaatcttatctagaattgtaatcagaattataatccccatgtgttgggggagggacctggtgggaggtgataggatcatgggggtggttcccccatgctgttctgatagtgagtgagttatcacgagatctgatggttttgtaagtggtggtttcccctgctcttctctcttgcctgccaccatgtaagatgtgcctggttccccttccgccatgattgtaagtttcctgaggcctcccccgccatgtggaactgtgagtcaattacacctctttcatttataaattaaaaaaaaaaaaaaaaaaa

SEQ ID NO: 65 - protein, 463 aa, Homo sapiens
  misc_feature (1)..(463): Siglec 9 FL
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 66 - DNA, 1737 bp, Homo sapiens
  misc_feature (1)..(1737): Siglec 9 FL
tagggcctcctctaagtcttgagcccgcagttcctgagagaagaaccctgaggaacagacgttccctcgcggccctggcacctctaaccccagacatgctgctgctgctgctgcccctgctctgggggagggagagggcggaaggacagacaagtaaactgctgacgatgcagagttccgtgacggtgcaggaaggcctgtgtgtccatgtgccctgctccttctcctacccctcgcatggctggatttaccctggcccagtagttcatggctactggttccgggaaggggccaatacagaccaggatgctccagtggccacaaacaacccagctcgggcagtgtgggaggagactcgggaccgattccacctccttggggacccacataccaagaattgcaccctgagcatcagagatgccagaagaagtgatgcggggagatacttctttcgtatggagaaaggaagtataaaatggaattataaacatcaccggctctctgtgaatgtgacagccttgacccacaggcccaacatcctcatcccaggcaccctggagtccggctgcccccagaatctgacctgctctgtgccctgggcctgtgagcaggggacaccccctatgatctcctggatagggacctccgtgtcccccctggacccctccaccacccgctcctcggtgctcaccctcatcccacagccccaggaccatggcaccagcctcacctgtcaggtgaccttccctggggccagcgtgaccacgaacaagaccgtccatctcaacgtgtcctacccgcctcagaacttgaccatgactgtcttccaaggagacggcacagtatccacagtcttgggaaatggctcatctctgtcactcccagagggccagtctctgcgcctggtctgtgcagttgatgcagttgacagcaatccccctgccaggctgagcctgagctggagaggcctgaccctgtgcccctcacagccctcaaacccgggggtgctggagctgccttgggtgcacctgagggatgcagctgaattcacctgcagagctcagaaccctctcggctctcagcaggtctacctgaacgtctccctgcagagcaaagccacatcaggagtgactcagggggtggtcgggggagctggagccacagccctggtcttcctgtccttctgcgtcatcttcgttgtagtgaggtcctgcaggaagaaatcggcaaggccagcagcgggcgtgggagatacgggcatagaggatgcaaacgctgtcaggggttcagcctctcaggggcccctgactgaaccttgggcagaagacagtcccccagaccagcctcccccagcttctgcccgctcctcagtgggggaaggagagctccagtatgcatccctcagcttccagatggtgaagccttgggactcgcggggacaggaggccactgacaccgagtactcggagatcaagatccacagatgagaaactgcagagactcaccctgattgagggatcacagcccctccaggcaagggagaagtcagaggctgattcttgtagaattaacagccctcaacgtgatgagctatgataacactatgaattatgtgcagagtgaaaagcacacaggctttagagtcaaagtatctcaaacctgaatccacactgtgccctcccttttatttttttaactaaaagacagacaaattcctaaaaaaaaaaaaaaaaaaaa

SEQ ID NO: 67 - protein, 122 aa, Homo sapiens
  misc_feature (1)..(122): Siglec 9 V-Set (23-144)
LTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTH

SEQ ID NO: 68 - protein, 118 aa, Homo sapiens
  misc_feature (1)..(118): Siglec 9 V-Set (Alt. boundary) (23-140)
LTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVT

SEQ ID NO: 69 - protein, 218 aa, Homo sapiens
  misc_feature (1)..(218): Siglec 9 V-Set + 1 C2-Set Domain (18-235)
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYP

SEQ ID NO: 70 - protein, 331 aa, Homo sapiens
  misc_feature (1)..(331): Siglec 9 V-Set Domain + 2 C2-Set Domains (Full ECD) (18-348)
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQG

SEQ ID NO: 71 - protein, 333 aa, Artificial Sequence
  Synthetic Siglec 9 Fragment (18-348) C158S, C295Y
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGSPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLYPSQPSNPGVLELPWVHLRDAAEFTCRAQNPLGSQQVYLNVSLQSKATSGVTQGAS

SEQ ID NO: 72 - protein, 333 aa, Artificial Sequence
  Synthetic Siglec 9 Fragment (18-348) C158S, C295Y, SAX
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFKMEKGSIKWNYKHHRLSVNVTALTHRPNILIPGTLESGSPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLYPSQPSNPGVLELPWVHLRDAAEFTCRAQNPLGSQQVYLNVSLQSKATSGVTQGAS

SEQ ID NO: 73 - protein, 560 aa, Artificial Sequence
  Synthetic Siglec 9 Fragment (18-348) C158S, C295Y / AS / hIgG1 Fc N297G
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGSPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLYPSQPSNPGVLELPWVHLRDAAEFTCRAQNPLGSQQVYLNVSLQSKATSGVTQGASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - DNA, 1680 bp, Artificial Sequence
  Synthetic Siglec 9 Fragment (18-348) C158S, C295Y / AS / hIgG1 Fc N297G
cagaccagcaagctgctgaccatgcagagcagcgtgaccgtgcaggagggcctgtgcgtgcatgtgccctgcagcttcagctaccccagccacggctggatctaccccggtcccgtagtgcacggctactggttcagggagggcgccaacaccgaccaggacgctcccgtggcaaccaacaaccccgccagggccgtgtgggaggagaccagggacaggttccacctgctgggcgacccccacaccaagaactgcaccctgagcatcagggacgccaggaggagcgacgccggcaggtacttcttcaggatggagaaggggtctatcaagtggaactacaagcaccaccggctgagcgtgaatgtgaccgccctgacccaccggcccaatatcctcatccccggcaccctggagagcggcagcccccagaatcttacctgcagcgtaccctgggcctgcgagcagggcacccctccaatgatcagctggatcggcaccagcgtgagccccctggaccctagtaccaccaggagcagcgtgctgaccctgatcccccagccccaggaccacggaaccagcctgacctgccaggtgaccttccccggagccagcgtaaccaccaacaagaccgtgcacctgaacgtgagctacccaccccaaaacctgaccatgaccgtgttccagggcgacggcacggtgagcaccgtactgggcaacggcagctctctgagcctgcccgagggccagagcttgcggctggtctgcgccgtggatgctgtggacagcaaccctcccgccaggctgagcctgagctggaggggcctgaccctgtaccccagccagcccagcaatcccggcgtgctggagctgccctgggttcacctgagggacgctgccgagttcacatgtagggcccagaaccccctgggctctcagcaggtgtacctgaacgtgtctcttcagagtaaggccaccagcggcgtgacccaaggagctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 75 - protein, 560 aa, Artificial Sequence
  Synthetic Siglec 9 Fragment (18-348) C158S, C295Y, SAX / AS / hIgG1 Fc N297G
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFKMEKGSIKWNYKHHRLSVNVTALTHRPNILIPGTLESGSPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLYPSQPSNPGVLELPWVHLRDAAEFTCRAQNPLGSQQVYLNVSLQSKATSGVTQGASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - DNA, 1680 bp, Artificial Sequence
  Synthetic Siglec 9 Fragment (18-348) C158S, C295Y, SAX / AS / hIgG1 Fc N297G
cagaccagcaagctgctgaccatgcagagcagcgtgaccgtgcaggagggcctgtgcgtgcatgtgccctgcagcttcagctaccccagccacggctggatctaccccggtcccgtagtgcacggctactggttcagggagggcgccaacaccgaccaggacgctcccgtggcaaccaacaaccccgccagggccgtgtgggaggagaccagggacaggttccacctgctgggcgacccccacaccaagaactgcaccctgagcatcagggacgccaggaggagcgacgccggcaggtacttcttcaagatggagaaggggtctatcaagtggaactacaagcaccaccggctgagcgtgaatgtgaccgccctgacccaccggcccaatatcctcatccccggcaccctggagagcggcagcccccagaatcttacctgcagcgtaccctgggcctgcgagcagggcacccctccaatgatcagctggatcggcaccagcgtgagccccctggaccctagtaccaccaggagcagcgtgctgaccctgatcccccagccccaggaccacggaaccagcctgacctgccaggtgaccttccccggagccagcgtaaccaccaacaagaccgtgcacctgaacgtgagctacccaccccaaaacctgaccatgaccgtgttccagggcgacggcacggtgagcaccgtactgggcaacggcagctctctgagcctgcccgagggccagagcttgcggctggtctgcgccgtggatgctgtggacagcaaccctcccgccaggctgagcctgagctggaggggcctgaccctgtaccccagccagcccagcaatcccggcgtgctggagctgccctgggttcacctgagggacgctgccgagttcacatgtagggcccagaaccccctgggctctcagcaggtgtacctgaacgtgtctcttcagagtaaggccaccagcggcgtgacccaaggagctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 77 - protein, 357 aa, Artificial Sequence
  Synthetic Siglec 9 Fragment (18-145) / AS / hIgG1 Fc (w/ monovalent mutations)
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGK

SEQ ID NO: 78 - DNA, 1071 bp, Artificial Sequence
  Synthetic Siglec 9 Fragment (18-145) / AS / hIgG1 Fc (w/ monovalent mutations)
cagaccagcaagctgctgaccatgcagagcagcgtgaccgtgcaggagggcctgtgcgtgcatgtgccctgcagcttcagctaccccagccacggctggatctaccccggtcccgtagtgcacggctactggttcagggagggcgccaacaccgaccaggacgctcccgtggcaaccaacaaccccgccagggccgtgtgggaggagaccagggacaggttccacctgctgggcgacccccacaccaagaactgcaccctgagcatcagggacgccaggaggagcgacgccggcaggtacttcttcaggatggagaaggggtctatcaagtggaactacaagcaccaccggctgagcgtgaatgtgaccgccctgacccaccgggctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcaacctgacctgcctggtcaaaggcttctatcccagcgacatcgccgtggagtgggagagcaatgggcagccggagaacaactacaagaccacgcctcccgtgctggactccgacggctccttcttcctcaatagcacgctcaccgtggacaagagcaggtggcagcaggggaacgtcttctcatgctccgtgatgcatgaggctctgcacaaccactacacgcagaagagcctctccctgtctccgggtaaa

SEQ ID NO: 79 - protein, 341 aa, Artificial Sequence
  Synthetic Siglec 9 Fragment (18-348) C158S, C295Y / His Tag
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGSPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLYPSQPSNPGVLELPWVHLRDAAEFTCRAQNPLGSQQVYLNVSLQSKATSGVTQGHHHHHHHHHH

SEQ ID NO: 80 - DNA, 1026 bp, Artificial Sequence
  Synthetic Siglec 9 Fragment (18-348) C158S, C295Y / His Tag
cagaccagcaagctgctgaccatgcagagcagcgtgaccgtgcaggagggcctgtgcgtgcatgtgccctgcagcttcagctaccccagccacggctggatctaccccggtcccgtagtgcacggctactggttcagggagggcgccaacaccgaccaggacgctcccgtggcaaccaacaaccccgccagggccgtgtgggaggagaccagggacaggttccacctgctgggcgacccccacaccaagaactgcaccctgagcatcagggacgccaggaggagcgacgccggcaggtacttcttcaagatggagaaggggtctatcaagtggaactacaagcaccaccggctgagcgtgaatgtgaccgccctgacccaccggcccaatatcctcatccccggcaccctggagagcggcagcccccagaatcttacctgcagcgtaccctgggcctgcgagcagggcacccctccaatgatcagctggatcggcaccagcgtgagccccctggaccctagtaccaccaggagcagcgtgctgaccctgatcccccagccccaggaccacggaaccagcctgacctgccaggtgaccttccccggagccagcgtaaccaccaacaagaccgtgcacctgaacgtgagctacccaccccaaaacctgaccatgaccgtgttccagggcgacggcacggtgagcaccgtactgggcaacggcagctctctgagcctgcccgagggccagagcttgcggctggtctgcgccgtggatgctgtggacagcaaccctcccgccaggctgagcctgagctggaggggcctgaccctgtaccccagccagcccagcaatcccggcgtgctggagctgccctgggttcacctgagggacgctgccgagttcacatgtagggcccagaaccccctgggctctcagcaggtgtacctgaacgtgtctcttcagagtaaggccaccagcggcgtgacccaaggacaccatcaccatcatcaccaccaccatcaccat

SEQ ID NO: 81 - protein, 432 aa, Artificial Sequence
  Synthetic Siglec 9 Fragment (146-348) / AS / hIgG1 Fc N297G
PNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - DNA, 1296 bp, Artificial Sequence
  Synthetic Siglec 9 Fragment (146-348) / AS / hIgG1 Fc N297G
cccaatatcctcatccccggcaccctggagagcggctgcccccagaatcttacctgcagcgtaccctgggcctgcgagcagggcacccctccaatgatcagctggatcggcaccagcgtgagccccctggaccctagtaccaccaggagcagcgtgctgaccctgatcccccagccccaggaccacggaaccagcctgacctgccaggtgaccttccccggagccagcgtaaccaccaacaagaccgtgcacctgaacgtgagctacccaccccaaaacctgaccatgaccgtgttccagggcgacggcacggtgagcaccgtactgggcaacggcagctctctgagcctgcccgagggccagagcttgcggctggtctgcgccgtggatgctgtggacagcaaccctcccgccaggctgagcctgagctggaggggcctgaccctgtgccccagccagcccagcaatcccggcgtgctggagctgccctgggttcacctgagggacgctgccgagttcacatgtagggcccagaaccccctgggctctcagcaggtgtacctgaacgtgtctcttcagagtaaggccaccagcggcgtgacccaaggagctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 83 - protein, 560 aa, Artificial Sequence
  Synthetic Siglec 9 Fragment (18-348) K131Q / AS / hIgG1 Fc N297G
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYQHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - DNA, 1680 bp, Artificial Sequence
  Synthetic Siglec 9 Fragment (18-348) K131Q / AS / hIgG1 Fc N297G
cagaccagcaagctgctgaccatgcagagcagcgtgaccgtgcaggagggcctgtgcgtgcatgtgccctgcagcttcagctaccccagccacggctggatctaccccggtcccgtagtgcacggctactggttcagggagggcgccaacaccgaccaggacgctcccgtggcaaccaacaaccccgccagggccgtgtgggaggagaccagggacaggttccacctgctgggcgacccccacaccaagaactgcaccctgagcatcagggacgccaggaggagcgacgccggcaggtacttcttcaggatggagaaggggtctatcaagtggaactaccagcaccaccggctgagcgtgaatgtgaccgccctgacccaccggcccaatatcctcatccccggcaccctggagagcggctgcccccagaatcttacctgcagcgtaccctgggcctgcgagcagggcacccctccaatgatcagctggatcggcaccagcgtgagccccctggaccctagtaccaccaggagcagcgtgctgaccctgatcccccagccccaggaccacggaaccagcctgacctgccaggtgaccttccccggagccagcgtaaccaccaacaagaccgtgcacctgaacgtgagctacccaccccaaaacctgaccatgaccgtgttccagggcgacggcacggtgagcaccgtactgggcaacggcagctctctgagcctgcccgagggccagagcttgcggctggtctgcgccgtggatgctgtggacagcaaccctcccgccaggctgagcctgagctggaggggcctgaccctgtgccccagccagcccagcaatcccggcgtgctggagctgccctgggttcacctgagggacgctgccgagttcacatgtagggcccagaaccccctgggctctcagcaggtgtacctgaacgtgtctcttcagagtaaggccaccagcggcgtgacccaaggagctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 85 - protein, 560 aa, Artificial Sequence
  Synthetic Siglec 9 Fragment (18-348) K100E, A315E / AS / hIgG1 Fc N297G
QTSKLLTMQSSVTVQEGLCVHVPCSFSYPSHGWIYPGPVVHGYWFREGANTDQDAPVATNNPARAVWEETRDRFHLLGDPHTENCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDEAEFTCRAQNPLGSQQVYLNVSLQSKATSGVTQGASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - DNA, 1680 bp, Artificial Sequence
  Synthetic Siglec 9 Fragment (18-348) K100E, A315E / AS / hIgG1 Fc N297G
cagaccagcaagctgctgaccatgcagagcagcgtgaccgtgcaggagggcctgtgcgtgcatgtgccctgcagcttcagctaccccagccacggctggatctaccccggtcccgtagtgcacggctactggttcagggagggcgccaacaccgaccaggacgctcccgtggcaaccaacaaccccgccagggccgtgtgggaggagaccagggacaggttccacctgctgggcgacccccacaccgagaactgcaccctgagcatcagggacgccaggaggagcgacgccggcaggtacttcttcaggatggagaaggggtctatcaagtggaactacaagcaccaccggctgagcgtgaatgtgaccgccctgacccaccggcccaatatcctcatccccggcaccctggagagcggctgcccccagaatcttacctgcagcgtaccctgggcctgcgagcagggcacccctccaatgatcagctggatcggcaccagcgtgagccccctggaccctagtaccaccaggagcagcgtgctgaccctgatcccccagccccaggaccacggaaccagcctgacctgccaggtgaccttccccggagccagcgtaaccaccaacaagaccgtgcacctgaacgtgagctacccaccccaaaacctgaccatgaccgtgttccagggcgacggcacggtgagcaccgtactgggcaacggcagctctctgagcctgcccgagggccagagcttgcggctggtctgcgccgtggatgctgtggacagcaaccctcccgccaggctgagcctgagctggaggggcctgaccctgtgccccagccagcccagcaatcccggcgtgctggagctgccctgggttcacctgagggacgaagccgagttcacatgtagggcccagaaccccctgggctctcagcaggtgtacctgaacgtgtctcttcagagtaaggccaccagcggcgtgacccaaggagctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 87 - protein, 697 aa, Homo sapiens
  misc_feature (1)..(697): Siglec 10 FL
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSNGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 88 - DNA, 3400 bp, Homo sapiens
  misc_feature (1)..(3400): Siglec 10 FL
gcccccaggagacccagaggacaactgggcaaggtgggccggagagtgtgggggaaggcaaaggagttctgtgagctcagcgtctgaagctcatttcatgcatcaggccccagggctcagcttccgccttcggcttccccttctgccaagagccctgagccactcacagcacgaccagagaacaggcctgtctcaggcaggccctgcgcctcctatgcggagatgctactgccactgctgctgtcctcgctgctgggcgggtcccaggctatggatgggagattctggatacgagtgcaggagtcagtgatggtgccggagggcctgtgcatctctgtgccctgctctttctcctacccccgacaagactggacagggtctaccccagcttatggctactggttcaaagcagtgactgagacaaccaagggtgctcctgtggccacaaaccaccagagtcgagaggtggaaatgagcacccggggccgattccagctcactggggatcccgccaaggggaactgctccttggtgatcagagacgcgcagatgcaggatgagtcacagtacttctttcgggtggagagaggaagctatgtgagatataatttcatgaacgatgggttctttctaaaagtaacagccctgactcagaagcctgatgtctacatccccgagaccctggagcccgggcagccggtgacggtcatctgtgtgtttaactgggcctttgaggaatgtccacccccttctttctcctggacgggggctgccctctcctcccaaggaaccaaaccaacgacctcccacttctcagtgctcagcttcacgcccagaccccaggaccacaacaccgacctcacctgccatgtggacttctccagaaagggtgtgagcgcacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctgtgcagtatcctccagagaacctgagagtgatggtttcccaagcaaacaggacagtcctggaaaaccttgggaacggcacgtctctcccagtactggagggccaaagcctgtgcctggtctgtgtcacacacagcagccccccagccaggctgagctggacccagaggggacaggttctgagcccctcccagccctcagaccccggggtcctggagctgcctcgggttcaagtggagcacgaaggagagttcacctgccacgctcggcacccactgggctcccagcacgtctctctcagcctctccgtgcactactccccgaagctgctgggcccctcctgctcctgggaggctgagggtctgcactgcagctgctcctcccaggccagcccggccccctctctgcgctggtggcttggggaggagctgctggaggggaacagcagccaggactccttcgaggtcacccccagctcagccgggccctgggccaacagctccctgagcctccatggagggctcagctccggcctcaggctccgctgtgaggcctggaacgtccatggggcccagagtggatccatcctgcagctgccagataagaagggactcatctcaacggcattctccaacggagcgtttctgggaatcggcatcacggctcttcttttcctctgcctggccctgatcatcatgaagattctaccgaagagacggactcagacagaaaccccgaggcccaggttctcccggcacagcacgatcctggattacatcaatgtggtcccgacggctggccccctggctcagaagcggaatcagaaagccacaccaaacagtcctcggacccctcttccaccaggtgctccctccccagaatcaaagaagaaccagaaaaagcagtatcagttgcccagtttcccagaacccaaatcatccactcaagccccagaatcccaggagagccaagaggagctccattatgccacgctcaacttcccaggcgtcagacccaggcctgaggcccggatgcccaagggcacccaggcggattatgcagaagtcaagttccaatgagggtctcttaggctttaggactgggacttcggctagggaggaaggtagagtaagaggttgaagataacagagtgcaaagtttccttctctccctctctctctctctttctctctctctctctctttctctctcttttaaaaaaacatctggccagggcacagtggctcacgcctgtaatcccagcactttgggaggttgaggtgggcagatcgcctgaggtcgggagttcgagaccagcctggccaacttggtgaaaccccgtctctactaaaaatacaaaaattagctgggcatggtggcaggcgcctgtaatcctacctacttgggaagctgaggcaggagaatcacttgaacctgggagacggaggttgcagtgagccaagatcacaccattgcacgccagcctgggcaacaaagcgagactccatctcaaaaaaaaaatcctccaaatgggttgggtgtctgtaatcccagcactttgggaggctaaggtgggtggattgcttgagcccaggagttcgagaccagcctgggcaacatggtgaaaccccatctctacaaaaaatacaaaacatagctgggcttggtggtgtgtgcctgtagtcccagctgtcagacatttaaaccagagcaactccatctggaataggagctgaataaaatgaggctgagacctactgggctgcattctcagacagtggaggcattctaagtcacaggatgagacaggaggtccgtacaagatacaggtcataaagactttgctgataaaacagattgcagtaaagaagccaaccaaatcccaccaaaaccaagttggccacgagagtgacctctggtcgtcctcactgctacactcctgacagcaccatgacagtttacaaatgccatggcaacatcaggaagttacccgatatgtcccaaaagggggaggaatgaataatccaccccttgtttagcaaataagcaagaaataaccataaaagtgggcaaccagcagctctaggcgctgctcttgtctatggagtagccattcttttgttcctttactttcttaataaacttgctttcaccttaaaaaaa

SEQ ID NO: 89 - protein, 533 aa, Homo sapiens
  misc_feature (1)..(533): Siglec 10 Fragment (18-550)
DGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSN

SEQ ID NO: 90 - protein, 770 aa, Artificial Sequence
  Synthetic Siglec 10 Fragment (18-550) / (G4S)2 / hIgG1 Fc N297G
DGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSNGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 2310 bp, Artificial Sequence
  Synthetic Siglec 10 Fragment (18-550) / (G4S)2 / hIgG1 Fc N297G
gacggcagattctggatcagagtgcaagagtccgtgatggtgcctgagggcctgtgtatcagcgtgccctgcagcttcagctaccccagacaggattggacaggcagcacaccagcctacggctactggtttaaggccgtgaccgaaaccaccaagggcgctccagtggccacaaaccaccagagcagagaagtggaaatgagcacccggggcagattccagctgacaggcgatcctgccaagggcaattgcagcctggtcatcagagatgcccagatgcaggacgagagccagtacttcttcagagtggaacggggcagctacgtgcggtacaactttatgaacgacggattcttcctgaaagtgacggccctgacacagaaacccgacgtgtacatccccgagacactggaacctggccagcctgtgacagtgatctgcgtgttcaactgggccttcgaggaatgccctccaccttccttttcttggactggcgccgctctgtctagccagggcacaaagcctaccaccagccactttagcgtgctgagcttcacccctagacctcaggaccacaacaccgacctgacctgccacgtggacttcagcagaaaaggcgtgtcagcccagagaaccgtgcggctgagagtggcctatgctcccagagatctcgtgatcagcatcagccgggacaacacccctgctctggaacctcagcctcagggcaatgtgccttacctggaagcccagaagggccagttcctgagactgctgtgtgccgccgattctcagcctcctgctacactgtcttgggtgctgcagaacagagtgctgagcagctctcacccttggggccctagaccactgggacttgaactgcctggtgtcaaggcaggcgacagcggcagatacacatgcagagccgagaacagactgggcagccagcagagagcactggatctgagcgtgcagtaccctcctgagaacctgagagtgatggtgtcccaggccaaccggaccgtgctggaaaatctcggcaatggcacaagcctgcctgtgctggaaggccagtctctgtgtctcgtgtgtgtgacccacagcagccctccagccagactctcttggacacagcgaggacaggtgctgtctcctagccagccttctgatcctggcgtcctggaactgcctagagtgcaggttgagcacgagggcgagttcacctgtcacgccagacatcctctgggctctcagcacgtttccctgagcctgtctgtgcactacagccctaagctgctgggccctagctgttcttgggaagccgaaggcctgcactgcagctgtagctctcaagcctctcctgctcctagcctgagatggtggctgggcgaagaactgctcgagggcaatagctcccaggacagcttcgaagtgaccccttcttctgccggaccttgggccaatagctctctgtctctgcacggcggactgagcagtggactgagactgagatgcgaggcctggaatgtgcacggtgctcagtctggctctatcctgcagctgcccgataagaagggcctgatcagcaccgccttcagcaacggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 92 - protein, 698 aa, Homo sapiens
  misc_feature (1)..(698): Siglec 11 FL
MVPGQAQPQSPEMLLLPLLLPVLGAGSLNKDPSYSLQVQRQVPVPEGLCVIVSCNLSYPRDGWDESTAAYGYWFKGRTSPKTGAPVATNNQSREVEMSTRDRFQLTGDPGKGSCSLVIRDAQREDEAWYFFRVERGSRVRHSFLSNAFFLKVTALTKKPDVYIPETLEPGQPVTVICVFNWAFKKCPAPSFSWTGAALSPRRTRPSTSHFSVLSFTPSPQDHDTDLTCHVDFSRKGVSAQRTVRLRVAYAPKDLIISISHDNTSALELQGNVIYLEVQKGQFLRLLCAADSQPPATLSWVLQDRVLSSSHPWGPRTLGLELRGVRAGDSGRYTCRAENRLGSQQQALDLSVQYPPENLRVMVSQANRTVLENLGNGTSLPVLEGQSLRLVCVTHSSPPARLSWTRWGQTVGPSQPSDPGVLELPPIQMEHEGEFTCHAQHPLGSQHVSLSLSVHYPPQLLGPSCSWEAEGLHCSCSSQASPAPSLRWWLGEELLEGNSSQGSFEVTPSSAGPWANSSLSLHGGLSSGLRLRCKAWNVHGAQSGSVFQLLPGKLEHGGGLGLGAALGAGVAALLAFCSCLVVFRVKICRKEARKRAAAEQDVPSTLGPISQGHQHECSAGSSQDHPPPGAATYTPGKGEEQELHYASLSFQGLRLWEPADQEAPSTTEYSEIKIHTGQPLRGPGFGLQLEREMSGMVPK

SEQ ID NO: 93 - DNA, 3183 bp, Homo sapiens
  misc_feature (1)..(3183): Siglec 11 FL
cgaggctcctcctctgtggatggtcactgcccctccaccaggcttcctgctggaggagtttccttcccagccaggccggcccagaagccagatggtcccgggacaggcccagccccagagcccagagatgctgctgctgcccctgctgctgcccgtgctgggggcggggtccctgaacaaggatcccagttacagtcttcaagtgcagaggcaggtgccggtgccggagggcctgtgtgtcatcgtgtcttgcaacctctcctacccccgggatggctgggacgagtctactgctgcttatggctactggttcaaaggacggaccagcccaaagacgggtgctcctgtggccactaacaaccagagtcgagaggtggaaatgagcacccgggaccgattccagctcactggggatcccggcaaagggagctgctccttggtgatcagagacgcgcagagggaggatgaggcatggtacttctttcgggtggagagaggaagccgtgtgagacatagtttcctgagcaatgcgttctttctaaaagtaacagccctgactaagaagcctgatgtctacatccccgagaccctggagcccgggcagccggtgacggtcatctgtgtgtttaactgggctttcaagaaatgtccagccccttctttctcctggacgggggctgccctctcccctagaagaaccagaccaagcacctcccacttctcagtgctcagcttcacgcccagcccccaggaccacgacaccgacctcacctgccatgtggacttctccagaaagggtgtgagcgcacagaggaccgtccgactccgtgtggcctatgcccccaaagaccttattatcagcatttcacatgacaacacgtcagccctggaactccagggaaacgtcatatatctggaagttcagaaaggccagttcctgcggctcctctgtgctgctgacagccagccccctgccacgctgagctgggtcctgcaggacagagtcctctcctcgtcccacccctggggccccagaaccctggggctggagctgcgtggggtaagggccggggattcagggcgctacacctgccgagcggagaacaggcttggctcccagcagcaagccctggacctctctgtgcagtatcctccagagaacctgagagtgatggtttcccaagcaaacaggacagtcctggaaaacctcgggaacggcacatccctcccggtcctggagggccaaagcctgcgcctggtctgtgtcacccacagcagccccccagccaggctgagctggacccggtggggacagaccgtgggcccctcccagccctcagaccccggggtcctggagctgccacccattcaaatggagcacgaaggagagttcacctgccacgctcagcaccctctgggctcccagcacgtctctctcagcctctccgtgcactaccctccacagctgctgggcccctcctgctcctgggaggctgagggtctgcactgcagctgctcctcccaggccagcccggccccctctctgcgctggtggcttggggaggagctgctggaggggaacagcagtcagggctccttcgaggtcacccccagctcagccgggccctgggccaacagctccctgagcctccatggagggctcagctccggcctcaggctccgctgtaaggcctggaacgtccacggggcccagagtggctctgtcttccagctgctaccagggaagctggagcatgggggaggacttggcctgggggctgccctgggagctggcgtcgctgccctgctcgctttctgttcctgccttgtcgtcttcagggtgaagatctgcaggaaggaagctcgcaagagggcagcagctgagcaggacgtgccctccaccctgggacccatctcccagggtcaccagcatgaatgctcggcaggcagctcccaagaccacccgcccccaggtgcagccacctacaccccggggaagggggaagagcaggagctccactatgcctccctcagcttccagggcctgaggctctgggagcctgcggaccaggaggcccccagcaccaccgagtactcggagatcaagatccacacaggacagcccctgaggggcccaggctttgggcttcaattggagagggagatgtcagggatggttccaaagtgaagaggtctccatggcaacaggacaccagcaagtgtgtgggagtcgcactggtgtgacggccagaactggactcagatttcagccccatccccaatgaagagcttgagtttgaagattatactttttttgagacagggtctgactctgtcctccaggccagagtccagtggtgcaatctcagctcactgtagcctcaacctgccaggttgaagtgagcctcccatttcagcctcccaagtagctgggactacaattgtgagccaccatgccaggctcattgttatatttttagtagagacagggttttgccatgtttccctggctggtctcagactcctgggctcaagcaatctgcccgcctctgcctcccaaagtgctgggattacagacgtgagccaccacagctggctgaagattatactttcaattcagagcgagtttgaagatgacactttgaggcatcgtgtctatggttcattactacagaagcttctctggatgtgtaaagcacaggaaaccaggcagaggaggcacagggtgctctccagaacgagaagccagctcctggagttgtttgctgcaactgccattccccgttgatgaccatgctcttccttcagaagagggagagtgagaggaccaagtccaagtggttcccatttgaacatttaaaaaaaaaaaaaaggctgggcatggtggctcacgcctgtaatctcaacactttgggaggctgaagtgggtggatcacaagtcaggagttcaagaccagcctgggcaagatggtgaaaccccatctctactaaaaatacaaaaattagccgggcatggtggcgggcgcctaaaatcccagctactcgggagactaggcagagaattggttgaacccgggaggtggaggttgcagtgagccgagatcgtcccactgcactccagcctgggcaacagagtgagactctgtttctaaataaataaatgaaaaaaaaaaaaaaaaaaaa

SEQ ID NO: 94 - protein, 534 aa, Homo sapiens
  misc_feature (1)..(534): Siglec 11 Fragment (28-561)
LNKDPSYSLQVQRQVPVPEGLCVIVSCNLSYPRDGWDESTAAYGYWFKGRTSPKTGAPVATNNQSREVEMSTRDRFQLTGDPGKGSCSLVIRDAQREDEAWYFFRVERGSRVRHSFLSNAFFLKVTALTKKPDVYIPETLEPGQPVTVICVFNWAFKKCPAPSFSWTGAALSPRRTRPSTSHFSVLSFTPSPQDHDTDLTCHVDFSRKGVSAQRTVRLRVAYAPKDLIISISHDNTSALELQGNVIYLEVQKGQFLRLLCAADSQPPATLSWVLQDRVLSSSHPWGPRTLGLELRGVRAGDSGRYTCRAENRLGSQQQALDLSVQYPPENLRVMVSQANRTVLENLGNGTSLPVLEGQSLRLVCVTHSSPPARLSWTRWGQTVGPSQPSDPGVLELPPIQMEHEGEFTCHAQHPLGSQHVSLSLSVHYPPQLLGPSCSWEAEGLHCSCSSQASPAPSLRWWLGEELLEGNSSQGSFEVTPSSAGPWANSSLSLHGGLSSGLRLRCKAWNVHGAQSGSVFQLLPGKLEHGGGLGL

SEQ ID NO: 95 - protein, 534 aa, Artificial Sequence
  Synthetic Siglec 11 Fragment (28-561) SAX
LNKDPSYSLQVQRQVPVPEGLCVIVSCNLSYPRDGWDESTAAYGYWFKGRTSPKTGAPVATNNQSREVEMSTRDRFQLTGDPGKGSCSLVIRDAQREDEAWYFFAVERGSRVRHSFLSNAFFLKVTALTKKPDVYIPETLEPGQPVTVICVFNWAFKKCPAPSFSWTGAALSPRRTRPSTSHFSVLSFTPSPQDHDTDLTCHVDFSRKGVSAQRTVRLRVAYAPKDLIISISHDNTSALELQGNVIYLEVQKGQFLRLLCAADSQPPATLSWVLQDRVLSSSHPWGPRTLGLELRGVRAGDSGRYTCRAENRLGSQQQALDLSVQYPPENLRVMVSQANRTVLENLGNGTSLPVLEGQSLRLVCVTHSSPPARLSWTRWGQTVGPSQPSDPGVLELPPIQMEHEGEFTCHAQHPLGSQHVSLSLSVHYPPQLLGPSCSWEAEGLHCSCSSQASPAPSLRWWLGEELLEGNSSQGSFEVTPSSAGPWANSSLSLHGGLSSGLRLRCKAWNVHGAQSGSVFQLLPGKLEHGGGLGL

SEQ ID NO: 96 - protein, 771 aa, Artificial Sequence
  Synthetic Siglec 11 Fragment (28-561) / (G4S)2 / hIgG1 Fc N297G
LNKDPSYSLQVQRQVPVPEGLCVIVSCNLSYPRDGWDESTAAYGYWFKGRTSPKTGAPVATNNQSREVEMSTRDRFQLTGDPGKGSCSLVIRDAQREDEAWYFFRVERGSRVRHSFLSNAFFLKVTALTKKPDVYIPETLEPGQPVTVICVFNWAFKKCPAPSFSWTGAALSPRRTRPSTSHFSVLSFTPSPQDHDTDLTCHVDFSRKGVSAQRTVRLRVAYAPKDLIISISHDNTSALELQGNVIYLEVQKGQFLRLLCAADSQPPATLSWVLQDRVLSSSHPWGPRTLGLELRGVRAGDSGRYTCRAENRLGSQQQALDLSVQYPPENLRVMVSQANRTVLENLGNGTSLPVLEGQSLRLVCVTHSSPPARLSWTRWGQTVGPSQPSDPGVLELPPIQMEHEGEFTCHAQHPLGSQHVSLSLSVHYPPQLLGPSCSWEAEGLHCSCSSQASPAPSLRWWLGEELLEGNSSQGSFEVTPSSAGPWANSSLSLHGGLSSGLRLRCKAWNVHGAQSGSVFQLLPGKLEHGGGLGLGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 2313 bp, Artificial Sequence
  Synthetic Siglec 11 Fragment (28-561) / (G4S)2 / hIgG1 Fc N297G
ctgaacaaggaccccagctatagcctgcaggtccagagacaggtgccagtgcctgaaggcctgtgtgtgatcgtgtcctgcaacctgagctaccccagagatggctgggatgagagcacagccgcctacggctactggttcaagggcagaacaagccccaaaacaggcgccccagtggccaccaacaaccagagcagagaagtggaaatgagcacccgggacagattccagctgacaggcgatcctggcaagggcagctgttctctggtcatcagagatgcccagagagaggacgaggcctggtacttcttcagagtggaacggggctctagagtgcggcacagctttctgagcaacgcattcttcctgaaagtgacggccctgaccaagaaacccgacgtgtacatccccgagacactggaacctggccagcctgtgacagtgatctgcgtgttcaactgggccttcaagaagtgccccgctcctagctttagctggacaggcgctgctctgagccccagaagaaccagacctagcaccagccacttcagcgtgctgagcttcaccccatctccacaggaccacgacaccgatctgacctgccacgtggacttcagcagaaagggcgtgtcagcccagagaaccgtcagactgagagtggcctacgctcccaaggacctgatcatcagcatcagccacgacaacacaagcgccctggaactgcagggcaacgtgatctacctggaagtgcagaagggccagttcctgagactgctgtgtgccgccgattctcagcctcctgctacactgtcttgggtgctgcaggacagagtgctgagcagcagtcatccttggggccctagaacactgggcctcgaactgagaggtgtcagagccggcgatagcggcagatacacatgcagagccgagaacagactgggctctcagcagcaggctctggatctgagcgtgcagtaccctcctgagaacctgagagtgatggtgtcccaggccaaccggaccgtgctggaaaatctcggcaatggcacaagcctgcctgtgctggaaggccagagtctgagactcgtgtgtgtgacccacagcagccctccagccagactgagttggacaagatggggccagacagtgggccctagccagccttctgatcctggcgttttggagctgcctcctatccagatggaacacgagggcgagttcacctgtcacgcccaacatcctctcggcagccagcatgtgtctctgagcctgtctgtgcactacccacctcagctgctgggcccttcttgttcttgggaagccgagggcctgcactgcagctgtagttctcaagcctctcctgctcctagcctgagatggtggctgggcgaagaactgctcgagggcaatagcagccagggcagctttgaagtgaccccttcttctgccggaccttgggccaatagctctctgtctctgcacggcggactgagcagtggactgagactgagatgcaaggcctggaatgtgcacggcgctcagagcggctctgtgtttcaactgctgcccggcaagctggaacatggcggaggacttggacttggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 98 - protein, 771 aa, Artificial Sequence
  Synthetic Siglec 11 Fragment (28-561) SAX / (G4S)2 / hIgG1 Fc N297G
LNKDPSYSLQVQRQVPVPEGLCVIVSCNLSYPRDGWDESTAAYGYWFKGRTSPKTGAPVATNNQSREVEMSTRDRFQLTGDPGKGSCSLVIRDAQREDEAWYFFAVERGSRVRHSFLSNAFFLKVTALTKKPDVYIPETLEPGQPVTVICVFNWAFKKCPAPSFSWTGAALSPRRTRPSTSHFSVLSFTPSPQDHDTDLTCHVDFSRKGVSAQRTVRLRVAYAPKDLIISISHDNTSALELQGNVIYLEVQKGQFLRLLCAADSQPPATLSWVLQDRVLSSSHPWGPRTLGLELRGVRAGDSGRYTCRAENRLGSQQQALDLSVQYPPENLRVMVSQANRTVLENLGNGTSLPVLEGQSLRLVCVTHSSPPARLSWTRWGQTVGPSQPSDPGVLELPPIQMEHEGEFTCHAQHPLGSQHVSLSLSVHYPPQLLGPSCSWEAEGLHCSCSSQASPAPSLRWWLGEELLEGNSSQGSFEVTPSSAGPWANSSLSLHGGLSSGLRLRCKAWNVHGAQSGSVFQLLPGKLEHGGGLGLGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - DNA, 2313 bp, Artificial Sequence
  Synthetic Siglec 11 Fragment (28-561) SAX / (G4S)2 / hIgG1 Fc N297G
ctgaacaaggaccccagctatagcctgcaggtccagagacaggtgccagtgcctgaaggcctgtgtgtgatcgtgtcctgcaacctgagctaccccagagatggctgggatgagagcacagccgcctacggctactggttcaagggcagaacaagccccaaaacaggcgccccagtggccaccaacaaccagagcagagaagtggaaatgagcacccgggacagattccagctgacaggcgatcctggcaagggcagctgttctctggtcatcagagatgcccagagagaggacgaggcctggtacttcttcgccgtggaacggggctctagagtgcggcacagctttctgagcaacgcattcttcctgaaagtgacggccctgaccaagaaacccgacgtgtacatccccgagacactggaacctggccagcctgtgacagtgatctgcgtgttcaactgggccttcaagaagtgccccgctcctagctttagctggacaggcgctgctctgagccccagaagaaccagacctagcaccagccacttcagcgtgctgagcttcaccccatctccacaggaccacgacaccgatctgacctgccacgtggacttcagcagaaagggcgtgtcagcccagagaaccgtcagactgagagtggcctacgctcccaaggacctgatcatcagcatcagccacgacaacacaagcgccctggaactgcagggcaacgtgatctacctggaagtgcagaagggccagttcctgagactgctgtgtgccgccgattctcagcctcctgctacactgtcttgggtgctgcaggacagagtgctgagcagcagtcatccttggggccctagaacactgggcctcgaactgagaggtgtcagagccggcgatagcggcagatacacatgcagagccgagaacagactgggctctcagcagcaggctctggatctgagcgtgcagtaccctcctgagaacctgagagtgatggtgtcccaggccaaccggaccgtgctggaaaatctcggcaatggcacaagcctgcctgtgctggaaggccagagtctgagactcgtgtgtgtgacccacagcagccctccagccagactgagttggacaagatggggccagacagtgggccctagccagccttctgatcctggcgttttggagctgcctcctatccagatggaacacgagggcgagttcacctgtcacgcccaacatcctctcggcagccagcatgtgtctctgagcctgtctgtgcactacccacctcagctgctgggcccttcttgttcttgggaagccgagggcctgcactgcagctgtagttctcaagcctctcctgctcctagcctgagatggtggctgggcgaagaactgctcgagggcaatagcagccagggcagctttgaagtgaccccttcttctgccggaccttgggccaatagctctctgtctctgcacggcggactgagcagtggactgagactgagatgcaaggcctggaatgtgcacggcgctcagagcggctctgtgtttcaactgctgcccggcaagctggaacatggcggaggacttggacttggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 100 - protein, 595 aa, Homo sapiens
  misc_feature (1)..(595): Siglec 12 FL
MLLLLLLLPPLLCGRVGAKEQKDYLLTMQKSVTVQEGLCVSVLCSFSYPQNGWTASDPVHGYWFRAGDHVSRNIPVATNNPARAVQEETRDRFHLLGDPQNKDCTLSIRDTRESDAGTYVFCVERGNMKWNYKYDQLSVNVTASQDLLSRYRLEVPESVTVQEGLCVSVPCSVLYPHYNWTASSPVYGSWFKEGADIPWDIPVATNTPSGKVQEDTHGRFLLLGDPQTNNCSLSIRDARKGDSGKYYFQVERGSRKWNYIYDKLSVHVTALTHMPTFSIPGTLESGHPRNLTCSVPWACEQGTPPTITWMGASVSSLDPTITRSSMLSLIPQPQDHGTSLTCQVTLPGAGVTMTRAVRLNISYPPQNLTMTVFQGDGTASTTLRNGSALSVLEGQSLHLVCAVDSNPPARLSWTWGSLTLSPSQSSNLGVLELPRVHVKDEGEFTCRAQNPLGSQHISLSLSLQNEYTGKMRPISGVTLGAFGGAGATALVFLYFCIIFVVVRSCRKKSARPAVGVGDTGMEDANAVRGSASQGPLIESPADDSPPHHAPPALATPSPEEGEIQYASLSFHKARPQYPQEQEAIGYEYSEINIPK

SEQ ID NO: 101 - DNA, 1788 bp, Homo sapiens
  misc_feature (1)..(1788): Siglec 12 FL
atgctactgctgctgctactgctgccacccctgctctgtgggagagtgggggctaaggaacagaaggattacctgctgacaatgcagaagtccgtgacggtgcaggagggcctgtgtgtctctgtgctttgctccttctcctacccccaaaatggctggactgcctccgatccagttcatggctactggttccgggcaggggaccatgtaagccggaacattccagtggccacaaacaacccagctcgagcagtgcaggaggagactcgggaccgattccacctccttggggacccacagaacaaggattgtaccctgagcatcagagacaccagagagagtgatgcagggacatacgtcttttgtgtagagagaggaaatatgaaatggaattataaatatgaccagctctctgtgaatgtgacagcgtcccaggacctactgtcaagatacaggctggaggtgccagagtcggtgactgtgcaggagggtctgtgtgtctctgtgccctgcagtgtcctttacccccattacaactggactgcctctagccctgtttatggatcctggttcaaggaaggggccgatataccatgggatattccagtggccacaaacaccccaagtggaaaagtgcaagaggatacccacggtcgattcctcctccttggggacccacagaccaacaactgctccctgagcatcagagatgccaggaagggggattcagggaagtactacttccaggtggagagaggaagcaggaaatggaactacatatatgacaagctctctgtgcatgtgacagccctgactcacatgcccaccttctccatcccggggaccctggagtctggccaccccaggaacctgacctgctctgtgccctgggcctgtgaacaggggacgccccccacgatcacctggatgggggcctccgtgtcctccctggaccccactatcactcgctcctcgatgctcagcctcatcccacagccccaggaccatggcaccagcctcacctgtcaggtgaccttgcctggggccggcgtgaccatgaccagggctgtccgactcaacatatcctatcctcctcagaacttgaccatgactgtcttccaaggagatggcacagcatccacaaccttgaggaatggctcggccctttcagtcctggagggccagtccctgcaccttgtctgtgctgtcgacagcaatccccctgccaggctgagctggacctgggggagcctgaccctgagcccctcacagtcctcgaaccttggggtgctggagctgcctcgagtgcatgtgaaggatgaaggggaattcacctgccgagctcagaaccctctaggctcccagcacatttccctgagcctctccctgcaaaacgagtacacaggcaaaatgaggcctatatcaggagtgacgctaggggcattcgggggagctggagccacagccctggtcttcctgtacttctgcatcatcttcgttgtagtgaggtcctgcaggaagaaatcggcaaggccagcagtgggcgtgggggatacaggcatggaggacgcaaacgctgtcaggggctcagcctctcagggacccctgattgaatccccggcagatgacagccccccacaccatgctccgccagccctggccaccccctccccagaggaaggagagatccagtatgcatccctcagcttccacaaagcgaggcctcagtacccacaggaacaggaggccatcggctatgagtactccgagatcaacatccccaagtga

SEQ ID NO: 102 - protein, 396 aa, Homo sapiens
  misc_feature (1)..(396): Siglec 14 FL
MLPLLLLPLLWGGSLQEKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQVKRQGAQVTTERTVQLNVSYAPQNLAISIFFRNGTGTALRILSNGMSVPIQEGQSLFLACTVDSNPPASLSWFREGKALNPSQTSMSGTLELPNIGAREGGEFTCRVQHPLGSQHLSFILSVQRSSSSCICVTEKQQGSWPLVLTLIRGALMGAGFLLTYGLTWIYYTRCGGPQQSRAERPG

SEQ ID NO: 103 - DNA, 1191 bp, Homo sapiens
  misc_feature (1)..(1191): Siglec 14 FL
atgctgcccctgctgctgctgcccctgctgtggggggggtccctgcaggagaagccagtgtacgagctgcaagtgcagaagtcggtgacggtgcaggagggcctgtgcgtccttgtgccctgctccttctcttacccctggagatcctggtattcctctcccccactctacgtctactggttccgggacggggagatcccatactacgctgaggttgtggccacaaacaacccagacagaagagtgaagccagagacccagggccgattccgcctccttggggatgtccagaagaagaactgctccctgagcatcggagatgccagaatggaggacacgggaagctatttcttccgcgtggagagaggaagggatgtaaaatatagctaccaacagaataagctgaacttggaggtgacagccctgatagagaaacccgacatccactttctggagcctctggagtccggccgccccacaaggctgagctgcagccttccaggatcctgtgaagcgggaccacctctcacattctcctggacggggaatgccctcagccccctggaccccgagaccacccgctcctcggagctcaccctcacccccaggcccgaggaccatggcaccaacctcacctgtcaggtgaaacgccaaggagctcaggtgaccacggagagaactgtccagctcaatgtctcctatgctccacagaacctcgccatcagcatcttcttcagaaatggcacaggcacagccctgcggatcctgagcaatggcatgtcggtgcccatccaggagggccagtccctgttcctcgcctgcacagttgacagcaacccccctgcctcactgagctggttccgggagggaaaagccctcaatccttcccagacctcaatgtctgggaccctggagctgcctaacataggagctagagagggaggggaattcacctgccgggttcagcatccgctgggctcccagcacctgtccttcatcctttctgtgcagagaagctcctcttcctgcatatgtgtaactgagaaacagcagggctcctggcccctcgtcctcaccctgatcaggggggctctcatgggggctggcttcctcctcacctatggcctcacctggatctactataccaggtgtggaggcccccagcagagcagggctgagaggcctggctga

SEQ ID NO: 104 - protein, 328 aa, Homo sapiens
  misc_feature (1)..(328): Siglec 15 FL
MEKSIWLLACLAWVLPTGSFVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGASTVALLLGALGFKALLLLGVLAARAARRRPEHLDTPDTPPRSQAQESNYENLSQMNPRSPPATMCSP

SEQ ID NO: 105 - DNA, 987 bp, Homo sapiens
  misc_feature (1)..(987): Siglec 15 FL
atggaaaagtccatctggctgctggcctgcttggcgtgggttctcccgacaggctcatttgtgagaactaaaatagatactacggagaacttgctcaacacagaggtgcacagctcgccagcgcagcgctggtccatgcaggtgccacccgaggtgagcgcggaggcaggcgacgcggcagtgctgccctgcaccttcacgcacccgcaccgccactacgacgggccgctgacggccatctggcgcgcgggcgagccctatgcgggcccgcaggtgttccgctgcgctgcggcgcggggcagcgagctctgccagacggcgctgagcctgcacggccgcttccggctgctgggcaacccgcgccgcaacgacctctcgctgcgcgtcgagcgcctcgccctggctgacgaccgccgctacttctgccgcgtcgagttcgccggcgacgtccatgaccgctacgagagccgccacggcgtccggctgcacgtgacagccgcgccgcggatcgtcaacatctcggtgctgcccagtccggctcacgccttccgcgcgctctgcactgccgaaggggagccgccgcccgccctcgcctggtccggcccggccctgggcaacagcttggcagccgtgcggagcccgcgtgagggtcacggccacctagtgaccgccgaactgcccgcactgacccatgacggccgctacacgtgtacggccgccaacagcctgggccgctccgaggccagcgtctacctgttccgcttccatggcgccagcggggcctcgacggtcgccctcctgctcggcgctctcggcttcaaggcgctgctgctgctcggggtcctggccgcccgcgctgcccgccgccgcccagagcatctggacaccccggacaccccaccacggtcccaggcccaggagtccaattatgaaaatttgagccagatgaacccccggagcccaccagccaccatgtgctcaccgtga

SEQ ID NO: 106 - protein, 244 aa, Homo sapiens
  misc_feature (1)..(244): Siglec 15 Fragment (20-263)
FVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGAST

SEQ ID NO: 107 - protein, 244 aa, Artificial Sequence
  Synthetic Siglec 15 Fragment (20-263) SAX
FVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVALHVTAAPRIVNISVLPSPAHAFRALCTAEGEPPPALAWSGPALGNSLAAVRSPREGHGHLVTAELPALTHDGRYTCTAANSLGRSEASVYLFRFHGASGAST

SEQ ID NO: 108 - protein, 481 aa, Artificial Sequence
  Synthetic Siglec 15 Fragment (20-263) / (G4S)2 / hIgG1 Fc N297G
FVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGASTGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - DNA, 1443 bp, Artificial Sequence
  Synthetic Siglec 15 Fragment (20-263) / (G4S)2 / hIgG1 Fc N297G
tttgtgcggaccaagatcgacaccaccgagaacctgctgaacaccgaggtgcactctagccctgctcagcggtggtctatgcaggtcccacctgaagtgtctgccgaagctggggatgctgctgtgctgccttgcacctttacacaccctcaccggcactacgatggccctctgacagccatttggagagccggcgaaccttatgccggaccacaggtgttcagatgcgctgccgctagaggctctgagctgtgtcagacagccctgtctctgcacggcagattcagactgctgggcaaccccagaagaaacgacctgagcctgagagtggaaagactggccctggccgacgacagacggtacttctgcagagtggaatttgccggcgacgtgcacgacagatacgagtctagacacggcgtgcggctgcacgttacagccgctcctagaatcgtgaacatcagcgtgctgccctctccagctcacgcctttagagccctgtgtacagccgaaggcgaacctcctcctgctcttgcttggagtggacccgctctgggaaattctctggccgccgtcagaagccctagagaaggacacggacacctggtcacagccgaactgcctgctctgacacacgacggcagatacacatgcacagccgccaatagcctgggcagatctgaggccagcgtgtacctgttcagattccatggcgccagcggcgcctctacaggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 110 - protein, 481 aa, Artificial Sequence
  Synthetic Siglec 15 Fragment (20-263) SAX / (G4S)2 / hIgG1 Fc N297G
FVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCAVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGASTGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - DNA, 1443 bp, Artificial Sequence
  Synthetic Siglec 15 Fragment (20-263) SAX / (G4S)2 / hIgG1 Fc N297G
tttgtgcggaccaagatcgacaccaccgagaacctgctgaacaccgaggtgcactctagccctgctcagcggtggtctatgcaggtcccacctgaagtgtctgccgaagctggggatgctgctgtgctgccttgcacctttacacaccctcaccggcactacgatggccctctgacagccatttggagagccggcgaaccttatgccggaccacaggtgttcagatgcgctgccgctagaggctctgagctgtgtcagacagccctgtctctgcacggcagattcagactgctgggcaaccccagaagaaacgacctgagcctgagagtggaaagactggccctggccgacgacagacggtacttctgcgccgtggaatttgccggcgacgtgcacgacagatacgagtctagacacggcgtgcggctgcacgttacagccgctcctagaatcgtgaacatcagcgtgctgccctctccagctcacgcctttagagccctgtgtacagccgaaggcgaacctcctcctgctcttgcttggagtggacccgctctgggaaattctctggccgccgtcagaagccctagagaaggacacggacacctggtcacagccgaactgcctgctctgacacacgacggcagatacacatgcacagccgccaatagcctgggcagatctgaggccagcgtgtacctgttcagattccatggcgccagcggcgcctctacaggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 112 - protein, 481 aa, Artificial Sequence
  Synthetic Mouse Siglec 15 Fragment / (G4S)2 / hIgG1 Fc N297G
FVRTKIDTTENLLNTEVHSSPAQRWSMQVPAEVSAAAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIINISVLPGPAHAFRALCTAEGEPPPALAWSGPALGNGSAAVPSSGQGHGHLVTAELPALNHDGRYTCTAANSLGRSEASVYLFRFHGASGASTGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - DNA, 1443 bp, Artificial Sequence
  Synthetic Mouse Siglec 15 Fragment / (G4S)2 / hIgG1 Fc N297G
ttcgtgcggaccaagatcgacaccaccgagaacctgctgaacaccgaggtgcactctagccctgctcagcggtggtctatgcaggtcccagctgaagtgtctgctgctgctggggatgctgctgtgctgccttgcacctttacacaccctcaccggcactacgatggccctctgacagccatttggagagccggcgaaccttatgccggaccacaggtgttcagatgcgctgccgctagaggctctgagctgtgtcagacagccctgtctctgcacggcagattcagactgctgggcaaccccagaagaaacgacctgagcctgagagtggaaagactggccctggccgacgacagacggtacttctgcagagtggaatttgccggcgacgtgcacgacagatacgagtctagacacggcgtgcggctgcacgttacagccgctcctagaatcatcaacatcagcgtgctgcccggacctgctcacgcttttagagccctgtgtacagccgagggcgaacctcctcctgctcttgcttggagtggacccgctctcggaaatggatctgccgccgttcctagctctggccaaggacacggacatctggtcacagccgaactgcccgctctgaaccacgacggcagatacacatgtaccgccgccaatagcctgggcagatctgaggctagcgtgtacctgttcagattccacggcgccagcggagcctctacaggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 114 - protein, 481 aa, Homo sapiens
  misc_feature (1)..(481): Siglec 16 FL
MLLLPLLLPVLGAGSLNKDPSYSLQVQRQVPVPEGLCVIVSCNLSYPRDGWDESTAAYGYWFKGWTSPKTGAPVATNNQSREVEMSTRDRFQLTGDPGKGSCSLVIRDAQREDEAWYFFRVERGSRVRHSFVNNLFFLKVTALTQKPDVYIPETLEPGQPVTVICVFNWAFKKCPAPSFSWTGAALSPRRTRPSTSHFSVLSFTPSPQDHDTDLTCHVDFSRKGVSAQRTVRLRVASLELQGNVIYLEVQKGQFLRLLCAADSQPPATLSWVLQDRVLSSSHPWGPRTLGLELRGVRAGDSGRYTCRAENRLGSQQRALDLSVQYPPENLRVMVSQANRTVLENLRNGTSLRVLEGQSLRLVCVTHSSPPARLSWTRWGQTVGPSQPSDPGVLELPRVQMEHEGEFTCHARHPLGSQRVSLSFSVHCKSGPMTGVVLVAVGEVAMKILLLCLCLILLRVRSCRRKAARAALGMEAADAVTD

SEQ ID NO: 115 - DNA, 3811 bp, Homo sapiens
  misc_feature (1)..(3811): Siglec 16 FL
agatggtcccgggacaggcccagccccagagcccagagatgctgctgctgcccctgctgctgcccgtgctgggggcggggtccctgaacaaggatcccagttacagtcttcaagtgcagaggcaggtgccggtgccggagggcctgtgtgtcatcgtgtcttgcaacctctcctacccccgggatggctgggacgagtctactgctgcttatggctactggttcaaaggatggaccagcccaaagacgggtgctcctgtggccactaacaaccagagtcgagaggtggaaatgagcacccgggaccgattccagctcactggggatcccggcaaagggagctgctccttggtgatcagagacgcgcagagggaggatgaggcatggtacttctttcgggtggagagaggaagccgtgtgagacatagtttcgtgaacaatttgttctttctaaaagtaacagccctgactcagaagcctgatgtctacatccccgagaccctggagcccgggcagccggtgacggtcatctgtgtgtttaactgggctttcaagaaatgtccagccccttctttctcctggacgggggctgccctctcccctagaagaaccagaccaagcacctcccacttctcagtgctcagcttcacgcccagcccccaggaccacgacaccgacctcacctgccatgtggacttctccagaaagggtgtgagcgcacagaggaccgtccgactccgtgtggcctccctggaactccagggaaacgtcatatatctggaagttcagaaaggccagttcctgcggctcctctgtgctgctgacagccagccccctgccacgctgagctgggtcctgcaggacagagtcctctcctcgtcccacccctggggccccagaaccctggggctggagctgcgtggggtaagggccggggattcagggcgctacacctgccgagcggagaacaggcttggctcccagcagcgagccctggacctctctgtgcagtatcctccagagaacctgagagtgatggtttcccaagcaaacaggacagtcctggaaaacctgaggaacggcacatccctccgggtcctggagggccaaagcctgcgtctggtctgtgtcacacacagcagccccccagccaggctgagctggacccggtggggacagaccgtgggcccctcccagccctcagaccctggggtcctggagctgcctcgggttcaaatggagcacgaaggagagttcacctgccacgctcggcacccgctgggctcccagcgcgtctctctcagcttctccgtgcactgcaaatcagggcccatgacgggggtggttctggtggctgttggggaggtggctatgaagatcctgcttctctgcctctgcctcatcctcctcagagtgaggtcttgcaggaggaaggcagcaagggcagcattgggcatggaggctgcagacgctgtcacggactaatctccagactccagactgcttccagatgcctcctcatccagttcctccacagtctgagcggccgtgtttcctctgcaggctttgcatggtctgtcccctgctggactctcctgatccctcctttccctgtcacccaacatctccccaaaccctccgggccaaggactctgcggctcttgacactttgcacgtgtagtttcttctcctgaaacacccttccctcctgccaaaccacgtctggaccctccttcaggttcactttttttttttttttttgagacacagtctcactttgtcacccaggctggagtgcagtggcgcgatctcggctcactgcaacctctgtctcccaggttcaagcgattctcatgtctcagcctcctgagtagctgggattataggcgcccgccaccacgcctggctcatttttgtatttttggtagagacagggtttcaccatgtcggccaggctggtcttgaactcccgacttcaggtgatccgccagcctcggcctcccaaagtggtggggttacatgtgtgagccaccgtgcccagccagctgtgtcttctttttgcaggatttacttacaaaattccttgcagctgcagtctgcgttccacccacatcctaatatggtgtttgcggtggtcacatccaacttccttcctcctccatccatggaacgtgcattagggaggaaccacacgcaccacgtgcaagatgagccagttccctgaacggccggccacggcgttaaccgcgctgctctgggctctgcacgtctcatctgctgaggcgcattactgcatctttcatgcagaacttgctttccatgcctcacatctccacatgtgtggggatatgtcctgagaaatgcacggttaggtgattctgtcgtgtgagcatccttgaatgtgcttacacagtcataaatggtacagcctaccacacgccgaggctatacggtatagcccattgctcctagactatacacctgcacggcatgttcctgtagtgaaagctgtaggcaattataacgcaatggataggttaaagacagagttttagtaaaaatatggcataataggccaggcgtggtggctcatgcctgtaatcccagcactttgggaggccaaggcaggcagatcacaaggtcaggagtttgagaccagcctggccaatatggtgaaacccagtctctactaaaaatacaaaaattagccgggtgtggtggcgggcgcctgtagtcccagctactcgggaggctgaggcagaagaatcatttgaaccaggaggtggaggttgcagtgagccgagatcgcatcactgcactccagcctgggcgacagagcaagactgtctcaaaaactaaaacaaaaaaacaccaccaccaccaccaatgtctgattaaatctaagccacggttccattttggggcaaggggcagggtgtgggtgtggtggacagagaacgggctccagccaccatcagctgcacgtcctggtagcacctgagccattcccagtctcctgtgctgggagatcatcatctaatcctgatgatgacgccctcctctcagagctcgggggatgagctgccaaaacccatggaggttcaaaacccgggctctggagacagaagacactgggctcagagacatggggccactgtccaggctttccctgggcctggacctgggctccttccactgtctgagcagggtcagcctgggctggctgtgctggttcaagagatgagggaggaggacacagcggttgccaggaccctgaggcactggactgggcactgccccaaggggcagaggatggaacccggtgttgatattgtgatgctgacccgtctgctcacctgatttgggtgctgaagggcagctgacccccctaatcttttatagacttgtgaaacgaaaataaataaaagtcaagctgggaacagcttagggcaaacctgcctcccattctattcaaagccacccctctgctcactgagatagatgcatgtctgatcacctcagttgggaaaggctaatcagaaactccaaagaatgcatctgttcatctctcacccatctgtgacctggaagccccctccccacttcgagtcttcctgcctttgcttcaagttttcccgcctttccaaatggaaccaaagtacttcttacatatagtgactcatgtctcaagtctccctaaaacgtataaaactaagctgtgccccaaccacgctgaccatgtcatcaggacctcctgaggctgtgtcacggcctgtgtcctcaaccttggcaaaataaactttctaaattaa

SEQ ID NO: 116 - protein, 418 aa, Homo sapiens
  misc_feature (1)..(418): Siglec 16 Fragment (15-434)
NKDPSYSLQVQRQVPVPEGLCVIVSCNLSYPRDGWDESTAAYGYWFKGRTSPKTGAPVATNNQSREVAMSTRDRFQLTGDPGKGSCSLVIRDAQREDEAWYFFRVERGSRVRHSFLSNAFFLKVTALTQKPDVYIPETLEPGQPVTVICVFNWAFKKCPAPSFSWTGAALSPRRTRPSTSHFSVLSFTPSPQDHDTDLTCHVDFSRKGVSAQRTVRLRVASLELQGNVIYLEVQKGQFLRLLCAADSQPPATLSWVLQDRVLSSSHPWGPRTLGLELPGVKAGDSGRYTCRAENRLGSQQRALDLSVQYPPENLRVMVSQANRTVLENLRNGTSLRVLEGQSLRLVCVTHSSPPARLSWTWGEQTVGPSQPSDPGVLQLPRVQMEHEGEFTCHARHPLGSQRVSLSFSVHCKSGPMTG

SEQ ID NO: 117 - protein, 655 aa, Artificial Sequence
  Synthetic Siglec 16 Fragment (15-434) (G4S)2 / hIgG1 Fc N297G
NKDPSYSLQVQRQVPVPEGLCVIVSCNLSYPRDGWDESTAAYGYWFKGRTSPKTGAPVATNNQSREVAMSTRDRFQLTGDPGKGSCSLVIRDAQREDEAWYFFRVERGSRVRHSFLSNAFFLKVTALTQKPDVYIPETLEPGQPVTVICVFNWAFKKCPAPSFSWTGAALSPRRTRPSTSHFSVLSFTPSPQDHDTDLTCHVDFSRKGVSAQRTVRLRVASLELQGNVIYLEVQKGQFLRLLCAADSQPPATLSWVLQDRVLSSSHPWGPRTLGLELPGVKAGDSGRYTCRAENRLGSQQRALDLSVQYPPENLRVMVSQANRTVLENLRNGTSLRVLEGQSLRLVCVTHSSPPARLSWTWGEQTVGPSQPSDPGVLQLPRVQMEHEGEFTCHARHPLGSQRVSLSFSVHCKSGPMTGGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - DNA, 1965 bp, Artificial Sequence
  Synthetic Siglec 16 Fragment (15-434) (G4S)2 / hIgG1 Fc N297G
aacaaggaccccagctacagcctgcaggtccagagacaggtgccagtgcctgaaggcctgtgtgtgatcgtgtcctgcaacctgagctaccccagagatggctgggatgagagcacagccgcctacggctactggttcaagggcagaacaagccccaaaacaggcgccccagtggccaccaacaaccagtctagagaagtggccatgagcacccgggacagattccagttgacaggcgatcctggcaagggcagctgtagcctggttatcagagatgcccagagagaggacgaggcctggtacttcttcagagtggaacggggctctagagtgcggcacagctttctgagcaacgcattcttcctgaaagtgacggccctgacacagaaacccgacgtgtacatccccgagacactggaacctggccagcctgtgacagtgatctgcgtgttcaactgggccttcaagaagtgccccgctcctagctttagctggacaggcgctgctctgagccccagaagaaccagacctagcaccagccacttcagcgtgctgagcttcaccccatctccacaggaccacgacaccgatctgacctgccacgtggacttcagcagaaagggcgtgtcagcccagagaaccgtcagactgagagtggccagtctcgagctgcagggcaacgtgatctacctggaagtgcagaagggccagttcctgagactgctgtgtgccgccgattctcagcctcctgctacactgtcttgggtgctgcaggacagagtgctgagcagcagtcatccttggggccctagaacactgggcctcgaattgcctggtgtcaaggccggcgatagcggcagatacacatgcagagccgagaacagactgggcagccagcagagagcactggatctgagcgtgcagtaccctcctgagaacctgagagtgatggtgtcccaggccaaccggaccgtgctggaaaacctgagaaatggcaccagcctgagggtgctcgagggacagtctctgagactcgtgtgtgtgacccacagcagccctccagccagactctcttggacatggggagagcagacagtgggccctagccagccttctgatcctggtgttctgcagctgccccgggtgcagatggaacacgaaggcgagtttacctgtcacgccagacatcctctgggctcccagagagtgtctctgagctttagcgtgcactgcaagagcggccctatgacaggaggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 119 - protein, 45 aa, Artificial Sequence
  Synthetic HSA-binding protein
LKEAKEKAIEELKKAGITSDYYFDLINKAKTVEGVNALKDEILKA

SEQ ID NO: 120 - protein, 560 aa, Artificial Sequence
  Synthetic Siglec 9 Fragment (18-348) C158S, C295Y / AS / hIgG1 Fc
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGSPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLYPSQPSNPGVLELPWVHLRDAAEFTCRAQNPLGSQQVYLNVSLQSKATSGVTQGASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - DNA, 1680 bp, Artificial Sequence
  Synthetic Siglec 9 Fragment (18-348) C158S, C295Y / AS / hIgG1 Fc
cagaccagcaagctgctgaccatgcagagcagcgtgaccgtgcaggagggcctgtgcgtgcatgtgccctgcagcttcagctaccccagccacggctggatctaccccggtcccgtagtgcacggctactggttcagggagggcgccaacaccgaccaggacgctcccgtggcaaccaacaaccccgccagggccgtgtgggaggagaccagggacaggttccacctgctgggcgacccccacaccaagaactgcaccctgagcatcagggacgccaggaggagcgacgccggcaggtacttcttcaggatggagaaggggtctatcaagtggaactacaagcaccaccggctgagcgtgaatgtgaccgccctgacccaccggcccaatatcctcatccccggcaccctggagagcggcagcccccagaatcttacctgcagcgtaccctgggcctgcgagcagggcacccctccaatgatcagctggatcggcaccagcgtgagccccctggaccctagtaccaccaggagcagcgtgctgaccctgatcccccagccccaggaccacggaaccagcctgacctgccaggtgaccttccccggagccagcgtaaccaccaacaagaccgtgcacctgaacgtgagctacccaccccaaaacctgaccatgaccgtgttccagggcgacggcacggtgagcaccgtactgggcaacggcagctctctgagcctgcccgagggccagagcttgcggctggtctgcgccgtggatgctgtggacagcaaccctcccgccaggctgagcctgagctggaggggcctgaccctgtaccccagccagcccagcaatcccggcgtgctggagctgccctgggttcacctgagggacgctgccgagttcacatgtagggcccagaaccccctgggctctcagcaggtgtacctgaacgtgtctcttcagagtaaggccaccagcggcgtgacccaaggagctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 122 - protein, 563 aa, Artificial Sequence
  Synthetic Siglec 9 Fragment (18-348) C158S, C295Y, SAX / EPKSS / hIgG1 Fc N297G
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFKMEKGSIKWNYKHHRLSVNVTALTHRPNILIPGTLESGSPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLYPSQPSNPGVLELPWVHLRDAAEFTCRAQNPLGSQQVYLNVSLQSKATSGVTQGEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - DNA, 1689 bp, Artificial Sequence
  Synthetic Siglec 9 Fragment (18-348) C158S, C295Y, SAX /  EPKSS / hIgG1 Fc N297G
cagaccagcaagctgctgaccatgcagagcagcgtgaccgtgcaggagggcctgtgcgtgcatgtgccctgcagcttcagctaccccagccacggctggatctaccccggtcccgtagtgcacggctactggttcagggagggcgccaacaccgaccaggacgctcccgtggcaaccaacaaccccgccagggccgtgtgggaggagaccagggacaggttccacctgctgggcgacccccacaccaagaactgcaccctgagcatcagggacgccaggaggagcgacgccggcaggtacttcttcaagatggagaaggggtctatcaagtggaactacaagcaccaccggctgagcgtgaatgtgaccgccctgacccaccggcccaatatcctcatccccggcaccctggagagcggcagcccccagaatcttacctgcagcgtaccctgggcctgcgagcagggcacccctccaatgatcagctggatcggcaccagcgtgagccccctggaccctagtaccaccaggagcagcgtgctgaccctgatcccccagccccaggaccacggaaccagcctgacctgccaggtgaccttccccggagccagcgtaaccaccaacaagaccgtgcacctgaacgtgagctacccaccccaaaacctgaccatgaccgtgttccagggcgacggcacggtgagcaccgtactgggcaacggcagctctctgagcctgcccgagggccagagcttgcggctggtctgcgccgtggatgctgtggacagcaaccctcccgccaggctgagcctgagctggaggggcctgaccctgtaccccagccagcccagcaatcccggcgtgctggagctgccctgggttcacctgagggacgctgccgagttcacatgtagggcccagaaccccctgggctctcagcaggtgtacctgaacgtgtctcttcagagtaaggccaccagcggcgtgacccaaggagagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 124 - protein, 481 aa, Artificial Sequence
  Synthetic Siglec 15 Fragment (20-263) / (G4S)2 / hIgG1 Fc
FVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGASTGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - DNA, 1443 bp, Artificial Sequence
  Synthetic Siglec 15 Fragment (20-263) / (G4S)2 / hIgG1 Fc
tttgtgcggaccaagatcgacaccaccgagaacctgctgaacaccgaggtgcactctagccctgctcagcggtggtctatgcaggtcccacctgaagtgtctgccgaagctggggatgctgctgtgctgccttgcacctttacacaccctcaccggcactacgatggccctctgacagccatttggagagccggcgaaccttatgccggaccacaggtgttcagatgcgctgccgctagaggctctgagctgtgtcagacagccctgtctctgcacggcagattcagactgctgggcaaccccagaagaaacgacctgagcctgagagtggaaagactggccctggccgacgacagacggtacttctgcagagtggaatttgccggcgacgtgcacgacagatacgagtctagacacggcgtgcggctgcacgttacagccgctcctagaatcgtgaacatcagcgtgctgccctctccagctcacgcctttagagccctgtgtacagccgaaggcgaacctcctcctgctcttgcttggagtggacccgctctgggaaattctctggccgccgtcagaagccctagagaaggacacggacacctggtcacagccgaactgcctgctctgacacacgacggcagatacacatgcacagccgccaatagcctgggcagatctgaggccagcgtgtacctgttcagattccatggcgccagcggcgcctctacaggcggaggtggaagtggcggaggcggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 126 - protein, 469 aa, Artificial Sequence
  Synthetic Siglec 3 Fragment (18-259) SAX / hIgG1 Fc N297G
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFKMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 566 aa, Artificial Sequence
  Synthetic Siglec 7 Fragment (19-357) / hIgG1 Fc N297G
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 566 aa, Artificial Sequence
  Synthetic Siglec 7 Fragment (19-357) SAX / hIgG1 Fc N297G
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFKMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 558 aa, Artificial Sequence
  Synthetic Siglec 9 Fragment (18-348) C158S, C295Y / hIgG1 Fc N297G
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGSPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLYPSQPSNPGVLELPWVHLRDAAEFTCRAQNPLGSQQVYLNVSLQSKATSGVTQGDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 558 aa, Artificial Sequence
  Synthetic Siglec 9 Fragment (18-348) C158S, C295Y, SAX / hIgG1 Fc N297G
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFKMEKGSIKWNYKHHRLSVNVTALTHRPNILIPGTLESGSPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLYPSQPSNPGVLELPWVHLRDAAEFTCRAQNPLGSQQVYLNVSLQSKATSGVTQGDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 355 aa, Artificial Sequence
  Synthetic Siglec 9 Fragment (18-145) / hIgG1 Fc (w/ monovalent mutations)
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLSPGK

SEQ ID NO: 132 - protein, 430 aa, Artificial Sequence
  Synthetic Siglec 9 Fragment (146-348) / hIgG1 Fc N297G
PNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 558 aa, Artificial Sequence
  Synthetic Siglec 9 Fragment (18-348) K131Q / hIgG1 Fc N297G
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYQHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 558 aa, Artificial Sequence
  Synthetic Siglec 9 Fragment (18-348) K100E, A315E / hIgG1 Fc N297G
QTSKLLTMQSSVTVQEGLCVHVPCSFSYPSHGWIYPGPVVHGYWFREGANTDQDAPVATNNPARAVWEETRDRFHLLGDPHTENCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDEAEFTCRAQNPLGSQQVYLNVSLQSKATSGVTQGDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 558 aa, Artificial Sequence
  Synthetic Siglec 9 Fragment (18-348) C158S, C295Y / AS / hIgG1 Fc
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGSPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLYPSQPSNPGVLELPWVHLRDAAEFTCRAQNPLGSQQVYLNVSLQSKATSGVTQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 22 aa, Homo sapiens
  misc_feature (1)..(22): N-terminal signal sequence
MDMRVPAQLLGLLLLWLPGARC

SEQ ID NO: 137 - protein, 4 aa, Homo sapiens
  misc_feature (1)..(4): C terminal lysosomal signal motif
YGTL

SEQ ID NO: 138 - protein, 481 aa, Homo sapiens
  misc_feature (1)..(481): Siglec 16 FL (uniprot A6NMB1)
MLLLPLLLPVLGAGSLNKDPSYSLQVQRQVPVPEGLCVIVSCNLSYPRDGWDESTAAYGYWFKGRTSPKTGAPVATNNQSREVAMSTRDRFQLTGDPGKGSCSLVIRDAQREDEAWYFFRVERGSRVRHSFLSNAFFLKVTALTQKPDVYIPETLEPGQPVTVICVFNWAFKKCPAPSFSWTGAALSPRRTRPSTSHFSVLSFTPSPQDHDTDLTCHVDFSRKGVSAQRTVRLRVASLELQGNVIYLEVQKGQFLRLLCAADSQPPATLSWVLQDRVLSSSHPWGPRTLGLELPGVKAGDSGRYTCRAENRLGSQQRALDLSVQYPPENLRVMVSQANRTVLENLRNGTSLRVLEGQSLRLVCVTHSSPPARLSWTWGEQTVGPSQPSDPGVLQLPRVQMEHEGEFTCHARHPLGSQRVSLSFSVHCKSGPMTGVVLVAVGEVAMKILLLCLCLILLRVRSCRRKAARAALGMEAADAVTD

SEQ ID NO: 139 - protein, 471 aa, Artificial Sequence
  Synthetic Siglec 3 Fragment (18-259) / AS / hIgG1 Fc N297G
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - DNA, 1413 bp, Artificial Sequence
  Synthetic Siglec 3 Fragment (18-259) / AS / hIgG1 Fc
gatcccaatttctggctgcaagtgcaagagtccgtgaccgtgcaagagggcctgtgtgtgctggtgccctgcaccttctttcaccccattccttactacgacaagaacagccctgtgcacggctactggtttagagagggcgccatcatcagcagagatagccctgtggccaccaacaagctggaccaagaggtgcaagaagagacacagggcagattcagactgctgggcgaccccagcagaaacaactgcagcctgtctatcgtggacgccaggcggagagacaacggcagctacttcttcaggatggaacggggcagcaccaagtacagctacaagagccctcagctgtccgtgcacgtgaccgacctgacacacagacccaagattctgatccccggcacactggaacctggccacagcaagaatctgacctgctccgtgtcctgggcctgcgaacagggaacccctcctatctttagctggctgagcgccgctcctacatctctgggccctagaacaacacacagcagcgtgctgatcatcacccctagacctcaggaccacggcaccaacctgacctgccaagtgaaatttgctggcgctggcgtgaccaccgagagaaccatccagctgaacgtgacctacgtgccacagaaccctaccaccggcatctttccaggcgacggctctggcaagcaagaaacaagagctggcgtggtgcacgctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 141 - protein, 654 aa, Artificial Sequence
  Synthetic Siglec 5 Fragment (17-441) / AS / hIgG1 Fc N297G
EKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQMKRQGAQVTTERTVQLNVSYAPQTITIFRNGIALEILQNTSYLPVLEGQALRLLCDAPSNPPAHLSWFQGSPALNATPISNTGILELRRVRSAEEGGFTCRAQHPLGFLQIFLNLSVYSLPQLLGPSCSWEAEGLHCRCSFRARPAPSLCWRLEEKPLEGNSSQGSFKVNSSSAGPWANSSLILHGGLSSDLKVSCKAWNIYGSQSGSVLLLQGRSNLGTGVVPAALASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - DNA, 1962 bp, Artificial Sequence
  Synthetic Siglec 5 Fragment (17-441) / AS / hIgG1 Fc N297G
gagaagcccgtgtatgagctgcaggtccagaaaagcgtgaccgtgcaagagggcctgtgtgtgctggtgccttgcagctttagctacccttggcggagctggtacagcagccctcctctgtacgtgtactggttcagagatggcgagatcccctactacgccgaggtggtggccacaaacaaccccgacagaagagtgaagcccgagacacagggcagattcagactgctgggcgacgtgcagaagaagaactgcagcctgagcatcggcgacgcccggatggaagataccggcagctacttcttcagagtggaacgcggcagggacgtgaagtacagctaccagcagaacaagctgaacctggaagtgacagccctgatcgagaagcctgacatccactttctggaacccctggaaagcggcagacccaccagactgtcttgtagcctgcctggcagctgtgaagctggccctccactgacctttagctggaccggcaatgccctgtctcctctggaccctgagacaaccagaagcagcgagctgaccctgacacctagacctgaggaccacggcaccaatctgacctgccagatgaagaggcagggcgcccaagtgaccaccgagagaacagtgcagctgaacgtgtcctacgctccccagaccatcaccatcttccggaacggaatcgccctggaaatcctgcagaacaccagctacctgcctgtgctggaaggccaggctctgagactgctgtgtgacgcccctagcaatcctcctgctcacctgagctggtttcagggcagcccagctctgaacgccacacctatcagcaacaccggcatcctggaactgagaagagtgcggagcgctgaggaaggcggcttcacttgtagagcccagcatcctctgggctttctgcagatcttcctgaacctgagcgtgtacagcctgccacagctgctgggacctagctgttcttgggaagccgagggcctgcactgccggtgtagctttagagctagacccgctcctagcctgtgttggcggctggaagagaaacctctggaaggcaatagcagccagggcagcttcaaagtgaacagcagctctgccggaccttgggccaatagctctctgattctgcacggcggcctgagcagcgatctgaaggtgtcatgcaaggcctggaacatctacggcagccagtctggctctgtgctgctgcttcagggaagaagcaatctcggcacaggcgtggtgcctgctgctctggctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 143 - protein, 654 aa, Artificial Sequence
  Synthetic Siglec 5 Fragment (17-441) SAX / AS / hIgG1 Fc N297G
EKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFAVERGRDVKYSYQQNKLNLEVTALIEKPDIHFLEPLESGRPTRLSCSLPGSCEAGPPLTFSWTGNALSPLDPETTRSSELTLTPRPEDHGTNLTCQMKRQGAQVTTERTVQLNVSYAPQTITIFRNGIALEILQNTSYLPVLEGQALRLLCDAPSNPPAHLSWFQGSPALNATPISNTGILELRRVRSAEEGGFTCRAQHPLGFLQIFLNLSVYSLPQLLGPSCSWEAEGLHCRCSFRARPAPSLCWRLEEKPLEGNSSQGSFKVNSSSAGPWANSSLILHGGLSSDLKVSCKAWNIYGSQSGSVLLLQGRSNLGTGVVPAALASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - DNA, 1962 bp, Artificial Sequence
  Synthetic Siglec 5 Fragment (17-441) SAX / AS / hIgG1 Fc N297G
gagaagcccgtgtatgagctgcaggtccagaaaagcgtgaccgtgcaagagggcctgtgtgtgctggtgccttgcagctttagctacccttggcggagctggtacagcagccctcctctgtacgtgtactggttcagagatggcgagatcccctactacgccgaggtggtggccacaaacaaccccgacagaagagtgaagcccgagacacagggcagattcagactgctgggcgacgtgcagaagaagaactgcagcctgagcatcggcgacgcccggatggaagataccggcagctacttcttcgccgtggaacgcggcagggacgtgaagtacagctaccagcagaacaagctgaacctggaagtgacagccctgatcgagaagcctgacatccactttctggaacccctggaaagcggcagacccaccagactgtcttgtagcctgcctggcagctgtgaagctggccctccactgacctttagctggaccggcaatgccctgtctcctctggaccctgagacaaccagaagcagcgagctgaccctgacacctagacctgaggaccacggcaccaatctgacctgccagatgaagaggcagggcgcccaagtgaccaccgagagaacagtgcagctgaacgtgtcctacgctccccagaccatcaccatcttccggaacggaatcgccctggaaatcctgcagaacaccagctacctgcctgtgctggaaggccaggctctgagactgctgtgtgacgcccctagcaatcctcctgctcacctgagctggtttcagggcagcccagctctgaacgccacacctatcagcaacaccggcatcctggaactgagaagagtgcggagcgctgaggaaggcggcttcacttgtagagcccagcatcctctgggctttctgcagatcttcctgaacctgagcgtgtacagcctgccacagctgctgggacctagctgttcttgggaagccgagggcctgcactgccggtgtagctttagagctagacccgctcctagcctgtgttggcggctggaagagaaacctctggaaggcaatagcagccagggcagcttcaaagtgaacagcagctctgccggaccttgggccaatagctctctgattctgcacggcggcctgagcagcgatctgaaggtgtcatgcaaggcctggaacatctacggcagccagtctggctctgtgctgctgcttcagggaagaagcaatctcggcacaggcgtggtgcctgctgctctggctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 145 - protein, 571 aa, Artificial Sequence
  Synthetic Siglec 14 Fragment (17-358) / AS / hIgG1 Fc N297G
EKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQVKRQGAQVTTERTVQLNVSYAPQNLAISIFFRNGTGTALRILSNGMSVPIQEGQSLFLACTVDSNPPASLSWFREGKALNPSQTSMSGTLELPNIGAREGGEFTCRVQHPLGSQHLSFILSVQRSSSSCICVTEKQQGSWPLVLASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - DNA, 1713 bp, Artificial Sequence
  Synthetic Siglec 14 Fragment (17-358) / AS / hIgG1 Fc N297G
gagaagcccgtgtatgagctgcaggtccagaaaagcgtgaccgtgcaagagggcctgtgtgtgctggtgccttgcagctttagctacccttggcggagctggtacagcagccctcctctgtacgtgtactggttcagagatggcgagatcccctactacgccgaggtggtggccacaaacaaccccgacagaagagtgaagcccgagacacagggcagattcagactgctgggcgacgtgcagaagaagaactgcagcctgagcatcggcgacgcccggatggaagataccggcagctacttcttcagagtggaacgcggcagggacgtgaagtacagctaccagcagaacaagctgaacctggaagtgacagccctgatcgagaagcctgacatccactttctggaacccctggaaagcggcagacccaccagactgtcttgtagcctgcctggcagctgtgaagctggccctccactgacctttagctggaccggcaatgccctgtctcctctggaccctgagacaaccagaagcagcgagctgaccctgacacctagacctgaggaccacggcaccaacctgacctgccaagtgaaaagacagggcgcccaagtgaccaccgagagaacagtgcagctgaacgtgtcctacgctccccagaacctggccatcagcatctttttcagaaacggcaccggcacagccctgcggatcctgtctaatggcatgagcgtgcccatccaagagggacagtctctgttcctggcctgcaccgtggactctaatcctccagctagcctgagctggtttagagagggcaaagccctgaatcctagccagaccagcatgagcggcaccctggaactgcctaatatcggcgctagagaaggcggcgagttcacctgtagagtgcagcaccctctgggcagccagcacctgagctttatcctgagcgtgcagagaagcagcagcagctgcatctgcgtgaccgagaaacagcaaggcagctggcctctggttctggctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 147 - protein, 571 aa, Artificial Sequence
  Synthetic Siglec 14 Fragment (17-358) SAX / AS / hIgG1 Fc N297G
EKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFAVERGRDVKYSYQQNKLNLEVTALIEKPDIHFLEPLESGRPTRLSCSLPGSCEAGPPLTFSWTGNALSPLDPETTRSSELTLTPRPEDHGTNLTCQVKRQGAQVTTERTVQLNVSYAPQNLAISIFFRNGTGTALRILSNGMSVPIQEGQSLFLACTVDSNPPASLSWFREGKALNPSQTSMSGTLELPNIGAREGGEFTCRVQHPLGSQHLSFILSVQRSSSSCICVTEKQQGSWPLVLASDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - DNA, 1713 bp, Artificial Sequence
  Synthetic Siglec 14 Fragment (17-358) SAX / AS / hIgG1 Fc N297G
gagaagcccgtgtatgagctgcaggtccagaaaagcgtgaccgtgcaagagggcctgtgtgtgctggtgccttgcagctttagctacccttggcggagctggtacagcagccctcctctgtacgtgtactggttcagagatggcgagatcccctactacgccgaggtggtggccacaaacaaccccgacagaagagtgaagcccgagacacagggcagattcagactgctgggcgacgtgcagaagaagaactgcagcctgagcatcggcgacgcccggatggaagataccggcagctacttcttcgccgtggaacgcggcagggacgtgaagtacagctaccagcagaacaagctgaacctggaagtgacagccctgatcgagaagcctgacatccactttctggaacccctggaaagcggcagacccaccagactgtcttgtagcctgcctggcagctgtgaagctggccctccactgacctttagctggaccggcaatgccctgtctcctctggaccctgagacaaccagaagcagcgagctgaccctgacacctagacctgaggaccacggcaccaacctgacctgccaagtgaaaagacagggcgcccaagtgaccaccgagagaacagtgcagctgaacgtgtcctacgctccccagaacctggccatcagcatctttttcagaaacggcaccggcacagccctgcggatcctgtctaatggcatgagcgtgcccatccaagagggacagtctctgttcctggcctgcaccgtggactctaatcctccagctagcctgagctggtttagagagggcaaagccctgaatcctagccagaccagcatgagcggcaccctggaactgcctaatatcggcgctagagaaggcggcgagttcacctgtagagtgcagcaccctctgggcagccagcacctgagctttatcctgagcgtgcagagaagcagcagcagctgcatctgcgtgaccgagaaacagcaaggcagctggcctctggttctggctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 149 - protein, 652 aa, Artificial Sequence
  Synthetic Siglec 5 Fragment (17-441) / hIgG1 Fc N297G
EKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQMKRQGAQVTTERTVQLNVSYAPQTITIFRNGIALEILQNTSYLPVLEGQALRLLCDAPSNPPAHLSWFQGSPALNATPISNTGILELRRVRSAEEGGFTCRAQHPLGFLQIFLNLSVYSLPQLLGPSCSWEAEGLHCRCSFRARPAPSLCWRLEEKPLEGNSSQGSFKVNSSSAGPWANSSLILHGGLSSDLKVSCKAWNIYGSQSGSVLLLQGRSNLGTGVVPAALDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 652 aa, Artificial Sequence
  Synthetic Siglec 5 Fragment (17-441) SAX / hIgG1 Fc N297G
EKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFAVERGRDVKYSYQQNKLNLEVTALIEKPDIHFLEPLESGRPTRLSCSLPGSCEAGPPLTFSWTGNALSPLDPETTRSSELTLTPRPEDHGTNLTCQMKRQGAQVTTERTVQLNVSYAPQTITIFRNGIALEILQNTSYLPVLEGQALRLLCDAPSNPPAHLSWFQGSPALNATPISNTGILELRRVRSAEEGGFTCRAQHPLGFLQIFLNLSVYSLPQLLGPSCSWEAEGLHCRCSFRARPAPSLCWRLEEKPLEGNSSQGSFKVNSSSAGPWANSSLILHGGLSSDLKVSCKAWNIYGSQSGSVLLLQGRSNLGTGVVPAALDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 569 aa, Artificial Sequence
  Synthetic Siglec 14 Fragment (17-358) SAX / hIgG1 Fc N297G
EKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFAVERGRDVKYSYQQNKLNLEVTALIEKPDIHFLEPLESGRPTRLSCSLPGSCEAGPPLTFSWTGNALSPLDPETTRSSELTLTPRPEDHGTNLTCQVKRQGAQVTTERTVQLNVSYAPQNLAISIFFRNGTGTALRILSNGMSVPIQEGQSLFLACTVDSNPPASLSWFREGKALNPSQTSMSGTLELPNIGAREGGEFTCRVQHPLGSQHLSFILSVQRSSSSCICVTEKQQGSWPLVLDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 19 aa, Mus musculus
  misc_feature (1)..(19): N-terminal signal sequence
MGWSCIILFLVATATGVHS

SEQ ID NO: 153 - protein, 19 aa, Homo sapiens
  misc_feature (1)..(19): N-terminal signal sequence
MEFGLSWLFLVAILKGVQC

SEQ ID NO: 154 - protein, 469 aa, Artificial Sequence
  Synthetic Siglec 3 Fragment (18-259) / hIgG1 Fc N297G
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 569 aa, Artificial Sequence
  Synthetic Siglec 14 Fragment (17-358) / hIgG1 Fc N297G
EKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQVKRQGAQVTTERTVQLNVSYAPQNLAISIFFRNGTGTALRILSNGMSVPIQEGQSLFLACTVDSNPPASLSWFREGKALNPSQTSMSGTLELPNIGAREGGEFTCRVQHPLGSQHLSFILSVQRSSSSCICVTEKQQGSWPLVLDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 321 aa, Artificial Sequence
  Synthetic Siglec 6 Fragment (27-347) SAX
QERRFQLEGPESLTVQEGLCVLVPCRLPTTLPASYYGYGYWFLEGADVPVATNDPDEEVQEETRGRFHLLWDPRRKNCSLSIRDARRRDNAAYFFKLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGV

SEQ ID NO: 157 - DNA, 963 bp, Artificial Sequence
  Synthetic Siglec 6 Fragment (27-347) SAX
caagagcggcggtttcagctggaaggacctgagagcctgacagtgcaagagggcctgtgtgtgctggtgccttgtagactgcctaccacactgcccgccagctactacggctacggctattggtttctggaaggcgccgatgtgcccgtggccaccaatgatcctgacgaggaagtgcaagaagagacacggggcagattccatctgctgtgggaccccagacggaagaactgcagcctgagcatcagagatgccagaaggcgggataacgccgcctacttcttcaagctgaagtccaagtggatgaagtacggctacaccagcagcaagctgagcgtcagagtgatggccctgacacacagacccaacatcagcatccccggcacactggaaagcggacaccctagcaatctgacctgcagcgtgccatgggtttgcgagcagggaacccctcctatcttctcctggatgtctgccgctcctacctctctgggccctagaacaacacagagcagcgtgctgaccatcacacccagacctcaggaccacagcaccaacctgacctgccaagtgacctttcctggcgctggcgtgaccatggaaagaaccatccagctcaacgtgtcctacgctccccagaaggtggccatcagcattttccagggcaacagcgccgccttcaagatcctgcagaataccagcagcctgcctgtgctggaaggccaggctctgagactgctgtgtgacgccgatggcaatcctcctgctcacctgagctggttccagggcttcccagctctgaacgccacacctatcagcaacaccggcgtgctggaactgcctcaagtgggatctgccgaggaaggcgacttcacctgtagagcccaacatcctctgggcagcctgcagatctccctgagcctgtttgtgcactggaagcccgaaggcagagctggcggagtg

SEQ ID NO: 158 - protein, 553 aa, Artificial Sequence
  Synthetic Siglec 6 Fragment (27-347) SAX / EPKSS / hIgG1 Fc N297G
QERRFQLEGPESLTVQEGLCVLVPCRLPTTLPASYYGYGYWFLEGADVPVATNDPDEEVQEETRGRFHLLWDPRRKNCSLSIRDARRRDNAAYFFKLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGVEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - DNA, 1659 bp, Artificial Sequence
  Synthetic Siglec 6 Fragment (27-347) SAX / EPKSS / hIgG1 Fc N297G
caagagcggcggtttcagctggaaggacctgagagcctgacagtgcaagagggcctgtgtgtgctggtgccttgtagactgcctaccacactgcccgccagctactacggctacggctattggtttctggaaggcgccgatgtgcccgtggccaccaatgatcctgacgaggaagtgcaagaagagacacggggcagattccatctgctgtgggaccccagacggaagaactgcagcctgagcatcagagatgccagaaggcgggataacgccgcctacttcttcaagctgaagtccaagtggatgaagtacggctacaccagcagcaagctgagcgtcagagtgatggccctgacacacagacccaacatcagcatccccggcacactggaaagcggacaccctagcaatctgacctgcagcgtgccatgggtttgcgagcagggaacccctcctatcttctcctggatgtctgccgctcctacctctctgggccctagaacaacacagagcagcgtgctgaccatcacacccagacctcaggaccacagcaccaacctgacctgccaagtgacctttcctggcgctggcgtgaccatggaaagaaccatccagctcaacgtgtcctacgctccccagaaggtggccatcagcattttccagggcaacagcgccgccttcaagatcctgcagaataccagcagcctgcctgtgctggaaggccaggctctgagactgctgtgtgacgccgatggcaatcctcctgctcacctgagctggttccagggcttcccagctctgaacgccacacctatcagcaacaccggcgtgctggaactgcctcaagtgggatctgccgaggaaggcgacttcacctgtagagcccaacatcctctgggcagcctgcagatctccctgagcctgtttgtgcactggaagcccgaaggcagagctggcggagtggagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 160 - protein, 232 aa, Artificial Sequence
  Synthetic hIgG1 Fc N297G with N-terminal EPKSS included.
EPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - DNA, 696 bp, Artificial Sequence
  Synthetic hIgG1 Fc N297G with N-terminal EPKSS included.
gagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggtagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

(19) United States
(12) Patent Application Publication
(10) Pub. No.: US 2024/0156979 A1
(43) Pub. Date: May 16, 2024

(54) ANTI-INFLAMMATORY SIGLEC PROTEINS AND METHODS OF MAKING AND USING SAME

(71) Applicant: Palleon Pharmaceuticals Inc., Waltham, MA (US)

(72) Inventors: Li Peng, Lexington, MA (US); Sujata B. Nerle, Somerville, MA (US); Zakir B. Siddiquee, Cambridge, MA (US)

(21) Appl. No.: 18/554,232

(22) PCT Filed: Apr. 15, 2022
(86) PCT No.: PCT/US2022/025087

§ 371 Date: Oct. 6, 2023

Related U.S. Application Data
(60) Provisional application No. 63/175,826, filed on Apr. 16, 2021.

(51) Int. Cl. A61K 47/68; A61K 38/17; A61P 37/06

(52) U.S. Cl. CPC A61K 47/6811; A61K 38/1732; A61P 37/06

(57) ABSTRACT

The invention relates generally to proteins comprising a recombinant Siglec extracellular domain (ECD), or a functional fragment or variant thereof, optionally containing a mutation that reduces sialic acid binding activity and/or conjugated to a serum half-life enhancer. The invention further relates to methods of using the proteins for treating an inflammatory disorder and/or an autoimmune disorder.

### CROSS-REFERENCE TO RELATED APPLICATIONS

This application claims the benefit of and priority to U.S. Provisional Patent Application No. 63/175,826, filed Apr. 16, 2021, the disclosure of which is hereby incorporated by reference in its entirety for all purposes.

### FIELD OF THE INVENTION

The invention relates generally to Siglec proteins, including Siglec extracellular domains (ECDs), and their use in treating inflammatory and/or autoimmune disorders.

### BACKGROUND

Siglecs (Sialic acid-binding immunoglobulin-type lectins) belong to a lectin-based family of cell surface protein receptors that bind to sialic acid, e.g., sialoglycans, and are predominantly expressed on cells of the hematopoietic system in a manner dependent on cell type and differentiation. Siglecs are Type I transmembrane proteins where the amino terminus is located in the extracellular space and the carboxy terminus is located in the cytosol. Each Siglec protein contains an N-terminal V-set immunoglobulin-like domain (Ig domain) that acts as the binding receptor for sialic acid. Siglecs are lectins, and are categorized into the group of I-type lectins because the lectin domain has a three-dimensional structure similar to an immunoglobulin fold. All Siglecs extend from the cell surface by means of intervening C2-set domains which have no binding activity. Siglecs differ in the number of these C2-set domains. As these proteins contain Ig-like domains, they are members of the Immunoglobulin superfamily (IgSF).

There are at least 14 different mammalian Siglecs, which together provide an array of different functions based on cell surface receptor-ligand interactions. Most Siglecs are classified as “inhibitory” as they contain an intracellular immunoreceptor tyrosine-based inhibitory motif (ITIM) and ITIM-like motifs in their cytoplasmic tail. On engagement by sialoglycans, ITIMs may become phosphorylated and recruit SH2 domain-containing protein tyrosine phosphatases, SHP1 and SHP2. These phosphatases can inhibit signaling pathways triggered in close proximity and thereby modulate diverse physiological responses depending on the cell type and Siglecs. A few Siglecs, including Siglecs-14, -15, and -16, are known as “activating” and signal through adaptor proteins such as DNAX-activating protein-10 (DAP-10) and DNAX-activating protein-12 (DAP-12) upon binding to sialoglycans. These Siglec-glycan interactions can mediate, among other things, cell adhesion and modulate immune cell functions.

Activation of T cells, for example, occurs through the simultaneous engagement of the T-cell receptor and a co-stimulatory molecule (e.g., CD28 or ICOS) on CD4+ T cells by the major histocompatibility complex (MHCII) peptide and co-stimulatory molecules on the antigen-presenting cell (APC). Both are required for production of an effective immune response. In the absence of co-stimulation, T-cell receptor signaling alone results in anergy. It is believed that the signaling pathways downstream from co-stimulatory molecules usually engage the PI3K pathway generating PIP3 at the plasma membrane and recruiting PH domain-containing signaling molecules like PDK1 that are essential for the activation of PKC-θ, and eventual IL-2 production. In addition, CD4+ cells are useful in the initial antigenic activation of naïve CD8 T cells, and sustaining memory CD8+ T cells in the aftermath of an acute infection. Therefore, activation of CD4+ T cells can be beneficial to the action of CD8+ T cells. However, an undesirable T-cell activation can result in the development of an inflammatory and/or autoimmune disorder in a subject.

Accordingly, there is a need in the art for compositions and methods for treating inflammatory and/or autoimmune disorders, for example, by reducing the number or activity of activated T cells in a subject with an inflammatory and/or autoimmune disorder.

### SUMMARY OF THE INVENTION

The invention is based, in part, upon the discovery that Siglec extracellular domains (ECDs) regardless of their “inhibitory” or “activating” cytoplasmic domains, including Siglec ECDs with reduced or no sialic acid binding activity, can act as a ligand and suppress immune responses (such as T cell activation) independent of sialoglycan interaction, by interacting with certain receptors on T cells potentially through protein-protein interaction. This discovery differs from the conventional understanding that Siglecs function as sialoglycan-sensing receptors and signal through their ITIM cytoplasmic domains or through association with DAP-10/DAP-12 to inhibit or activate certain immune functions. As discussed herein, the Siglec ECDs, which include functional fragments and variants of Siglec ECDs, can suppress the activation and/or activity of certain immune cells, e.g., T cells. Accordingly, in certain embodiments, a protein comprising a Siglec ECD, or a functional fragment or variant thereof as described herein, can be administered to a subject in need thereof to treat an inflammatory and/or autoimmune disorder, such as fibrosis and arthritis.

Accordingly, in one aspect, the invention provides a pharmaceutical composition comprising a Siglec extracellular domain (ECD), or a functional fragment or variant thereof, conjugated to a serum half-life enhancer that increases the serum half-life of the Siglec ECD when administered to a subject, wherein the Siglec ECD comprises at least one (e.g., 1, 2, 3 or 4) mutation that reduces or abolishes sialic acid binding activity. In certain embodiments, the at least one mutation results in the Siglec ECD having less than 50% (e.g., less than 40%, less than 30%, less than 20%, less than 10%, less than 5%, less than 3%, less than 1%, or less than 0.5%) of the sialic acid binding activity of a corresponding Siglec ECD lacking the mutation, e.g., a corresponding wild-type Siglec ECD.

In certain embodiments, the Siglec ECD is selected from a human Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, Siglec-15, and Siglec-16 ECD. In certain embodiments, the Siglec ECD is selected from a human Siglec-1, Siglec-4, Siglec-6, Siglec-9, Siglec-11, and Siglec-15 ECD. In certain embodiments, the Siglec ECD is selected from a human Siglec-1, Siglec-4, Siglec-6, Siglec-9, and Siglec-11 ECD.

In certain embodiments,
(a) the Siglec ECD is a human Siglec-1 ECD, and the mutation is present in the region from amino acid 106 to 134 of SEQ ID NO: 15 (wild-type human Siglec-1);
(b) the Siglec ECD is a human Siglec-2 ECD, and the mutation is present in the region from amino acid 110 to 135 of SEQ ID NO: 23 (wild-type human Siglec-2);
(c) the Siglec ECD is a human Siglec-3 ECD, and the mutation is present in the region from amino acid 109 to 137 of SEQ ID NO: 25 (wild-type human Siglec-3);
(d) the Siglec ECD is a human Siglec-4 ECD, and the mutation is present in the region from amino acid 108 to 133 of SEQ ID NO: 33 (wild-type human Siglec-4);
(e) the Siglec ECD is a human Siglec-5 ECD, and the mutation is present in the region from amino acid 109 to 138 of SEQ ID NO: 41 (wild-type human Siglec-5);
(f) the Siglec ECD is a human Siglec-6 ECD, and the mutation is present in the region from amino acid 112 to 140 of SEQ ID NO: 43 (wild-type human Siglec-6);
(g) the Siglec ECD is a human Siglec-7 ECD, and the mutation is present in the region from amino acid 114 to 142 of SEQ ID NO: 51 (wild-type human Siglec-7);
(h) the Siglec ECD is a human Siglec-8 ECD, and the mutation is present in the region from amino acid 115 to 149 of SEQ ID NO: 63 (wild-type human Siglec-8);
(i) the Siglec ECD is a human Siglec-9 ECD, and the mutation is present in the region from amino acid 110 to 138 of SEQ ID NO: 65 (wild-type human Siglec-9);
(j) the Siglec ECD is a human Siglec-10 ECD, and the mutation is present in the region from amino acid 109 to 138 of SEQ ID NO: 87 (wild-type human Siglec-10);
(k) the Siglec ECD is a human Siglec-11 ECD, and the mutation is present in the region from amino acid 122 to 151 of SEQ ID NO: 92 (wild-type human Siglec-11);
(l) the Siglec ECD is a human Siglec-12 ECD, and the mutation is present in the region from amino acid 122 to 151 of SEQ ID NO: 100 (wild-type human Siglec-12);
(m) the Siglec ECD is a human Siglec-14 ECD, and the mutation is present in the region from amino acid 109 to 138 of SEQ ID NO: 102 (wild-type human Siglec-14);
(n) the Siglec ECD is a human Siglec-15 ECD, and the mutation is present in the region from amino acid 133 to 161 of SEQ ID NO: 104 (wild-type human Siglec-15); or
(o) the Siglec ECD is a human Siglec-16 ECD, and the mutation is present in the region from amino acid 110 to 139 of SEQ ID NO: 114 (wild-type human Siglec-16).

In certain embodiments,
(a) the Siglec ECD is a human Siglec-1 ECD, and the mutation is a substitution of an arginine residue at a position corresponding to position 116 of wild-type human Siglec-1 (e.g., R116);
(b) the Siglec ECD is a human Siglec-2 ECD, and the mutation is a substitution of an arginine residue at a position corresponding to position 120 of wild-type human Siglec-2 (e.g., R120);
(c) the Siglec ECD is a human Siglec-3 ECD, and the mutation is a substitution of an arginine residue at a position corresponding to position 119 of wild-type human Siglec-3 (e.g., R119);
(d) the Siglec ECD is a human Siglec-4 ECD, and the mutation is a substitution of an arginine residue at a position corresponding to position 118 of wild-type human Siglec-4 (e.g., R118);
(e) the Siglec ECD is a human Siglec-5 ECD, and the mutation is a substitution of an arginine residue at a position corresponding to position 119 of wild-type human Siglec-5 (e.g., R119);
(f) the Siglec ECD is a human Siglec-6 ECD, and the mutation is a substitution of an arginine residue at a position corresponding to position 122 of wild-type human Siglec-6 (e.g., R122);
(g) the Siglec ECD is a human Siglec-7 ECD, and the mutation is a substitution of an arginine residue at a position corresponding to position 124 of wild-type human Siglec-7 (e.g., R124);
(h) the Siglec ECD is a human Siglec-8 ECD, and the mutation is a substitution of an arginine residue at a position corresponding to position 109 of wild-type human Siglec-8 (e.g., R128);
(i) the Siglec ECD is a human Siglec-9 ECD, and the mutation is a substitution of an arginine residue at a position corresponding to position 120 of wild-type human Siglec-9 (e.g., R120);
(j) the Siglec ECD is a human Siglec-10 ECD, and the mutation is a substitution of an arginine residue at a position corresponding to position 119 of wild-type human Siglec-10 (e.g., R119);
(k) the Siglec ECD is a human Siglec-11 ECD, and the mutation is a substitution of an arginine residue at a position corresponding to position 132 of wild-type human Siglec-11 (e.g., R132);
(l) the Siglec ECD is a human Siglec-12 ECD, and the mutation is a substitution of an arginine residue at a position corresponding to position 125 of wild-type human Siglec-12 (e.g., R125);
(m) the Siglec ECD is a human Siglec-14 ECD, and the mutation is a substitution of an arginine residue at a position corresponding to position 119 of wild-type human Siglec-14 (e.g., R119);
(n) the Siglec ECD is a human Siglec-15 ECD, and the mutation is a substitution of an arginine residue at a position corresponding to position 143 of wild-type human Siglec-15 (e.g., R143); or
(o) the Siglec ECD is a human Siglec-16 ECD, and the mutation is a substitution of an arginine residue at a position corresponding to position 120 of wild-type human Siglec-16 (e.g., R120).

In another aspect, the disclosure relates to a pharmaceutical composition comprising a Siglec extracellular domain (ECD), or a functional fragment or variant thereof, conjugated to a serum half-life enhancer that increases the serum half-life of the Siglec ECD when administered to a subject, wherein the Siglec ECD is selected from a human Siglec-1, Siglec-2, Siglec-3, Siglec-6, Siglec-7, Siglec-8, Siglec-10, Siglec-11, Siglec-12, Siglec-14, and Siglec-16 ECD. In certain embodiments, the Siglec ECD is selected from a human Siglec-1, Siglec-6, and Siglec-11 ECD.

In certain embodiments, the Siglec ECD, or the functional fragment or variant thereof, and the serum half-life enhancer are covalently linked together in a fusion protein. In certain embodiments, the Siglec ECD, or the functional fragment or variant thereof, and the serum half-life enhancer are chemically conjugated together. In certain embodiments, the serum half-life enhancer is selected from an immunoglobulin Fc domain, transferrin, albumin, XTEN, a homo-amino acid polymer (HAP), a proline-alanine-serine polymer (PAS), an elastin-like peptide (ELP), albumin binding domain, carboxy-terminal peptide (CTP), gelatin-like protein (GLK) fusion, and a polyethylene glycol (PEG). In certain embodiments, the serum half-life enhancer is an immunoglobulin Fc domain. In certain embodiments, the immunoglobulin Fc domain is derived from a human IgG1, IgG2, IgG3, IgG4, IgA1, IgA2, IgD, IgE, or IgM Fe domain. In certain embodiments, al composition of claim 14, wherein the immunoglobulin Fc domain is derived from a human IgG1, IgG2, IgG3, or IgG4 Fc domain. In certain embodiments, the immunoglobulin Fc domain is derived from a human IgG1 Fc domain. In certain embodiments, the Siglec ECD, or functional fragment or variant thereof, conjugated to the serum half-life enhancer is present as a dimer.

In certain embodiments, the fusion protein comprises an amino acid sequence selected from the group consisting of SEQ ID NOs: 19, 21, 31, 37, 39, 47, 49, 59, 61, 73, 75, 77, 81, 83, 85, 90, 96, 98, 108, 110, 117, 120, 122, 124, 126-135, 139, 141, 143, 145, 147, 149-151, 154, 155, and 158, or a functional fragment thereof.

In certain embodiments, the pharmaceutical composition, optionally comprising a pharmaceutically acceptable carrier, is disposed in a sterile container (e.g., a bottle or vial). In certain embodiments, the pharmaceutical composition is lyophilized in the sterile container. In certain embodiments, the pharmaceutical composition is present as a solution in the sterile container. In certain embodiments, the sterile container has a label disposed thereon identifying the pharmaceutical composition contained in the container.

In certain embodiments, the disclosure relates to a method of treating an inflammatory and/or autoimmune disorder in a subject in need thereof, the method comprising administering to the subject a pharmaceutical composition as described herein.

In another aspect, the disclosure relates to a method of treating an inflammatory disorder and/or an autoimmune disorder in a subject in need thereof, the method comprising administering to the subject a Siglec extracellular domain (ECD), or a functional fragment or variant thereof, wherein the Siglec ECD comprises at least one (e.g., 1, 2, 3, or 4) mutation that reduces sialic acid binding activity. In certain embodiments, the at least one mutation results in the Siglec ECD, or the functional fragment or variant thereof, having less than 50% (e.g., less than 40%, less than 30%, less than 20%, less than 10%, less than 5%, less than 3%, less than 1%, or less than 0.5%) of the sialic acid binding activity of a corresponding wild-type Siglec ECD.

In certain embodiments, the Siglec ECD is selected from a human Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, Siglec-15, and Siglec-16 ECD. In certain embodiments, the Siglec ECD is selected from a human Siglec-1, Siglec-4, Siglec-6, Siglec-9, Siglec-11, and Siglec-15 ECD. In certain embodiments, the Siglec ECD is selected from a human Siglec-1, Siglec-4, Siglec-6, Siglec-9, and Siglec-11 ECD.

In certain embodiments:
(a) the Siglec ECD is a human Siglec-1 ECD, and the mutation is present in the region from amino acid 106 to 134 of SEQ ID NO: 15 (wild-type human Siglec-1);
(b) the Siglec ECD is a human Siglec-2 ECD, and the mutation is present in the region from amino acid 110 to 135 of SEQ ID NO: 23 (wild-type human Siglec-2);
(c) the Siglec ECD is a human Siglec-3 ECD, and the mutation is present in the region from amino acid 109 to 137 of SEQ ID NO: 25 (wild-type human Siglec-3);
(d) the Siglec ECD is a human Siglec-4 ECD, and the mutation is present in the region from amino acid 108 to 133 of SEQ ID NO: 33 (wild-type human Siglec-4);
(e) the Siglec ECD is a human Siglec-5 ECD, and the mutation is present in the region from amino acid 109 to 138 of SEQ ID NO: 41 (wild-type human Siglec-5);
(f) the Siglec ECD is a human Siglec-6 ECD, and the mutation is present in the region from amino acid 112 to 140 of SEQ ID NO: 43 (wild-type human Siglec-6);
(g) the Siglec ECD is a human Siglec-7 ECD, and the mutation is present in the region from amino acid 114 to 142 of SEQ ID NO: 51 (wild-type human Siglec-7);
(h) the Siglec ECD is a human Siglec-8 ECD, and the mutation is present in the region from amino acid 115 to 149 of SEQ ID NO: 63 (wild-type human Siglec-8);
(i) the Siglec ECD is a human Siglec-9 ECD, and the mutation is present in the region from amino acid 110 to 138 of SEQ ID NO: 65 (wild-type human Siglec-9);
(j) the Siglec ECD is a human Siglec-10 ECD, and the mutation is present in the region from amino acid 109 to 138 of SEQ ID NO: 87 (wild-type human Siglec-10);
(k) the Siglec ECD is a human Siglec-11 ECD, and the mutation is present in the region from amino acid 122 to 151 of SEQ ID NO: 92 (wild-type human Siglec-11);
(l) the Siglec ECD is a human Siglec-12 ECD, and the mutation is present in the region from amino acid 122 to 151 of SEQ ID NO: 100 (wild-type human Siglec-12);
(m) the Siglec ECD is a human Siglec-14 ECD, and the mutation is present in the region from amino acid 109 to 138 of SEQ ID NO: 102 (wild-type human Siglec-14);
(n) the Siglec ECD is a human Siglec-15 ECD, and the mutation is present in the region from amino acid 133 to 161 of SEQ ID NO: 104 (wild-type human Siglec-15); or
(o) the Siglec ECD is a human Siglec-16 ECD, and the mutation is present in the region from amino acid 110 to 139 of SEQ ID NO: 114 (wild-type human Siglec-16).

In certain embodiments:
(a) the Siglec ECD is a human Siglec-1 ECD, and the mutation is a substitution of an arginine residue at a position corresponding to position 116 of wild-type human Siglec-1 (e.g., R116);
(b) the Siglec ECD is a human Siglec-2 ECD, and the mutation is a substitution of an arginine residue at a position corresponding to position 120 of wild-type human Siglec-2 (e.g., R120);
(c) the Siglec ECD is a human Siglec-3 ECD, and the mutation is a substitution of an arginine residue at a position corresponding to position 119 of wild-type human Siglec-3 (e.g., R119);
(d) the Siglec ECD is a human Siglec-4 ECD, and the mutation is a substitution of an arginine residue at a position corresponding to position 118 of wild-type human Siglec-4 (e.g., R118);
(e) the Siglec ECD is a human Siglec-5 ECD, and the mutation is a substitution of an arginine residue at a position corresponding to position 119 of wild-type human Siglec-5 (e.g., R119);
(f) the Siglec ECD is a human Siglec-6 ECD, and the mutation is a substitution of an arginine residue at a position corresponding to position 122 of wild-type human Siglec-6 (e.g., R122);
(g) the Siglec ECD is a human Siglec-7 ECD, and the mutation is a substitution of an arginine residue at a position corresponding to position 124 of wild-type human Siglec-7 (e.g., R124);
(h) the Siglec ECD is a human Siglec-8 ECD, and the mutation is a substitution of an arginine residue at a position corresponding to position 109 of wild-type human Siglec-8 (e.g., R128);
(i) the Siglec ECD is a human Siglec-9 ECD, and the mutation is a substitution of an arginine residue at a position corresponding to position 120 of wild-type human Siglec-9 (e.g., R120);
(j) the Siglec ECD is a human Siglec-10 ECD, and the mutation is a substitution of an arginine residue at a position corresponding to position 119 of wild-type human Siglec-10 (e.g., R119);
(k) the Siglec ECD is a human Siglec-11 ECD, and the mutation is a substitution of an arginine residue at a position corresponding to position 132 of wild-type human Siglec-11 (e.g., R132);
(l) the Siglec ECD is a human Siglec-12 ECD, and the mutation is a substitution of an arginine residue at a position corresponding to position 125 of wild-type human Siglec-12 (e.g., R125);
(m) the Siglec ECD is a human Siglec-14 ECD, and the mutation is a substitution of an arginine residue at a position corresponding to position 119 of wild-type human Siglec-14 (e.g., R119);
(n) the Siglec ECD is a human Siglec-15 ECD, and the mutation is a substitution of an arginine residue at a position corresponding to position 143 of wild-type human Siglec-15 (e.g., R143); or
(o) the Siglec ECD is a human Siglec-16 ECD, and the mutation is a substitution of an arginine residue at a position corresponding to position 120 of wild-type human Siglec-16 (e.g., R120).

In another aspect, the disclosure relates to a method of treating an inflammatory disorder and/or an autoimmune disorder in a subject in need thereof, the method comprising administering to the subject a Siglec extracellular domain (ECD), or a functional fragment or variant thereof, wherein the Siglec ECD is selected from a human Siglec-1, Siglec-2, Siglec-3, Siglec-6, Siglec-7, Siglec-8, Siglec-10, Siglec-11, Siglec-12, Siglec-14, and Siglec-16 ECD.

In certain embodiments, the Siglec ECD, or the functional fragment or variant thereof, comprises a mutation that reduces sialic acid binding activity.

In certain embodiments, the mutation results in the Siglec ECD, or the functional fragment or variant thereof, having less than 50% (e.g., less than 40%, less than 30%, less than 20%, less than 10%, less than 5%, less than 3%, less than 1%, or less than 0.5%) of the sialic acid binding activity of a corresponding Siglec ECD without the mutation, e.g., a corresponding wild-type Siglec ECD.

In certain embodiments:
(a) the Siglec ECD is a human Siglec-1 ECD, and the mutation is present in the region from amino acid 106 to 134 of SEQ ID NO: 15 (wild-type human Siglec-1);
(b) the Siglec ECD is a human Siglec-2 ECD, and the mutation is present in the region from amino acid 110 to 135 of SEQ ID NO: 23 (wild-type human Siglec-2);
(c) the Siglec ECD is a human Siglec-3 ECD, and the mutation is present in the region from amino acid 109 to 137 of SEQ ID NO: 25 (wild-type human Siglec-3);
(d) the Siglec ECD is a human Siglec-6 ECD, and the mutation is present in the region from amino acid 112 to 140 of SEQ ID NO: 43 (wild-type human Siglec-6);
(e) the Siglec ECD is a human Siglec-7 ECD, and the mutation is present in the region from amino acid 114 to 142 of SEQ ID NO: 51 (wild-type human Siglec-7);
(f) the Siglec ECD is a human Siglec-8 ECD, and the mutation is present in the region from amino acid 115 to 149 of SEQ ID NO: 63 (wild-type human Siglec-8);
(g) the Siglec ECD is a human Siglec-10 ECD, and the mutation is present in the region from amino acid 109 to 138 of SEQ ID NO: 87 (wild-type human Siglec-10);
(h) the Siglec ECD is a human Siglec-11 ECD, and the mutation is present in the region from amino acid 122 to 151 of SEQ ID NO: 92 (wild-type human Siglec-11);
(i) the Siglec ECD is a human Siglec-12 ECD, and the mutation is present in the region from amino acid 122 to 151 of SEQ ID NO: 100 (wild-type human Siglec-12);
(j) the Siglec ECD is a human Siglec-14 ECD, and the mutation is present in the region from amino acid 109 to 138 of SEQ ID NO: 102 (wild-type human Siglec-14);
(k) the Siglec ECD is a human Siglec-16 ECD, and the mutation is present in the region from amino acid 110 to 139 of SEQ ID NO: 114 (wild-type human Siglec-16).

In certain embodiments:
(a) the Siglec ECD is a human Siglec-1 ECD, and the mutation is a substitution of an arginine residue at a position corresponding to position 116 of wild-type human Siglec-1 (e.g., R116);
(b) the Siglec ECD is a human Siglec-2 ECD, and the mutation is a substitution of an arginine residue at a position corresponding to position 120 of wild-type human Siglec-2 (e.g., R120);
(c) the Siglec ECD is a human Siglec-3 ECD, and the mutation is a substitution of an arginine residue at a position corresponding to position 119 of wild-type human Siglec-3 (e.g., R119);
(d) the Siglec ECD is a human Siglec-6 ECD, and the mutation is a substitution of an arginine residue at a position corresponding to position 122 of wild-type human Siglec-6 (e.g., R122);
(e) the Siglec ECD is a human Siglec-7 ECD, and the mutation is a substitution of an arginine residue at a position corresponding to position 124 of wild-type human Siglec-7 (e.g., R124);
(f) the Siglec ECD is a human Siglec-8 ECD, and the mutation is a substitution of an arginine residue at a position corresponding to position 109 of wild-type human Siglec-8 (e.g., R128);
(g) the Siglec ECD is a human Siglec-10 ECD, and the mutation is a substitution of an arginine residue at a position corresponding to position 119 of wild-type human Siglec-10 (e.g., R119);
(h) the Siglec ECD is a human Siglec-11 ECD, and the mutation is a substitution of an arginine residue at a position corresponding to position 132 of wild-type human Siglec-11 (e.g., R132);
(i) the Siglec ECD is a human Siglec-12 ECD, and the mutation is a substitution of an arginine residue at a position corresponding to position 125 of wild-type human Siglec-12 (e.g., R125);
(j) the Siglec ECD is a human Siglec-14 ECD, and the mutation is a substitution of an arginine residue at a position corresponding to position 119 of wild-type human Siglec-14 (e.g., R119); or
(k) the Siglec ECD is a human Siglec-16 ECD, and the mutation is a substitution of an arginine residue at a position corresponding to position 120 of wild-type human Siglec-16 (e.g., R120).

In certain embodiments, the Siglec ECD, or the functional fragment or variant thereof, is conjugated to a serum half-life enhancer. In certain embodiments, the Siglec ECD, or the functional fragment or variant thereof, and the serum half-life enhancer are covalently linked together in a fusion protein. In certain embodiments, the Siglec ECD, or the functional fragment or variant thereof, and serum half-life enhancer are chemically conjugated together. In certain embodiments, the serum half-life enhancer is selected from an immunoglobulin Fc domain, transferrin, albumin, XTEN, a homo-amino acid polymer (HAP), a proline-alanine-serine polymer (PAS), an elastin-like peptide (ELP), albumin binding domain, carboxy-terminal peptide (CTP), gelatin-like protein (GLK) fusion, and a polyethylene glycol (PEG). In certain embodiments, the serum half-life enhancer is an immunoglobulin Fe domain. In certain embodiments, the immunoglobulin Fe domain is derived from a human IgG1, IgG2, IgG3, IgG4, IgA1, IgA2, IgD, IgE, or IgM Fe domain. In certain embodiments, the immunoglobulin Fe domain is derived from a human IgG1, IgG2, IgG3, or IgG4 Fe domain. In certain embodiments, the immunoglobulin Fe domain is derived from a human IgG1 Fe domain.

In certain embodiments, the fusion protein comprises an amino acid sequence selected from the group consisting of SEQ ID NOs: 19, 21, 31, 37, 39, 47, 49, 59, 61, 73, 75, 77, 81, 83, 85, 90, 96, 98, 108, 110, 117, 120, 122, 124, 126-135, 139, 141, 143, 145, 147, 149-151, 154, 155, and 158, or a functional fragment thereof.

In certain embodiments, the Siglec ECD or the Siglec ECD and the serum half-life enhancer is present as a dimer.

These and other aspects and features of the invention are described in the following detailed description and claims.

### DESCRIPTION OF THE DRAWINGS

The invention can be more completely understood with reference to the following drawings, in which:

FIG. 1 depicts a schematic representation of certain exemplary native Siglec proteins, including murine proteins, human proteins, and a group of “conserved” protein that are common between murine and human Siglecs.

FIG. 2A depicts a schematic representation of ITIM-containing Siglec molecules that function like a “PD-1-like receptor” and wherein the ITIM-containing Siglecs function as receptors and suppress immune function by binding to sialoglycan on a target cell, e.g., a cancer cell. FIG. 2B depicts a schematic representation of Siglec molecules that function like a “PD-L1-like ligand” regardless of their intracellular domains being “inhibitory” (ITIM) or “activating” (DAP-10/12) and wherein such Siglecs function as ligands and suppress immune responses by binding to currently unidentified receptor(s) on T cells in a sialoglycan-independent manner.

FIG. 3 is a schematic depiction of an exemplary Siglec ECD conjugated to a serum half-life enhancer (e.g., an Fc domain), where, as depicted, the exemplary Siglec ECD contains a Siglec N-terminal V-set immunoglobulin-like domain and two C2-set domains.

FIG. 4A depicts an alignment of human Siglec proteins showing a conserved sialic acid binding domain. Conserved residues are highlighted, and the speckled bar along the top of the alignment corresponds to speckled beta-turn motifs present in the proteins.

FIG. 4B depicts three-dimensional models of Siglec-7, Siglec-9, and Siglec-6 V-set domains. The Siglec-7 and Siglec-9 models show sialic acid biding to Siglec-7 and the beta-turn motifs are shown as speckled.

FIG. 5 depicts bar charts showing interferon-γ (IFN-γ) expression by primary human T cells (day 7-8 of culture) treated with Sig-1-HIS, Sig-2-HIS, Sig-3-Fc, Sig-4-Fc, Sig-5-Fc, Sig-6-Fc, Sig-7-Fc-1G (“Sig-7-1G-Fc”), Sig-8-Fc, Sig-9-Fc, Sig-9-Fc-1G (“Sig-9-1G-Fc”), Sig-9-SAX-Fc-1G (“Sig-9-SAX-1G”), Sig-10-Fc, Sig-11-Fc, Sig-14-Fc, Sig-15-Fc, or PDL1-Fc at the indicated concentrations (0.3 or 0.15 μM). Results are shown for two different donors (FIG. 5A and FIG. 5B).

FIG. 6 depicts bar charts showing IFN-γ expression by primary human T cells (day 7-8 of culture) treated with Sig-1-HIS, Sig-2-HIS, Sig-3-Fc, Sig-4-Fc, Sig-5-Fc, Sig-6-Fc, Sig-7-Fc-1G (“Sig-7-1G-Fc”), Sig-8-Fc, Sig-9-Fc, Sig-9-Fc-1G (“Sig-9-1G-Fc”), Sig-9-SAX-Fc-1G (“Sig-9-SAX-1G”), Sig-10-Fc, Sig-11-Fc, Sig-14-Fc, Sig-15-Fc, or PDL1-Fc at the indicated concentrations (0.1 or 0.05 μM). Results are shown for two different donors (FIG. 6A and FIG. 6B).

FIG. 7 depicts graphs showing ForteBio octet binding of purified Siglec-9 ECD constructs to an anti-Siglec-9 antibody. Results are shown for Sig-9-Fc (FIG. 7A), Sig-9-Fc-1G (FIG. 7B), and Sig-9-SAX-Fc-1G (FIG. 7C). Sig-15-Fc-1G was used as a negative control (FIG. 7D).

FIG. 8 depicts bar charts showing the expression of cytokines IFN-γ (FIG. 8A), granzyme A (FIG. 8B), granzyme B (FIG. 8C), soluble Fas ligand (FIG. 8D), and perform (FIG. 8E) by primary human T cells (day 7-8 of culture) treated with Sig-9-Fc-1G (“Sig-9-1G”), Sig-9-SAX-Fc-1G (“Sig-9-SAX-1G”), Sig-4-Fc (filtered once after thawing; “Sig-4-1filter”), Sig-4-Fc (filtered twice after thawing; “Sig-4-2filter”), Sig-4-Fc (tested after thawing without filtration; “Sig-4-Freezer”), Sig-4-SAX-Fc-1G (“Sig-4-SAX-1G”), Sig-6-Fc-1G (“Sig-6-1G”), Sig-6-SAX-Fc-1G (“Sig-6-SAX-1G”), Sig-11-Fc-1G (“Sig-11-1G”), or Sig-11-SAX-Fc-1G (“Sig-11-SAX-1G”).

FIG. 9 depicts bar charts showing IFN-γ secretion (as a measure of T cell activation) in enriched primary Pan-T cells (FIG. 9A) and cultured activated T cells (FIG. 9B) treated with Sig-9-Fc-1G or Sig-9-SAX-Fc-1G (“Sig-9-Fc-1G-SAX”). Sig-9-Fc-1G and Sig-9-SAX-Fc-1G each suppressed T cell activation.

FIG. 10 depicts bar charts showing TNF-α secretion (as a measure of T cell activation) in enriched primary Pan-T cells (FIG. 10A) and cultured activated T cells (FIG. 10B) treated with Sig-9-Fc-1G and Sig-9-SAX-Fc-1G (“Sig-9-Fc-1G-SAX”). Sig-9-Fc-1G and Sig-9-SAX-Fc-1G suppressed T cell activation.

FIG. 11 depicts bar charts showing NFAT activation as measured by luminescence (RLU) in Jurkat cells treated with the indicated Siglec ECD constructs at the indicated concentration (concentrations in μM are shown in parentheses). FIG. 11A shows inhibition of NFAT activation in Jurkat cells treated with Sig-9-Fc-1G and Sig-9-SAX-Fc-1G (“Sig-9-Fc-1G-SAX”). FIG. 11B shows inhibition of NFAT activation in Jurkat cells treated with Sig-11-Fc-1G (“Siglec-11-Fc”) and Sig-11-SAX-Fc-1G (“Sig-11-Fc-SAX”). FIG. 11C shows inhibition of NFAT activation in Jurkat cells treated with Sig-15-Fc-1G. ConA=Concanavalin A; PAL-001=Isotype control IgG1 antibody.

FIG. 12 depicts bar charts showing the expression of IFN-γ (FIG. 12A), TNF-α (FIG. 12B), IL-10 (FIG. 12C), granzyme A (FIG. 12D), granzyme B (FIG. 12E) and perforin (FIG. 12F) in activated T cells treated with Sig-9-SAX-Fc-1G (“Sig-9-SAX-1G”), Sig-6-SAX-Fc-1G (“Sig-6-SAX-1G”), Sig-11-SAX-Fc-1G (“Sig-11-SAX-1G”), Sig-15-SAX-Fc-1G (“Sig-15-SAX-1G”), PDL-1-Fc and PDL-1-His. The Siglec ECD constructs suppressed Th1 cytokine and cytotoxicity molecule expression.

FIG. 13 depicts bar charts showing the expression of IL-12p40 and IL-10 in macrophages treated with Sig-9-Fc, Sig-9-Fc-1G (“Sig-9-TG”), Sig-9-SAX-Fc-1G (“Sig-9-SAX-1G”), Sig-15-Fc, Sig-15-Fc-1G (“Sig-15-1G”), or Sig-15-SAX-Fc-1G (“Sig-15-SAX-1G”). Results are shown for primary human M1 macrophages derived from culturing on Ultra Low plates (FIG. 13A and FIG. 13B), a mix of non-treated M1 and M2 primary human macrophages (FIG. 13C and FIG. 13D), and treated M2 like primary human macrophages (FIG. 13E and FIG. 13F). IL-12p40 expression is shown in FIG. 13A, FIG. 13C, and FIG. 13E. IL-10 expression is shown in FIG. 13B, FIG. 13D, and FIG. 13F. The Siglec ECD constructs suppressed expression of IL-12p40 and enhanced expression of IL-10. Unstim=unstimulated; LPS=lipopolysaccharide; Inf-G=IFN-γ.

FIG. 14 depicts a scatter plot showing the effects of Sig-9-Fc (“Siglec-9-Fc”), Sig-9-Fc-1G (“Siglec-9-1G”), Sig-9-SAX-Fc-1G (“Siglec-9-SAX-1G”), Sig-15-Fc-1G (“Siglec-15-1G”), or mSig-15-SAX-Fc-1G (“mSiglec-1G”) in a mouse IPF (idiopathic pulmonary fibrosis) model, as demonstrated by hydroxyproline levels in flash frozen lung lobes. Bleo=bleomycin.

FIG. 15 depicts the effects of Sig-9-Fc (“Siglec-9-Fc”), Sig-9-Fc-1G (“Siglec-9-1G”), Sig-9-SAX-Fc-1G (“Siglec-9-SAX-1G”), Sig-15-Fc-1G (“Siglec-15-1G”), or mSig-15-SAX-Fc-1G (“mSiglec-1G”) in a mouse CAIA (collagen antibody induced arthritis) model.

FIG. 15A depicts a graph showing the clinical score (CS) over time and FIG. 15B depicts a bar chart showing an Area Under the Curve (AUC) analysis of the results shown in FIG. 15A.

FIG. 16A is a graph showing secretion of IFN-γ following treatment of human T cells from a first donor with Sig-6-SAX-1G or with an IgG1 N297G isotype control (“IgG1-1G”). The IC50 (μM) of each construct is shown below the figure. FIG. 16B shows results for a second donor.

FIG. 17 depicts a flow chart summarizing a human macrophage polarization assay protocol used to assess activity of Sig-6-SAX-1G.

FIGS. 18A and 18B are bar graphs showing secretion of IL-12p40 from either M1 human macrophages (FIG. 18A) or M2 human macrophages (FIG. 18B) following treatment with the indicated concentration of Sig-6-SAX-1G or an IgG1 N297G isotype control (“IgG-1G”).

FIGS. 19A and 19B are bar graphs showing secretion of IL-10 from either M1 human macrophages (FIG. 19A) or M2 human macrophages (FIG. 19B) following treatment with the indicated concentration of Sig-6-SAX-1G or an IgG1 N297G isotype control (“IgG-1G).

FIGS. 20A and 20B are bar graphs showing secretion of IL-23 from either M1 human macrophages (FIG. 20A) or M2 human macrophages (FIG. 20B) following treatment with the indicated concentration of Sig-6-SAX-1G or an IgG1 N297G isotype control (“IgG-1G”).

FIGS. 21A and 21B are bar graphs showing CD64 surface expression from either M1 human macrophages (FIG. 21A) or from M2 human macrophages (FIG. 21B) following treatment with the indicated concentration of Sig-6-SAX-1G or an IgG1 N297G isotype control (“IgG-1G”). Unstimulated (“Unstim”) and stimulated (“LPS+Inf-G”) macrophages that were not treated with Sig-6-SAX-1G or IgG-1G were used as additional controls.

### DETAILED DESCRIPTION

The invention is based, in part, upon the discovery that certain Siglec extracellular domains (ECDs) regardless of their “inhibitory” or “activating” cytoplasmic domains, including Siglec ECDs with reduced or no sialic acid binding activity, can act as a ligand and suppress immune responses (such as T cell activation) independent of sialoglycan interaction, by potentially interacting with certain receptors on T cells through protein-protein interaction. This discovery differs from the conventional understanding that Siglecs function as sialoglycan-sensing receptors and signal through their ITIM cytoplasmic domains or through association with DAP-10/DAP-12 to inhibit or activate certain immune functions. As discussed herein, Siglec ECDs, which include functional fragments and variants of Siglec ECDs can suppress the activation and/or activity of certain immune cells, e.g., T cells. In particular, it has been discovered that certain Siglec ECDs, even with reduced or no sialic acid binding activity, can function like “PD-L1-like molecules,” that bind to receptors on certain immune cells, e.g., T-cells, potentially via protein-protein interactions, to down regulate, suppress or prevent the upregulation of immune cell function, e.g., T-cell function, thereby mediating an anti-inflammatory effect. The invention provides, among other things, compositions, e.g., pharmaceutical compositions, comprising a Siglec extracellular domain (ECD) that can mediate an anti-inflammatory effect. Furthermore, a protein comprising a Siglec ECD, or a functional fragment or variant thereof as discussed herein, can be administered to a subject in need thereof to treat an inflammatory and/or autoimmune disorder such as fibrosis and arthritis.

### I. Siglecs and Siglec Biology

Siglecs (Sialic acid-binding immunoglobulin-type lectins) are cell surface proteins that bind sialic acid. Siglecs comprise a lectin family of surface receptors that bind to sialoglycans and are predominantly expressed on cells of the hematopoietic system in a manner dependent on cell type and differentiation. There are at least 15 different mammalian Siglecs, which together provide an array of different functions based on cell surface receptor-ligand interactions. These receptor-glycan interactions can mediate, among other things, cell adhesion and cell signaling.

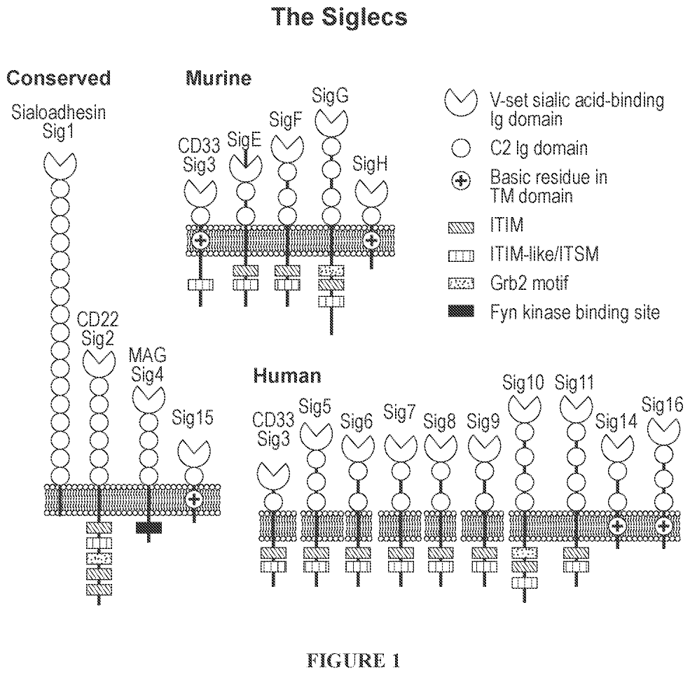

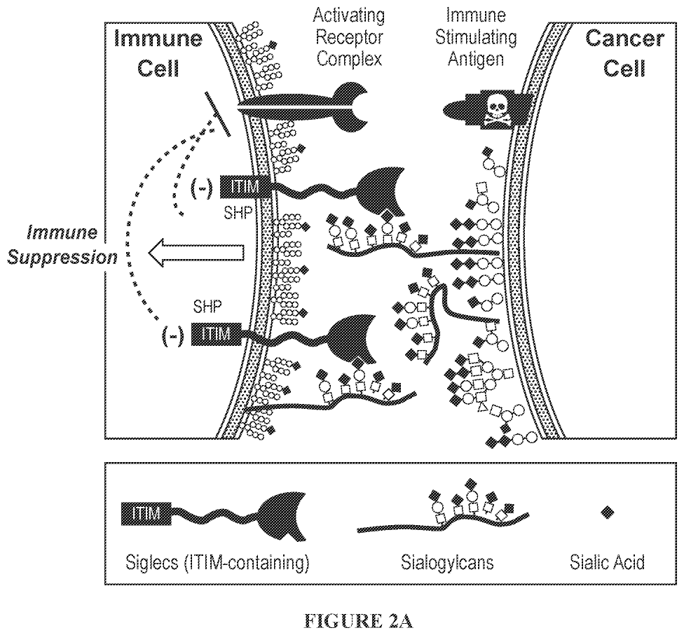

FIG. 1 depicts certain exemplary Siglecs, which include various domains present in human Siglecs, murine Siglecs, and certain Siglecs conserved between humans and mice. Siglecs are Type I transmembrane proteins where the amino terminus is located in the extracellular space and the carboxy terminus is located in the cytosol. As shown in FIG. 1, each Siglec contains an N-terminal V-set immunoglobulin-like domain (Ig domain) that acts as the binding receptor for sialic acid. Siglecs are classified as I-type lectins because the lectin domain is in the form of an immunoglobulin fold. Siglecs extend from the cell surface by means of intervening C2-set domains which do not bind to sialic acid. As shown in FIG. 1, various Siglecs differ in the number of these C2-set domains. Given that these proteins contain Ig domains, they are members of the Immunoglobulin superfamily (IgSF).

Most Siglecs, and in particular the CD33-like Siglecs, contain immunoreceptor tyrosine-based inhibitory motifs (ITIMs) in their cytoplasmic domains (see, FIG. 1), which act to down-regulate signaling pathways involving phosphorylation, such as those induced by immunoreceptor tyrosine-based activation motifs (ITAMs). Siglecs 3, 7 and 9 are highly and constitutively expressed on macrophages, dendritic cells, and NK cells, and are now known to be induced on T-cells, in particular, effector T-cells.

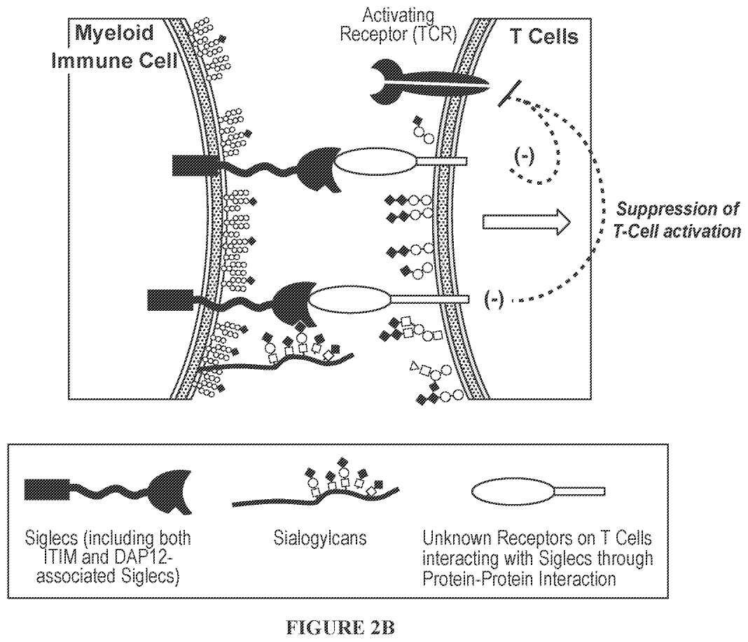

As shown in FIG. 2A, most CD33-like Siglecs are believed to interfere with cellular signaling via their ITIM-containing cytoplasmic domains, thereby inhibiting immune cell activation. In this context, these Siglecs function like “PD-1-like receptors”. For example, and without wishing to be bound by theory, these Siglecs function like receptors on an immune cell which, when bound to their ligands on a cancer cell (e.g., hypersialylated tumor glycans) and on the immune cell itself (e.g., sialylated immune cis-ligands), recruit inhibitory proteins, such as SHP phosphatases, via their ITIM domains. The tyrosine amino acids contained within the ITIM domain become phosphorylated upon ligand binding and act as docking sites for SH2 domain-containing proteins like SHP phosphatases. This leads to de-phosphorylation of cellular proteins, and down-regulation of activating signaling pathways in the immune cell, thereby suppressing immune cell activity and allowing cancer cells to evade the immune system. The alleviation of this suppression can be used to treat a variety of disorders such as cancer.

Although immune suppression by Siglecs is believed to be mediated by their ITIM-containing cytoplasmic domain upon the extracellular domain's recognition of sialic acid binding, it has now been surprisingly discovered that Siglec extracellular domains (ECDs) with reduced or no sialic acid binding activity, can reduce immune system activation and/or dysregulation regardless of the nature of their cytoplasmic domains. Without wishing to be bound by theory, it is believed that, in addition to sialic acid-mediated immune suppression of certain immune cells, certain Siglecs can also act through protein-protein interactions in a sialic acid-independent pathway as a ligand for a currently unidentified receptor, on other immune cells, e.g., T-cells, to reduce immune activation. As shown in FIG. 2B, certain Siglecs can function like a “PD-L1-like ligand” to suppress the function of certain immune cells, e.g., T-cells, via a receptor disposed on the immune cells, e.g., T-cells, in a sialic acid-independent manner to down regulate the inflammatory activity of the immune cells. The down regulation of this anti-inflammatory activity can be used to treat a variety of disorders such as inflammatory and autoimmune disorders.

Accordingly, in certain embodiments, the disclosure relates to the use of a Siglec ECD, including a Siglec ECD with reduced or no sialic acid binding activity, to suppress the function of certain immune cells, e.g., the activity of T-cells. Suppression of T-cell activity can be useful to treat diseases or disorders characterized by hyperactivation or dysregulation of the immune system, such as inflammatory and/or autoimmune disorders.

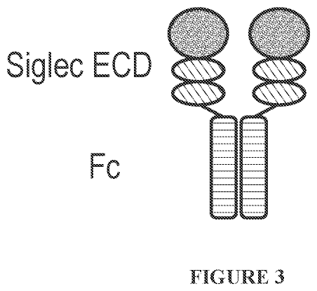

Further, the disclosure relates to a protein comprising a Siglec ECD, or a functional fragment or variant thereof, conjugated to a serum half-life enhancer. In certain embodiments, the Siglec ECD comprises a mutation that reduces sialic acid binding activity. An exemplary, engineered Siglec-based protein is shown schematically in FIG. 3 where a dimer of Siglec ECDs is conjugated to a serum half-life enhancer (e.g., an Fc fragment comprising two polypeptides, each comprising a portion of an immunoglobulin hinge region and immunoglobulin CH2 and CH3 domains, where the two polypeptides are covalently linked through disulfide bonds between cysteine residues present in the respective hinge region sequences). Each exemplary Siglec ECD contains a Siglec N-terminal V-set immunoglobulin-like domain and two C2-set domains. In certain embodiments, the disclosure provides Siglec proteins that contain one or more mutations to reduce or eliminate sialic acid binding activity and a serum half-life extender.

The disclosure further relates to pharmaceutical compositions comprising such proteins and methods of administering such proteins to a subject in need thereof to treat an inflammatory and/or autoimmune disorder. In certain embodiments, the pharmaceutical compositions further comprise a pharmaceutically acceptable carrier.

An amino acid sequence of an exemplary human Siglec-1 protein is provided in SEQ ID NO: 15 (NCBI Reference Sequence: NP_075556.1) and a DNA sequence encoding an exemplary human Siglec-1 protein is provided in SEQ ID NO: 16 (NCBI Reference Sequence: NM_023068.3). An amino acid sequence of an exemplary human Siglec-2 protein is provided in SEQ ID NO: 23 (NCBI Reference Sequence: NP_001762.2) and a DNA sequence encoding an exemplary human Siglec-2 protein is provided in SEQ ID NO: 24 (NCBI Reference Sequence: NM_001771.3). An amino acid sequence of an exemplary human Siglec-3 protein is provided in SEQ ID NO: 25 (NCBI Reference Sequence: NP_001763.3) and a DNA sequence encoding an exemplary human Siglec-3 protein is provided in SEQ ID NO: 26 (NCBI Reference Sequence: NM_001772.3). An amino acid sequence of an exemplary human Siglec-4 protein is provided in SEQ ID NO: 33 (NCBI Reference Sequence: NP_002352.1) and a DNA sequence encoding an exemplary human Siglec-4 protein is provided in SEQ ID NO: 34 (NCBI Reference Sequence: NM_002361.3). An amino acid sequence of an exemplary human Siglec-5 protein is provided in SEQ ID NO: 41 (NCBI Reference Sequence: NP_003821.1) and a DNA sequence encoding an exemplary human Siglec-5 protein is provided in SEQ ID NO: 42 (NCBI Reference Sequence: NM_003830). An amino acid sequence of an exemplary human Siglec-6 protein is provided in SEQ ID NO: 43 (NCBI Reference Sequence: NP_001236.4) and a DNA sequence encoding an exemplary human Siglec-6 protein is provided in SEQ ID NO: 44 (NCBI Reference Sequence: NM_198845.5). An amino acid sequence of an exemplary human Siglec-7 protein is provided in SEQ ID NO: 51 (NCBI Reference Sequence: NP_055200.1) and a DNA sequence encoding an exemplary human Siglec-7 protein is provided in SEQ ID NO: 52 (NCBI Reference Sequence: NM_014385.3). An amino acid sequence of an exemplary human Siglec-8 protein is provided in SEQ ID NO: 63 (NCBI Reference Sequence: NP_055257.2) and a DNA sequence encoding an exemplary human Siglec-8 protein is provided in SEQ ID NO: 64 (NCBI Reference Sequence: NM_014442.2). An amino acid sequence of an exemplary human Siglec-9 protein is provided in SEQ ID NO: 65 (NCBI Reference Sequence: NP_055256.1) and a DNA sequence encoding an exemplary human Siglec-9 protein is provided in SEQ ID NO: 66 (NCBI Reference Sequence: NM_014441.2). An amino acid sequence of an exemplary human Siglec-10 protein is provided in SEQ ID NO: 87 (NCBI Reference Sequence: NP_149121.2) and a DNA sequence encoding an exemplary human Siglec-10 protein is provided in SEQ ID NO: 88 (NCBI Reference Sequence: NM_033130.4). An amino acid sequence of an exemplary human Siglec-11 protein is provided in SEQ ID NO: 92 (NCBI Reference Sequence: NP_443116.2) and a DNA sequence encoding an exemplary human Siglec-11 protein is provided in SEQ ID NO: 93 (NCBI Reference Sequence: NM_052884.2). An amino acid sequence of an exemplary human Siglec-12 protein is provided in SEQ ID NO: 100 (NCBI Reference Sequence: NP_443729.1) and a DNA sequence encoding an exemplary human Siglec-12 protein is provided in SEQ ID NO: 101 (NCBI Reference Sequence: NM_053003.3). An amino acid sequence of an exemplary human Siglec-14 protein is provided in SEQ ID NO: 102 (NCBI Reference Sequence: NP_001092082.1) and a DNA sequence encoding an exemplary human Siglec-14 protein is provided in SEQ ID NO: 103 (NCBI Reference Sequence: NM_001098612.1). An amino acid sequence of an exemplary human Siglec-15 protein is provided in SEQ ID NO: 104 (NCBI Reference Sequence: NP_998767.1) and a DNA sequence encoding an exemplary human Siglec-15 protein is provided in SEQ ID NO: 105 (NCBI Reference Sequence: NM_213602.2). An amino acid sequence of an exemplary human Siglec-16 protein is provided in SEQ ID NO: 114 (NCBI Reference Sequence: NP_001335293.2) and a DNA sequence encoding an exemplary human Siglec-16 protein is provided in SEQ ID NO: 115 (NCBI Reference Sequence: NM_001348364.2).

a. Siglec Extracellular Domains (ECD)

In certain embodiments described herein, the disclosure relates to a Siglec extracellular domain (ECD). An amino acid sequence of an exemplary human Siglec-1 ECD is provided at amino acid residues 20-1642 of SEQ ID NO: 15. In certain embodiments, the Siglec-1 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 20-1642 of SEQ ID NO: 15.

An amino acid sequence of an exemplary human Siglec-2 ECD is provided at amino acid residues 20-688 of SEQ ID NO: 23. In certain embodiments, the Siglec-2 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 20-688 of SEQ ID NO: 23.

An amino acid sequence of an exemplary human Siglec-3 ECD is provided at amino acid residues 18-259 of SEQ ID NO: 25 (i.e., SEQ ID NO: 29). In certain embodiments, the Siglec-3 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 18-259 of SEQ ID NO: 25 (i.e., SEQ ID NO: 29).

An amino acid sequence of an exemplary human Siglec-4 ECD is provided in amino acid residues 20-516 SEQ ID NO: 33. In certain embodiments, the Siglec-4 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 20-516 of SEQ ID NO: 33.

An amino acid sequence of an exemplary human Siglec-5 ECD is provided at amino acid residues 17-441 of SEQ ID NO: 41. In certain embodiments, the Siglec-5 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 17-441 of SEQ ID NO: 41.

An amino acid sequence of an exemplary human Siglec-6 ECD is provided in amino acid residues 27-347 of SEQ ID NO: 43 (i.e., SEQ ID NO: 45). In certain embodiments, the Siglec-6 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 27-347 of SEQ ID NO: 43 (i.e., SEQ ID NO: 45).

An amino acid sequence of an exemplary human Siglec-7 ECD is provided at amino acid residues 19-357 of SEQ ID NO: 51 (i.e., SEQ ID NO: 56). In certain embodiments, the Siglec-7 extracellular domain comprises a Siglec-7 V-Set immunoglobulin-like domain and 2 Siglec-7 C2-Set domains as provided at amino acid residues 19-357 of SEQ ID NO: 51 (i.e., SEQ ID NO: 56). In certain embodiments, the Siglec-7 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 19-357 of SEQ ID NO: 51 (i.e., SEQ ID NO: 56).

An amino acid sequence of an exemplary human Siglec-8 ECD is provided at amino acid residues 17-364 of SEQ ID NO: 63. In certain embodiments, the Siglec-8 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 17-364 of SEQ ID NO: 63.

An amino acid sequence of an exemplary human Siglec-9 ECD is provided at amino acid residues 18-348 of SEQ ID NO: 65 (i.e., SEQ ID NO: 70). In certain embodiments, the Siglec-9 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 18-348 of SEQ ID NO: 65 (i.e., SEQ ID NO: 70).

An amino acid sequence of an exemplary human Siglec-10 ECD is provided at amino acid residues 18-550 of SEQ ID NO: 87. In certain embodiments, the Siglec-10 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 18-550 of SEQ ID NO: 87.

An amino acid sequence of an exemplary human Siglec-11 ECD is provided at amino acid residues 28-561 of SEQ ID NO: 92 (i.e., SEQ ID NO: 94). In certain embodiments, the Siglec-11 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 28-561 of SEQ ID NO: 92 (i.e., SEQ ID NO: 94).

An amino acid sequence of an exemplary human Siglec-12 ECD is provided at amino acid residues 19-482 of SEQ ID NO: 100. In certain embodiments, the Siglec-12 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 19-482 of SEQ ID NO: 100.

An amino acid sequence of an exemplary human Siglec-14 ECD is provided at amino acid residues 17-358 of SEQ ID NO: 102. In certain embodiments, the Siglec-14 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 17-358 of SEQ ID NO: 102.

An amino acid sequence of an exemplary human Siglec-15 ECD is provided at amino acid residues 20-263 of SEQ ID NO: 104 (i.e., SEQ ID NO: 106). In certain embodiments, the Siglec-15 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 20-263 of SEQ ID NO: 104 (i.e., SEQ ID NO: 106).

An amino acid sequence of an exemplary human Siglec-16 ECD is provided at amino acid residues 15-434 of SEQ ID NO: 114 and at amino acid residues 15-434 of SEQ ID NO: 138. In certain embodiments, the Siglec-15 ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 15-434 of SEQ ID NO: 114 or amino acid residues 15-434 of SEQ ID NO: 138.

b. Siglec Extracellular Domain (ECD) Fragments

As used herein, the term “functional fragment” of a Siglec extracellular domain (ECD) refers to fragment of a Siglec ECD that retains, for example, at least 20%, at least 30%, at least 40%, at least 50%, at least 60%, at least 70%, at least 80%, at least 90%, at least 95%, or 100% of the Siglec ECD activity of the corresponding full-length, naturally occurring Siglec ECD. Siglec ECD activity may be assayed by any method known in the art, including, for example, by measuring one or more criteria indicative of the inhibition of immune cell activity, such as (1) the inhibition of NFAT activation a Jurkat Luciferase cell assay as described in Example 3; (2) reduced hydroxyproline in a mouse idiopathic pulmonary fibrosis (IPF) model as described in Example 4; (3) reduced clinical score in a mouse CAIA (Collagen Antibody Induced Arthritis) model; and (4) reduced IFN-γ secretion from activated T cells. In certain embodiments, Siglec ECD activity does not include sialic acid binding activity. In certain embodiments, the functional fragment comprises at least 50, at least 75, at least 100, at least 125, or at least 150 consecutive amino acids present in a full-length, naturally occurring Siglec ECD.

In certain embodiments, the functional fragment of a Siglec ECD comprises a Siglec-1 V-set immunoglobulin-like domain, e.g., amino acid residues 21-136 of SEQ ID NO: 15 or amino acid residues 20-320 of SEQ ID NO: 15 (i.e., SEQ ID NO: 17). In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 21-136 of SEQ ID NO: 15 or amino acid residues 20-320 of SEQ ID NO: 15.

In certain embodiments, the functional fragment of a Siglec ECD comprises a Siglec-2 V-set immunoglobulin-like domain, e.g., amino acid residues 24-122 of SEQ ID NO: 23. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 24-122 of SEQ ID NO: 23.

In certain embodiments, the functional fragment of a Siglec ECD comprises a Siglec-3 V-set immunoglobulin-like domain, e.g., amino acid residues 23-139 of SEQ ID NO: 25 (i.e., SEQ ID NO: 27) or amino acids 23-227 of SEQ ID NO: 25 (i.e., SEQ ID NO: 28. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 23-139 of SEQ ID NO: 25 or amino acid residues 18-259 of SEQ ID NO: 25.

In certain embodiments, the functional fragment of a Siglec ECD comprises a Siglec-4 V-set immunoglobulin-like domain, e.g., amino acid residues 22-139 of SEQ ID NO: 33. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 22-139 of SEQ ID NO: 33.

In certain embodiments, the functional fragment of a Siglec ECD comprises a Siglec-5 V-set immunoglobulin-like domain, e.g., amino acid residues 21-140 of SEQ ID NO: 41. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 21-140 of SEQ ID NO: 41.

In certain embodiments, the functional fragment of a Siglec ECD comprises a Siglec-6 V-set immunoglobulin-like domain, e.g., amino acid residues 31-141 of SEQ ID NO: 43 or amino acid residues 27-347 of SEQ ID NO: 43. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 31-141 of SEQ ID NO: 43 or amino acid residues 27-347 of SEQ ID NO: 43.

In certain embodiments, the functional fragment of a Siglec ECD comprises a Siglec-7 V-set immunoglobulin-like domain, e.g., amino acid residues 26-144 of SEQ ID NO: 51 (i.e., SEQ ID NO: 53) or amino acid residues 31-122 of SEQ ID NO: 51 (i.e., SEQ ID NO: 54). In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 26-144 of SEQ ID NO: 51 (i.e., SEQ ID NO: 53) or amino acid residues 31-122 of SEQ ID NO: 51 (i.e., SEQ ID NO: 54). In certain embodiments, the functional fragment of a Siglec ECD comprises a Siglec-7 V-Set immunoglobulin-like domain and 1 Siglec-7 C2-Set domain, e.g., amino acid residues 19-239 of SEQ ID NO: 51 (i.e., SEQ ID NO: 55). In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 19-239 of SEQ ID NO: 51 (i.e., SEQ ID NO: 55).

In certain embodiments, the functional fragment of a Siglec ECD comprises a Siglec-8 V-set immunoglobulin-like domain, e.g., amino acid residues 27-151 of SEQ ID NO: 63. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 27-151 of SEQ ID NO: 63.

In certain embodiments, the functional fragment of a Siglec ECD comprises a Siglec-9 V-set immunoglobulin-like domain, e.g., amino acid residues 23-144 of SEQ ID NO: 65 (also provided as SEQ ID NO: 67), amino acid residues 23-140 of SEQ ID NO: 65 (also provided as SEQ ID NO: 68), and amino acid residues 18-341 of SEQ ID NO: 65. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 23-144 of SEQ ID NO: 65 (also provided as SEQ ID NO: 67), amino acid residues 23-140 of SEQ ID NO: 65 (also provided as SEQ ID NO: 68), or amino acid residues 18-341 of SEQ ID NO: 65. In certain embodiments, the functional fragment of a Siglec ECD comprises a Siglec-9 V-Set immunoglobulin-like domain and 1 Siglec-9 C2-Set domain, e.g., SEQ ID NO: 69. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 69. In certain embodiments, the functional fragment of a Siglec ECD comprises amino acid residues 18-145 or 146-348 of SEQ ID NO: 65. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 18-145 or 146-348 of SEQ ID NO: 65.

In certain embodiments, the functional fragment of a Siglec ECD comprises a Siglec-10 V-set immunoglobulin-like domain, e.g., amino acid residues 23-140 of SEQ ID NO: 87. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 23-140 of SEQ ID NO: 87.

In certain embodiments, the functional fragment of a Siglec ECD comprises a Siglec-11 V-set immunoglobulin-like domain, e.g., amino acid residues 34-153 of SEQ ID NO: 92. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 34-153 of SEQ ID NO: 92.

In certain embodiments, the functional fragment of a Siglec ECD comprises a Siglec-12 V-set immunoglobulin-like domain, e.g., amino acid residues 24-142 of SEQ ID NO: 100. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 24-142 of SEQ ID NO: 100.

In certain embodiments, the functional fragment of a Siglec ECD comprises a Siglec-14 V-set immunoglobulin-like domain, e.g., amino acid residues 21-140 of SEQ ID NO: 102. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 21-140 of SEQ ID NO: 102.

In certain embodiments, the functional fragment of a Siglec ECD comprises a Siglec-15 V-set immunoglobulin-like domain, e.g., amino acid residues 44-150 of SEQ ID NO: 104. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 44-150 of SEQ ID NO: 104.

In certain embodiments, the functional fragment of a Siglec ECD comprises a Siglec-16 domain, e.g., amino acid residues 15-434 of SEQ ID NO: 114. In certain embodiments, the functional fragment of a Siglec ECD comprises an amino acid sequence having 80%, 85%, 86%, 87%, 88%, 89%, 90%, 91%, 92%, 93%, 94%, 95%, 96%, 97%, 98%, or 99% sequence identity to amino acid residues 15-434 of SEQ ID NO: 114.

In certain embodiments, the functional fragment of a Siglec ECD comprises a Siglec-3 V-Set immunoglobulin-like domain, e.g., SEQ ID NO: 27, a Siglec-6 V-Set immunoglobulin-like domain, e.g., amino acid residues 31-141 of SEQ ID NO: 43, a Siglec-7 V-Set immunoglobulin-like domain, e.g., SEQ ID NO: 53 or SEQ ID NO: 54, or a Siglec-9 V-Set immunoglobulin-like domain, e.g., SEQ ID NO: 67 or SEQ ID NO: 68. In certain embodiments, the functional fragment of a Siglec ECD comprises a Siglec-3 V-Set immunoglobulin-like domain and 1 Siglec-3 C2-Set domain, e.g., SEQ ID NO: 28. In certain embodiments, the functional fragment of a Siglec ECD comprises a Siglec-6 V-Set immunoglobulin-like domain and 1 Siglec-6 C2-Set domain, e.g., amino acid residues 31-233 of SEQ ID NO: 43. In certain embodiments, the functional fragment of a Siglec ECD comprises a Siglec-6 V-Set immunoglobulin domain and 2 Siglec-6 C2-Set domains, e.g., amino acid residues 27-347 of SEQ ID NO: 43. In certain embodiments, the functional fragment of a Siglec ECD comprises a Siglec-7 V-Set immunoglobulin-like domain and 1 Siglec-7 C2-Set domain, e.g., SEQ ID NO: 55. In certain embodiments, the functional fragment of a Siglec ECD comprises a Siglec-9 V-Set immunoglobulin-like domain and 1 Siglec-9 C2-Set domain, e.g., SEQ ID NO: 69. In certain embodiments, the functional fragment of a Siglec ECD comprises a Siglec-7 V-Set immunoglobulin-like domain and 2 Siglec-7 C2-Set domains, e.g., SEQ ID NO: 56. In certain embodiments, the functional fragment of a Siglec ECD comprises a Siglec-9 V-Set immunoglobulin-like domain and 2 Siglec-9 C2-Set domains, e.g., SEQ ID NO: 70.

c. Siglec Extracellular Domain (ECD) Variants

As used herein, the term “variant” of a Siglec extracellular domain (ECD) refers to variant of a Siglec ECD or a functional fragment thereof that retains, for example, at least 20%, at least 30%, at least 40%, at least 50%, at least 60%, at least 70%, at least 80%, at least 90%, at least 95%, or 100% of the Siglec ECD activity of the corresponding full-length, naturally occurring Siglec ECD. Siglec ECD activity may be assayed by any method known in the art, including, for example, by measuring one or more criteria indicative of the inhibition of immune cell activity, such as (1) the inhibition of NFAT activation a Jurkat Luciferase cell assay as described in Example 3; (2) reduced hydroxyproline in a mouse idiopathic pulmonary fibrosis (IPF) model as described in Example 4; and (3) reduced clinical score in a mouse CAIA (Collagen Antibody Induced Arthritis) model. In certain embodiments, Siglec ECD activity does not include sialic acid binding.

In certain embodiments, the variant of a Siglec ECD comprises a substitution of at least one wild-type cysteine residue. For example, in certain embodiments, the variant of a Siglec ECD is from human Siglec-9, and the variant of a Siglec ECD comprises a substitution of a cysteine residue at a position corresponding to position 141 of wild-type human Siglec-9, e.g., the cysteine residue at a position corresponding to position 141 of wild-type human Siglec-9 is substituted by serine (C141S). In certain embodiments, the variant of a Siglec ECD is from human Siglec-9, and the variant of a Siglec ECD comprises a substitution of a cysteine residue at a position corresponding to position 278 of wild-type human Siglec-9, e.g., the cysteine residue at a position corresponding to position 278 of wild-type human Siglec-9 is substituted by threonine (C278T).

In certain embodiments, the variant of a Siglec ECD comprises a conservative substitution relative to a Siglec ECD sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix). In certain embodiments, the Siglec ECD comprises 1, 2, 3, 4, 5, 6, 7, 8, 9, or 10 conservative substitutions.

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; —W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

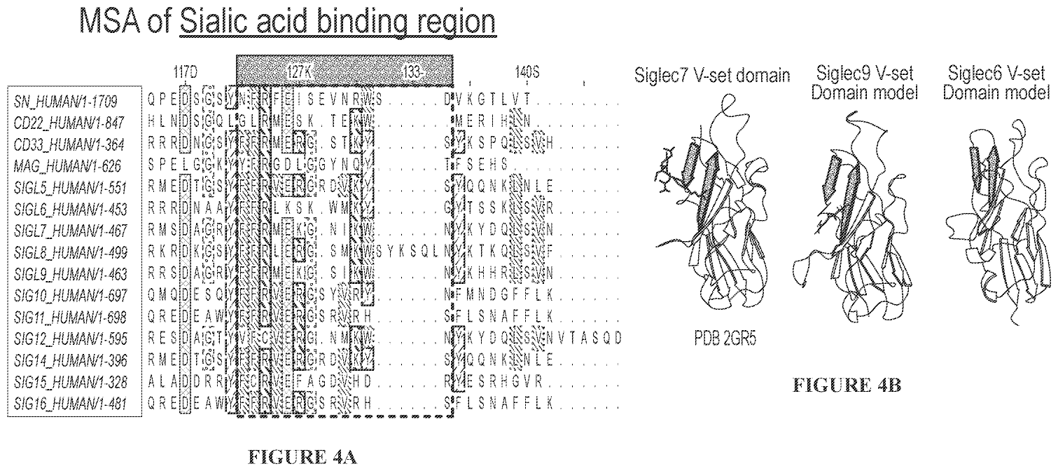

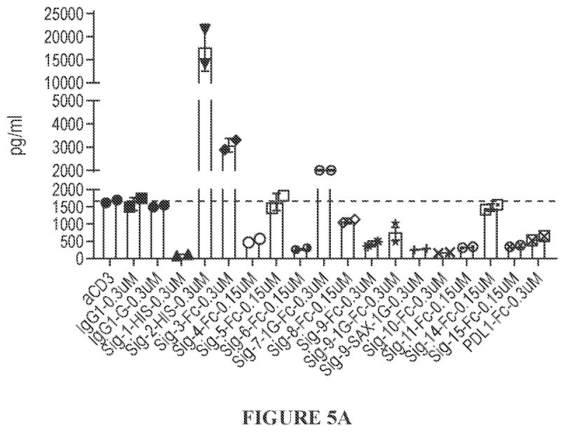

In certain embodiments, the Siglec ECD, or a functional fragment or variant thereof, comprises a mutation that reduces sialic acid binding activity. The mutation can include a deletion, substitution, insertion, or any combination thereof. In certain embodiments, the mutation is a substitution of an alanine (A) for an amino acid present in a wild-type Siglec sequence. FIG. 4A provides an alignment of the sialic acid binding region of human Siglecs (Siglec-1 (“SN”), Siglec-2 (“CD22”), Siglec-3 (“CD33”), Siglec-4 (“MAG”), Siglec-5, Siglec-6, Siglec-7, Siglec-8, Siglec-9, Siglec-10, Siglec-11, Siglec-12, Siglec-14, and Siglec-15. As shown in FIG. 4A, the sialic acid binding region is conserved among Siglec proteins. The conserved region includes residues that make up beta turn motifs (see region beneath the red bar at the top of the alignment). FIG. 4B depicts structural models of Siglec-7, Siglec-9, and Siglec-6 V-set domains. The Siglec-7 and Siglec-9 models show sialic acid binding to each Siglec, and all three models show the beta-turn motifs in the region of the sialic binding residues. The amino acids shown in the alignment in FIG. 4A can contribute to sialic acid binding.

In certain embodiments, the mutation that reduces sialic acid binding is present in the sialic acid binding region shown in FIG. 4A, a region corresponding to, for example, amino acids 110-138 of SEQ ID NO: 65 (human Siglec-9). In certain embodiments, a mutation that reduces sialic acid binding is in at least one conserved residue as shown in FIG. 4. For example, the at least one conserved residue may include a residue that corresponds to amino acid 113, amino acid 115, any one of amino acids 117 to 124, any one of amino acids 126-128, amino acid 130, amino acid 135, and amino acid 137 of SEQ ID NO: 65 (human Siglec-9).

In certain embodiments, the Siglec ECD, or a functional fragment or variant thereof, is a human Siglec-1 ECD, and the mutation is present in the region from amino acid 106 to 134 of SEQ ID NO: 15 (wild-type human Siglec-1). In certain embodiments, the mutation is present in the region from amino acid 114 to 127 of SEQ ID NO: 15 (wild-type human Siglec-1). In certain embodiments, the mutation is present in the region from amino acid 113 to 116 of SEQ ID NO: 15 (wild-type human Siglec-1). In certain embodiments, the mutation is present at D109, G111, Y113, F115, R116, E118, or W125 of SEQ ID NO: 15 (wild-type human Siglec-1). In certain embodiments, the mutation is present at 158, Y60, N114, R124, or D127 of SEQ ID NO: 15 (wild-type human Siglec-1). In certain embodiments, the mutation is a substitution corresponding to a substitution of I58D, Y60D, N114E, R124D, or D127R. In certain embodiments, the mutation is a substitution or deletion of an arginine residue at a position corresponding to position 116 of wild-type human Siglec-1 (e.g., R116). In certain embodiments, the Siglec ECD comprises or consists of SEQ ID NO: 18.

In certain embodiments, the Siglec ECD, or a functional fragment or variant thereof, is a human Siglec-2 ECD, and the mutation is present in the region from amino acid 110 to 135 of SEQ ID NO: 23 (wild-type human Siglec-2). In certain embodiments, the mutation is present in the region from amino acid 118 to 128 of SEQ ID NO: 23 (wild-type human Siglec-2). In certain embodiments, the mutation is present in the region from amino acid 117 to 120 of SEQ ID NO: 23 (wild-type human Siglec-2). In certain embodiments, the mutation is present at D113, G115, R120, E122, K127, W128, or L134 of SEQ ID NO: 23 (wild-type human Siglec-2). In certain embodiments, the mutation is present at G118, E126, W128, E130 of SEQ ID NO: 23 (wild-type human Siglec-2). In certain embodiments, the mutation is a substitution or deletion of an arginine residue at a position corresponding to position 120 of wild-type human Siglec-2 (R120), e.g., R120A or R120K.

In certain embodiments, the Siglec ECD, or a functional fragment or variant thereof, is a human Siglec-3 ECD, and the mutation is present in the region from amino acid 109 to 137 of SEQ ID NO: 25 (wild-type human Siglec-3). In certain embodiments, the mutation is present in the region from amino acid 117 to 128 of SEQ ID NO: 25 (wild-type human Siglec-3). In certain embodiments, the mutation is present in the region from amino acid 117 to 120 of SEQ ID NO: 25 (wild-type human Siglec-3). In certain embodiments, the mutation is present at D112, G114, Y116, F117, F118, R119, E121, R122, G123, K126, Y127, Y129, L134, or V136 of SEQ ID NO: 25 (wild-type human Siglec-3). In certain embodiments, the mutation is a substitution or deletion of an arginine residue at a position corresponding to position 119 of wild-type human Siglec-3 (R119), e.g., R119A or R119K. In certain embodiments, the Siglec ECD comprises or consists of SEQ ID NO: 30.

In certain embodiments, the Siglec ECD, or a functional fragment or variant thereof, is a human Siglec-4 ECD, and the mutation is present in the region from amino acid 108 to 133 of SEQ ID NO: 33 (wild-type human Siglec-4). In certain embodiments, the mutation is present in the region from amino acid 116 to 128 of SEQ ID NO: 33 (wild-type human Siglec-4). In certain embodiments, the mutation is present in the region from amino acid 115 to 118 of SEQ ID NO: 33 (wild-type human Siglec-4). In certain embodiments, the mutation is present at G113, Y115, F117, R118, G122, or Y127 of SEQ ID NO: 33 (wild-type human Siglec-4). In certain embodiments, the mutation is a substitution or deletion of an arginine residue at a position corresponding to position 118 of wild-type human Siglec-4 (R118), e.g., RT18A. In certain embodiments, the Siglec ECD comprises or consists of SEQ ID NO: 36.

In certain embodiments, the Siglec ECD, or a functional fragment or variant thereof, is a human Siglec-5 ECD, and the mutation is present in the region from amino acid 109 to 138 of SEQ ID NO: 41 (wild-type human Siglec-5). In certain embodiments, the mutation is present in the region from amino acid 117 to 129 of SEQ ID NO: 41 (wild-type human Siglec-5). In certain embodiments, the mutation is present in the region from amino acid 116-119 of SEQ ID NO: 41 (wild-type human Siglec-5). In certain embodiments, the mutation is present at D112, G114, Y116, F117, F118, R119, V120, E121, R122, G123, R124, V126, K127, Y128, Y130, or L135 of SEQ ID NO: 41 (wild-type human Siglec-5). In certain embodiments, the mutation is a substitution or deletion of an arginine residue at a position corresponding to position 124 of wild-type human Siglec-5 (R119), e.g., R119A.

In certain embodiments, the Siglec ECD, or a functional fragment or variant thereof, is a human Siglec-6 ECD, and the mutation is present in the region from amino acid 112 to 140 of SEQ ID NO: 43 (wild-type human Siglec-6). In certain embodiments, the mutation is present in the region from amino acid 120 to 131 of SEQ ID NO: 43 (wild-type human Siglec-6). In certain embodiments, the mutation is present in the region from amino acid 119-122 of SEQ ID NO: 43 (wild-type human Siglec-6). In certain embodiments, the mutation is present at D115, Y119, F120, F121, R122, K129, Y130, Y132, L137, or V139 of SEQ ID NO: 43 (wild-type human Siglec-6). In certain embodiments, the mutation is a substitution or deletion of an arginine residue at a position corresponding to position 122 of wild-type human Siglec-6 (R122), e.g., R122A, R122G, or R122K. In certain embodiments, the Siglec ECD comprises or consists of SEQ ID NO: 46 or SEQ ID NO: 156.

In certain embodiments, the Siglec ECD, or a functional fragment or variant thereof, is a human Siglec-7 ECD, and the mutation is present in the region from amino acid 114 to 142 of SEQ ID NO: 51 (wild-type human Siglec-7). In certain embodiments, the mutation is present in the region from amino acid 122 to 133 of SEQ ID NO: 51 (wild-type human Siglec-7). In certain embodiments, the mutation is present in the region from amino acid 121 to 124 of SEQ ID NO: 51 (wild-type human Siglec-7). In certain embodiments, the mutation is present at D117, G119, Y121, F122, F123, R124, E126, G128, K131, W132, Y134, L139, or V141 of SEQ ID NO: 51 (wild-type human Siglec-7). In certain embodiments, the mutation is a substitution or deletion of an arginine residue at a position corresponding to position 124 of wild-type human Siglec-7 (R124), e.g., R124A or R124K. In certain embodiments, the Siglec ECD comprises or consists of SEQ ID NO: 58.

In certain embodiments, the Siglec ECD, or a functional fragment or variant thereof, is a human Siglec-8 ECD, and the mutation is present in the region from amino acid 115 to 149 of SEQ ID NO: 63 (wild-type human Siglec-8). In certain embodiments, the mutation is present in the region from amino acid 123 to 140 of SEQ ID NO: 63 (wild-type human Siglec-8). In certain embodiments, the mutation is present in the region from amino acid 122 to 125 of SEQ ID NO: 63 (wild-type human Siglec-8). In certain embodiments, the mutation is present at D118, G120, Y122, F123, F124, R125, E127, R128, G129, K132, W133, Y141, L146, or V148 of SEQ ID NO: 63 (wild-type human Siglec-8). In certain embodiments, the mutation is a substitution or deletion of an arginine residue at a position corresponding to position 128 of wild-type human Siglec-8 (R128), e.g., R128A.

In certain embodiments, the Siglec ECD, or a functional fragment or variant thereof, is a human Siglec-9 ECD, and the mutation is present in the region from amino acid 110 to 138 of SEQ ID NO: 65 (wild-type human Siglec-9). In certain embodiments, the mutation is present in the region from amino acid 118 to 130 of SEQ ID NO: 65 (wild-type human Siglec-9). In certain embodiments, the mutation is present in the region from amino acid 117 to 120 of SEQ ID NO: 65 (wild-type human Siglec-9). In certain embodiments, the mutation is present at D113, G115, Y117, F118, F119, R120, E122, G124, K127, W128, Y130, L135, or V137 of SEQ ID NO: 65 (wild-type human Siglec-9). In certain embodiments, the mutation that reduces sialic acid binding is a substitution or deletion of an arginine residue at a position corresponding to position 120 of wild-type human Siglec-9 (R120), e.g., R120A or R120K. In certain embodiments, the Siglec ECD comprises or consists of SEQ ID NO: 72.

In certain embodiments, the Siglec ECD, or a functional fragment or variant thereof, is a human Siglec-10 ECD, and the mutation is present in the region from amino acid 109 to 138 of SEQ ID NO: 87 (wild-type human Siglec-10). In certain embodiments, the mutation is present in the region from amino acid 117 to 129 of SEQ ID NO: 87 (wild-type human Siglec-10). In certain embodiments, the mutation is present in the region from amino acid 116 to 119 of SEQ ID NO: 87 (wild-type human Siglec-10). In certain embodiments, the mutation is present at D112, Y116, F117, F118, R119, V120, E121, R122, G123, V126, or Y128 of SEQ ID NO: 87 (wild-type human Siglec-10). In certain embodiments, the mutation is a substitution or deletion of an arginine residue at a position corresponding to position 119 of wild-type human Siglec-10 (R119), e.g., R119E or R119A.

In certain embodiments, the Siglec ECD, or a functional fragment or variant thereof, is a human Siglec-11 ECD, and the mutation is present in the region from amino acid 122 to 151 of SEQ ID NO: 92 (wild-type human Siglec-11). In certain embodiments, the mutation is present in the region from amino acid 130 to 142 of SEQ ID NO: 92 (wild-type human Siglec-11). In certain embodiments, the mutation is present in the region from amino acid 129 to 132 of SEQ ID NO: 92 (wild-type human Siglec-11). In certain embodiments, the mutation is present at D125, Y129, F130, F131, R132, V133, E134, R135, G136, or V139 of SEQ ID NO: 92 (wild-type human Siglec-11). In certain embodiments, the mutation is a substitution or deletion of an arginine residue at a position corresponding to position 132 of wild-type human Siglec-11 (R132), e.g., R132A. In certain embodiments, the Siglec ECD comprises or consists of SEQ ID NO: 95.

In certain embodiments, the Siglec ECD, or a functional fragment or variant thereof, is a human Siglec-12 ECD, and the mutation is present in the region from amino acid 122 to 151 of SEQ ID NO: 100 (wild-type human Siglec-12). In certain embodiments, the mutation is present in the region from amino acid 120 to 131 of SEQ ID NO: 100 (wild-type human Siglec-12). In certain embodiments, the mutation is present in the region from amino acid 119 to 122 of SEQ ID NO: 100 (wild-type human Siglec-12). In certain embodiments, the mutation is present at D115, G117, Y119, F121, V123, E124, R125, G126, K129, W130, Y132, L137, or V139 of SEQ ID NO: 37 (wild-type human Siglec-12). In certain embodiments, the mutation is a substitution or deletion of an arginine residue at a position corresponding to position 125 of wild-type human Siglec-12 (R125), e.g., R125A.

In certain embodiments, the Siglec ECD, or a functional fragment or variant thereof, is a human Siglec-14 ECD, and the mutation is present in the region from amino acid 109 to 138 of SEQ ID NO: 102 (wild-type human Siglec-14). In certain embodiments, the mutation is present in the region from amino acid 117 to 129 of SEQ ID NO: 102 (wild-type human Siglec-14). In certain embodiments, the mutation is present in the region from amino acid 116 to 119 of SEQ ID NO: 102 (wild-type human Siglec-14). In certain embodiments, the mutation is present at D112, G114, Y116, F117, F1l18, R119, V120, E121, R122, G123, V126, K127, Y128, Y130, or L135 of SEQ ID NO: 102 (wild-type human Siglec-14). In certain embodiments, the mutation is a substitution or deletion of an arginine residue at a position corresponding to position 119 of wild-type human Siglec-14 (R119), e.g., RT19A.

In certain embodiments, the Siglec ECD, or a functional fragment or variant thereof, is a human Siglec-15 ECD, and the mutation is present in the region from amino acid 133 to 161 of SEQ ID NO: 104 (wild-type human Siglec-15). In certain embodiments, the mutation is present in the region from amino acid 141 to 153 of SEQ ID NO: 104 (wild-type human Siglec-15). In certain embodiments, the mutation is present in the region from amino acid 140 to 143 of SEQ ID NO: 104 (wild-type human Siglec-15). In certain embodiments, the mutation is present at D136, Y140, F141, R143, V144, E145, V150, or Y154 of SEQ ID NO: 104 (wild-type human Siglec-15). In certain embodiments, the mutation is a substitution or deletion of an arginine residue at a position corresponding to position 143 of wild-type human Siglec-15 (R143), e.g., R143A. In certain embodiments, the Siglec ECD comprises or consists of SEQ ID NO: 107.

In certain embodiments, the Siglec ECD, or a functional fragment or variant thereof, is a human Siglec-16 ECD, and the mutation is present in the region from amino acid 110 to 139 of SEQ ID NO: 114 or SEQ ID NO: 138 (wild-type human Siglec-16). In certain embodiments, the mutation is present in the region from amino acid 118 to 130 of SEQ ID NO: 114 or SEQ ID NO: 138 (wild-type human Siglec-16). In certain embodiments, the mutation is present in the region from amino acid 117 to 120 of SEQ ID NO: 114 or SEQ ID NO: 138 (wild-type human Siglec-16). In certain embodiments, the mutation is present at D113, Y117, F118, F119, R120, V121, E122, R123, G124, or V127 of SEQ ID NO: 114 or SEQ ID NO: 138 (wild-type human Siglec-16). In certain embodiments, the mutation is a substitution or deletion of an arginine residue at a position corresponding to position 120 of wild-type human Siglec-16 (R120), e.g., R120A.

### II. Serum Half-Life Enhancer

As used herein, a “serum half-life enhancer” refers to a moiety that can be associated with (e.g., conjugated to) a Siglec extracellular domain (ECD), or a functional fragment or variant thereof, to enhance its circulating half-life in the serum of a subject. In certain embodiments, a serum half-life enhancer can be selected from an Fc domain (see, e.g., Beck et al. (2011) MABS 4:1015-28), albumin (e.g., human serum albumin (HSA), see, Weimer et al. (2013) Recombinant albumin fusion proteins. In: Schmidt S, editor. Fusion protein technologies for biopharmaceuticals: applications and challenges. Hoboken: Wiley; 2013, p. 297-323), albumin binding domain (e.g., an HSA binder, see Walker et al. (2013) Albumin-binding fusion proteins in the development of novel long-acting therapeutics. In: Schmidt S, editor. Fusion protein technologies for biopharmaceuticals: applications and challenges. Hoboken: Wiley; 2013, p. 325-43), transferrin (see Kim et al. (2010) J. PHARMACOL. EXP. THER. 334:682-92), XTEN (also called recombinant PEG or “rPEG”, see Schellenberger et al. (2009) NAT. BIOTECHNOL. 27:1186-90), a homo-amino acid polymer (HAP, see Schlapschy et al. (2007) PROTEIN ENG. DES. SEL. 20:273-84)), a proline-alanine-serine polymer (PAS, see Schlapschy et al. (2013) PROTEIN ENG. DES. SEL. 26:489-501), an elastin-like peptide (ELP, see Floss et al. (2013) Fusion protein technologies for biopharmaceuticals: applications and challenges, p. 372-98), carboxy-terminal peptide (CTP, Duijkers et al. (2002) HUM. REPROD. 17:1987-93)), gelatin-like protein (GLK, Huang et al. (2010) EUR. J. PHARM. BIOPHARM. 72:435-41), and a polyethylene glycol (PEG).

Suitable serum half-life enhancers also include a variety of polymers, such as those described in U.S. Pat. No. 7,842,789. For example, block copolymers of polyoxyethylene and polyoxypropylene (Pluronics); polymethacrylates; carbomers; and branched or unbranched polysaccharides which comprise the saccharide monomers such as D-mannose, D- and L-galactose, fucose, fructose, D-xylose, L-arabinose, and D-glucuronic acid can be used. In other embodiments, the serum half-life enhancer can be a hydrophilic polyvinyl polymer such as polyvinyl alcohol and polyvinylpyrrolidone (PVP)-type polymers. The serum half-life enhancer can be a functionalized polyvinylpyrrolidone, for example, carboxy or amine functionalized on one (or both) ends of the polymer (as available from PolymerSource). Alternatively, the serum half-life enhancer can include Poly N-(2-hydroxypropyl)methacrylamide (HPMA), or functionalized HPMA (amine, carboxy, etc.), Poly(N-isopropylacrylamide) or functionalized poly(N-isopropylacrylamide).

In one embodiment, a Siglec extracellular domain (ECD), or a functional fragment or variant thereof, is covalently attached to a naturally long-half-life polypeptide or protein such as an Fe domain (Beck et al., supra), transferrin (Kim et al., supra), or albumin (Weimer et al., supra) to form a fusion protein, either by genetic fusion (i.e., production of recombinant fusion protein) or by chemical conjugation.

In another embodiment, a Siglec extracellular domain (ECD), or a functional fragment or variant thereof, is covalently attached to an inert polypeptide such as an XTEN (also called recombinant PEG or “rPEG”, see Schellenberger, supra), a homo amino acid polymer (HAP, see Schlapschy et al. (2007), supra), a proline-alanine-serine-polymer (PAS, see Schlapschy et al., (2013), supra), an elastin-like peptide (ELP, see Floss et al., supra), or gelatin-like protein (GLK, Huang et al., supra) to form a fusion protein, either by genetic fusion (i.e., production of recombinant fusion protein) or by chemical conjugation. Inert polypeptides function, among other things, to increase the size and hydrodynamic radius of a Siglec extracellular domain (ECD), or a functional fragment or variant thereof, thereby to extend half-life. In certain embodiments, an XTEN polypeptide has a length from about 25 amino acids to about 1500 amino acids (e.g., from about 25 amino acids to about 100 amino acids, from about 25 amino acids to about 250 amino acids, from about 25 amino acids to about 500 amino acids, from about 25 amino acids to about 750 amino acids, from about 25 amino acids to about 1,000 amino acids, from about 25 amino acids to about 1250 amino acids, from about 100 amino acids to about 250 amino acids, from about 100 amino acids to about 250 amino acids, from about 100 amino acids to about 500 amino acids, from about 100 amino acids to about 750 amino acids, from about 100 amino acids to about 1,000 amino acids, from about 100 amino acids to about 1250 amino acids, from about 100 amino acids to about 1,500 amino acids, from about 250 amino acids to about 1250 amino acids, from about 250 amino acids to about 1,000 amino acids, from about 250 amino acids to about 750 amino acids, from about 250 amino acids to about 500 amino acids, from about 500 amino acids to about 750 amino acids, from about 500 amino acids to about 1000 amino acids, from about 500 amino acids to about 1,250 amino acids, from about 500 amino acids to about 1,500 amino acids, from about 750 amino acids to about 1000 amino acids, from about 750 amino acids to about 1250 amino acids, from about 750 amino acids to about 1500 amino acids, from about 1,000 amino acids to about 1,250 amino acids, from about 1000 amino acids to about 1,500 amino acids, or from about 1,250 amino acids to about 1,500 amino acids.

In certain embodiments, a Siglec extracellular domain (ECD), or a functional fragment or variant thereof, is chemically conjugated to a repeat chemical moiety such as PEG or hyaluronic acid (see, Mero et al. (2013) CARB. POLYMERS 92:2163-70), which increases the hydrodynamic radius of the Siglec extracellular domain (ECD), or the functional fragment or variant thereof, thereby to extend half-life. Alternatively, a Siglec extracellular domain (ECD), or a functional fragment or variant thereof, is itself polysialylated or covalently attached to a negatively charged, highly sialylated protein (e.g., carboxy-terminal peptide (CTP), of chorionic gonadotropin (CG) β-chain, see, Duijkers et al. (2002) HUM REPROD 17:1987-93).

In certain embodiments, a Siglec extracellular domain (ECD), or a functional fragment or variant thereof, is conjugated to a serum half-life enhancer that is not and Fc domain and/or is not PEG.

In general, the serum half-life enhancer may have a molecular weight from about 2 kDa to about 5 kDa, from about 2 kDa to about 10 kDa, from about 2 kDa to about 20 kDa, from about 2 kDa to about 30 kDa, from about 2 kDa to about 40 kDa, from about 2 kDa to about 50 kDa, from about 2 kDa to about 60 kDa, from about 2 kDa to about 70 kDa, from about 2 kDa to about 80 kDa, from about 2 kDa to about 90 kDa, from about 2 kDa to about 100 kDa, from about 2 kDa to about 150 kDa, from about 5 kDa to about 10 kDa, from about 5 kDa to about 20 kDa, from about 5 kDa to about 30 kDa, from about 5 kDa to about 40 kDa, from about 5 kDa to about 50 kDa, from about 5 kDa to about 60 kDa, from about 5 kDa to about 70 kDa, from about 5 kDa to about 80 kDa, from about 5 kDa to about 90 kDa, from about 5 kDa to about 100 kDa, from about 5 kDa to about 150 kDa, from about 10 kDa to about 20 kDa, from about 10 kDa to about 30 kDa, from about 10 kDa to about 40 kDa, from about 10 kDa to about 50 kDa, from about 10 kDa to about 60 kDa, from about 10 kDa to about 70 kDa, from about 10 kDa to about 80 kDa, from about 10 kDa to about 90 kDa, from about 10 kDa to about 100 kDa, from about 10 kDa to about 150 kDa, from about 20 kDa to about 30 kDa, from about 20 kDa to about 40 kDa, from about 20 kDa to about 50 kDa, from about 20 kDa to about 60 kDa, from about 20 kDa to about 70 kDa, from about 20 kDa to about 80 kDa, from about 20 kDa to about 90 kDa, from about 20 kDa to about 100 kDa, from about 20 kDa to about 150 kDa, from about 30 kDa to about 40 kDa, from about 30 kDa to about 50 kDa, from about 30 kDa to about 60 kDa, from about 30 kDa to about 70 kDa, from about 30 kDa to about 80 kDa, from about 30 kDa to about 90 kDa, from about 30 kDa to about 100 kDa, from about 30 kDa to about 150 kDa, from about 40 kDa to about 50 kDa, from about 40 kDa to about 60 kDa, from about 40 kDa to about 70 kDa, from about 40 kDa to about 80 kDa, from about 40 kDa to about 90 kDa, from about 40 kDa to about 100 kDa, from about 40 kDa to about 150 kDa, from about 50 kDa to about 60 kDa, from about 50 kDa to about 70 kDa, from about 50 kDa to about 80 kDa, from about 50 kDa to about 90 kDa, from about 50 kDa to about 100 kDa, from about 50 kDa to about 150 kDa, from about 60 kDa to about 70 kDa, from about 60 kDa to about 80 kDa, from about 60 kDa to about 90 kDa, from about 60 kDa to about 100 kDa, from about 60 kDa to about 150 kDa, from about 70 kDa to about 80 kDa, from about 70 kDa to about 90 kDa, from about 70 kDa to about 100 kDa, from about 70 kDa to about 150 kDa, from about 80 kDa to about 90 kDa, from about 80 kDa to about 100 kDa, from about 80 kDa to about 150 kDa, from about 90 kDa to about 100 kDa, from about 90 kDa to about 150 kDa, or from about 100 kDa to about 150 kDa.

Methods for making and using the foregoing serum half-life enhancers are known in the art. See also, e.g., Strohl (2015) BIODRUGS 29:215-239. It is contemplated that one or more Siglec extracellular domain (ECD), or functional fragments or variants thereof, may be covalently bound to one or more (for example, 2, 3, 4, 5, 6, 8, 9, 10 or more) serum half-life enhancers.

In certain embodiments, the serum half-life of a Siglec extracellular domain (ECD), or a functional fragment or variant thereof, conjugated to a serum half-life enhancer is at least 24, 36, 48, or 60 hours.

In certain embodiments, an XTEN polypeptide is linked, e.g., conjugated, to a Siglec extracellular domain (ECD), or a functional fragment or variant thereof. In certain embodiments, an XTEN polypeptide is linked, e.g., conjugated, to a Siglec-1 ECD, or a functional fragment or variant thereof. In certain embodiments, an XTEN polypeptide is linked, e.g., conjugated, to a Siglec-2 ECD, or a functional fragment or variant thereof. In certain embodiments, an XTEN polypeptide is linked, e.g., conjugated, to a Siglec-3 ECD, or a functional fragment or variant thereof. In certain embodiments, an XTEN polypeptide is linked, e.g., conjugated, to a Siglec-4 ECD, or a functional fragment or variant thereof. In certain embodiments, an XTEN polypeptide is linked, e.g., conjugated, to a Siglec-5 ECD, or a functional fragment or variant thereof. In certain embodiments, an XTEN polypeptide is linked, e.g., conjugated, to a Siglec-6 ECD, or a functional fragment or variant thereof. In certain embodiments, an XTEN polypeptide is linked, e.g., conjugated, to a Siglec-7 ECD, or a functional fragment or variant thereof. In certain embodiments, XTEN polypeptide is linked, e.g., conjugated, to a Siglec-8 ECD, or a functional fragment or variant thereof. In certain embodiments, an XTEN polypeptide is linked, e.g., conjugated, to a Siglec-9 ECD, or a functional fragment or variant thereof. In certain embodiments, an XTEN polypeptide is linked, e.g., conjugated, to a Siglec-10 ECD, or a functional fragment or variant thereof. In certain embodiments, XTEN polypeptide is linked, e.g., conjugated, to a Siglec-11 ECD, or a functional fragment or variant thereof. In certain embodiments, an XTEN polypeptide is linked, e.g., conjugated, to a Siglec-12 ECD, or a functional fragment or variant thereof. In certain embodiments, an XTEN polypeptide is linked, e.g., conjugated, to a Siglec-14 ECD, or a functional fragment or variant thereof. In certain embodiments, an XTEN polypeptide is linked, e.g., conjugated, to a Siglec-15 ECD, or a functional fragment or variant thereof. In certain embodiments, an XTEN polypeptide is linked, e.g., conjugated, to a Siglec-16 ECD, or a functional fragment or variant thereof.

In certain embodiments, a homo-amino acid polymer (HAP) is linked, e.g., conjugated, to a Siglec extracellular domain (ECD), or a functional fragment or variant thereof. In certain embodiments, a HAP is linked, e.g., conjugated, to a Siglec-1 domain ECD, or a functional fragment or variant thereof. In certain embodiments, a HAP is linked, e.g., conjugated, to a Siglec-2 ECD, or a functional fragment or variant thereof. In certain embodiments, a HAP is linked, e.g., conjugated, to a Siglec-3 ECD, or a functional fragment or variant thereof. In certain embodiments, a HAP is linked, e.g., conjugated, to a Siglec-4 ECD, or a functional fragment or variant thereof. In certain embodiments, a HAP is linked, e.g., conjugated, to a Siglec-5 ECD, or a functional fragment or variant thereof. In certain embodiments, a HAP is linked, e.g., conjugated, to a Siglec-6 ECD, or a functional fragment or variant thereof. In certain embodiments, a HAP is linked, e.g., conjugated, to a Siglec-7 ECD, or a functional fragment or variant thereof. In certain embodiments, a HAP is linked, e.g., conjugated, to a Siglec-8 ECD, or a functional fragment or variant thereof. In certain embodiments, a HAP is linked, e.g., conjugated, to a Siglec-9 ECD, or a functional fragment or variant thereof. In certain embodiments, a HAP is linked, e.g., conjugated, to a Siglec-10 ECD, or a functional fragment or variant thereof. In certain embodiments, a HAP is linked, e.g., conjugated, to a Siglec-11 ECD, or a functional fragment or variant thereof. In certain embodiments, a HAP is linked, e.g., conjugated, to a Siglec-12 ECD, or a functional fragment or variant thereof. In certain embodiments, a HAP is linked, e.g., conjugated, to a Siglec-14 ECD, or a functional fragment or variant thereof. In certain embodiments, a HAP is linked, e.g., conjugated, to a Siglec-15 ECD, or a functional fragment or variant thereof. In certain embodiments, a HAP is linked, e.g., conjugated, to a Siglec-16 ECD, or a functional fragment or variant thereof.

In certain embodiments, an elastin-like polypeptide (ELP) is linked, e.g., conjugated, to a Siglec extracellular domain (ECD) or a functional fragment or variant thereof. In certain embodiments, an ELP is linked, e.g., conjugated, to a Siglec-1 ECD, or a functional fragment or variant thereof. In certain embodiments, an ELP is linked, e.g., conjugated, to a Siglec-2 ECD, or a functional fragment or variant thereof. In certain embodiments, an ELP is linked, e.g., conjugated, to a Siglec-3 ECD, or a functional fragment or variant thereof. In certain embodiments, an ELP is linked, e.g., conjugated, to a Siglec-4 ECD, or a functional fragment or variant thereof. In certain embodiments, an ELP is linked, e.g., conjugated, to a Siglec-5 ECD, or a functional fragment or variant thereof. In certain embodiments, an ELP is linked, e.g., conjugated, to a Siglec-6 ECD, or a functional fragment or variant thereof. In certain embodiments, an ELP is linked, e.g., conjugated, to a Siglec-7 ECD, or a functional fragment or variant thereof. In certain embodiments, an ELP is linked, e.g., conjugated, to a Siglec-8 ECD, or a functional fragment or variant thereof. In certain embodiments, an ELP is linked, e.g., conjugated, to a Siglec-9 ECD, or a functional fragment or variant thereof. In certain embodiments, an ELP is linked, e.g., conjugated, to a Siglec-10 ECD, or a functional fragment or variant thereof. In certain embodiments, an ELP is linked, e.g., conjugated, to a Siglec-11 ECD, or a functional fragment or variant thereof. In certain embodiments, an ELP is linked, e.g., conjugated, to a Siglec-12 ECD, or a functional fragment or variant thereof. In certain embodiments, an ELP is linked, e.g., conjugated, to a Siglec-14 ECD, or a functional fragment or variant thereof. In certain embodiments, an ELP is linked, e.g., conjugated, to a Siglec-15 ECD, or a functional fragment or variant thereof. In certain embodiments, an ELP is linked, e.g., conjugated, to a Siglec-16 ECD, or a functional fragment or variant thereof.

In certain embodiments, a proline-alanine-serine (PAS) polypeptide is linked, e.g., conjugated, to a Siglec extracellular domain (ECD) or a functional fragment or variant thereof. In certain embodiments, a PAS polypeptide is linked, e.g., conjugated, to a Siglec-1 ECD, or a functional fragment or variant thereof. In certain embodiments, a PAS polypeptide is linked, e.g., conjugated, to a Siglec-2 ECD, or a functional fragment or variant thereof. In certain embodiments, a PAS polypeptide is linked, e.g., conjugated, to a Siglec-3 ECD, or a functional fragment or variant thereof. In certain embodiments, a PAS polypeptide is linked, e.g., conjugated, to a Siglec-4 ECD, or a functional fragment or variant thereof. In certain embodiments, a PAS polypeptide is linked, e.g., conjugated, to a Siglec-5 ECD, or a functional fragment or variant thereof. In certain embodiments, a PAS polypeptide is linked, e.g., conjugated, to a Siglec-6 ECD, or a functional fragment or variant thereof. In certain embodiments, a PAS polypeptide is linked, e.g., conjugated, to a Siglec-7 ECD, or a functional fragment or variant thereof. In certain embodiments, a PAS polypeptide is linked, e.g., conjugated, to a Siglec-8 ECD, or a functional fragment or variant thereof. In certain embodiments, a PAS polypeptide is linked, e.g., conjugated, to a Siglec-9 ECD, or a functional fragment or variant thereof. In certain embodiments, a PAS polypeptide is linked, e.g., conjugated, to a Siglec-10 ECD, or a functional fragment or variant thereof. In certain embodiments, a PAS polypeptide is linked, e.g., conjugated, to a Siglec-11 ECD, or a functional fragment or variant thereof. In certain embodiments, a PAS polypeptide is linked, e.g., conjugated, to a Siglec-12 ECD, or a functional fragment or variant thereof. In certain embodiments, a PAS polypeptide is linked, e.g., conjugated, to a Siglec-14 ECD, or a functional fragment or variant thereof. In certain embodiments, a PAS polypeptide is linked, e.g., conjugated, to a Siglec-15 ECD, or a functional fragment or variant thereof. In certain embodiments, a PAS polypeptide is linked, e.g., conjugated, to a Siglec-16 ECD, or a functional fragment or variant thereof.

In certain embodiments, a gelatin-like protein (GLK) polymer is linked, e.g., conjugated, to a Siglec extracellular domain (ECD) or a functional fragment or variant thereof. In certain embodiments, a GLK polymer is linked, e.g., conjugated, to a Siglec-1 ECD, or a functional fragment or variant thereof. In certain embodiments, a GLK polymer is linked, e.g., conjugated, to a Siglec-2 ECD, or a functional fragment or variant thereof. In certain embodiments, a GLK polymer is linked, e.g., conjugated, to a Siglec-3 ECD, or a functional fragment or variant thereof. In certain embodiments, a GLK polymer is linked, e.g., conjugated, to a Siglec-4 ECD, or a functional fragment or variant thereof. In certain embodiments, a GLK polymer is linked, e.g., conjugated, to a Siglec-5 ECD, or a functional fragment or variant thereof. In certain embodiments, a GLK polymer is linked, e.g., conjugated, to a Siglec-6 ECD, or a functional fragment or variant thereof. In certain embodiments, a GLK polymer is linked, e.g., conjugated, to a Siglec-7 ECD, or a functional fragment or variant thereof. In certain embodiments, a GLK polymer is linked, e.g., conjugated, to a Siglec-8 ECD, or a functional fragment or variant thereof. In certain embodiments, a GLK polymer is linked, e.g., conjugated, to a Siglec-9 ECD, or a functional fragment or variant thereof. In certain embodiments, a GLK polymer is linked, e.g., conjugated, to a Siglec-10 ECD, or a functional fragment or variant thereof. In certain embodiments, a GLK polymer is linked, e.g., conjugated, to a Siglec-11 ECD, or a functional fragment or variant thereof. In certain embodiments, a GLK polymer is linked, e.g., conjugated, to a Siglec-12 ECD, or a functional fragment or variant thereof. In certain embodiments, a GLK polymer is linked, e.g., conjugated, to a Siglec-14 ECD, or a functional fragment or variant thereof. In certain embodiments, a GLK polymer is linked, e.g., conjugated, to a Siglec-15 ECD, or a functional fragment or variant thereof. In certain embodiments, a GLK polymer is linked, e.g., conjugated, to a Siglec-16 ECD, or a functional fragment or variant thereof.

In certain embodiments, a carboxy-terminal peptide (CTP) of chorionic gonadotropin (CG) β-chain is linked, e.g., covalently bound, to a Siglec extracellular domain (ECD) or a functional fragment or variant thereof. In certain embodiments, a CTP is linked, e.g., covalently bound, to a Siglec-1 ECD, or a functional fragment or variant thereof. In certain embodiments, a CTP is linked, e.g., covalently bound, to a Siglec-2 ECD, or a functional fragment or variant thereof. In certain embodiments, a CTP is linked, e.g., covalently bound, to a Siglec-3 ECD, or a functional fragment or variant thereof. In certain embodiments, a CTP is linked, e.g., covalently bound, to a Siglec-4 ECD, or a functional fragment or variant thereof. In certain embodiments, a CTP is linked, e.g., covalently bound, to a Siglec-5 ECD, or a functional fragment or variant thereof. In certain embodiments, a CTP is linked, e.g., covalently bound to a Siglec-6 ECD, or a functional fragment or variant thereof. In certain embodiments, a CTP is linked, e.g., covalently bound, to a Siglec-7 ECD, or a functional fragment or variant thereof. In certain embodiments, a CTP is linked, e.g., covalently bound, to a Siglec-8 ECD, or a functional fragment or variant thereof. In certain embodiments, a CTP is linked, e.g., covalently bound, to a Siglec-9 ECD, or a functional fragment or variant thereof. In certain embodiments, a CTP is linked, e.g., covalently bound, to a Siglec-10 ECD, or a functional fragment or variant thereof. In certain embodiments, a CTP is linked, e.g., covalently bound, to a Siglec-11 ECD, or a functional fragment or variant thereof. In certain embodiments, a CTP is linked, e.g., covalently bound, to a Siglec-12 ECD, or a functional fragment or variant thereof. In certain embodiments, a CTP is linked, e.g., covalently bound, to a Siglec-14 ECD, or a functional fragment or variant thereof. In certain embodiments, a CTP is linked, e.g., covalently bound, to a Siglec-15 ECD, or a functional fragment or variant thereof. In certain embodiments, a CTP is linked, e.g., covalently bound, to a Siglec-16 ECD, or a functional fragment or variant thereof.

a. Fc Domains

In certain embodiments, the serum half-life enhancer comprises an immunoglobulin Fe domain. As used herein, unless otherwise indicated, the term “immunoglobulin Fe domain” or “Fe domain” or “Fc” refers to a fragment of an immunoglobulin heavy chain constant region which, either alone or in combination with a second immunoglobulin Fe domain, or unconjugated or conjugated to a Siglec extracellular domain (ECD), or a functional fragment or variant thereof, is capable of binding to an Fc receptor. An immunoglobulin Fe domain may include, e.g., immunoglobulin CH2 and CH3 domains. An immunoglobulin Fe domain may include, e.g., immunoglobulin CH2 and CH3 domains and an immunoglobulin hinge region. For example, as used herein, an “Fc” or “Fe domain” can refer to a polypeptide comprising a CH2 domain, a CH3 domain, and optionally a hinge or a portion thereof. This polypeptide can bind (e.g., dimerize) to another polypeptide comprising a CH2 domain, a CH3 domain, and optionally a hinge or a portion thereof, wherein the dimer is capable of binding to an Fc receptor. Boundaries between immunoglobulin hinge regions, CH2, and CH3 domains are well known in the art, and can be found, e.g., in the PROSITE database (available on the world wide web at prosite.expasy.org).

In certain embodiments, the immunoglobulin Fe domain is derived from a human IgG1, IgG2, IgG3, IgG4, IgA1, IgA2, IgD, IgE, and IgM Fe domain. A single amino acid substitution (S228P according to Kabat numbering; designated IgG4Pro) may be introduced to abolish the heterogeneity observed in recombinant IgG4 antibody. See Angal, S. et al. (1993) MOL. IMMUNOL. 30:105-108.

Depending upon the circumstances, the immunoglobulin Fe domain can be derived from a human IgG1 isotype or another isotype that elicits antibody-dependent cell-mediated cytotoxicity (ADCC) and/or complement mediated cytotoxicity (CDC). In certain embodiments, the immunoglobulin Fe domain is derived from a human IgG1 isotype (e.g., SEQ ID NO: 1 and SEQ ID NO: 5). Alternatively, the immunoglobulin Fe domain can be derived from a human IgG4 isotype or another isotype that elicits little or no antibody-dependent cell-mediated cytotoxicity (ADCC) and/or complement mediated cytotoxicity (CDC). In certain embodiments, the immunoglobulin Fe domain is derived from a human IgG4 isotype.

In certain embodiments where a Siglec extracellular domain (ECD), or a functional fragment or variant thereof, is conjugated to an Fe domain, the Fe domain can have a “knob-into-hole” type format. The “knob” part is engineered by replacing a small amino acid with a larger one, which fits into a “hole”, which is engineered by replacing a large amino acid with a smaller one. The “knob-into-hole” format enhances heterodimer formation but doesn't suppress homodimer formation. Several approaches to promote heterodimerization have been described, for example in International (PCT) Publication Nos. WO96/27011, WO98/050431, WO2007/110205, WO2007/147901, WO2009/089004, WO2010/129304, WO2011/90754, WO2011/143545, WO2012/058768, WO2013/157954, and WO2013/096291, and European Patent Publication No. EP1870459. Typically, in the approaches known in the art, the CH3 domain of the first heavy chain and the CH3 domain of the second heavy chain are both engineered in a complementary manner so that the heavy chain comprising one engineered CH3 domain can no longer homodimerize with another heavy chain of the same structure (e.g. a CH3-engineered first heavy chain can no longer homodimerize with another CH3-engineered first heavy chain; and a CH3-engineered second heavy chain can no longer homodimerize with another CH3-engineered second heavy chain). Thereby the heavy chain comprising one engineered CH3 domain is forced to heterodimerize with another heavy chain comprising the CH3 domain, which is engineered in a complementary manner. As a result, the CH3 domain of the first heavy chain and the CH3 domain of the second heavy chain are engineered in a complementary manner by amino acid substitutions, such that the first heavy chain and the second heavy chain are forced to heterodimerize, whereas the first heavy chain and the second heavy chain can no longer homodimerize (e.g., for steric reasons).

Depending upon the certain circumstances, e.g., when the molecule is a heterodimer, the immunoglobulin Fc domain comprises either a “knob” mutation, e.g., T366Y, or a “hole” mutation, e.g., Y407T, for heterodimerization with a second polypeptide (residue numbers according to EU numbering, Kabat, E. A., et al. (1991) SEQUENCES OF PROTEINS OF IMMUNOLOGICAL INTEREST, FIFTH EDITION, U.S. Department of Health and Human Services, NIH Publication No. 91-3242). For example, in certain constructs, the immunoglobulin Fc domain is derived from a human IgG1 Fc domain and comprises a Y407T mutation (e.g., the Fc domain comprises SEQ ID NO: 2 and/or SEQ ID NO: 7). In certain other constructs, the immunoglobulin Fc domain is derived from a human IgG1 Fc domain and comprises a T366Y mutation (e.g., the Fe domain comprises SEQ ID NO: 3 and/or SEQ ID NO: 6).

In addition, it is understood that the immunoglobulin Fe domain can be modified to prevent to glycosylation of the Fe domain. For example, in certain constructs, the immunoglobulin Fe domain is derived from a human IgG1 Fe domain and comprises a mutation to prevent glycosylation, for example, a mutation at position N297, for example, an N297A or N297G mutation (residue numbers according to EU numbering, Kabat, E. A., et al., supra). For example, in certain constructs, the Fe domain comprises SEQ ID NO: 4 and/or SEQ ID NO: 8.

In certain embodiments, an immunoglobulin Fe domain comprises a modified hinge. For example, in certain embodiments, the Fe domain is derived from a human IgG1 Fe domain and comprises a mutation at, e.g., C220. In certain embodiments, the Fe domain comprises a C220S mutation (residue numbers according to EU numbering, Kabat, E. A., et al., supra). In certain embodiments, the Fe domain comprises SEQ ID NO: 12. In certain embodiments, the Fe domain comprises SEQ ID NO: 1 and SEQ ID NO: 12, wherein SEQ ID NO: 12 is fused to the N-terminus of SEQ ID NO: 1. In certain embodiments, the Fe domain comprises SEQ ID NO: 2 and SEQ ID NO: 12, wherein SEQ ID NO: 12 is fused to the N-terminus of SEQ ID NO: 2. In certain embodiments, the Fe domain comprises SEQ ID NO: 3 and SEQ ID NO: 12, wherein SEQ ID NO: 12 is fused to the N-terminus of SEQ ID NO: 3. In certain embodiments, the Fe domain comprises SEQ ID NO: 4 and SEQ ID NO: 12, wherein SEQ ID NO: 12 is fused to the N-terminus of SEQ ID NO: 4. In certain embodiments, the Fe domain comprises SEQ ID NO: 160.

In certain embodiments, an Fe domain is linked, e.g., conjugated, to a Siglec extracellular domain (ECD) or a functional fragment or variant thereof. In certain embodiments, an Fe domain is linked, e.g., conjugated, to a Siglec-1 ECD, or a functional fragment or variant thereof. In certain embodiments, an Fe domain is linked, e.g., conjugated, to a Siglec-2 ECD, or a functional fragment or variant thereof. In certain embodiments, an Fe domain is linked, e.g., conjugated, to a Siglec-3 ECD, or a functional fragment or variant thereof. In certain embodiments, an Fe domain is linked, e.g., conjugated, to a Siglec-4 ECD, or a functional fragment or variant thereof. In certain embodiments, an Fe domain is linked, e.g., conjugated, to a Siglec-5 ECD, or a functional fragment or variant thereof. In certain embodiments, an Fe domain is linked, e.g., conjugated, to a Siglec-6 ECD, or a functional fragment or variant thereof. In certain embodiments, an Fe domain is linked, e.g., conjugated, to a Siglec-7 ECD, or a functional fragment or variant thereof. In certain embodiments, an Fe domain is linked, e.g., conjugated, to a Siglec-8 ECD, or a functional fragment or variant thereof. In certain embodiments, an Fe domain is linked, e.g., conjugated, to a Siglec-9 ECD, or a functional fragment or variant thereof. In certain embodiments, an Fe domain is linked, e.g., conjugated, to a Siglec-10 ECD, or a functional fragment or variant thereof. In certain embodiments, an Fe domain is linked, e.g., conjugated, to a Siglec-11 ECD, or a functional fragment or variant thereof. In certain embodiments, an Fe domain is linked, e.g., conjugated, to a Siglec-12 ECD, or a functional fragment or variant thereof. In certain embodiments, an Fe domain is linked, e.g., conjugated, to a Siglec-14 ECD, or a functional fragment or variant thereof. In certain embodiments, an Fe domain is linked, e.g., conjugated, to a Siglec-15 ECD, or a functional fragment or variant thereof. In certain embodiments, an Fe domain is linked, e.g., conjugated, to a Siglec-16 ECD, or a functional fragment or variant thereof.

b. Polyethylene Glycol (PEG)

In addition or in the alternative, the serum half-life enhancer can be polyethylene glycol (PEG) or derivative thereof (e.g., alkoxy polyethylene glycol, for example, methoxypolyethylene glycol, ethoxypolyethylene glycol and the like). In one embodiment, the Siglec extracellular domain (ECD), or a functional fragment or variant thereof, as described herein, can be covalently attached to at least one PEG having an actual MW of at least about 20,000 D. In another embodiment, the Siglec extracellular domain (ECD), or a functional fragment or variant thereof, is covalently attached to at least one PEG having an actual MW of at least about 30,000 D. In another embodiment, the Siglec extracellular domain (ECD), or a functional fragment or variant thereof, is covalently attached to at least one PEG having an actual MW of at least about 40,000 D. In certain embodiments, the PEG is methoxyPEG(5000)-succinimidylpropionate (mPEG-SPA), methoxyPEG(5000)-succinimidylsuccinate (mPEG-SS). Such PEGS are commercially available from Nektar Therapeutics or SunBiowest or LaysanBio or NOF. It is contemplated that the PEG may be branched, or Y-shaped, as available from JenKem USA or NOF, or comb-shaped, or synthesized by coupling two or more PEGs to a small molecule such as glutamic acid.

The omega position of PEG may include a hydroxyl group or a methoxy group and the PEG may also contain an amino group in the omega position. Such an amino group can in turn be coupled to a variety of agents. In certain embodiments, the serum half-life enhancer can be a pegylated poly-L-lysine or a pegylated poly-D-lysine.

Attachment sites on a Siglec extracellular domain (ECD), or a functional fragment or variant thereof, for a PEG or a derivative thereof include the N-terminal amino group and epsilon amino groups found on lysine residues, as well as other amino, imino, carboxyl, sulfhydryl, hydroxyl or other hydrophilic groups. PEG may be covalently bonded directly to the Siglec extracellular domain (ECD), or a functional fragment or variant thereof, with or without the known use of a multifunctional (ordinarily bifunctional) crosslinking agent using chemistries and used in the art. For example, the PEG modifier can be conjugated to the Siglec extracellular domain (ECD), or a functional fragment or variant thereof, by using a thiol reactive cross linker and then reacting with a thiol group on the PEG. In certain embodiments, sulfhydryl groups can be derivatized by coupling to maleimido-substituted PEG (e.g., alkoxy-PEG amine plus sulfosuccinimidyl 4-(N-maleimidomethyl)cyclohexane-1-carboxylate), or PEG-maleimide commercially available from Shearwater Polymers, Inc., Huntsville, Ala.).

In certain embodiments, PEG or a derivative thereof is linked, e.g., covalently attached, to a Siglec extracellular domain, (ECD) or a functional fragment or variant thereof. In certain embodiments, PEG or a derivative thereof is linked, e.g., covalently attached, to a Siglec-1 ECD, or a functional fragment or variant thereof. In certain embodiments, PEG or a derivative thereof is linked, e.g., covalently attached, to a Siglec-2 ECD, or a functional fragment or variant thereof. In certain embodiments, PEG or a derivative thereof is linked, e.g., covalently attached, to a Siglec-3 ECD, or a functional fragment or variant thereof. In certain embodiments, PEG or a derivative thereof is linked, e.g., covalently attached, to a Siglec-4 ECD, or a functional fragment or variant thereof. In certain embodiments, PEG or a derivative thereof is linked, e.g., covalently attached, to a Siglec-5 ECD, or a functional fragment or variant thereof. In certain embodiments, PEG or a derivative thereof is linked, e.g., covalently attached, to a Siglec-6 ECD, or a functional fragment or variant thereof. In certain embodiments, PEG or a derivative thereof is linked, e.g., covalently attached, to a Siglec-7 ECD, or a functional fragment or variant thereof. In certain embodiments, PEG or a derivative thereof is linked, e.g., covalently attached, to a Siglec-8 ECD, or a functional fragment or variant thereof. In certain embodiments, PEG or a derivative thereof is linked, e.g., covalently attached, to a Siglec-9 ECD, or a functional fragment or variant thereof. In certain embodiments, PEG or a derivative thereof is linked, e.g., covalently attached, to a Siglec-10 ECD, or a functional fragment or variant thereof. In certain embodiments, PEG or a derivative thereof is linked, e.g., covalently attached, to a Siglec-11 ECD, or a functional fragment or variant thereof. In certain embodiments, PEG or a derivative thereof is linked, e.g., covalently attached, to a Siglec-12 ECD, or a functional fragment or variant thereof. In certain embodiments, PEG or a derivative thereof is linked, e.g., covalently attached, to a Siglec-14 ECD, or a functional fragment or variant thereof. In certain embodiments, PEG or a derivative thereof is linked, e.g., covalently attached, to a Siglec-15 ECD, or a functional fragment or variant thereof. In certain embodiments, PEG or a derivative thereof is linked, e.g., covalently attached, to a Siglec-16 ECD, or a functional fragment or variant thereof.

c. Human Serum Albumin (HSA) and HSA Binders

Human serum albumin (HSA) (molecular mass ˜67 kDa) is the most abundant protein in plasma, present at about 50 mg/mL (600 μM), and has a half-life of around 20 days in humans. HSA serves to maintain plasma pH, contributes to colloidal blood pressure, functions as carrier of many metabolites and fatty acids, and serves as a major drug transport protein in plasma.

In certain embodiments, the serum half-life enhancer can be human serum albumin (HSA) or an HSA-binding peptide (see, e.g., PCT Publication Nos. WO2013128027A1 and WO2014140358A1). The neonatal Fc receptor (FcRn) appears to be involved in prolonging the life-span of albumin in circulation (see, Chaudhury et al. (2003) J. EXP. MED., 3: 315-22). Albumin and IgG bind noncooperatively to distinct sites of FcRn and form a tri-molecular (see id.). Binding of human FcRn to HSA and to human IgG is pH dependent, stronger at acidic pH and weaker at neutral or physiological pH (see id.). This observation suggests that proteins and protein complexes containing albumin, similar to those containing IgG (particularly Fc), are protected from degradation through pH-sensitive interaction with FcRn (see id.). Using surface plasmon resonance (SPR) to measure the capacity of individual HSA domains to bind immobilized soluble human FcRn, it has been shown that FcRn and albumin interact via the D-III domain of albumin in a pH-dependent manner, on a site distinct from the IgG binding site (see, Chaudhury et al. (2006) BIOCHEM. 45:4983-90 and PCT Publication No. WO2008068280A1).

Exemplary HSA-binding proteins are known in the art. For example, U.S. Patent Application Publication No. US20130316952A1 discloses a polypeptide that binds serum albumin having the amino acid sequence of LKEAKEKAIEELKKAGITSDYYFDLINKAKTVEGVNALKDEILKA (SEQ ID NO: 119). In certain embodiments, the HSA-binding protein is an HSA-specific antibody, derivative, or HSA-binding fragment thereof. Additional exemplary polypeptides that bind HSA are described in U.S. Pat. Nos. 8,188,223, and 9,284,361, PCT Publication Nos. WO2017085172, and WO2018050833, and Dennis et al. (2002) J. BIOL. CHEM., 277: 35035-43; Jacobs et al. (2015) PROTEIN ENG. DES. SEL., 28: 385-93; and Zorzi et al. (2017) NAT. COMMUN., 8: 16092.

In certain embodiments, the half-life-extender can be an HSA-binding moiety. HSA has multiple fatty acid binding sites and, in certain embodiments, the HSA-binding moiety may be a fatty acid moiety that is conjugated or linked to the Siglec extracellular domain or fragment thereof (e.g., the Siglec extracellular domain or fragment thereof may be acylated or lipidated). Without wishing to be bound by theory, a linked fatty acid or lipid moiety is thought to enhance protein half-life by facilitating reversible binding to HSA. Methods of generating acylated proteins are known in the art including, e.g., as described in PCT Publication No. WO2000055119 and in U.S. Pat. No. 8,791,236.

In certain embodiments, HSA or an HSA-binding protein or HSA-binding moiety is linked, e.g., conjugated, to a Siglec extracellular domain (ECD), or a functional fragment or variant thereof. In certain embodiments, HSA or an HSA-binding protein or HSA-binding moiety is linked, e.g., conjugated, to a Siglec-1 ECD, or a functional fragment or variant thereof. In certain embodiments, HSA or an HSA-binding protein or HSA-binding moiety is linked, e.g., conjugated, to a Siglec-2 ECD, or a functional fragment or variant thereof. In certain embodiments, HSA or an HSA-binding protein or HSA-binding moiety is linked, e.g., conjugated, to a Siglec-3 ECD, or a functional fragment or variant thereof. In certain embodiments, HSA or an HSA-binding protein or HSA-binding moiety is linked, e.g., conjugated, to a Siglec-4 ECD, or a functional fragment or variant thereof. In certain embodiments, HSA or an HSA-binding protein or HSA-binding moiety is linked, e.g., conjugated, to a Siglec-5 ECD, or a functional fragment or variant thereof. In certain embodiments, HSA or an HSA-binding protein or HSA-binding moiety is linked, e.g., conjugated, to a Siglec-6 ECD, or a functional fragment or variant thereof. In certain embodiments, HSA or an HSA-binding protein or HSA-binding moiety is linked, e.g., conjugated, to a Siglec-7 ECD, or a functional fragment or variant thereof. In certain embodiments, HSA or an HSA-binding protein or HSA-binding moiety is linked, e.g., conjugated, to a Siglec-8 ECD, or a functional fragment or variant thereof. In certain embodiments, HSA or an HSA-binding protein or HSA-binding moiety is linked, e.g., conjugated, to a Siglec-9 ECD, or a functional fragment or variant thereof. In certain embodiments, HSA or an HSA-binding protein or HSA-binding moiety is linked, e.g., conjugated, to a Siglec-10 ECD, or a functional fragment or variant thereof. In certain embodiments, HSA or an HSA-binding protein or HSA-binding moiety is linked, e.g., conjugated, to a Siglec-11 ECD, or a functional fragment or variant thereof. In certain embodiments, HSA or an HSA-binding protein or HSA-binding moiety is linked, e.g., conjugated, to a Siglec-12 ECD, or a functional fragment or variant thereof. In certain embodiments, HSA or an HSA-binding protein or HSA-binding moiety is linked, e.g., conjugated, to a Siglec-14 ECD, or a functional fragment or variant thereof. In certain embodiments HSA or an HSA-binding protein or HSA-binding moiety is linked, e.g., conjugated, to a Siglec-15 ECD, or a functional fragment or variant thereof. In certain embodiments, HSA or an HSA-binding protein or HSA-binding moiety is linked, e.g., conjugated, to a Siglec-16 ECD, or a functional fragment or variant thereof.

d. Transferrin

Transferrin is a high molecular weight protein (molecular mass ˜76 kDa) that is normally present at a high concentration in human serum (approximately 3-4 mg/mL). Transferrin has a half-life of approximately 14 to 17 days in humans in its glycosylated form, or approximately 7-10 days in its non-glycosylated form. Transferrin binds circulating iron ions in a pH-dependent manner and mediates their transport throughout the body and into cells via interaction with its receptor. When iron-loaded transferrin is bound to its cell surface receptor, the receptor:transferrin complex is endocytosed, and the transition to the low-pH of the endosome triggers the release of iron ions. The naturally long half-life of transferrin is thought to be a function both of a recycling mechanism that returns endocytosed transferrin to circulation, in addition to the protein's large size.

In certain embodiments, the half-life extender is transferrin or a fragment thereof. Transferrin fusion proteins are known in the art, e.g., those described in U.S. Pat. Nos. 5,672,683, 5,977,307, and 7,176,278. In certain embodiments, the half-life extender is a protein having affinity for transferrin, such as an anti-transferrin antibody or derivative thereof, or transferrin-binding fragment thereof. Exemplary transferrin-binding proteins are known in the art, such as those described in U.S. patent application Ser. No. 16/755,268.

In certain embodiments, transferrin or a transferrin-binding protein is linked, e.g., conjugated, to a Siglec extracellular domain (ECD), or a functional fragment or variant thereof. In certain embodiments, transferrin or a transferrin-binding protein is linked, e.g., conjugated, to a Siglec-1 ECD, or a functional fragment or variant thereof. In certain embodiments, transferrin or a transferrin-binding protein is linked, e.g., conjugated, to a Siglec-2 ECD, or a functional fragment or variant thereof. In certain embodiments, transferrin or a transferrin-binding protein is linked, e.g., conjugated, to a Siglec-3 ECD, or a functional fragment or variant thereof. In certain embodiments, transferrin or a transferrin-binding protein is linked, e.g., conjugated, to a Siglec-4 ECD, or a functional fragment or variant thereof. In certain embodiments, transferrin or a transferrin-binding protein is linked, e.g., conjugated, to a Siglec-5 ECD, or a functional fragment or variant thereof. In certain embodiments, transferrin or a transferrin-binding protein is linked, e.g., conjugated, to a Siglec-6 ECD, or a functional fragment or variant thereof. In certain embodiments, transferrin or a transferrin-binding protein is linked, e.g., conjugated, to a Siglec-7 ECD, or a functional fragment or variant thereof. In certain embodiments, transferrin or a transferrin-binding protein is linked, e.g., conjugated, to a Siglec-8 ECD, or a functional fragment or variant thereof. In certain embodiments, transferrin or a transferrin-binding protein is linked, e.g., conjugated, to a Siglec-9 ECD, or a functional fragment or variant thereof. In certain embodiments, transferrin or a transferrin-binding protein is linked, e.g., conjugated, to a Siglec-10 ECD, or a functional fragment or variant thereof. In certain embodiments, transferrin or a transferrin-binding protein is linked, e.g., conjugated, to a Siglec-11 ECD, or a functional fragment or variant thereof. In certain embodiments, transferrin or a transferrin-binding protein is linked, e.g., conjugated, to a Siglec-12 ECD, or a functional fragment or variant thereof. In certain embodiments, transferrin or a transferrin-binding protein is linked, e.g., conjugated, to a Siglec-14 ECD, or a functional fragment or variant thereof. In certain embodiments transferrin or a transferrin-binding protein is linked, e.g., conjugated, to a Siglec-15 ECD, or a functional fragment or variant thereof. In certain embodiments, transferrin or a transferrin-binding protein is linked, e.g., conjugated, to a Siglec-16 ECD, or a functional fragment or variant thereof.

### III. Linkers

It is understood that, depending upon the circumstances, the Siglec ECD, or a functional fragment or variant thereof, can be linked or fused directly to a serum half-life enhancer (e.g., an immunoglobulin Fc domain). Alternatively, the Siglec ECD, or a functional fragment or variant thereof, can be covalently bound to the serum half-life enhancer by a linker.

The linker may couple, with one or more amino acids (natural, unnatural or a combination thereof), the Siglec ECD, or a functional fragment or variant thereof, to the serum half-life enhancer where the amino acid (e.g., a cysteine amino acid) may be introduced by site-directed mutagenesis. The linker may include one or more unnatural amino acids. It is contemplated that, in certain circumstances, a linker containing for example, one or more sulfhydryl reactive groups (e.g., a maleimide) may covalently link a cysteine in the Siglec ECD, or a functional fragment or variant thereof, or in the serum half-life enhancer that is a naturally occurring cysteine residue or is the product of site-specific mutagenesis.

The linker may be a cleavable linker or a non-cleavable linker. Optionally or in addition, the linker may be a flexible linker or an inflexible linker.

The linker should be a length sufficiently long to allow the Siglec ECD, or a functional fragment or variant thereof, and the serum half-life enhancer to be linked without steric hindrance from one another and sufficiently short to retain the intended activity of the fusion protein. The linker preferably is sufficiently hydrophilic to avoid or minimize instability of the fusion protein. The linker preferably is sufficiently hydrophilic to avoid or minimize insolubility of the fusion protein. The linker should be sufficiently stable in vivo (e.g., it is not cleaved by serum, enzymes, etc.) to permit the fusion protein to be operative in vivo.

The linker may be from about 1 angstroms (Å) to about 150 Å in length, or from about 1 Å to about 120 Å in length, or from about 5 Å to about 110 Å in length, or from about 10 Å to about 100 Å in length. The linker may be greater than about 2, 3, 4, 5, 6, 7, 8, 9, 10, 11, 12, 13, 14, 15, 16, 17, 18, 19, 20, 25, 27, 30 or greater angstroms in length and/or less than about 110, 100, 90, 85, 80, 75, 70, 65, 60, 55, 50, 45, 43, 42, 41, 40, 39, 38, 37, 36, 35, 34, 33, 32, 31, or fewer A in length. Furthermore, the linker may be about 5, 10, 15, 20, 25, 30, 35, 40, 45, 50, 55, 60, 65, 70, 75, 80, 85, 90, 95, 100, 110, and 120 Å in length.

In certain embodiments, the linker comprises a polypeptide linker that connects or fuses the Siglec ECD to the serum half-life enhancer (e.g., immunoglobulin Fc domain). For example, it is contemplated that a gene encoding a Siglec ECD linked directly or indirectly (for example, via an amino acid containing linker) to a serum half-life enhancer can be created and expressed using conventional recombinant DNA technologies. When a linker is employed, the linker may comprise hydrophilic amino acid residues, such as Gln, Ser, Gly, Glu, Pro, His and Arg. In certain embodiments, the linker is a peptide containing 1-25 amino acid residues, 1-20 amino acid residues, 2-15 amino acid residues, 3-10 amino acid residues, 3-7 amino acid residues, 4-25 amino acid residues, 4-20 amino acid residues, 4-15 amino acid residues, 4-10 amino acid residues, 5-25 amino acid residues, 5-20 amino acid residues, 5-15 amino acid residues, or 5-10 amino acid residues. Exemplary linkers include glycine and serine-rich linkers, e.g., (GlyGlyPro)ₙ (SEQ ID NO: 13) or (GlyGlyGlyGlySer)ₙ (SEQ ID NO: 14), where n is 1-5. In certain embodiments, the linker comprises, consists, or consists essentially of GGGGS (SEQ ID NO: 10). In certain embodiments, the linker comprises, consists, or consists essentially of GGGGSGGGGS (SEQ ID NO: 9). In certain embodiments, the linker comprises, consists, or consists essentially of GGGGSGGGGSGGGGS (SEQ ID NO: 11). In certain embodiments, the linker comprises, consists, or consists essentially of EPKSS (SEQ ID NO: 12). Additional exemplary linker sequences are disclosed, e.g., in George et al. (2003) PROTEIN ENGINEERING 15:871-879, and U.S. Pat. Nos. 5,482,858 and 5,525,491.

### IV. Proteins Comprising a Siglec Extracellular Domain (ECD) Conjugated to a Serum Half-Life Enhancer

In certain embodiments, the disclosure provides a protein comprising a Siglec ECD, or a functional fragment or variant thereof, conjugated to a serum half-life enhancer. The protein can comprise any Siglec ECD, or any functional fragment or variant thereof, disclosed herein and any serum half-life enhancer disclosed herein. The Siglec ECD, or a functional fragment or variant thereof, and the serum half-life enhancer can be fused directly, or can comprise any linker as disclosed herein. All combinations of Siglec ECDs, or functional fragments or variants thereof, linker, and serum half-life enhancer are contemplated herein.

In certain embodiments, the protein comprises a Siglec ECD selected from a human Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, Siglec-15, and Siglec-16 ECD, or a functional fragment or variant thereof. In certain embodiments, the protein comprises a Siglec ECD selected from a human Siglec-1, Siglec-4, Siglec-6, Siglec-9, Siglec-11, and Siglec-15 ECD, or a functional fragment or variant thereof. In certain embodiments, the Siglec ECD is selected from a human Siglec-1, Siglec-4, Siglec-6, Siglec-9, and Siglec-11 ECD, or a functional fragment or variant thereof.

In certain embodiments, the protein comprises a Siglec ECD selected from a human Siglec-1, Siglec-2, Siglec-3, Siglec-6, Siglec-7, Siglec-8, Siglec-10, Siglec-11, Siglec-12, Siglec-14, and Siglec-16 ECD, or a functional fragment or variant thereof. In certain embodiments, the protein comprises a Siglec ECD selected from a human Siglec-1, Siglec-6, and Siglec-11 ECD, or a functional fragment or variant thereof.

In certain embodiments, the protein comprises a Siglec-1 ECD, or a functional fragment or variant thereof, conjugated, optionally via a linker, to an Fc domain. In certain embodiments, the Siglec-1 ECD comprises or consists of amino acids 20-230 of SEQ ID NO: 15 (SEQ ID NO: 17). In certain embodiments, the Siglec-1 ECD comprises or consists of amino acids 20-230 of SEQ ID NO: 15 with an R119A substitution (SEQ ID NO: 18). In certain embodiments, the linker comprises or consists of a GGGGSGGGGS (SEQ ID NO: 9) linker or a EPKSS (SEQ ID NO: 12) linker. In certain embodiments, the Fc domain is a human IgG1 Fc domain, optionally with a N297G mutation. In certain embodiments, the protein comprises SEQ ID NO: 19. In certain embodiments, the protein is encoded by SEQ ID NO: 20. In certain embodiments, the protein comprises SEQ ID NO: 21, or a functional fragment thereof. In certain embodiments, the protein is encoded by SEQ ID NO: 22. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 19 or SEQ ID NO: 21. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 20 or SEQ ID NO: 22.

In certain embodiments, the protein comprises a Siglec-2 ECD, or a functional fragment or variant thereof, conjugated, optionally via a linker, to an Fc domain. In certain embodiments, the Siglec-2 ECD comprises or consists of amino acids 24-122 or 20-688 of SEQ ID NO: 23. In certain embodiments, the Siglec-1 ECD comprises or consists of amino acids 20-688 of SEQ ID NO: 23 with an R120A substitution. In certain embodiments, the linker comprises or consists of a GGGGSGGGGS (SEQ ID NO: 9) linker or a EPKSS (SEQ ID NO: 12) linker. In certain embodiments, the Fc domain is a human IgG1 Fc domain, optionally with a N297G mutation.

In certain embodiments, the protein comprises a Siglec-3 ECD, or a functional fragment or variant thereof, conjugated, optionally via a linker, to an Fe domain. In certain embodiments, the Siglec-3 ECD comprises or consists of amino acids 18-259 of SEQ ID NO: 25 (SEQ ID NO: 29). In certain embodiments, the Siglec-3 ECD comprises or consists of amino acids 18-259 of SEQ ID NO: 25 with an R119K substitution (SEQ ID NO: 30). In certain embodiments, the linker comprises or consists of a GGGGSGGGGS (SEQ ID NO: 9) linker or a EPKSS (SEQ ID NO: 12) linker. In certain embodiments, the Fc domain is a human IgG1 Fc domain, optionally with a N297G mutation. In certain embodiments, the protein comprises SEQ ID NO: 31, SEQ ID NO: 126, SEQ ID NO: 139, or SEQ ID NO: 154, or a functional fragment thereof. In certain embodiments, the protein is encoded by SEQ ID NO: 32 or SEQ ID NO: 140. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 31, SEQ ID NO: 126, SEQ ID NO: 139, or SEQ ID NO: 154. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 32 or SEQ ID NO: 140.

In certain embodiments, the protein comprises a Siglec-4 ECD, or a functional fragment or variant thereof, conjugated, optionally via a linker, to an Fc domain. In certain embodiments, the Siglec-4 ECD comprises or consists of amino acids 20-516 of SEQ ID NO: 33 (SEQ ID NO: 35). In certain embodiments, the Siglec-4 ECD comprises or consists of amino acids 20-516 of SEQ ID NO: 33 with an R118A substitution (SEQ ID NO: 36). In certain embodiments, the linker comprises or consists of a GGGGSGGGGS (SEQ ID NO: 9) linker or a EPKSS (SEQ ID NO: 12) linker. In certain embodiments, the Fc domain is a human IgG1 Fc domain, optionally with a N297G mutation. In certain embodiments, the protein comprises SEQ ID NO: 37, or a functional fragment thereof. In certain embodiments, the protein is encoded by SEQ ID NO: 38. In certain embodiments, the protein comprises SEQ ID NO: 39. In certain embodiments, the protein is encoded by SEQ ID NO: 40. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 37 or SEQ ID NO: 39. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 38 or SEQ ID NO: 40.

In certain embodiments, the protein comprises a Siglec-5 ECD, or a functional fragment or variant thereof, conjugated, optionally via a linker, to an Fe domain. In certain embodiments, the Siglec-5 ECD comprises or consists of amino acid residues 21-140 or 17-441 of SEQ ID NO: 41. In certain embodiments, the Siglec-5 ECD comprises or consists of amino acid residues 17-441 of SEQ ID NO: 41 with an R119A substitution. In certain embodiments, the linker comprises or consists of a GGGGSGGGGS (SEQ ID NO: 9) linker or a EPKSS (SEQ ID NO: 12) linker. In certain embodiments, the Fc domain is a human IgG1 Fc domain, optionally with a N297G mutation. In certain embodiments, the protein comprises SEQ ID NO: 141, SEQ ID NO: 143, SEQ ID NO: 149, or SEQ ID NO: 150, or a functional fragment thereof. In certain embodiments, the protein is encoded by SEQ ID NO: 142 or SEQ ID NO: 144. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 141, SEQ ID NO: 143, SEQ ID NO: 149, or SEQ ID NO: 150. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 142 or SEQ ID NO: 144.

In certain embodiments, the protein comprises a Siglec-6 ECD, or a functional fragment or variant thereof, conjugated, optionally via a linker, to an Fc domain. In certain embodiments, the Siglec-6 ECD comprises or consists of amino acids 27-347 of SEQ ID NO: 43 (SEQ ID NO: 45). In certain embodiments, the Siglec-6 ECD comprises or consists of amino acids 27-347 of SEQ ID NO: 43 with an R122A substitution (SEQ ID NO: 46). In certain embodiments, the Siglec-6 ECD comprises or consists of amino acids 27-347 of SEQ ID NO: 43 with an R122K mutation (SEQ ID NO: 156). In certain embodiments, the linker comprises or consists of a GGGGSGGGGS (SEQ ID NO: 9) linker or a EPKSS (SEQ ID NO: 12) linker. In certain embodiments, the Fc domain is a human IgG1 Fc domain, optionally with a N297G mutation. In certain embodiments, the protein comprises SEQ ID NO: 47, or a functional fragment thereof. In certain embodiments, the protein is encoded by SEQ ID NO: 48. In certain embodiments, the protein comprises SEQ ID NO: 49, or a functional fragment thereof. In certain embodiments, the protein is encoded by SEQ ID NO: 50. In certain embodiments, the protein comprises SEQ ID NO: 158, or a functional fragment thereof. In certain embodiments, the protein is encoded by SEQ ID NO: 159. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 47, SEQ ID NO: 49, or SEQ ID NO: 158. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 48, SEQ ID NO: 50, or SEQ ID NO: 159.

In certain embodiments, the protein comprises a Siglec-7 ECD, or a functional fragment or variant thereof, conjugated, optionally via a linker, to an Fc domain. In certain embodiments, the Siglec-7 ECD comprises or consists of amino acids 19-357 of SEQ ID NO: 51 (SEQ ID NO: 57). In certain embodiments, the Siglec-7 ECD comprises or consists of amino acids 19-357 of SEQ ID NO: 51 with an R124K substitution (SEQ ID NO: 58). In certain embodiments, the linker comprises or consists of a GGGGSGGGGS (SEQ ID NO: 9) linker or a EPKSS (SEQ ID NO: 12) linker. In certain embodiments, the Fc domain is a human IgG1 Fc domain, optionally with a N297G mutation. In certain embodiments, the protein comprises SEQ ID NO: 59, SEQ ID NO: 61, SEQ ID NO: 127, or SEQ ID NO: 128, or a functional fragment thereof. In certain embodiments, the protein is encoded by SEQ ID NO: 60 or SEQ ID NO: 62. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 59, SEQ ID NO: 61, SEQ ID NO: 127, or SEQ ID NO: 128. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 60 or SEQ ID NO: 62.

In certain embodiments, the protein comprises a Siglec-8 ECD, or a functional fragment or variant thereof, conjugated, optionally via a linker, to an Fc domain. In certain embodiments, the Siglec-8 ECD comprises or consists of amino acids 17-364 or 27-151 of SEQ ID NO: 63. In certain embodiments, the Siglec-7 ECD comprises or consists of amino acids 17-364 or 27-151 of SEQ ID NO: 51 with an R128A substitution (SEQ ID NO: 58). In certain embodiments, the linker comprises or consists of a GGGGSGGGGS (SEQ ID NO: 9) linker or a EPKSS (SEQ ID NO: 12) linker. In certain embodiments, the Fc domain is a human IgG1 Fc domain, optionally with a N297G mutation.

In certain embodiments, the protein comprises a Siglec-9 ECD, or a functional fragment or variant thereof, conjugated, optionally via a linker, to an Fc domain. In certain embodiments, the Siglec-9 ECD comprises or consists of amino acids 18-145, 18-348, or 146-348 of SEQ ID NO: 65. In certain embodiments, the Siglec-9 ECD comprises a mutation at one or more of K100, K131, C158, C295, or A315, for example, K100E, K131Q, C158S, C295Y, or A315E. In certain embodiments, the Siglec-9 ECD comprises C158 and C295 mutations (e.g., C158S and C295Y). In certain embodiments, the Siglec-9 ECD comprises a K131 (e.g., K131Q) mutation. In certain embodiments, the Siglec-9 ECD comprises K100 and A315 (e.g., K100E and A315E) mutations. In certain embodiments, the Siglec-9 ECD comprises a mutation at R120, e.g., a R120A or an R120K mutation. In certain embodiments, the Siglec-9 ECD comprises or consists of amino acids 18-348 of SEQ ID NO: 65, optionally with C158S and C295Y mutations (SEQ ID NO: 71). In certain embodiments, the Siglec-9 ECD comprises or consists of amino acids 18-348 of SEQ ID NO: 65, optionally with C158S and C295Y mutations, and an R120K substitution (SEQ ID NO: 72). In certain embodiments, the Siglec-9 ECD comprises or consists of amino acids 18-348 of SEQ ID NO: 65, optionally with a K131Q mutation. In certain embodiments, the Siglec-9 ECD comprises or consists of amino acids 18-348 of SEQ ID NO: 65, optionally with a K131Q mutation mutations, and an R120K substitution. In certain embodiments, the Siglec-9 ECD comprises or consists of amino acids 18-348 of SEQ ID NO: 65, optionally with K100E and A315E mutations. In certain embodiments, the Siglec-9 ECD comprises or consists of amino acids 18-348 of SEQ ID NO: 65, optionally with K100E and A315E mutations, and an R120K substitution. In certain embodiments, the linker comprises or consists of a GGGGSGGGGS (SEQ ID NO: 9) linker or a EPKSS (SEQ ID NO: 12) linker. In certain embodiments, the Fc domain is a human IgG1 Fc domain, optionally with a N297G mutation. In certain embodiments, the protein comprises SEQ ID NO: 73, 75, 77, 81, 83, 85, 120, 122, or 129-135, or a functional fragment thereof. In certain embodiments, the protein is encoded by SEQ ID NO: 74, 76, 78, 82, 84, 86, 121, or 123. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 73, 75, 77, 81, 83, 85, 120, 122, or 129-135. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 74, 76, 78, 82, 84, 86, 121, or 123.

In certain embodiments, the protein comprises a Siglec-10 ECD, or a functional fragment or variant thereof, conjugated, optionally via a linker, to an Fc domain. In certain embodiments, the Siglec-10 ECD comprises or consists of amino acids 18-550 of SEQ ID NO: 87 (SEQ ID NO: 89). In certain embodiments, the Siglec-10 ECD comprises or consists of amino acids 18-550 of SEQ ID NO: 87 with an R119A substitution. In certain embodiments, the linker comprises or consists of a GGGGSGGGGS (SEQ ID NO: 9) linker or a EPKSS (SEQ ID NO: 12) linker. In certain embodiments, the Fe domain is a human IgG1 Fe domain, optionally with a N297G mutation. In certain embodiments, the protein comprises SEQ ID NO: 90, or a functional fragment thereof. In certain embodiments, the protein is encoded by SEQ ID NO: 91. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 90. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 91.

In certain embodiments, the protein comprises a Siglec-11 ECD, or a functional fragment or variant thereof, conjugated, optionally via a linker, to an Fc domain. In certain embodiments, the Siglec-11 ECD comprises or consists of amino acids 28-561 of SEQ ID NO: 92 (SEQ ID NO: 94). In certain embodiments, the Siglec-11 ECD comprises or consists of amino acids 28-561 of SEQ ID NO: 92 with an R132A substitution (SEQ ID NO: 95). In certain embodiments, the linker comprises or consists of a GGGGSGGGGS (SEQ ID NO: 9) linker or a EPKSS (SEQ ID NO: 12) linker. In certain embodiments, the Fc domain is a human IgG1 Fc domain, optionally with a N297G mutation. In certain embodiments, the protein comprises SEQ ID NO: 96 or SEQ ID NO: 98, or a functional fragment thereof. In certain embodiments, the protein is encoded by SEQ ID NO: 97 or SEQ ID NO: 99. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 96 or SEQ ID NO: 98. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 97 or SEQ ID NO: 99.

In certain embodiments, the protein comprises a Siglec-12 ECD, or a functional fragment or variant thereof, conjugated, optionally via a linker, to an Fc domain. In certain embodiments, the Siglec-12 ECD comprises or consists of amino acids 19-482 or 24-142 of SEQ ID NO: 100. In certain embodiments, the Siglec-12 ECD comprises or consists of amino acids 19-482 or 24-142 of SEQ ID NO: 100 with a mutation in the region from amino acid 122-151 of SEQ ID NO: 100. In certain embodiments, the linker comprises or consists of a GGGGSGGGGS (SEQ ID NO: 9) linker or a EPKSS (SEQ ID NO: 12) linker. In certain embodiments, the Fe domain is a human IgG1 Fe domain, optionally with a N297G mutation.

In certain embodiments, the protein comprises a Siglec-14 ECD, or a functional fragment or variant thereof, conjugated, optionally via a linker, to an Fe domain. In certain embodiments, the Siglec-14 ECD comprises or consists of amino acids 17-359 or 21-140 of SEQ ID NO: 102. In certain embodiments, the Siglec-14 ECD comprises or consists of amino acids 17-359 or 21-140 of SEQ ID NO: 102 with an R119A substitution. In certain embodiments, the linker comprises or consists of a GGGGSGGGGS (SEQ ID NO: 9) linker or a EPKSS (SEQ ID NO: 12) linker. In certain embodiments, the Fe domain is a human IgG1 Fe domain, optionally with a N297G mutation. In certain embodiments, the protein comprises SEQ ID NO: 145, SEQ ID NO: 147, SEQ ID NO: 151, or SEQ ID NO: 155, or a functional fragment thereof. In certain embodiments, the protein is encoded by SEQ ID NO: 146 or SEQ ID NO: 148. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 145, SEQ ID NO: 147, SEQ ID NO: 151, SEQ ID NO: 155. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 146 or SEQ ID NO: 148.

In certain embodiments, the protein comprises a Siglec-15 ECD, or a functional fragment or variant thereof, conjugated, optionally via a linker, to an Fe domain. In certain embodiments, the Siglec-15 ECD comprises or consists of amino acids 20-263 of SEQ ID NO: 104 (SEQ ID NO: 106). In certain embodiments, the Siglec-15 ECD comprises or consists of amino acids 20-263 of SEQ ID NO: 104 with a substitution at an amino acid corresponding to R143 (e.g., R143A) of SEQ ID NO: 104. In certain embodiments, the Siglec-15 ECD comprises or consists of amino acids 20-263 of SEQ ID NO: 104 with an R143A substitution (SEQ ID NO: 107). In certain embodiments, the linker comprises or consists of a GGGGSGGGGS (SEQ ID NO: 9) linker or a EPKSS (SEQ ID NO: 12) linker. In certain embodiments, the Fe domain is a human IgG1 Fe domain, optionally with a N297G mutation. In certain embodiments, the protein comprises SEQ ID NO: 108, SEQ ID NO: 110, or SEQ ID NO: 124, or a functional fragment thereof. In certain embodiments, the protein is encoded by SEQ ID NO: 109, SEQ ID NO: 111, or SEQ ID NO: 125. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 108, SEQ ID NO: 110, or SEQ ID NO: 124. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 109, SEQ ID NO: 111, or SEQ ID NO: 125.

In certain embodiments, the protein comprises a Siglec-16 ECD, or a functional fragment or variant thereof, conjugated, optionally via a linker, to an Fc domain. In certain embodiments, the Siglec-16 ECD comprises or consists of amino acids 15-434 of SEQ ID NO: 114 (SEQ ID NO: 116). In certain embodiments, the Siglec-16 ECD comprises or consists of amino acids 15-434 of SEQ ID NO: 114 with an R120A substitution. In certain embodiments, the linker comprises or consists of a GGGGSGGGGS (SEQ ID NO: 9) linker or a EPKSS (SEQ ID NO: 12) linker. In certain embodiments, the Fc domain is a human IgG1 Fc domain, optionally with a N297G mutation. In certain embodiments, the protein comprises SEQ ID NO: 117, or a functional fragment thereof. In certain embodiments, the protein is encoded by SEQ ID NO: 118. In certain embodiments, the protein comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO: 117. In certain embodiments, the protein is encoded by a nucleotide sequence having 80%, 85%, 86%, 87%, 88%, 89%, 90%, 91%, 92%, 93%, 94%, 95%, 96%, 97%, 98%, or 99% sequence identity to SEQ ID NO: 118.

### V. Methods of Making Siglec Extracellular Domain (ECD) Conjugated to a Serum Half-Life Enhancer

Methods for producing a Siglec extracellular domain (ECD), or a functional fragment or variant thereof, are known in the art. For example, DNA molecules encoding a Siglec extracellular domain (ECD), or a functional fragment or variant thereof and, if appropriate, a protein-based serum half-life enhancer (e.g., a peptide that can dimerize to produce an Fc domain) either linked directly or via a peptide linker, can be synthesized chemically or by recombinant DNA methodologies. The sequences of interest can be cloned by conventional hybridization techniques or polymerase chain reaction (PCR) techniques, using the appropriate synthetic nucleic acid primers. The resulting DNA molecules encoding the Siglec extracellular domain (ECD), or a functional fragment or variant thereof, and optionally, the serum half-life extender and optional linker, can be ligated to other appropriate nucleotide sequences, including, for example, expression control sequences, to produce conventional gene expression constructs (i.e., expression vectors) encoding the desired antibodies. Production of defined gene constructs is within routine skill in the art.

Nucleic acids encoding a desired Siglec extracellular domain (ECD), or a functional fragment or variant thereof, can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the Siglec extracellular domain (ECD), or a functional fragment or variant thereof.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. The gene construct can be introduced into eukaryotic host cells using conventional techniques.

A polypeptide comprising the components described herein can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags. In certain embodiments, in order to express a protein, e.g., a Siglec extracellular domain (ECD), or a functional fragment or variant thereof, as a secreted protein, a native N-terminal signal sequence of the protein is replaced, e.g., with MDMRVPAQLLGLLLLWLPGARC (SEQ ID NO: 136), MGWSCIILFLVATATGVHS (SEQ ID NO: 152), or MEFGLSWLFLVAILKGVQC (SEQ ID NO: 153). In certain embodiments, to express a protein, e.g., a fusion protein, as a secreted protein, an N-terminal signal sequence, e.g., MDMRVPAQLLGLLLLWLPGARC (SEQ ID NO: 136), MGWSCIILFLVATATGVHS (SEQ ID NO: 152), or MEFGLSWLFLVAILKGVQC (SEQ ID NO: 153) is added. Additional exemplary N-terminal signal sequences include signal sequences from interleukin-2, CD-5, IgG kappa light chain, trypsinogen, serum albumin, and prolactin. In certain embodiments, in order to express a protein, e.g., a Siglec extracellular domain (ECD), or a functional fragment or variant thereof, as a secreted protein, a C terminal lysosomal signal motif, e.g., YGTL (SEQ ID NO: 137) is removed.

### VI. Pharmaceutical Compositions

For therapeutic use, a Siglec extracellular domain (ECD), or a functional fragment or variant thereof, optionally conjugated to a serum half-life enhancer, preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-methacrylate), ethylene vinyl acetate, or poly-D(−)-3-hydroxybutyric acid. Sustained release compositions may also include liposomes that can be prepared by any of several methods known in the art.

Pharmaceutical compositions containing a Siglec extracellular domain (ECD), or a functional fragment or variant thereof, optionally conjugated to a serum half-life enhancer, as disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, it is contemplated that the constructs derived herein can be administered by IV infusion. Alternatively, it is contemplated that the constructs can be administered by intratumoral injection.

Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The compositions described herein may be administered locally or systemically. It is contemplated that the compositions described herein are generally administered by parenteral administration. Preparations for parenteral administration include sterile aqueous or non-aqueous solutions, suspensions, and emulsions. In certain embodiments, the pharmaceutical composition is administered subcutaneously or maybe administered intravenously, e.g., via intravenous infusion.

Generally, a therapeutically effective amount of active component, for example, a Siglec extracellular domain (ECD), or a functional fragment or variant thereof, optionally conjugated to a serum half-life enhancer, is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the active component, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the Siglec extracellular domain (ECD), or a functional fragment or variant thereof, optionally conjugated to a serum half-life enhancer, and the disease being treated. Exemplary dosing frequencies are once per day, once per week and once every two weeks. In certain embodiments, a Siglec extracellular domain (ECD), or a functional fragment or variant thereof, optionally conjugated to a serum half-life enhancer, is lyophilized, and then reconstituted in buffered saline, at the time of administration.

### VII. Therapeutic Uses

The compositions and methods disclosed herein can be used to treat various forms of immune system disorders in a subject. The present invention provides methods for decreasing an unwanted immune or inflammatory response in a subject, by administering to the subject an effective amount of a Siglec ECD, or a functional fragment or variant thereof, optionally conjugated to a serum half-life enhancer, wherein the Siglec ECD, or the functional fragment or variant thereof, reduces the unwanted immune or inflammatory response in the subject. In one embodiment, the method is used to decrease the number of T cells, e.g., CD4 T cells and/or CD8 T cells, in a subject. In another embodiment, the method is used to decrease the activity of T cells, e.g., CD4 T cells and/or CD8 T cells, in a subject. In certain embodiments, the T cells are hyperactive. Siglec ECDs of the present invention act primarily on activated T cells with little observed effects on non-activated T cells.

Activation of T cells occurs through the simultaneous engagement of a T-cell receptor and a co-stimulatory molecule (like CD28, or ICOS) on CD4+ T cells by the major histocompatibility complex (MHCII) peptide and co-stimulatory molecules on the APC. Both are required for production of an effective immune response. In the absence of co-stimulation, T cell receptor signaling alone results in anergy. The signaling pathways downstream from co-stimulatory molecules usually engages the PI3K pathway generating PIP3 at the plasma membrane and recruiting PH domain containing signaling molecules like PDK1 that are essential for the activation of PKC-θ, and eventual IL-2 production. Optimal CD8+ T cell response relies on CD4+ signaling. CD4+ cells are useful in the initial antigenic activation of naïve CD8 T cells, and sustaining memory CD8+ T cells in the aftermath of an acute infection. Therefore, activation of CD4+ T cells can be beneficial to the action of CD8+ T cells. However, the unwanted immune or inflammatory activated T cell response can result in an inflammatory or autoimmune disorder.

Thus, the invention provides a method of treating an inflammatory disorder and/or an autoimmune disorder in a subject. The method comprises administering to the subject an effective amount of a protein construct described herein either alone or in a combination with another therapeutic agent to treat the inflammatory disorder and/or the autoimmune disorder in the subject. The term “effective amount” as used herein refers to the amount of an active agent (e.g., Siglec extracellular domain (ECD), or a functional fragment or variant thereof, optionally conjugated to a serum half-life enhancer, according to the present invention) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of inflammatory disorders include, but are not limited to: acute respiratory distress syndrome (ARDS), acute lung injury (ALI), alcoholic liver disease, allergic inflammation of the skin, lungs, and gastrointestinal tract, allergic rhinitis, ankylosing spondylitis, asthma (allergic and non-allergic), atopic dermatitis (also known as atopic eczema), atherosclerosis, celiac disease, chronic obstructive pulmonary disease (COPD), chronic respiratory distress syndrome (CRDS), colitis, dermatitis, diabetes, eczema, endocarditis, fatty liver disease, fibrosis (e.g., idiopathic pulmonary fibrosis, scleroderma, kidney fibrosis, and scarring), food allergies (e.g., allergies to peanuts, eggs, dairy, shellfish, tree nuts, etc.), gastritis, gout, hepatic steatosis, hepatitis, inflammation of body organs including joint inflammation including joints in the knees, limbs or hands, inflammatory bowel disease (IBD) (including Crohn's disease or ulcerative colitis), intestinal hyperplasia, irritable bowel syndrome, juvenile rheumatoid arthritis, liver disease, metabolic syndrome, multiple sclerosis, myasthenia gravis, neurogenic lung edema, nephritis (e.g., glomerular nephritis), non-alcoholic fatty liver disease (NAFLD) (including non-alcoholic steatosis and non-alcoholic steatohepatitis (NASH)), obesity, prostatitis, psoriasis, psoriatic arthritis, rheumatoid arthritis, sarcoidosis sinusitis, splenitis, seasonal allergies, sepsis, systemic lupus erythematosus, uveitis, and UV-induced skin inflammation.

Examples of autoimmune diseases or disorders include, but are not limited to: arthritis, including rheumatoid arthritis, acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis chronica progrediente, arthritis deformans, polyarthritis chronica primaria, reactive arthritis, and ankylosing spondylitis; inflammatory hyperproliferative skin diseases; psoriasis, such as plaque psoriasis, gutatte psoriasis, pustular psoriasis, and psoriasis of the nails; atopy, including atopic diseases such as hay fever and Job's syndrome; dermatitis, including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis; x-linked hyper IgM syndrome; allergic intraocular inflammatory diseases; urticaria, such as chronic allergic urticaria, chronic idiopathic urticaria, and chronic autoimmune urticaria; myositis; polymyositis/dermatomyositis; juvenile dermatomyositis; toxic epidermal necrolysis; scleroderma, including systemic scleroderma; sclerosis, such as systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis; neuromyelitis optica (NMO); inflammatory bowel disease (IBD), including Crohn's disease, autoimmune-mediated gastrointestinal diseases, colitis, ulcerative colitis, colitis ulcerosa, microscopic colitis, collagenous colitis, colitis polyposa, necrotizing enterocolitis, transmural colitis, and autoimmune inflammatory bowel disease; bowel inflammation; pyoderma gangrenosum; erythema nodosum; primary sclerosing cholangitis; respiratory distress syndrome, including adult or acute respiratory distress syndrome (ARDS); meningitis; inflammation of all or part of the uvea; iritis; choroiditis; an autoimmune hematological disorder; rheumatoid spondylitis; rheumatoid synovitis; hereditary angioedema; cranial nerve damage, as in meningitis; herpes gestationis; pemphigoid gestationis; pruritis scroti; autoimmune premature ovarian failure; sudden hearing loss due to an autoimmune condition; IgE-mediated diseases, such as anaphylaxis and allergic and atopic rhinitis; encephalitis, such as Rasmussen's encephalitis and limbic and/or brainstem encephalitis; uveitis, such as anterior uveitis, acute anterior uveitis, granulomatous uveitis, nongranulomatous uveitis, phacoantigenic uveitis, posterior uveitis, or autoimmune uveitis; glomerulonephritis (GN) with and without nephrotic syndrome, such as chronic or acute glomerulonephritis, primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN; proliferative nephritis; autoimmune polyglandular endocrine failure; balanitis, including balanitis circumscripta plasmacellularis; balanoposthitis; erythema annulare centrifugum; erythema dyschromicum perstans; eythema multiform; granuloma annulare; lichen nitidus; lichen sclerosus et atrophicus; lichen simplex chronicus; lichen spinulosus; lichen planus; lamellar ichthyosis; epidermolytic hyperkeratosis; premalignant keratosis; pyoderma gangrenosum; allergic conditions and responses; allergic reaction; eczema, including allergic or atopic eczema, asteatotic eczema, dyshidrotic eczema, and vesicular palmoplantar eczema; asthma, such as asthma bronchiale, bronchial asthma, and auto-immune asthma; conditions involving infiltration of T cells and chronic inflammatory responses; immune reactions against foreign antigens such as fetal A-B-O blood groups during pregnancy; chronic pulmonary inflammatory disease; autoimmune myocarditis; leukocyte adhesion deficiency; lupus, including lupus nephritis, lupus cerebritis, pediatric lupus, non-renal lupus, extra-renal lupus, discoid lupus and discoid lupus erythematosus, alopecia lupus, systemic lupus erythematosus (SLE), cutaneous SLE, subacute cutaneous SLE, neonatal lupus syndrome (NLE), and lupus erythematosus disseminatus; juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, and diabetic large-artery disorder; immune responses associated with acute and delayed hypersensitivity mediated by cytokines and T-lymphocytes; tuberculosis; sarcoidosis; granulomatosis, including lymphomatoid granulomatosis; Wegener's granulomatosis; agranulocytosis; vasculitides, including vasculitis, large-vessel vasculitis, polymyalgia rheumatica and giant-cell (Takayasu's) arteritis, medium-vessel vasculitis, Kawasaki's disease, polyarteritis nodosa/periarteritis nodosa, microscopic polyarteritis, immunovasculitis, CNS vasculitis, cutaneous vasculitis, hypersensitivity vasculitis, necrotizing vasculitis, systemic necrotizing vasculitis, ANCA-associated vasculitis, Churg-Strauss vasculitis or syndrome (CSS), and ANCA-associated small-vessel vasculitis; temporal arteritis; aplastic anemia; autoimmune aplastic anemia; Coombs positive anemia; Diamond Blackfan anemia; hemolytic anemia or immune hemolytic anemia, including autoimmune hemolytic anemia (AIHA), pernicious anemia (anemia perniciosa); Addison's disease; pure red cell anemia or aplasia (PRCA); Factor VIII deficiency; hemophilia A; autoimmune neutropenia; pancytopenia; leukopenia; diseases involving leukocyte diapedesis; CNS inflammatory disorders; multiple organ injury syndrome, such as those secondary to septicemia, trauma or hemorrhage; antigen-antibody complex-mediated diseases; antiglomerular basement membrane disease; anti-phospholipid antibody syndrome; allergic neuritis; Behcet's disease/syndrome; Castleman's syndrome; Goodpasture's syndrome; Reynaud's syndrome; Sjogren's syndrome; Stevens-Johnson syndrome; pemphigoid, such as pemphigoid bullous and skin pemphigoid, pemphigus, pemphigus vulgaris, pemphigus foliaceus, pemphigus mucus-membrane pemphigoid, and pemphigus erythematosus; autoimmune polyendocrinopathies; Reiter's disease or syndrome; thermal injury; preeclampsia; an immune complex disorder, such as immune complex nephritis, and antibody-mediated nephritis; polyneuropathies; chronic neuropathy, such as IgM polyneuropathies and IgM-mediated neuropathy; thrombocytopenia (as developed by myocardial infarction patients, for example), including thrombotic thrombocytopenic purpura (TTP), post-transfusion purpura (PTP), heparin-induced thrombocytopenia, autoimmune or immune-mediated thrombocytopenia, idiopathic thrombocytopenic purpura (ITP), and chronic or acute ITP; scleritis, such as idiopathic cerato-scleritis, and episcleritis; autoimmune disease of the testis and ovary including, autoimmune orchitis and oophoritis; primary hypothyroidism; hypoparathyroidism; autoimmune endocrine diseases, including thyroiditis, autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes, autoimmune polyglandular syndromes, and polyglandular endocrinopathy syndromes; paraneoplastic syndromes, including neurologic paraneoplastic syndromes; Lambert-Eaton myasthenic syndrome or Eaton-Lambert syndrome; stiff-man or stiff-person syndrome; encephalomyelitis, such as allergic encephalomyelitis, encephalomyelitis allergica, and experimental allergic encephalomyelitis (EAE); myasthenia gravis, such as thymoma-associated myasthenia gravis; cerebellar degeneration; neuromyotonia; opsoclonus or opsoclonus myoclonus syndrome (OMS); sensory neuropathy; multifocal motor neuropathy; Sheehan's syndrome; hepatitis, including autoimmune hepatitis, chronic hepatitis, lupoid hepatitis, giant-cell hepatitis, chronic active hepatitis, and autoimmune chronic active hepatitis; lymphoid interstitial pneumonitis (LIP); bronchiolitis obliterans (non-transplant) vs NSIP; Guillain-Barre syndrome; Berger's disease (IgA nephropathy); idiopathic IgA nephropathy; linear IgA dermatosis; acute febrile neutrophilic dermatosis; subcorneal pustular dermatosis; transient acantholytic dermatosis; cirrhosis, such as primary biliary cirrhosis and pneumonocirrhosis; autoimmune enteropathy syndrome; Celiac or Coeliac disease; celiac sprue (gluten enteropathy); refractory sprue; idiopathic sprue; cryoglobulinemia; amylotrophic lateral sclerosis (ALS; Lou Gehrig's disease); coronary artery disease; autoimmune ear disease, such as autoimmune inner ear disease (AIED); autoimmune hearing loss; polychondritis, such as refractory or relapsed or relapsing polychondritis; pulmonary alveolar proteinosis; Cogan's syndrome/nonsyphilitic interstitial keratitis; Bell's palsy; Sweet's disease/syndrome; rosacea autoimmune; zoster-associated pain; amyloidosis; a non-cancerous lymphocytosis; a primary lymphocytosis, including monoclonal B cell lymphocytosis (e.g., benign monoclonal gammopathy and monoclonal gammopathy of undetermined significance, MGUS); peripheral neuropathy; channelopathies, such as epilepsy, migraine, arrhythmia, muscular disorders, deafness, blindness, periodic paralysis, and channelopathies of the CNS; autism; inflammatory myopathy; focal or segmental or focal segmental glomerulosclerosis (FSGS); endocrine opthalmopathy; uveoretinitis; chorioretinitis; autoimmune hepatological disorder; fibromyalgia; multiple endocrine failure; Schmidt's syndrome; adrenalitis; gastric atrophy; presenile dementia; demyelinating diseases, such as autoimmune demyelinating diseases and chronic inflammatory demyelinating polyneuropathy; Dressler's syndrome; alopecia areata; alopecia totalis; CREST syndrome (calcinosis, Raynaud's phenomenon, esophageal dysmotility, sclerodactyly, and telangiectasia); male and female autoimmune infertility (e.g., due to anti-spermatozoan antibodies); mixed connective tissue disease; Chagas' disease; rheumatic fever; recurrent abortion; farmer's lung; erythema multiforme; post-cardiotomy syndrome; Cushing's syndrome; bird-fancier's lung; allergic granulomatous angiitis; benign lymphocytic angiitis; Alport's syndrome; alveolitis, such as allergic alveolitis and fibrosing alveolitis; interstitial lung disease; transfusion reaction; leprosy; malaria; Samter's syndrome; Caplan's syndrome; endocarditis; endomyocardial fibrosis; diffuse interstitial pulmonary fibrosis; interstitial lung fibrosis; pulmonary fibrosis; idiopathic pulmonary fibrosis; cystic fibrosis; endophthalmitis; erythema elevatum et diutinum; erythroblastosis fetalis; eosinophilic fasciitis; Shulman's syndrome; Felty's syndrome; flariasis; cyclitis, such as chronic cyclitis, heterochronic cyclitis, iridocyclitis (acute or chronic), or Fuch's cyclitis; Henoch-Schonlein purpura; sepsis; endotoxemia; pancreatitis; thyroxicosis; Evan's syndrome; autoimmune gonadal failure; Sydenham's chorea; post-streptococcal nephritis; thromboangitis ubiterans; thyrotoxicosis; tabes dorsalis; choroiditis; giant-cell polymyalgia; chronic hypersensitivity pneumonitis; keratoconjunctivitis sicca; epidemic keratoconjunctivitis; idiopathic nephritic syndrome; minimal change nephropathy; benign familial and ischemia-reperfusion injury; transplant organ reperfusion; retinal autoimmunity; joint inflammation; bronchitis; chronic obstructive airway/pulmonary disease; silicosis; aphthae; aphthous stomatitis; arteriosclerotic disorders; aspermiogenese; autoimmune hemolysis; Boeck's disease; cryoglobulinemia; Dupuytren's contracture; endophthalmia phacoanaphylactica; enteritis allergica; erythema nodo sum leprosum; idiopathic facial paralysis; febris rheumatica; Hamman-Rich's disease; sensoneural hearing loss; haemoglobinuria paroxysmatica; hypogonadism; ileitis regionalis; leucopenia; mononucleosis infectiosa; traverse myelitis; primary idiopathic myxedema; nephrosis; ophthalmia symphatica; orchitis granulomatosa; pancreatitis; polyradiculitis acuta; pyoderma gangrenosum; Quervain's thyreoiditis; acquired splenic atrophy; non-malignant thymoma; vitiligo; toxic-shock syndrome; food poisoning; conditions involving infiltration of T cells; leukocyte-adhesion deficiency; immune responses associated with acute and delayed hypersensitivity mediated by cytokines and T-lymphocytes; diseases involving leukocyte diapedesis; multiple organ injury syndrome; antigen-antibody complex-mediated diseases; antiglomerular basement membrane disease; allergic neuritis; autoimmune polyendocrinopathies; oophoritis; primary myxedema; autoimmune atrophic gastritis; sympathetic ophthalmia; rheumatic diseases; mixed connective tissue disease; nephrotic syndrome; insulitis; polyendocrine failure; autoimmune polyglandular syndrome type I; adult-onset idiopathic hypoparathyroidism (AOIH); cardiomyopathy such as dilated cardiomyopathy; epidermolisis bullosa acquisita (EBA); hemochromatosis; myocarditis; nephrotic syndrome; primary sclerosing cholangitis; purulent or nonpurulent sinusitis; acute or chronic sinusitis; ethmoid, frontal, maxillary, or sphenoid sinusitis; an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils; anaphylaxis; seronegative spondyloarthritides; polyendocrine autoimmune disease; sclerosing cholangitis; chronic mucocutaneous candidiasis; Bruton's syndrome; transient hypogammaglobulinemia of infancy; Wiskott-Aldrich syndrome; ataxia telangiectasia syndrome; angiectasis; autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization; allergic hypersensitivity disorders; glomerulonephritides; reperfusion injury; ischemic reperfusion disorder; reperfusion injury of myocardial or other tissues; lymphomatous tracheobronchitis; inflammatory dermatoses; dermatoses with acute inflammatory components; multiple organ failure; bullous diseases; renal cortical necrosis; acute purulent meningitis or other central nervous system inflammatory disorders; ocular and orbital inflammatory disorders; granulocyte transfusion-associated syndromes; cytokine-induced toxicity; narcolepsy; acute serious inflammation; chronic intractable inflammation; pyelitis; endarterial hyperplasia; peptic ulcer; valvulitis; and endometriosis.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more additional therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g., a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodilator, a statin, an anti-inflammatory agent (e.g., methotrexate), or an NSAID. In certain embodiments, the additional therapy may include a combination of therapeutics of different classes.

The invention also provides a method of decreasing the expression of HLA-DR, CD86, CD83, IFNγ, IL-1b, IL-12 (e.g., IL-12p40), TNFα, IL-17A, IL-2, IL-23, or IL-6 in a cell, tissue, or subject. The method comprises contacting the cell, tissue, or subject with an effective amount of a Siglec extracellular domain (ECD), or a functional fragment or variant thereof, optionally conjugated to a serum half-life enhancer, disclosed herein. In certain embodiments, the cell is selected from a dendritic cell and a peripheral blood mononuclear cell (PBMC).

In certain embodiments, expression of HLA-DR, CD86, CD83, IFNγ, IL-1b, IL-10, TNFα, IL-17A, IL-2, or IL-6 in the cell, tissue, or subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with a construct described herein. Gene expression may be measured by any suitable method known in the art, for example, by ELISA, or by Luminex multiplex assays.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This Example describes the effects of Siglec extracellular domains (ECDs) on cytokine expression in activated T cells.

His-tagged versions of Siglec-1 and Siglec-2 ECDs (referred to as Sig-1-HIS, Sig-2-HIS) and Fc-fusions of Siglec-4, Siglec-5, Siglec-6, Siglec-7, Siglec-8, Siglec-10, Siglec-11 and Siglec-14 ECDs (referred to as Sig-4-Fc, Sig-5-Fc, Sig-6-Fc, Sig-8-Fc, Sig-10-Fc, Sig-11-Fc, and Sig-14-Fc) were purchased from R&D Systems through Fisher Scientific. Catalog nos. were 5197SL050 (Sig-1-HIS), 10-191-SL050 (Sig-2-HIS), 89-40M-G050 (Sig-4-Fc), 1072SL050 (Sig-5-Fc), 2859SL050 (Sig-6-Fc), 90-45S-1050 (Sig-8-Fc), 2130-SL (Sig-10-Fc), 3258SL050 (Sig-11-Fc), and 4905SL050 (Sig-14-Fc).

Isotype controls (IgG1 and IgG1-G), Fc-fusions of Siglec-3, Siglec-7, Siglec-9, and Siglec-15 ECDs (referred to as Sig-3-Fe, Sig-7-Fc-1G, Sig-9-Fe, Sig-9-Fc-1G, and Sig-15-Fc), an Fc-fusion of a sialic acid loss of binding (SAX) mutant (R120A) of Siglec-9 ECD (referred to as Sig-9-SAX-Fc-1G), and an Fc-fusion of the PDL1 ECD (referred to as PDL1-Fc) were recombinantly expressed and purified. Briefly, proteins were expressed in a 200 mL transfection of Expi293 human cells using the pFusion mammalian expression vector, purified using Protein A chromatography, followed by dialysis into PBS, pH 7.2. All Fc-fusions included a human IgG1 Fc (IgG1-G, Sig-7-Fc-1G, Sig-9-Fc-1G, and Sig-9-SAX-Fc-1G included a human IgG1 Fc with an N297G mutation). Purified proteins were assayed for endotoxin and characterized for purity by SDS-PAGE and SEC-HPLC.

Human PBMCs were thawed and enriched for T cells using the STEMCELL T isolation kit (Catalog: 19051). T cells were stimulated with anti-CD3 (OKT3 clone) and anti-CD28 (clone CD28.2) antibodies at a final concentration of 1 μg/ml in complete RPMI media (supplemented with 10% heat-inactivated FBS, non-essential amino acids, and sodium pyruvate). On day 2, floating cells were collected and re-plated in fresh complete RPMI media and anti-CD3 and anti-CD28 antibodies were replenished at 1 μg/mL to stimulate cells continuously. On day 4 or 5, cells were spun down and resuspended in fresh RPMI media (10% complete) and supplied with low levels of IL-2 (5 ng/mL or ˜50 IU). On day 7, U-bottom well plates were coated with anti-CD3 antibody (OKT-3 clone) at 1 μg/mL and incubated on a shaker for 2 hours. Activated T cells were harvested, centrifuged, resuspended in fresh media, and counted. Siglec ECDs (as described above) were diluted and added to the plate at the desired concentration. 200,000 activated T cells were added to each well to a final volume of 200 μL. Plates were incubated overnight, and the supernatants were harvested for analysis the next day. Cytokine concentration was measured using a Biolegend LEGENDplex kit (Catalog No. 741035).

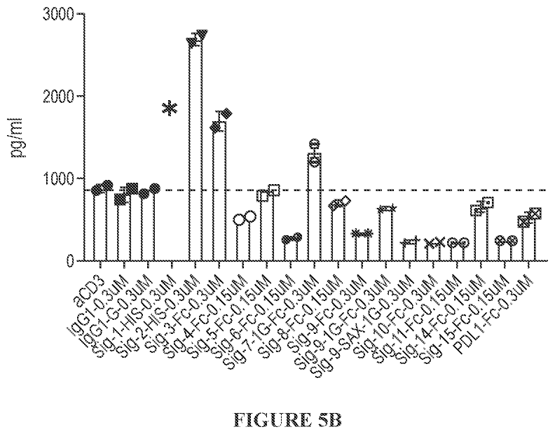

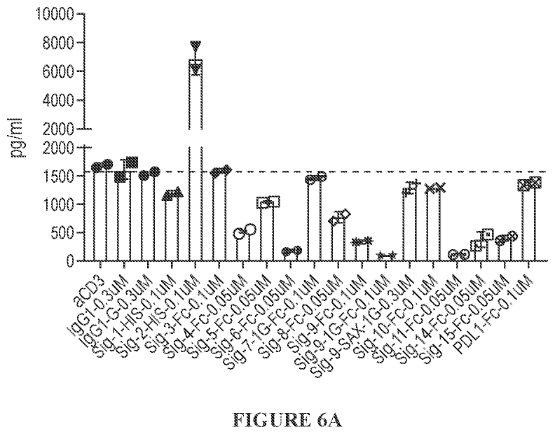

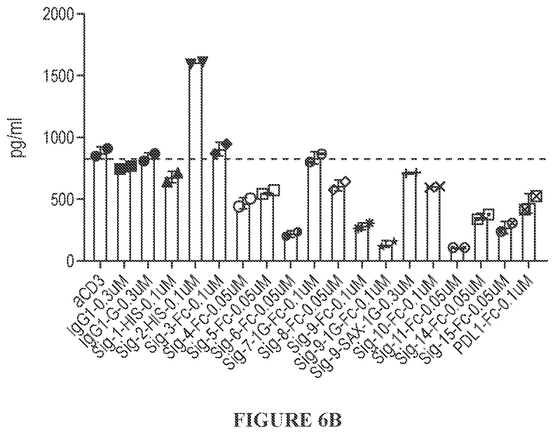

FIG. 5 depicts IFN-γ expression by primary human T cells (day 7-8 of culture) treated with the indicated Siglec ECD constructs at 0.3 or 0.15 μM. Results for two different donors are shown in FIG. 5A and FIG. 5B. FIG. 6 depicts IFN-γ expression by primary human T cells (day 7-8 of culture) treated with the indicated Siglec ECD constructs at 0.1 or 0.05 μM. Results for two different donors are shown in FIG. 6A and FIG. 6B.

As shown in FIG. 5 and FIG. 6, constructs including ECDs from Siglecs-1, -4, -6, -9, -11, and -15 (specifically, Sig-1-HIS, Sig-4-Fe, Sig-6-Fe, Sig-9-Fe, Sig-9-Fc-1G, Sig-9-SAX-Fc-1G, Sig-11-Fe, and Sig-15-Fc) as well as PDL-1 showed consistent and dose-dependent suppression of IFN-γ expression. Notably, suppression of IFN-γ expression was observed for Sig-9-SAX-Fc-1G, which included a mutation to alanine of an arginine required for sialic acid binding (R120A). Sig-2-HIS increased IFN-γ expression.

Testing of Siglec ECD constructs for modulation of expression of other cytokines, including IP-10, IL-4, IL-6, TNF-α, IL-17A, IL-10, IL-17A, IL-2, IL-6, IL-4, IL-10, granzyme A-B, and perform, gave similar results as for IFN-γ. IL-2, free active TGF-β, IL-8, MCP-1 and IL-1β showed a low level of basal cytokine expression, and therefore could not be used to assay modulation by Siglec ECD constructs. TNF-α expression by primary human T cells treated with the indicated Siglec ECD constructs at the indicated concentrations is shown in TABLE 1 and TABLE 2. TABLE 1 and TABLE 2 show the percent change in TNF-α expression relative to an untreated (stimulation only) control, and each table represents results for an independent donor. As shown in TABLE 1 and TABLE 2, Siglec ECD constructs had a dose-dependent effect on TNF-α expression.

TABLE 1

| | | | | | | | | | Sig-9- | | |
|---|---|---|---|---|---|---|---|---|---|---|---|
| | | | | | | | | Sig- | SAX- | | |
| Conc | Sig-1- | Sig-2- | Sig- | Sig- | Sig- | Sig- | Sig- | 9-Fc- | Fc- | Sig- | Sig- |
| (μM) | HIS | HIS | 4-Fc | 5-Fc | 6-Fc | 8-Fc | 9-Fc | 1G- | 1G- | 11-Fc | 14-Fc |
| 0.016 | 13.4 | 3.4 | −21.4 | −19.0 | −21.5 | — | −15.0 | −22.8 | −33.1 | −16.9 | −15.7 |
| 0.033 | 37.3 | 17.5 | −31.4 | −27.7 | −34.0 | — | −19.8 | −40.1 | −52.0 | −28.9 | −23.7 |
| 0.063 | 82.3 | 54.0 | −36.6 | −43.4 | −57.2 | — | −41.4 | −81.8 | −76.4 | −56.6 | −32.4 |
| 0.125 | 62.4 | 137.7 | −34.2 | −56.1 | −86.1 | −10.8 | −59.0 | −90.8 | −88.9 | −87.5 | −63.8 |
| 0.250 | −51.4 | 215.6 | −46.2 | −62.1 | −91.1 | −16.5 | −65.8 | −95.0 | −90.8 | −99.1 | −80.8 |
| 0.500 | — | 397.4 | −83.1 | −62.1 | −80.5 | −27.4 | −71.6 | −95.0 | −90.8 | −99.7 | −80.8 |

TABLE 2

| | | | | | | | | | Sig-9- | | |
|---|---|---|---|---|---|---|---|---|---|---|---|
| | | | | | | | | Sig- | SAX- | | |
| Conc | Sig-1- | Sig-2- | Sig- | Sig- | Sig- | Sig- | Sig- | 9-Fc- | Fc- | Sig- | Sig- |
| (μM) | HIS | HIS | 4-Fc | 5-Fc | 6-Fc | 8-Fc | 9-Fc | 1G- | 1G- | 11-Fc | 14-Fc |
| 0.016 | −16.9 | −11.4 | −27.2 | −20.2 | −53.0 | — | −21.1 | −20.6 | −27.0 | −53.1 | −20.4 |
| 0.033 | −38.2 | −40.7 | −54.8 | −8.2 | −58.7 | — | −31.5 | −28.2 | −40.4 | −54.7 | −12.1 |
| 0.063 | −9.1 | −29.1 | −57.9 | −32.3 | −59.4 | — | −58.0 | −78.0 | −77.8 | −65.3 | −38.0 |
| 0.125 | 17.9 | 4.2 | −63.5 | −47.2 | −80.2 | −12.2 | −70.4 | −88.4 | −84.2 | −79.9 | −66.3 |
| 0.250 | 17.8 | 72.1 | −62.3 | −51.3 | −93.7 | −10.4 | −75.3 | −90.0 | −87.7 | −94.2 | −77.8 |
| 0.500 | — | 114.5 | −53.1 | −51.3 | −92.7 | −16.9 | −80.0 | −90.0 | −87.7 | −98.6 | −77.8 |

### Example 2

This Example describes the construction of Siglec ECD constructs and determination of the Siglec ECD construct's binding properties.

Siglec ECD constructs described in this Example are depicted in TABLE 3, which include Fc fusion proteins with wild-type Siglec ECD domains and Fc fusion proteins with mutant sialic acid loss of binding (SAX) Siglec ECD domains.

Briefly, proteins indicated in TABLE 4 were expressed in a 200 mL transfection of Expi293 human cells using the pFusion mammalian expression vector, purified using Protein A chromatography, followed by dialysis into PBS, pH 7.2. Purified proteins were assayed for endotoxin and characterized for purity by SDS-PAGE and SEC-HPLC. Yield and percent purity are indicated in TABLE 4.

TABLE 3

| | Amino Acid | Nuc | | | |
|---|---|---|---|---|---|
| | SEQ | SEQ | Description | | |
| Construct | ID NO | ID NO | Siglec ECD | Linker | Fc |
| Sig-1-VsetC2-Fc-1G | 19 | 20 | Human Siglec-1 | (G₄S)₂ | Human |
| | | | (Residues 20-320) | | IgG1 Fc (N297G) |
| Sig-1-VsetC2-SAX- | 21 | 22 | Human Siglec-1 SAX | (G₄S)₂ | Human |
| Fc-1G | | | (Residues 20-320; R116A) | | IgG1 Fc (N297G) |
| Sig-3-Fc-1G | 139 | 140 | Human Siglec-3 | | Human |
| | | | (Residues 18-259) | | IgG1 Fc (N297G) |
| Sig-3-SAX-Fc-1G | 31 | 32 | Human Siglec-3 SAX | | Human |
| | | | (Residues 18-259, R119K) | | IgG1 Fc (N297G) |
| Sig-4-Fc-1G | 37 | 38 | Human Siglec-4 | (G₄S)₂ | Human |
| | | | (Residues 20-516) | | IgG1 Fc (N297G) |
| Sig-4-SAX-Fc-1G | 39 | 40 | Human Siglec-4 SAX | (G₄S)₂ | Human |
| | | | (Residues 20-516, R118A) | | IgG1 Fc (N297G) |
| Sig-5-Fc-1G | 141 | 142 | Human Siglec-5 | | Human |
| | | | (Residues 17-441) | | IgG1 Fc (N297G) |
| Sig-5-SAX-Fc-1G | 143 | 144 | Human Siglec-5 SAX | | Human |
| | | | (Residues 17-441, R119A) | | IgG1 Fc (N297G) |
| Sig-6-Fc-1G | 47 | 48 | Human Siglec-6 | (G₄S)₂ | Human |
| | | | (Residues 27-347) | | IgG1 Fc (N297G) |
| Sig-6-SAX-Fc-1G | 49 | 50 | Human Siglec-6 SAX | (G₄S)₂ | Human |
| | | | (Residues 27-347, R122A) | | IgG1 Fc (N297G) |
| Sig-7-Fc-1G | 59 | 60 | Human Siglec-7 | | Human |
| | | | (Residues 19-357) | | IgG1 Fc (N297G) |
| Sig-7-SAX-Fc-1G | 61 | 62 | Human Siglec-7 SAX | | Human |
| | | | (Residues 19-357, R124K) | | IgG1 Fc (N297G) |
| Sig-9-Fc | 120 | 121 | Human Siglec-9 | | Human |
| | | | (Residues 18-348; | | IgG1 Fc |
| | | | C158S, C295Y) | | |
| Sig-9-Fc-1G | 73 | 74 | Human Siglec-9 | | Human |
| | | | (Residues 18-348; | | IgG1 Fc (N297G) |
| | | | C158S, C295Y) | | |
| Sig-9-SAX-Fc-1G | 75 | 76 | Human Siglec-9 SAX | | Human |
| | | | (Residues 18-348; | | IgG1 Fc (N297G) |
| | | | C158S, C295Y, R120K) | | |
| Sig-9-SAX-Fc-1G | 122 | 123 | Human Siglec-9 SAX | EPKSS | Human |
| (Modified Hinge) | | | (Residues 18-348; | | IgG1 Fc (N297G) |
| | | | C158S, C295Y, R120K) | | |
| Sig-9-His | 79 | 80 | Human Siglec-9 | | |
| | | | (Residues 18-348; | | |
| | | | C158S, C295Y) | | |
| Sig-10-Fc-1G | 90 | 91 | Human Siglec-10 | (G₄S)₂ | Human |
| | | | (Residues 18-550) | | IgG1 Fc (N297G) |
| Sig-11-Fc-1G | 96 | 97 | Human Siglec-11 | (G₄S)₂ | Human |
| | | | (Residues 28-561) | | IgG1 Fc (N297G) |
| Sig-11-SAX-Fc-1G | 98 | 99 | Human Siglec-11 SAX | (G₄S)₂ | Human |
| | | | (Residues 28-561, R132A) | | IgG1 Fc (N297G) |
| Sig-14-Fc-1G | 145 | 146 | Human Siglec-14 | | Human |
| | | | (Residues 17-358) | | IgG1 Fc (N297G) |
| Sig-14-SAX-Fc-1G | 147 | 148 | Human Siglec-14 SAX | | Human |
| | | | (Residues 17-358, R119A) | | IgG1 Fc (N297G) |
| Sig-15-Fc | 124 | 125 | Human Siglec-15 | (G₄S)₂ | Human |
| | | | (Residues 20-263) | | IgG1 Fc |
| Sig-15-Fc-1G | 108 | 109 | Human Siglec-15 | (G₄S)₂ | Human |
| | | | (Residues 20-263) | | IgG1 Fc (N297G) |
| mSig-15-Fc-1G | 112 | 113 | Mouse Siglec-15 ECD | (G₄S)₂ | Human |
| | | | | | IgG1 Fc (N297G) |
| Sig-15-SAX-Fc-1G | 110 | 111 | Human Siglec-15 SAX | (G₄S)₂ | Human |
| | | | (Residues 20-263, R143A) | | IgG1 Fc (N297G) |
| Sig-16-Fc-1G | 117 | 118 | Human Siglec-16 | (G₄S)₂ | Human |
| | | | (Residues 15-434) | | IgG1 Fc (N297G) |
| Sig-9-SNP3-Fc-1G | 83 | 84 | Human Siglec-9 | (G₄S)₂ | Human |
| | | | (Residues 18-348; K131Q) | | IgG1 Fc (N297G) |
| Sig-9-SNP6-Fc-1G | 85 | 86 | Human Siglec-9 | (G₄S)₂ | Human |
| | | | (Residues 18-348; | | IgG1 Fc (N297G) |
| | | | K100E, A315E) | | |

TABLE 4

| Construct | Yield (mg/L) | Purity (%) |
|---|---|---|
| Sig-1-VsetC2-Fc-1G | 1.2 | 11 |
| Sig-1-VsetC2-SAX-Fc-1G | 2 | 30 |
| Sig-3-SAX-Fc-1G | 120 | 82 |
| Sig-4-Fc-1G | 24 | 93 |
| Sig-4-SAX-Fc-1G | 93.6 | 97 |
| Sig-6-Fc-1G | 201.6 | 81 |
| Sig-6-SAX-Fc-1G | 97.2 | 45 |
| Sig-7-Fc-1G | 210 | 95 |
| Sig-7-SAX-Fc-1G | 128 | 99 |
| Sig-9-Fc-1G | 225 | 94 |
| Sig-9-SAX-Fc-1G | 220 | 100 |
| Sig-10-Fc-1G | 15 | 55 |
| Sig-11-Fc-1G | 6.44 | 38.5 |
| Sig-11-SAX-Fc-1G | 7.02 | 35 |
| Sig-15-Fc-1G | 188 | 100 |
| mSig-15-Fc-1G | 88.4 | 96 |
| Sig-15-SAX-Fc-1G | 270 | 93 |
| Sig-16-Fc-1G | 4.42 | 14 |

Siglec ECD constructs were characterized with respect to binding to (i) sialic acid probes, (ii) cell surface sialic acid, (iii) human Fc γ receptor, and (iv) monoclonal antibodies.

### Siglec ECD Binding to a Sialic Acid Probe

Siglec ECD constructs were characterized using an ELISA to determine binding to a biotinylated sialic acid probe.

Briefly, the probe (Neu5Aca2-3Galβ1-4(Fucα1-3)(6-HSO₃)GlcNAcβ-PAA-biotin; GlycoTech) was diluted in ELISA buffer prior to loading on wells coated with the appropriate Siglec ECD construct. Probe binding was detected using HRP-conjugated streptavidin. The plate was developed with 3,3′,5,5′-Tetramethylbenzidine (TMB), and absorbance at 450 nm was read using a plate reader.

Results for Sig-7-Fc-1G, Sig-7-SAX-Fc-1G, Sig-9-Fc-1G, and Sig-9-SAX-Fc-1G are shown in TABLE 5, with Y indicating dose-dependent binding above background. As expected, Siglec ECD constructs with unmodified (wild-type) ECDs (Sig-7-Fc-1G and Sig-9-Fc-1G) demonstrated binding to the α2,3 sialic acid probe, while no binding was observed for the SAX Siglec ECD constructs (Sig-7-SAX-Fc-1G and Sig-9-SAX-Fc-1G). Sig-15-SAX-Fc-1G was shown not to bind an α2,6 probe from GlycoTech utilizing the same protocol.

TABLE 5

| Construct | SA Probe Binding |
|---|---|
| Sig-7-Fc-1G | Y (2-3) |
| Sig-7-SAX-Fc-1G | N (2-3) |
| Sig-9-Fc-1G | Y (2-3) |
| Sig-9-SAX-Fc-1G | N (2-3) |
| Sig-15-SAX-Fc-1G | N (2-6) |

### Siglec ECD Binding to Cell Surface Sialic Acid

The K562 human leukemia cell line was used to analyze the ability of Siglec ECD constructs to bind sialic acid on cell surfaces.

Briefly, cells were incubated with the appropriate Siglec ECD construct for 30-60 minutes at 4° C. Cells were washed and incubated with AF647-labeled goat anti-human IgG for 30 minutes at 4° C. Cells were washed and analyzed by flow cytometry. The results for Sig-3-Fc-1G, Sig-3-SAX-Fc-1G, Sig-7-Fc-1G, Sig-7-SAX-Fc-1G, Sig-9-Fc-1G, Sig-9-SAX-Fc-1G, Sig-15-Fc-1G, and Sig-15-SAX-Fc-1G are shown in TABLE 6, with Y indicating specific concentration-dependent cell binding above background, and N indicating no binding or binding similar to isotype control. As expected, Sig-3-Fc-1G, Sig-7-Fc-1G, Sig-9-Fc-1G, and Sig-15-Fc-1G demonstrated binding to K562 cells while no binding was observed for Sig-3-SAX-Fc-1G, Sig-7-SAX-Fc-1G, Sig-9-SAX-Fc-1G, and Sig-15-SAX-Fc-1G.

TABLE 6

| Construct | K562 Binding |
|---|---|
| Sig-3-Fc-1G | Y |
| Sig-3-SAX-Fc-1G | N |
| Sig-7-Fc-1G | Y |
| Sig-7-SAX-Fc-1G | N |
| Sig-9-Fc-1G | Y |
| Sig-9-SAX-Fc-1G | N |
| Sig-15-Fc-1G | Y |
| Sig-15-SAX-Fc-1G | N |

### Siglec ECD Binding to Monoclonal Antibodies

Binding of purified Siglec ECD constructs to anti-Siglec antibodies was measured by ForteBio Octet.

Briefly, Siglec ECD constructs were captured on an AHC (Anti-human IgG-Capture) Biosensor. Siglec-specific antibodies for Siglec-6 (R&D Systems; MA2859), Siglec-9 (R&D Systems MAB1139), Siglec-10 (Thermo Fisher Scientific; MA5-28237), Siglec-11 (R&D Systems; MAB3258), and Siglec-15 (a monoclonal antibody derived from a 5G12 variant having a hVH1 heavy chain and hVL3 light chain as described in WO2018/057735) were titrated from 100 nM in a 2× series dilution. The signal was subtracted with buffer reference and aligned to the baseline. KD, Kon and Koff values were generated using a 1:1 fitting model. The binding kinetics for the following Siglec ECD constructs were determined: Sig-6-Fc-1G, Sig-6-SAX-Fc-1G, Sig-9-Fc, Sig-9-Fc-1G, Sig-9-SAX-Fc-1G, Sig-11-Fc-1G, Sig-11-SAX-Fc-1G, Sig-15-Fc-1G, and Sig-15-SAX-Fc-1G. All the ForteBio Octet assays used standard/conventional settings.

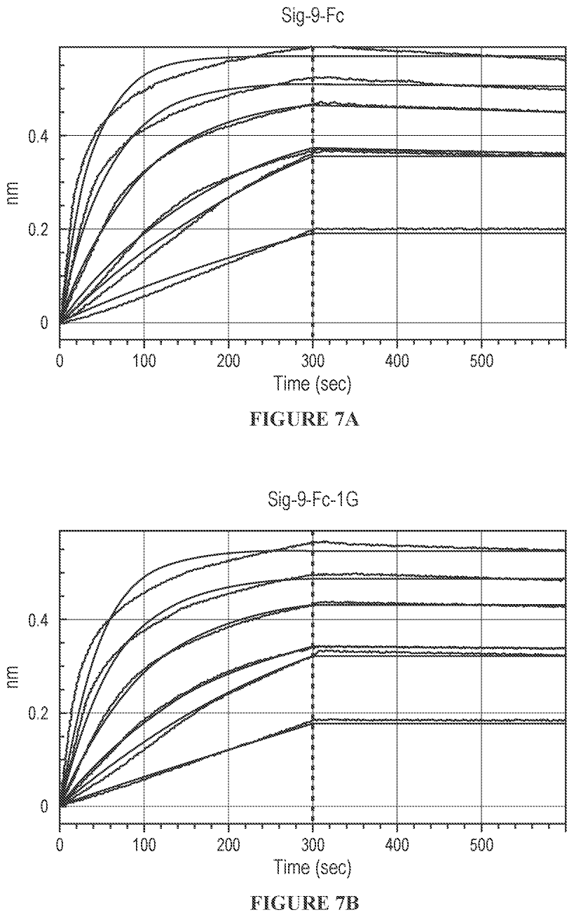

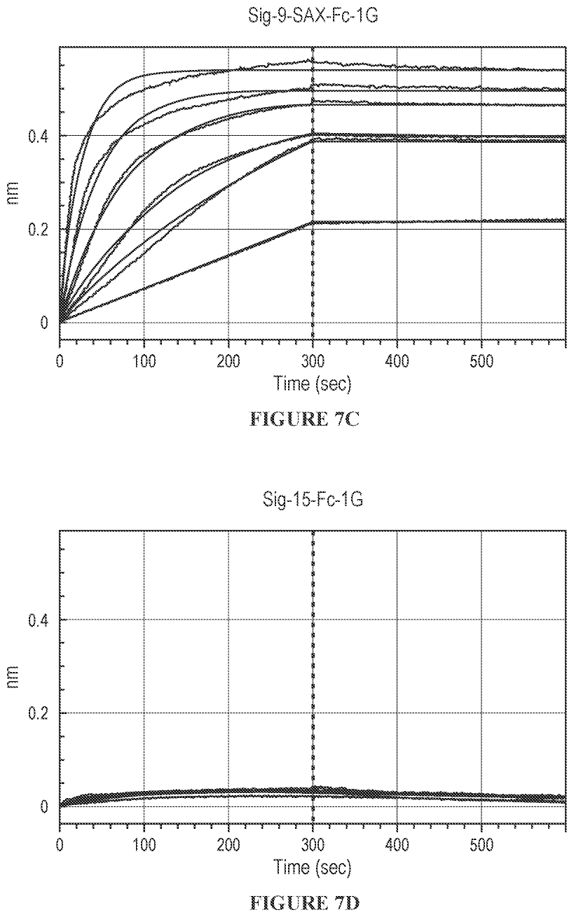

Results for all Siglec ECD constructs tested are summarized in TABLE 7 (Y indicates binding) and binding curves for Siglec-9 based ECD constructs are shown in FIG. 7. FIG. 7 depicts the results for Sig-9-Fc (FIG. 7A), Sig-9-Fc-1G (FIG. 7B) and Sig-9-SAX-Fc-1G (FIG. 7C) binding to a Sig-9 specific antibody, with binding affinities below 10 nM. Sig-15-Fc-1G was used as a negative control (FIG. 7D), with no binding observed even at 100 nM. It is believed that these results suggest that the Siglec ECD constructs, including those with SAX mutations, are folded correctly.

TABLE 7

| Construct | mAb Binding |
|---|---|
| Sig-6-Fc-1G | Y |
| Sig-6-SAX-Fc-1G | Y |
| Sig-9-Fc | Y |
| Sig-9-Fc-1G | Y |
| Sig-9-SAX-Fc-1G | Y |
| Sig-11-Fc-1G | Y |
| Sig-11-SAX-Fc-1G | Y |
| Sig-15-Fc-1G | Y |
| Sig-15-SAX-Fc-1G | Y |

### Siglec ECD Binding to Human Fcγ Receptor

Binding of purified Siglec ECD constructs to human Fcγ receptor 1 was measured using ForteBio octet.

Briefly, recombinant human Fcγ receptor 1 (Acros Biosystem) was captured on an anti-penta His (HIS1K) biosensor, and Siglec ECD constructs were titrated for binding. Data are summarized in TABLE 8 (N indicates no binding). As expected, no binding was observed for Siglec ECD constructs containing a N297G IgG1 Fc (Fc-1G). In contrast, constructs with wild type IgG1 Fc (Fc) bound with 10 to 100 nM affinities

TABLE 8

| Construct | Hu Fcγ Receptor Binding |
|---|---|
| Sig-7-Fc-1G | N |
| Sig-9-Fc-1G | N |
| Sig-10-Fc-1G | N |
| Sig-15-Fc-1G | N |
| mSig-15-Fc-1G | N |

### Example 3

This Example describes testing of Siglec ECD constructs (including Siglec Sialic acid loss of binding (“SAX”) ECD constructs) for their ability to suppress immune cells.

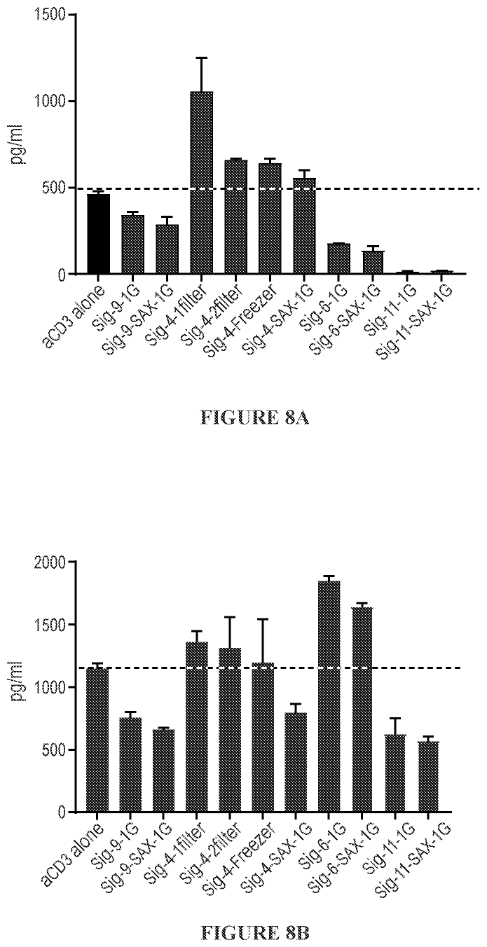

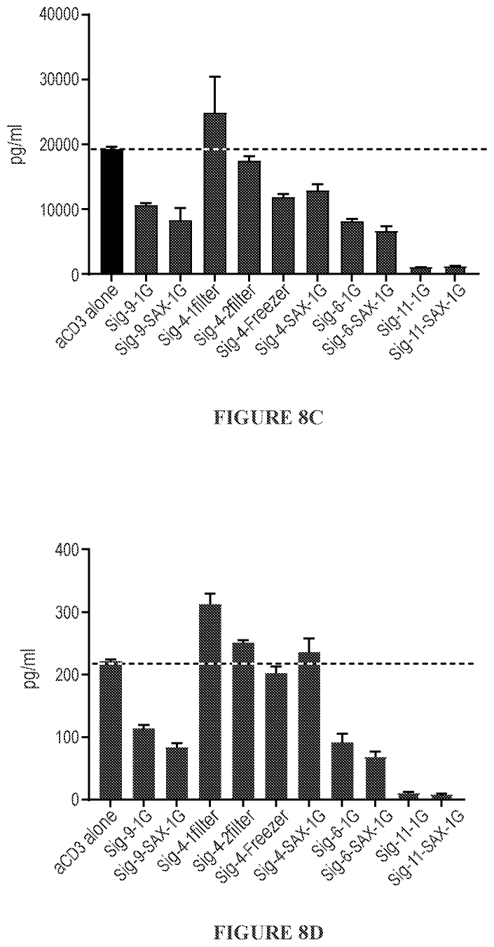

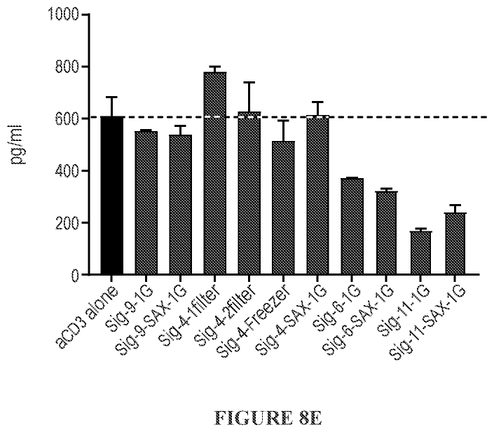

The ability of Siglec ECD constructs to suppress activated T cells was analyzed. Activated T cells were prepared and assayed as described in Example 1. Siglec ECD constructs were prepared as described in Examples 1 or 2. FIG. 8 depicts cytokine expression by primary human T cells (day 7-8) in the presence of the indicated Siglec ECD constructs. Depicted is IFN-γ (FIG. 8A), Granzyme B (FIG. 8B), Granzyme B (FIG. 8C), soluble Fas Ligand (FIG. 8D), and Perform (FIG. 8E) expression. As can be seen in FIG. 8, Siglec-6, Siglec-9, and Siglec-11 ECD based constructs, including SAX versions, demonstrated consistent suppression of cytokine expression following anti-CD3 antibody stimulation of primary human T cells, while results for Siglec-4 based ECD constructs were inconsistent.

Further results are summarized in TABLE 9 (Y indicates statistically significant suppression of IFN-γ expression relative to isotype control; N indicates no statistically significant suppression of IFN-γ expression relative to isotype control). Siglec-5, Siglec-14, and Siglec-15 based ECD constructs also demonstrated consistent suppression of cytokine expression following anti-CD3 antibody stimulation of primary human T cells. Siglec-3, Siglec-7, and Siglec-10 based ECD constructs, including SAX versions, demonstrated no or little suppression of cytokine expression.

TABLE 9

| Construct | T-cells |
|---|---|
| Sig-3-Fc-1G | N |
| Sig-3-SAX-Fc-1G | N |
| Sig-4-Fc-1G | N |
| Sig-4-SAX-Fc-1G | N |
| Sig-5-Fc-1G | Y |
| Sig-5-SAX-Fc-1G | Y |
| Sig-6-Fc-1G | Y |
| Sig-6-SAX-Fc-1G | Y |
| Sig-7-Fc-1G | N |
| Sig-7-SAX-Fc-1G | N |
| Sig-9-Fc-1G | Y |
| Sig-9-SAX-Fc-1G | Y |
| Sig-10-Fc-1G | N |
| Sig-11-Fc-1G | Y |
| Sig-11-SAX-Fc-1G | Y |
| Sig-14-Fc-1G | Y |
| Sig-14-SAX-Fc-1G | Y |
| Sig-15-Fc-1G | Y |
| Sig-15-SAX-Fc-1G | Y |

The ability of Siglec ECD constructs to suppress primary Pan-T cells was also analyzed. Activated T cells were prepared as described in Example 1. Primary Pan-T cells were prepared by serially culturing in the presence of aCD3 and aCD28 antibodies at a concentration of 1 μg/mL. Cells were passaged and replenished on days 2 and 4. The cells were rested for 3 days in low IL-2 (5 ng/mL). On day 7 or 8 cells were ready to use for functional assays. Siglec ECD constructs were prepared as described in Examples 1 or 2.

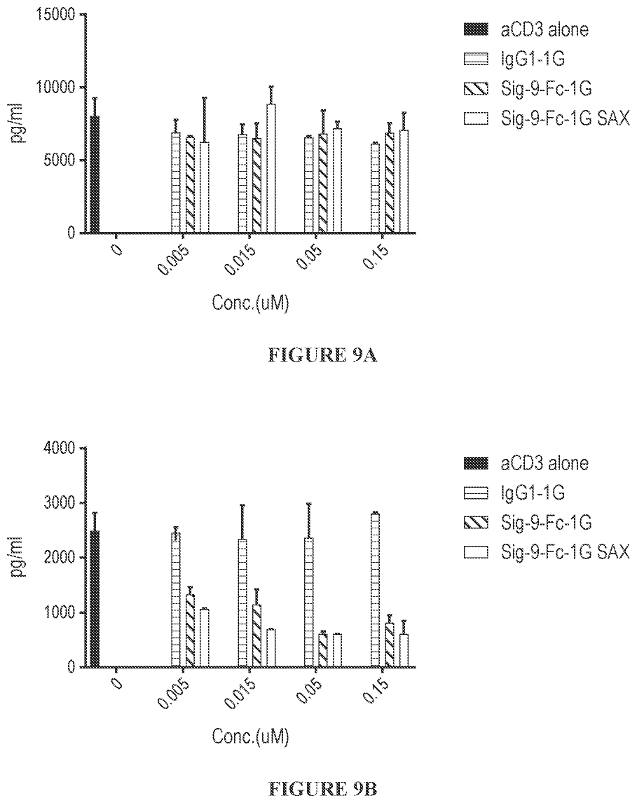

FIG. 9 depicts the effect of Sig-9-Fc-1G and Sig-9-SAX-Fc-1G on enriched primary Pan-T cells (FIG. 9A) and cultured activated T cells (FIG. 9B) as measured by IFN-γ secretion. Suppression by the Siglec ECD constructs was only seen for activated T cells. Again, both the wild type Siglec and Siglec SAX constructs had equal potency.

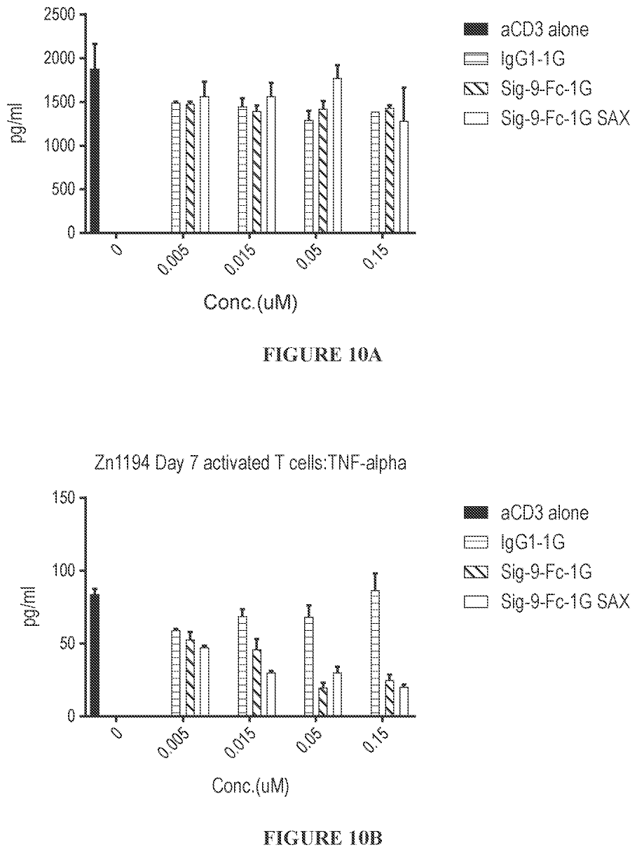

FIG. 10 depicts the effect of Sig-9-Fc-1G and Sig-9-SAX-Fc-1G on enriched primary Pan-T cells (FIG. 10A) and cultured activated T cells (FIG. 10B) as measured by TNF-α secretion. Suppression by the Siglec ECD constructs was only seen for activated T cells. Again, both the wild type Siglec and Siglec SAX constructs had equal potency.

A Jurkat-based luciferase cell assay was used to further analyze the ability of Siglec ECD constructs to suppress T Cell activation (Jurkat-Lucia™ NFAT Cells; InvivoGen). Jurkat-Lucia NFAT cells were derived from human immortalized Jurkat T cells by stable integration of an NFAT-inducible Lucia luciferase reporter construct. NFAT proteins are a family of transcription factors important in T cell activation. Jurkat-Lucia NFAT cells induce the activation of NFAT in response to PMA and T-lymphocyte antigens, such as concanavalin A (ConA) or phyto hemagglutinin (PHA).

Assays were conducted as follows. Jurkat-Lucia NFAT cells were grown in complete IMDM medium (10% FBS) to 70-80% confluence passaging 1 or 2 times with the occasional addition of the selective antibiotic zeocin for induction of NFAT-Lucia. Once sufficient cell numbers were obtained, they were centrifuged at 300×g for 5 minutes. Supernatant was removed, and cells were counted using trypan blue. Cells were resuspended at 2×10\hat{ }6 cells/ml in pre-warmed media. Siglec ECD constructs were prepared as described in Examples 1 or 2. 10 μL of a Siglec ECD construct stock solution at the desired concentration was added into each appropriate well in a 96-well assay plate. Each assay condition was run in triplicate. Con A alone without any Siglec ECD construct was used as a positive control, and no con A was used as a negative control. 180 μL of the cell suspension was added to the plate. 10 μL of a con A stock was added into each appropriate well to a final concentration of 25 μg/mL. Plates were incubated at 37° C. for 18-20 hours. A luciferase substrate (Quanti-Luc) was added after 18-20 hours following manufacturer instructions to generate a luminescent signal, and relative light units (RLUs) were measured.

FIG. 11 depicts the effects of the indicated Siglec ECD constructs in the Jurkat Luciferase cell assay. FIG. 11A depicts Sig-9-Fc-1G and Sig-9-SAX-Fc-1G induced inhibition of NFAT activation in the Jurkat Luciferase assay, FIG. 11B depicts Sig-11-Fc-1G and Sig-11-SAX-Fc-1G induced inhibition of NFAT activation in the Jurkat Luciferase assay, and FIG. 11C depicts Sig-15-Fc-1G induced inhibition of NFAT activation in the Jurkat Luciferase assay.

Recombinant Siglec-ECD constructs derived from human Siglec-5, -6, -9, -11, -14, and -15 along with their SAX counterparts were also tested in the Jurkat Luciferase cell assay. A summary of these results is shown in TABLE 10 (Y indicates a statistically significant suppression of luciferase activity as compared to isotype control). The Siglec-5 and Siglec-14 ECD constructs demonstrated dose dependent suppression in the 1 to 0.03 μM range.

TABLE 10

| Construct | Jurkat Lucia |
|---|---|
| Sig-5-Fc-1G | Y |
| Sig-5-SAX-Fc-1G | Y |
| Sig-6-Fc-1G | N |
| Sig-6-SAX-Fc-1G | N |
| Sig-9-Fc-1G | Y |
| Sig-9-SAX-Fc-1G | Y |
| Sig-11-Fc-1G | Y |
| Sig-11-SAX-Fc-1G | Y |
| Sig-14-Fc-1G | Y |
| Sig-14-SAX-Fc-1G | Y |
| Sig-15-Fc-1G | Y |
| Sig-15-SAX-Fc-1G | Y |

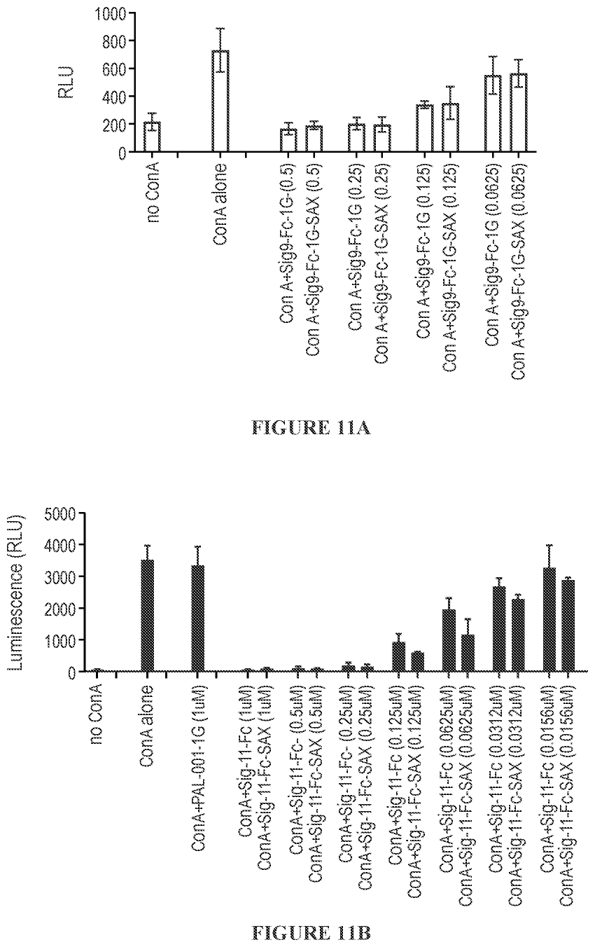

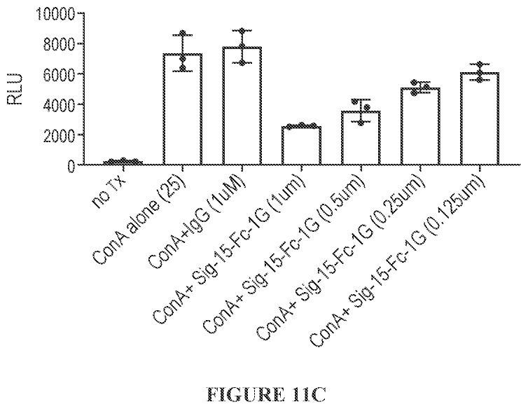

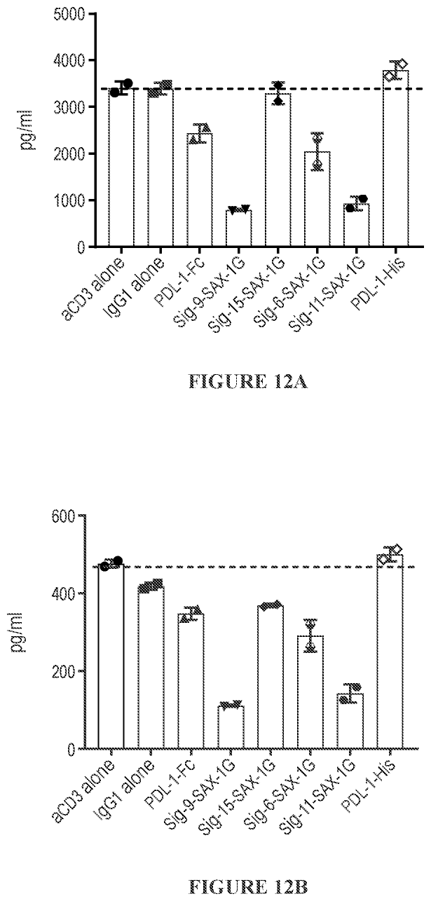

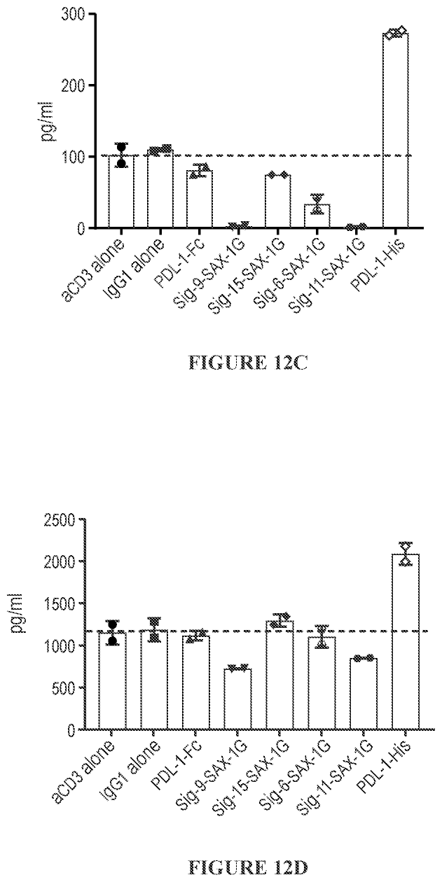

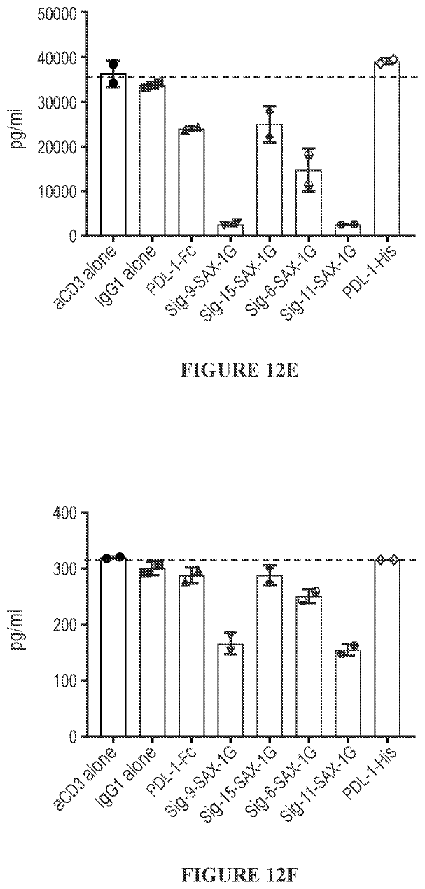

Siglec ECD constructs were also tested for their ability to modulate Th1 cytokine expression, as described in Example 1. Siglec ECD constructs were prepared as described in Examples 1 and 2. Also tested were PDL-1-Fc (as described in Example 1) and a His-tagged PDL-1 (PDL-1-His). As shown in FIG. 12, Sig-9-SAX-Fc-1G, Sig-6-SAX-Fc-1G, Sig-11-SAX-Fc-1G, and Sig-15-SAX-Fc-1G suppressed expression of cytokines and cytotoxicity molecules in activated T cells. Shown are levels of IFN-γ (FIG. 12A), TNF-α (FIG. 12B), IL-10 (FIG. 12C), Granzyme A (FIG. 12D), Granzyme B (FIG. 12E) and Perform (FIG. 12F).

Siglec ECD constructs were also tested for their ability to suppress primary human macrophages. Siglec ECD constructs were prepared as described in Examples 1 or 2. Human PBMCs were isolated from leukopaks and monocytes were isolated using Miltenyi kits (CD14 microbeads, Human, Miltenyi Cat #130-050-201). Monocytes were differentiated in 20% FBS RPMI media supplied with non-essential amino acids (Gibco) and sodium pyruvate (Gibco) for 6 days. Cells were supplied with M-CSF at 50 ng/ml (R&D Systems). Different plate surfaces were used for generating different macrophage phenotypes: M1-like (Ultra Low), M2-like (untreated) and a mix of M1+M2 (treated with plasma). On day 3, half medium was changed into fresh medium and supplied with 2× M-CSF conc. On day 6, cells were harvested using Accutase (Gibco) and the scraping method. Cells were re-plated in a 96-well flat-bottom plate at 25,000 cells/well in 100 μL media supplemented with 10 ng/mL M-CSF. The next day, cells were changed into fresh 10% complete RPMI medium and Siglec ECD test articles were added at the desired concentration. The cells were stimulated with LPS (25 ng/mL; InVivoGen) and IFN-γ (10 ng/mL; R&D Systems). Cells were incubated at 37° C. overnight. Cells and supernatant were harvested after 16-18 hours of incubation for analysis. IL-12p40 and IL-10 expression was measured using a Biolegend LEGENDplex kit (Catalog No. 740502).

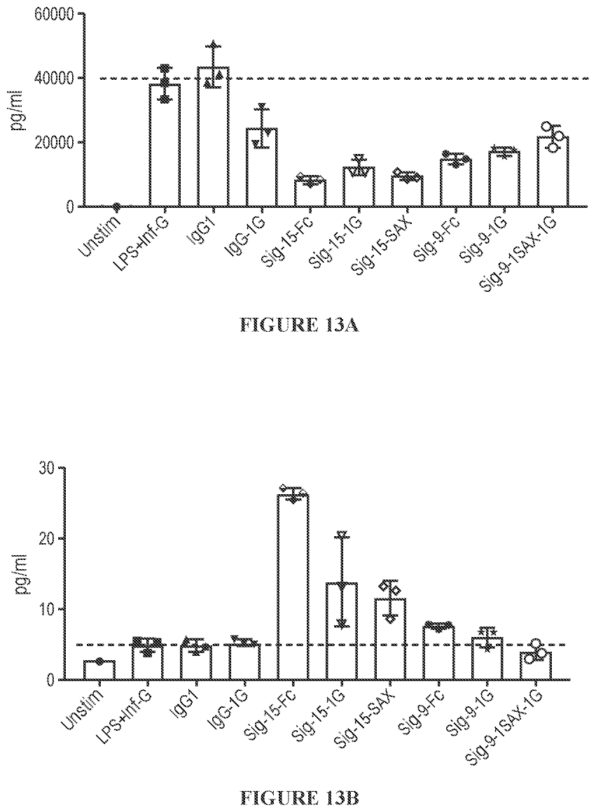

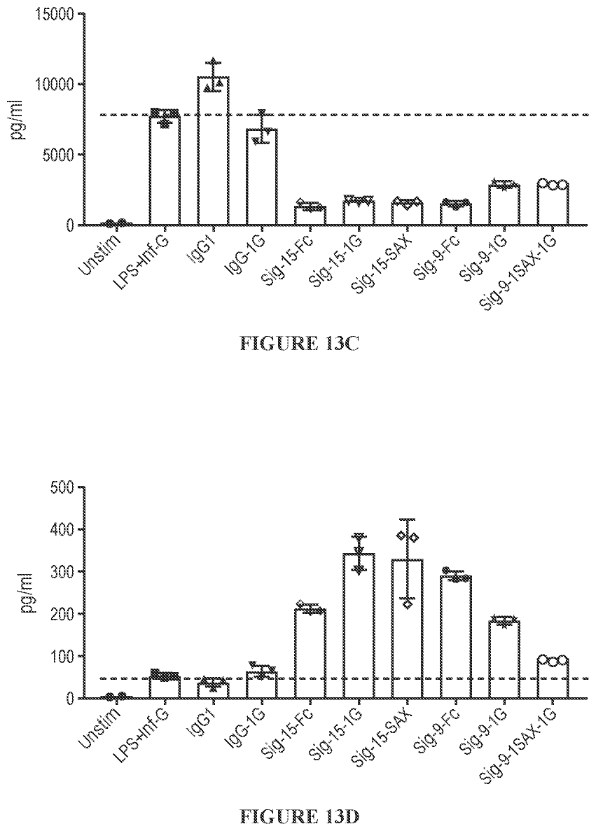

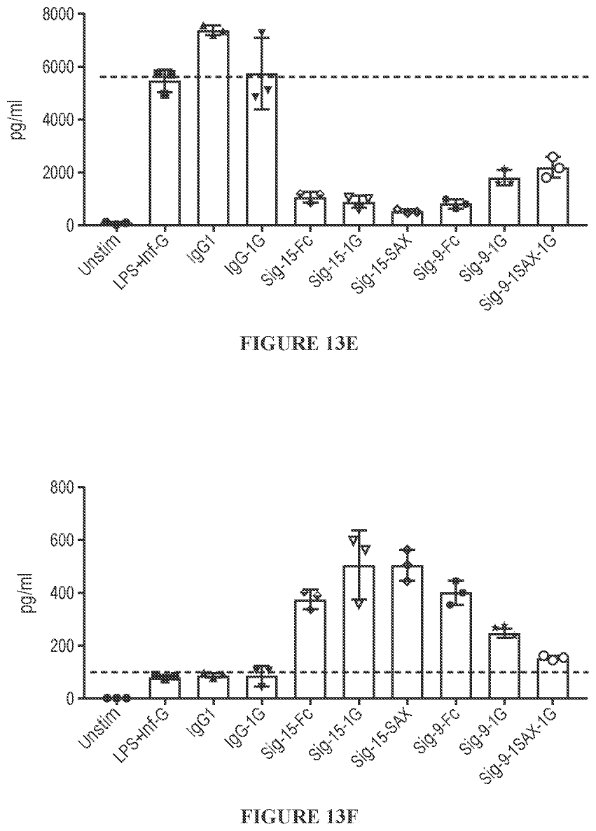

FIG. 13 depicts IL-12p40 and IL-10 expression in cells treated with Sig-9-Fc, Sig-9-Fc-1G, Sig-9-SAX-Fc-1G, Sig-15-Fc, Sig-15-Fc-1G, and Sig-15-SAX-Fc-1G. Results are shown for primary human M1 macrophages Ultra Low (FIG. 13A and FIG. 13B), untreated primary human macrophages (FIG. 13C and FIG. 13D), and in treated primary human macrophages (FIG. 13E and FIG. 13F).

Siglec-9 and Siglec-15 based ECD constructs, including their SAX variants, demonstrated suppression of IL-12p40 in all three macrophages types tested. An increase in IL-10 was also observed for Siglec-9 and Siglec-15 based ECD constructs, including their SAX variants in all three macrophage types. Results are also summarized in TABLE 11 (Y indicates a statistically significant suppression of IL-12p40 and increase in IL-10 as compared to isotype control).

TABLE 11

| Construct | Mφ |
|---|---|
| Sig-9-Fc-1G | Y |
| Sig-9-SAX-Fc-1G | Y |
| Sig-15-Fc-1G | Y |
| Sig-15-SAX-Fc-1G | Y |

### Example 4

This example describes the effects of Siglec ECD constructs in in vivo studies.

A mouse IPF (idiopathic pulmonary fibrosis) model study was conducted at Aragen Inc. C57bl6 mice were grouped (10 mice/group) and inoculated with a bleomycin dose orally. A control group of 5 mice was left without the bleomycin dose. The test articles were Sig-9-Fc, Sig-9-Fc-1G, Sig-9-SAX-Fc-1G, Sig-15-SAX-Fc-1G, and mSig-15-Fc-1G. The test articles were dosed at 10 mg/kg at 2 doses/week after day 7. A total of 4 doses were delivered IP. The study ran until day 21, after which mice were sacrificed for analysis. In-life harvest included lung weight, body weight, and leukocyte count. Flash frozen lung lobes (left side) were analyzed for hydroxyproline readout, which serves as qualitative representation of collagen deposits in the lungs (collagen has approximately 15% hydroxyproline).

FIG. 14 depicts the effects of Siglec ECD constructs in the mouse IPF (Idiopathic pulmonary fibrosis) model. A robust reduction in hydroxyproline read was seen with all the treatment groups as compared to vehicle.

A second in vivo study analyzed Siglec ECD constructs in a mouse CAIA (Collagen Antibody Induced Arthritis) model. The study was conducted at Charles River-Montreal. BalbC mice were randomized into groups (10 mice/group) and inoculated with collagen antibody cocktail arthritomab on day 0. The arthritis model was accelerated by an LPS boost on day 6. The test articles were Sig-9-Fc, Sig-9-Fc-1G, Sig-9-SAX-Fc-1G, Sig-15-SAX-Fc-1G and mSig-15-Fc-1G. 4 doses of test article were administered at 10 mg/kg dose. The first dose was given two hours before arthritomab inoculation and subsequent doses were given on a two dose per week schedule. The disease erosive arthritis was seen after the LPS boost. The study ran for 29 days total. Clinical score, body weight and paw thickness were recorded as readouts in intervals. Dexamethasone at 0.3 mg/kg was used as a positive control in the study.

FIG. 15 depicts the effects of Siglec ECD constructs in the mouse CAIA model. FIG. 15A depicts a clinical score over time while FIG. 15B depicts an Area Under the Curve (AUC) analysis. Sig-9-SAX-Fc-1G and mSig-15-Fc-1G caused a clinically significant reduction in clinical score. Unlike for dexamethasone, body weight was fully recovered at the end of the study in the Siglec ECD treated animals.

### Example 5

This Example describes the effects of a Siglec-6 ECD fusion construct on cytokine expression in activated T cells.

The Siglec ECD fusion construct described in this Example is summarized in TABLE 12. The fusion protein (Sig-6-SAX-1G) had (i) a mutant Siglec-6 ECD with a sialic acid loss-of-binding domain (SAX) mutation (R122K) and (ii) a human IgG1 Fc domain having an N297G mutation removing an N-linked glycosylation site. The fusion protein was prepared as described in Example 2.

TABLE 12

| | Amino | | | |
|---|---|---|---|---|
| | Acid | Nuc | | |
| | SEQ | SEQ | Description | |
| Construct | ID NO | ID NO | Siglec ECD | Fc |
| Sig-6- | 158 | 159 | Human Siglec-6 SAX | Human |
| SAX-1G | | | (Residues | IgG1 Fc (N297G) |
| | | | (Residues 27-347; | (Modified Hinge) |
| | | | R122K) | |

On day 1, human PBMCs were thawed and enriched for T cells using the STEMCELL T isolation kit (Catalog: 19051). T cells were stimulated with anti-CD3 (clone OKT3) and anti-CD28 (clone CD28.2) antibodies at a final concentration of 1 μg/ml in complete RPMI media (supplemented with 10% heat-inactivated FBS, non-essential amino acids, and sodium pyruvate). On day 2, floating cells were collected and re-plated in fresh complete RPMI media and anti-CD3 and anti-CD28 antibodies were replenished at 1 μg/mL to stimulate cells continuously. On day 4 or 5, cells were spun down and resuspended in fresh RPMI media (10% complete) and supplied with low levels of IL-2 (5 ng/mL or ˜50 IU). On day 7, U-bottom well plates were coated with anti-CD3 antibody (clone OKT3) at 1 μg/mL and incubated on a shaker for 2 hours. Activated T cells were harvested, centrifuged, resuspended in fresh media, and counted. Sig-6-SAX-1G was diluted and added to each well at the desired concentration. An IgG1 antibody with an N297G mutation (IgG1-1G) was used as an isotype control. Then approximately 200,000 activated T cells were added to each well to a final volume of 200 μL. Cultures were incubated overnight, and the supernatants were harvested for analysis the next day. Cytokine concentration was measured using a Biolegend LEGENDplex kit (Catalog No. 741035).

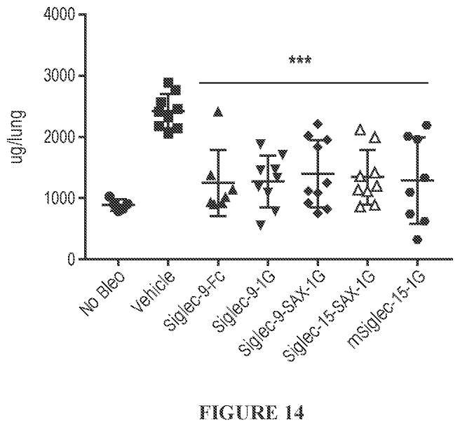

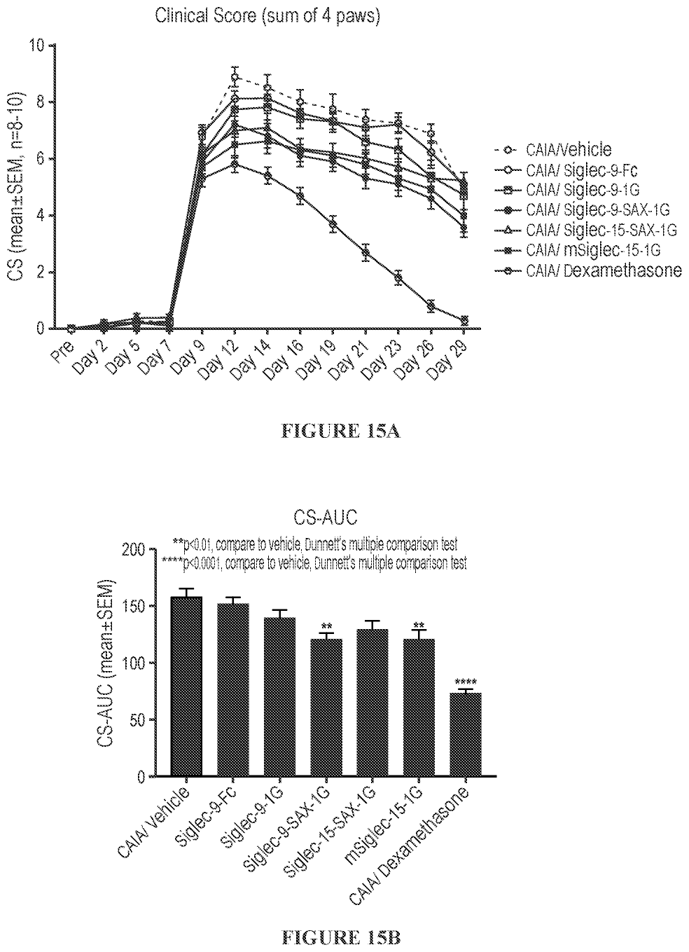

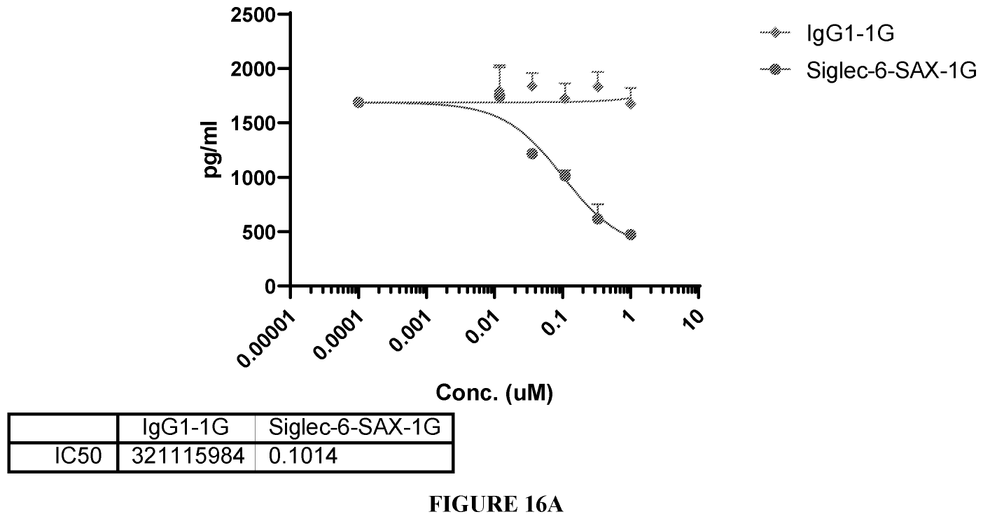

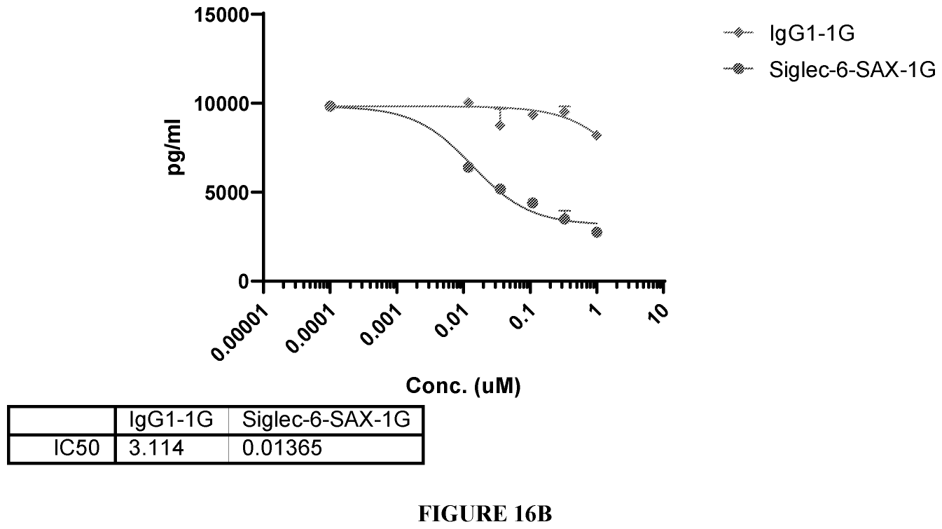

FIG. 16 depicts secretion of IFN-γ from human T cells treated with Sig-6-SAX-1G. FIG. 16A and FIG. 16B depict the results from two individual donors. The IC50 (μM) for Sig-6-SAX-1G is shown below each figure. Sig-6-SAX-1G exhibited dose-dependent inhibition of IFN-γ secretion from activated T cells, thus demonstrating the construct's immunosuppressive activity.

### Example 6

This Example describes the effects of a Siglec-6 extracellular domain (ECD) fusion protein on cytokine expression in M1 and M2 polarized human macrophages.

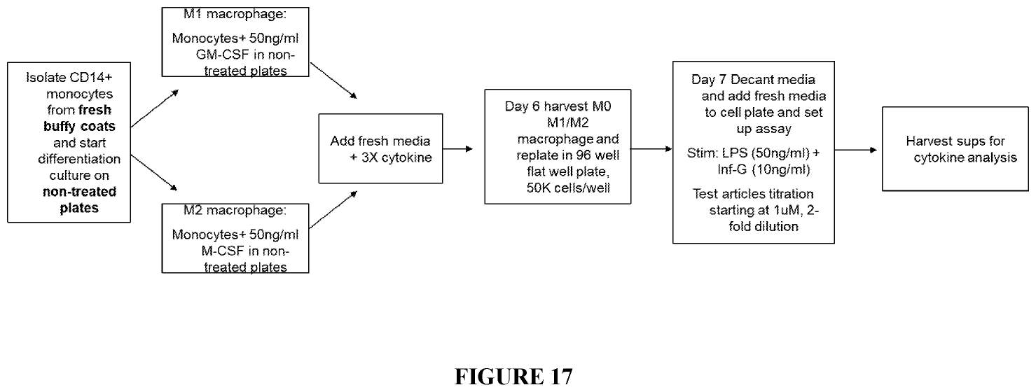

FIG. 17 summarizes a human macrophage polarization assay described in this Example. Briefly, M1 or M2 macrophages were generated using CD14+ monocytes isolated from fresh or frozen human PBMCs. M1 macrophages were generated by culturing monocytes in 50 ng/ml GM-CSF, whereas M2 macrophages were generated by culturing monocytes in presence of 50 ng/mL M-CSF. The media and cytokines were replenished every 2 or 3 days until day 6. On day 6, macrophages were harvested using Accutase (Innovative Cell Technologies) and gentle scraping. Harvested macrophages were re-seeded in a flat 96-well plate at approximately 50,000 cells/well in RPMI 1640 media (supplemented with 10% FBS, non-essential amino acids, and sodium pyruvate). On the next day, media was decanted and replaced with fresh media, and the test article (Sig-6-SAX-1G) was diluted and added to the culture at the desired concentration. IgG-1G was used as an isotype control. Cells were stimulated via overnight incubation with 50 ng/mL LPS and 10 ng/mL IFN-γ. The next day, supernatants were harvested for cytokine analysis and cells were processed for the identification of cell-surface markers.

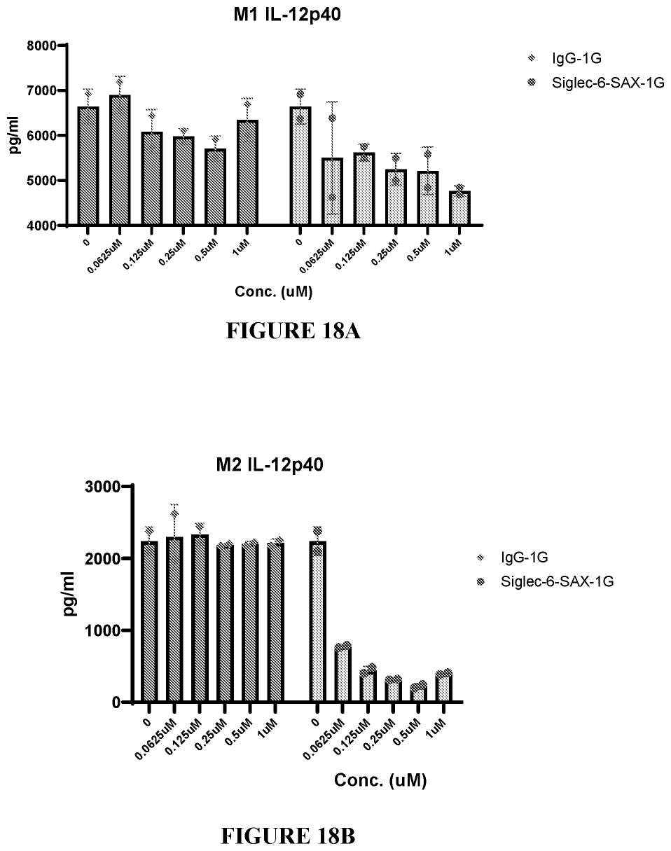

FIG. 18 depicts the effect of Sig-6-SAX-1G on IL-12p40 secretion from either M1 human macrophages (FIG. 18A) or M2 human macrophages (FIG. 18B). Sig-6-SAX-1G treatment inhibited secretion of IL-12p40 from both M1 and M2 macrophages, and especially from M2 macrophages.

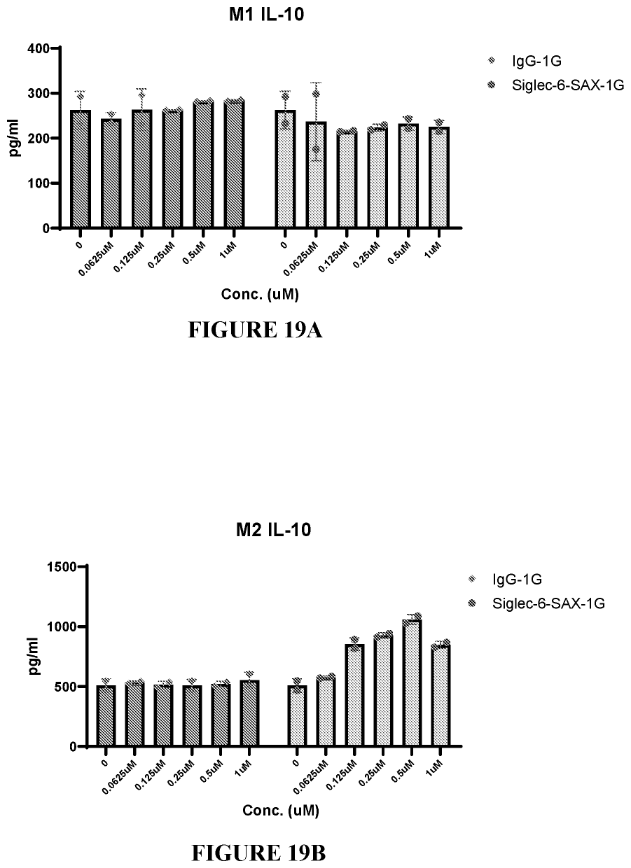

FIG. 19 depicts the effect of Sig-6-SAX-1G on IL-10 secretion from either M1 human macrophages (FIG. 19A) or M2 human macrophages (FIG. 19B). Sig-6-SAX-1G treatment stimulated secretion of the anti-inflammatory cytokine IL10 from M2 macrophages.

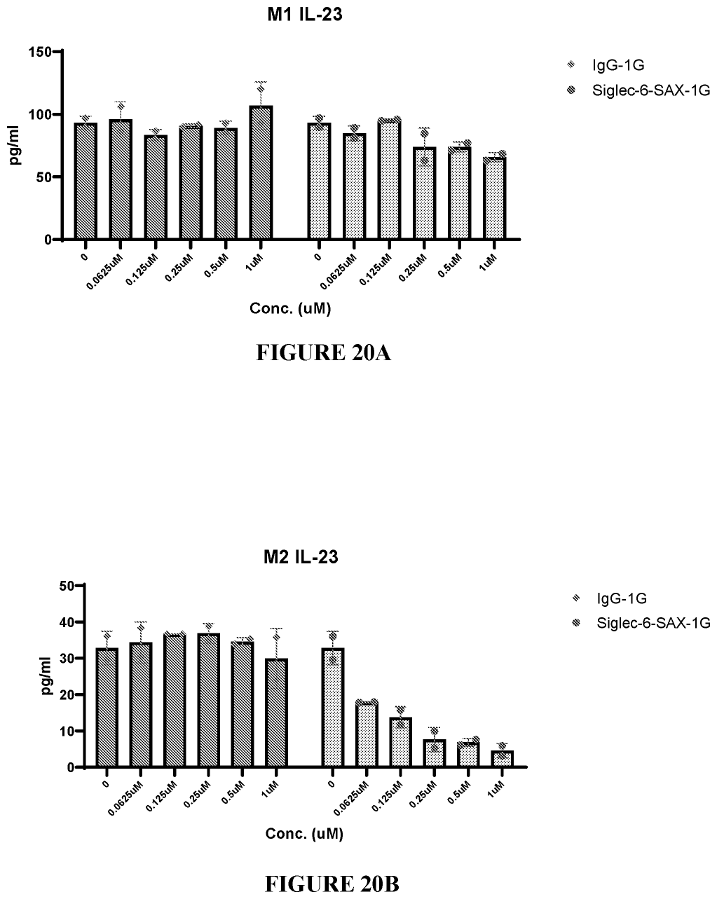

FIG. 20 depicts the effect of Sig-6-SAX-1G on IL-23 secretion from either M1 human macrophages (FIG. 20A) or M2 human macrophages (FIG. 20B). Sig-6-SAX-1G treatment inhibited secretion of IL-23 from both M1 and M2 macrophages, and especially from M2 macrophages.

A second human macrophage polarization assay was used to assess activity of Sig-6-SAX-1G on cell surface markers. This polarization protocol was identical to the one described hereinabove and summarized in FIG. 17, except that the CD14+ monocytes were started on plasma-treated plates on Day 1. TABLE 13 summarizes the cell staining panel used for flow cytometry analysis of M1 and M2 human macrophages following treatment with test articles. Untreated macrophages (stimulated or unstimulated) and IgG-1G-treated macrophages were used as controls.

TABLE 13

| Marker | Channel |
|---|---|
| CD64/Fc gamma-R-I | FITC |
| Siglec-9 | BV421 |
| CD163 | BV510 |
| CD80 | BV650 |
| CD86 | BV786 |
| HLA-DR (Class-II) | PE-Cy-7 |
| Live/dead dye | APC-Cy-7 |

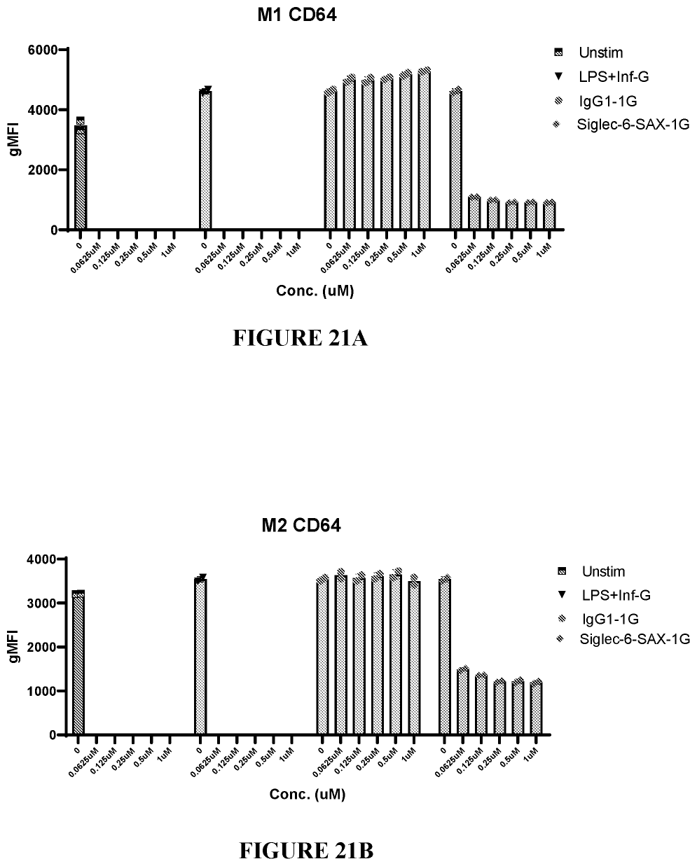

FIG. 21 depicts the effect of Sig-6-SAX-1G on CD64 surface expression for M1 human macrophages (FIG. 21A) and M2 human macrophages (FIG. 21B). For both M1 and M2 macrophages, Sig-6-SAX-1G treatment reduced macrophage CD64 surface expression, thus demonstrating the immunosuppressive activity of the construct.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A pharmaceutical composition comprising a Siglec extracellular domain (ECD), or a functional fragment or variant thereof, conjugated to a serum half-life enhancer that increases the serum half-life of the Siglec ECD when administered to a subject, wherein the Siglec ECD comprises a mutation that reduces sialic acid binding activity.

2. The pharmaceutical composition of claim 1, wherein the mutation results in the Siglec ECD having less than 50% (e.g., less than 40%, less than 30%, less than 20%, less than 10%, less than 5%, less than 3%, less than 1%, or less than 0.5%) of the sialic acid binding activity of a corresponding Siglec ECD without the mutation(s).

3. The pharmaceutical composition of claim 1 or 2, wherein the Siglec ECD is selected from a human Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, Siglec-15, and Siglec-16 ECD.

4. The pharmaceutical composition of any one of claims 1-3, wherein the Siglec ECD is selected from a human Siglec-1, Siglec-4, Siglec-6, Siglec-9, Siglec-11, and Siglec-15 ECD.

5. The pharmaceutical composition of any one of claims 1-4, wherein the Siglec ECD is selected from a human Siglec-1, Siglec-4, Siglec-6, Siglec-9, and Siglec-11 ECD.

6. The pharmaceutical composition of any one of claims 1-5, wherein:
(a) the Siglec ECD is a human Siglec-1 ECD, and the mutation is present in the region from amino acid 106 to 134 of SEQ ID NO: 15 (wild-type human Siglec-1);
(b) the Siglec ECD is a human Siglec-2 ECD, and the mutation is present in the region from amino acid 110 to 135 of SEQ ID NO: 23 (wild-type human Siglec-2);
(c) the Siglec ECD is a human Siglec-3 ECD, and the mutation is present in the region from amino acid 109 to 137 of SEQ ID NO: 25 (wild-type human Siglec-3);
(d) the Siglec ECD is a human Siglec-4 ECD, and the mutation is present in the region from amino acid 108 to 133 of SEQ ID NO: 33 (wild-type human Siglec-4);
(e) the Siglec ECD is a human Siglec-5 ECD, and the mutation is present in the region from amino acid 109 to 138 of SEQ ID NO: 41 (wild-type human Siglec-5);
(f) the Siglec ECD is a human Siglec-6 ECD, and the mutation is present in the region from amino acid 112 to 140 of SEQ ID NO: 43 (wild-type human Siglec-6);
(g) the Siglec ECD is a human Siglec-7 ECD, and the mutation is present in the region from amino acid 114 to 142 of SEQ ID NO: 51 (wild-type human Siglec-7);
(h) the Siglec ECD is a human Siglec-8 ECD, and the mutation is present in the region from amino acid 115 to 149 of SEQ ID NO: 63 (wild-type human Siglec-8);
(i) the Siglec ECD is a human Siglec-9 ECD, and the mutation is present in the region from amino acid 110 to 138 of SEQ ID NO: 65 (wild-type human Siglec-9);
(j) the Siglec ECD is a human Siglec-10 ECD, and the mutation is present in the region from amino acid 109 to 138 of SEQ ID NO: 87 (wild-type human Siglec-10);
(k) the Siglec ECD is a human Siglec-11 ECD, and the mutation is present in the region from amino acid 122 to 151 of SEQ ID NO: 92 (wild-type human Siglec-11);
(l) the Siglec ECD is a human Siglec-12 ECD, and the mutation is present in the region from amino acid 122 to 151 of SEQ ID NO: 100 (wild-type human Siglec-12);
(m) the Siglec ECD is a human Siglec-14 ECD, and the mutation is present in the region from amino acid 109 to 138 of SEQ ID NO: 102 (wild-type human Siglec-14);
(n) the Siglec ECD is a human Siglec-15 ECD, and the mutation is present in the region from amino acid 133 to 161 of SEQ ID NO: 104 (wild-type human Siglec-15); or
(o) the Siglec ECD is a human Siglec-16 ECD, and the mutation is present in the region from amino acid 110 to 139 of SEQ ID NO: 114 (wild-type human Siglec-16).

7. The pharmaceutical composition of any one of claims 1-5, wherein:
(a) the Siglec ECD is a human Siglec-1 ECD, and the mutation is a substitution of an arginine residue at a position corresponding to position 116, of wild-type human Siglec-1 (e.g., R116);
(b) the Siglec ECD is a human Siglec-2 ECD, and the mutation is a substitution of an arginine residue at a position corresponding to position 120 of wild-type human Siglec-2 (e.g., R120);
(c) the Siglec ECD is a human Siglec-3 ECD, and the mutation is a substitution of an arginine residue at a position corresponding to position 119 of wild-type human Siglec-3 (e.g., R119);
(d) the Siglec ECD is a human Siglec-4 ECD, and the mutation is a substitution of an arginine residue at a position corresponding to position 118 of wild-type human Siglec-4 (e.g., R118);
(e) the Siglec ECD is a human Siglec-5 ECD, and the mutation is a substitution of an arginine residue at a position corresponding to position 119 of wild-type human Siglec-5 (e.g., R119);
(f) the Siglec ECD is a human Siglec-6 ECD, and the mutation is a substitution of an arginine residue at a position corresponding to position 122 of wild-type human Siglec-6 (e.g., R122);
(g) the Siglec ECD is a human Siglec-7 ECD, and the mutation is a substitution of an arginine residue at a position corresponding to position 124 of wild-type human Siglec-7 (e.g., R124);
(h) the Siglec ECD is a human Siglec-8 ECD, and the mutation is a substitution of an arginine residue at a position corresponding to position 109 of wild-type human Siglec-8 (e.g., R128);
(i) the Siglec ECD is a human Siglec-9 ECD, and the mutation is a substitution of an arginine residue at a position corresponding to position 120 of wild-type human Siglec-9 (e.g., R120);
(j) the Siglec ECD is a human Siglec-10 ECD, and the mutation is a substitution of an arginine residue at a position corresponding to position 119 of wild-type human Siglec-10 (e.g., R119);
(k) the Siglec ECD is a human Siglec-11 ECD, and the mutation is a substitution of an arginine residue at a position corresponding to position 132 of wild-type human Siglec-11 (e.g., R132);
(l) the Siglec ECD is a human Siglec-12 ECD, and the mutation is a substitution of an arginine residue at a position corresponding to position 125 of wild-type human Siglec-12 (e.g., R125);
(m) the Siglec ECD is a human Siglec-14 ECD, and the mutation is a substitution of an arginine residue at a position corresponding to position 119 of wild-type human Siglec-14 (e.g., R119);
(n) the Siglec ECD is a human Siglec-15 ECD, and the mutation is a substitution of an arginine residue at a position corresponding to position 143 of wild-type human Siglec-15 (e.g., R143); or
(o) the Siglec ECD is a human Siglec-16 ECD, and the mutation is a substitution of an arginine residue at a position corresponding to position 120 of wild-type human Siglec-16 (e.g., R120).

8. A pharmaceutical composition comprising a Siglec extracellular domain (ECD), or a functional fragment or variant thereof, conjugated to a serum half-life enhancer that increases the serum half-life of the Siglec ECD when administered to a subject, wherein the Siglec ECD is selected from a human Siglec-1, Siglec-2, Siglec-3, Siglec-6, Siglec-7, Siglec-8, Siglec-10, Siglec-11, Siglec-12, Siglec-14, and Siglec-16 ECD.

9. The pharmaceutical composition of claim 8, wherein the Siglec ECD is selected from a human Siglec-1, Siglec-6, and Siglec-11 ECD.

10. The pharmaceutical composition of any one of claims 1-9, wherein the Siglec ECD, or the functional fragment or variant thereof, and the serum half-life enhancer are covalently linked together in a fusion protein.

11. The pharmaceutical composition of claim 10, wherein the fusion protein comprises an amino acid sequence selected from the group consisting of SEQ ID NOs: 19, 21, 31, 37, 39, 47, 49, 59, 61, 73, 75, 77, 81, 83, 85, 90, 96, 98, 108, 110, 117, 120, 122, 124, 126-135, 139, 141, 143, 145, 147, 149-151, 154, 155, and 158, or a functional fragment thereof.

12. The pharmaceutical composition of any one of claims 1-9, wherein the Siglec ECD, or the functional fragment or variant thereof, and the serum half-life enhancer are chemically conjugated together.

13. The pharmaceutical composition of any one of claims 1-10 and 12, wherein the serum half-life enhancer is selected from an immunoglobulin Fc domain, transferrin, albumin, XTEN, a homo-amino acid polymer (HAP), a proline-alanine-serine polymer (PAS), an elastin-like peptide (ELP), albumin binding domain, carboxy-terminal peptide (CTP), gelatin-like protein (GLK), and a polyethylene glycol (PEG).

14. The pharmaceutical composition of claim 13, wherein the serum half-life enhancer is an immunoglobulin Fc domain.

15. The pharmaceutical composition of claim 14, wherein the immunoglobulin Fc domain is derived from a human IgG1, IgG2, IgG3, IgG4, IgA1, IgA2, IgD, IgE, or IgM Fc domain.

16. The pharmaceutical composition of claim 14, wherein the immunoglobulin Fc domain is derived from a human IgG1, IgG2, IgG3, or IgG4 Fc domain.

17. The pharmaceutical composition of claim 16, wherein the immunoglobulin Fc domain is derived from a human IgG1 Fc domain.

18. The pharmaceutical composition of any one of claims 1-17, wherein the Siglec ECD, or the functional fragment or variant thereof, conjugated to the serum half-life enhancer is present as a dimer.

19. The pharmaceutical composition of any one of claims 1-18, wherein the pharmaceutical composition, which optionally further comprises a pharmaceutically acceptable carrier, is disposed in a sterile container (e.g., a bottle or vial).

20. The pharmaceutical composition of claim 19, wherein the pharmaceutical composition is lyophilized in the sterile container.

21. The pharmaceutical composition of claim 19, wherein the pharmaceutical composition is present as a solution in the sterile container.

22. The pharmaceutical composition of any one of claims 17-21, wherein the sterile container has a label disposed thereon identifying the pharmaceutical composition contained in the container.

23. A method of treating an inflammatory and/or autoimmune disorder in a subject in need thereof, the method comprising administering to the subject the pharmaceutical composition of any one of claims 1-22.

24. A method of treating an inflammatory disorder and/or an autoimmune disorder in a subject in need thereof, the method comprising administering to the subject a Siglec extracellular domain (ECD), or a functional fragment or variant thereof, wherein the Siglec ECD comprises a mutation that reduces sialic acid binding activity.

25. The method of claim 24, wherein the mutation results in the Siglec ECD, or the functional fragment or variant thereof, having less than 50% (e.g., less than 40%, less than 30%, less than 10%, less than 5%, less than 3%, less than 1%, or less than 0.5%) of the sialic acid binding activity of a corresponding Siglec ECD without the mutation.

26. The method of claim 24 or 25, wherein the Siglec ECD is selected from a human Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, Siglec-15, and Siglec-16 ECD.

27. The method of any one of claims 24-26, wherein the Siglec ECD is selected from a human Siglec-1, Siglec-4, Siglec-6, Siglec-9, Siglec-11, and Siglec-15 ECD.

28. The method of any one of claims 24-27, wherein the Siglec ECD is selected from a human Siglec-1, Siglec-4, Siglec-6, Siglec-9, and Siglec-11 ECD.

29. The method of any one of claims 24-28, wherein:
(a) the Siglec ECD is a human Siglec-1 ECD, and the mutation is present in the region from amino acid 106 to 134 of SEQ ID NO: 15 (wild-type human Siglec-1);
(b) the Siglec ECD is a human Siglec-2 ECD, and the mutation is present in the region from amino acid 110 to 135 of SEQ ID NO: 23 (wild-type human Siglec-2);
(c) the Siglec ECD is a human Siglec-3 ECD, and the mutation is present in the region from amino acid 109 to 137 of SEQ ID NO: 25 (wild-type human Siglec-3);
(d) the Siglec ECD is a human Siglec-4 ECD, and the mutation is present in the region from amino acid 108 to 133 of SEQ ID NO: 33 (wild-type human Siglec-4);
(e) the Siglec ECD is a human Siglec-5 ECD, and the mutation is present in the region from amino acid 109 to 138 of SEQ ID NO: 41 (wild-type human Siglec-5);
(f) the Siglec ECD is a human Siglec-6 ECD, and the mutation is present in the region from amino acid 112 to 140 of SEQ ID NO: 43 (wild-type human Siglec-6);
(g) the Siglec ECD is a human Siglec-7 ECD, and the mutation is present in the region from amino acid 114 to 142 of SEQ ID NO: 51 (wild-type human Siglec-7);
(h) the Siglec ECD is a human Siglec-8 ECD, and the mutation is present in the region from amino acid 115 to 149 of SEQ ID NO: 63 (wild-type human Siglec-8);
(i) the Siglec ECD is a human Siglec-9 ECD, and the mutation is present in the region from amino acid 110 to 138 of SEQ ID NO: 65 (wild-type human Siglec-9);
(j) the Siglec ECD is a human Siglec-10 ECD, and the mutation is present in the region from amino acid 109 to 138 of SEQ ID NO: 87 (wild-type human Siglec-10);
(k) the Siglec ECD is a human Siglec-11 ECD, and the mutation is present in the region from amino acid 122 to 151 of SEQ ID NO: 92 (wild-type human Siglec-11);
(l) the Siglec ECD is a human Siglec-12 ECD, and the mutation is present in the region from amino acid 122 to 151 of SEQ ID NO: 100 (wild-type human Siglec-12);
(m) the Siglec ECD is a human Siglec-14 ECD, and the mutation is present in the region from amino acid 109 to 138 of SEQ ID NO: 102 (wild-type human Siglec-14);
(n) the Siglec ECD is a human Siglec-15 ECD, and the mutation is present in the region from amino acid 133 to 161 of SEQ ID NO: 104 (wild-type human Siglec-15); or
(o) the Siglec ECD is a human Siglec-16 ECD, and the mutation is present in the region from amino acid 110 to 139 of SEQ ID NO: 114 (wild-type human Siglec-16).

30. The method of any one of claims 23-27, wherein:
(a) the Siglec ECD is a human Siglec-1 ECD, and the mutation is a substitution of an arginine residue at a position corresponding to position 116 of wild-type human Siglec-1 (e.g., R116);
(b) the Siglec ECD is a human Siglec-2 ECD, and the mutation is a substitution of an arginine residue at a position corresponding to position 120 of wild-type human Siglec-2 (e.g., R120);
(c) the Siglec ECD is a human Siglec-3 ECD, and the mutation is a substitution of an arginine residue at a position corresponding to position 119 of wild-type human Siglec-3 (e.g., R119);
(d) the Siglec ECD is a human Siglec-4 ECD, and the mutation is a substitution of an arginine residue at a position corresponding to position 118 of wild-type human Siglec-4 (e.g., R118);
(e) the Siglec ECD is a human Siglec-5 ECD, and the mutation is a substitution of an arginine residue at a position corresponding to position 119 of wild-type human Siglec-5 (e.g., R119);
(f) the Siglec ECD is a human Siglec-6 ECD, and the mutation is a substitution of an arginine residue at a position corresponding to position 122 of wild-type human Siglec-6 (e.g., R122);
(g) the Siglec ECD is a human Siglec-7 ECD, and the mutation is a substitution of an arginine residue at a position corresponding to position 124 of wild-type human Siglec-7 (e.g., R124);
(h) the Siglec ECD is a human Siglec-8 ECD, and the mutation is a substitution of an arginine residue at a position corresponding to position 109 of wild-type human Siglec-8 (e.g., R128);
(i) the Siglec ECD is a human Siglec-9 ECD, and the mutation is a substitution of an arginine residue at a position corresponding to position 120 of wild-type human Siglec-9 (e.g., R120);
(j) the Siglec ECD is a human Siglec-10 ECD, and the mutation is a substitution of an arginine residue at a position corresponding to position 119 of wild-type human Siglec-10 (e.g., R119);
(k) the Siglec ECD is a human Siglec-11 ECD, and the mutation is a substitution of an arginine residue at a position corresponding to position 132 of wild-type human Siglec-11 (e.g., R132);
(l) the Siglec ECD is a human Siglec-12 ECD; and the mutation is a substitution of an arginine residue at a position corresponding to position 125 of wild-type human Siglec-12 (e.g., R125);
(m) the Siglec ECD is a human Siglec-14 ECD, and the mutation is a substitution of an arginine residue at a position corresponding to position 119 of wild-type human Siglec-14 (e.g., R119);
(n) the Siglec ECD is a human Siglec-15 ECD, and the mutation is a substitution of an arginine residue at a position corresponding to position 143 of wild-type human Siglec-15 (e.g., R143); or
(o) the Siglec ECD is a human Siglec-16 ECD, and the mutation is a substitution of an arginine residue at a position corresponding to position 120 of wild-type human Siglec-16 (e.g., R120).

31. A method of treating an inflammatory disorder and/or an autoimmune disorder in a subject in need thereof, the method comprising administering to the subject a Siglec extracellular domain (ECD), or a functional fragment or variant thereof, wherein the Siglec ECD is selected from a human Siglec-1, Siglec-2, Siglec-3, Siglec-6, Siglec-7, Siglec-8, Siglec-10, Siglec-11, Siglec-12, Siglec-14, and Siglec-16 ECD.

32. The method of claim 31, wherein the Siglec ECD, or the functional fragment or variant thereof, comprises a mutation that reduces sialic acid binding activity.

33. The method of claim 32, wherein the mutation results in the Siglec ECD, or the functional fragment or variant thereof, having less than 50% (e.g., less than 40%, less than 30%, less than 20%, less than 10%, less than 5%, less than 3%, less than 1%, or less than 0.5%) of the sialic acid binding activity of a corresponding Siglec ECD without the mutation.

34. The method of any one of claims 31-33, wherein:
(a) the Siglec ECD is a human Siglec-1 ECD, and the mutation is present in the region from amino acid 106 to 134 of SEQ ID NO: 15 (wild-type human Siglec-1);
(b) the Siglec ECD is a human Siglec-2 ECD, and the mutation is present in the region from amino acid 110 to 135 of SEQ ID NO: 23 (wild-type human Siglec-2);
(c) the Siglec ECD is a human Siglec-3 ECD, and the mutation is present in the region from amino acid 109 to 137 of SEQ ID NO: 25 (wild-type human Siglec-3);
(d) the Siglec ECD is a human Siglec-6 ECD, and the mutation is present in the region from amino acid 112 to 140 of SEQ ID NO: 43 (wild-type human Siglec-6);
(e) the Siglec ECD is a human Siglec-7 ECD, and the mutation is present in the region from amino acid 114 to 142 of SEQ ID NO: 51 (wild-type human Siglec-7);
(f) the Siglec ECD is a human Siglec-8 ECD, and the mutation is present in the region from amino acid 115 to 149 of SEQ ID NO: 63 (wild-type human Siglec-8);
(g) the Siglec ECD is a human Siglec-10 ECD, and the mutation is present in the region from amino acid 109 to 138 of SEQ ID NO: 87 (wild-type human Siglec-10);
(h) the Siglec ECD is a human Siglec-11 ECD, and the mutation is present in the region from amino acid 122 to 151 of SEQ ID NO: 92 (wild-type human Siglec-11);
(i) the Siglec ECD is a human Siglec-12 ECD, and the mutation is present in the region from amino acid 122 to 151 of SEQ ID NO: 100 (wild-type human Siglec-12);
(j) the Siglec ECD is a human Siglec-14 ECD, and the mutation is present in the region from amino acid 109 to 138 of SEQ ID NO: 102 (wild-type human Siglec-14); or
(k) the Siglec ECD is a human Siglec-16 ECD, and the mutation is present in the region from amino acid 110 to 139 of SEQ ID NO: 114 (wild-type human Siglec-16).

35. The method of any one of claims 31-33, wherein:
(a) the Siglec ECD is a human Siglec-1 ECD, and the mutation is a substitution of an arginine residue at a position corresponding to position 116 of wild-type human Siglec-1 (e.g., R116);
(b) the Siglec ECD is a human Siglec-2 ECD, and the mutation is a substitution of an arginine residue at a position corresponding to position 120 of wild-type human Siglec-2 (e.g., R120);
(c) the Siglec ECD is a human Siglec-3 ECD, and the mutation is a substitution of an arginine residue at a position corresponding to position 119 of wild-type human Siglec-3 (e.g., R119);
(d) the Siglec ECD is a human Siglec-6 ECD, and the mutation is a substitution of an arginine residue at a position corresponding to position 122 of wild-type human Siglec-6 (e.g., R122);
(e) the Siglec ECD is a human Siglec-7 ECD, and the mutation is a substitution of an arginine residue at a position corresponding to position 124 of wild-type human Siglec-7 (e.g., R124);
(f) the Siglec ECD is a human Siglec-8 ECD, and the mutation is a substitution of an arginine residue at a position corresponding to position 109 of wild-type human Siglec-8 (e.g., R128);
(g) the Siglec ECD is a human Siglec-10 ECD, and the mutation is a substitution of an arginine residue at a position corresponding to position 119 of wild-type human Siglec-10 (e.g., R119);
(h) the Siglec ECD is a human Siglec-11 ECD, and the mutation is a substitution of an arginine residue at a position corresponding to position 132 of wild-type human Siglec-11 (e.g., R132);
(i) the Siglec ECD is a human Siglec-12 ECD; and the mutation is a substitution of an arginine residue at a position corresponding to position 125 of wild-type human Siglec-12 (e.g., R125);
(j) the Siglec ECD is a human Siglec-14 ECD, and the mutation is a substitution of an arginine residue at a position corresponding to position 119 of wild-type human Siglec-14 (e.g., R119); or
(k) the Siglec ECD is a human Siglec-16 ECD, and the mutation is a substitution of an arginine residue at a position corresponding to position 120 of wild-type human Siglec-16 (e.g., R120).

36. The method of any one of claims 31-35, wherein the Siglec ECD, or the functional fragment or variant thereof, is conjugated to a serum half-life enhancer.

37. The method of claim 36, wherein the Siglec ECD, or the functional fragment or variant thereof, and the serum half-life enhancer are covalently linked together in a fusion protein.

38. The method of claim 37, wherein the fusion protein comprises an amino acid sequence selected from the group consisting of SEQ ID NOs: 19, 21, 31, 37, 39, 47, 49, 59, 61, 73, 75, 77, 81, 83, 85, 90, 96, 98, 108, 110, 117, 120, 122, 124, 126-135, 139, 141, 143, 145, 147, 149-151, 154, 155, and 158, or a functional fragment thereof.

39. The method of claim 36, wherein the Siglec ECD, or the functional fragment or variant thereof, and serum half-life enhancer are chemically conjugated together.

40. The method of any one of claims 36-37 or 39, wherein the serum half-life enhancer is selected from an immunoglobulin Fc domain, transferrin, albumin, XTEN, a homo-amino acid polymer (HAP), a proline-alanine-serine polymer (PAS), an elastin-like peptide (ELP), albumin binding domain, carboxy-terminal peptide (CTP), gelatin-like protein (GLK), and a polyethylene glycol (PEG).

41. The method of claim 40, wherein the serum half-life enhancer is an immunoglobulin Fc domain.

42. The method of claim 41, wherein the immunoglobulin Fc domain is derived from a human IgG1, IgG2, IgG3, IgG4, IgA1, IgA2, IgD, IgE, or IgM Fc domain.

43. The method of claim 42, wherein the immunoglobulin Fc domain is derived from a human IgG1, IgG2, IgG3, or IgG4 Fc domain.

44. The method of claim 43, wherein the immunoglobulin Fe domain is derived from a human IgG1 Fe domain.

45. The method of any one of claims 31-44, wherein the Siglec ECD or the Siglec ECD and serum half-life enhancer is present as a dimer.
